(19) United States
(12) Patent Application Publication
(10) Pub. No.: US 2021/0163928 A1
(43) Pub. Date: Jun. 3, 2021

(54) MESSENGER RNA COMPRISING FUNCTIONAL RNA ELEMENTS

(71) Applicant: ModernaTX, Inc., Cambridge, MA (US)

(72) Inventors: David REID, Somerville, MA (US); Caroline KÖHRER, Cambridge, MA (US); Ruchi JAIN, Brookline, MA (US); Melissa J. MOORE, Cambridge, MA (US); Scott DONOVAN, Braintree, MA (US); Aaron LARSEN, Cambridge, MA (US); Vladimir PRESNYAK, Manchester, NH (US)

(21) Appl. No.: 17/041,332

(22) PCT Filed: Apr. 11, 2019
(86) PCT No.: PCT/US2019/027089

§ 371 Date: Sep. 24, 2020

Related U.S. Application Data
(60) Provisional application No. 62/769,739, filed on Nov. 20, 2018.
(60) Provisional application No. 62/667,849, filed on May 7, 2018.
(60) Provisional application No. 62/656,213, filed on Apr. 11, 2018.

(51) Int. Cl. C12N 15/11; A61K 48/00; C12Q 1/6897

(52) U.S. Cl. CPC C12N 15/11; A61K 48/0066; C12N 2830/50; C12N 2840/102; C12Q 1/6897

(57) ABSTRACT

The present disclosure provides messenger RNAs (mRNAs) having chemical and/or structural modifications, including RNA elements and/or modified nucleotides, in particular C-rich or CG-rich elements, which provide a desired translational regulatory activity to the mRNA.

### RELATED APPLICATIONS

This application claims the benefit of U.S. Provisional patent Application Ser. No. 62/656,213 filed Apr. 11, 2018; U.S. Provisional patent Application Ser. No. 62/667,849 filed May 7, 2018; and U.S. Provisional patent Application Ser. No. 62/769,739 filed Nov. 20, 2018. The entire contents of the above-referenced patent applications are incorporated herein by this reference.

### BACKGROUND

Administration of a synthetic and/or in vitro-generated mRNA that structurally resembles natural mRNA can result in the controlled production of therapeutic proteins or peptides via the endogenous and constitutively-active translation machinery (e.g. ribosomes) that exists within a patient's own cells. In recent years, the development and use of mRNA as a therapeutic agent has demonstrated potential for treatment of numerous diseases and for the development of novel approaches in regenerative medicine and vaccination (Sahin et al., (2014) Nat Rev Drug Discov 13(10):759-780; Stanton et al (2017) RNA Therapeutics. Topics in Medicinal Chemistry, vol 27).

It is recognized that the control and regulation of mRNA translation is an important development component in order for this class of drugs to establish the desired therapeutic effect. There exists a need to develop mRNA with improved therapeutic effect.

### SUMMARY OF THE INVENTION

The present disclosure provides messenger RNAs (mRNAs) having chemical and/or structural modifications, including RNA elements and/or modified nucleotides, which provide a desired translational regulatory activity to the mRNA. In one aspect, the mRNAs of the disclosure comprise modifications that reduce leaky scanning of 5′ UTRs by the cellular translation machinery. Leaky scanning can result in the bypass of the desired initiation codon that begins the open reading frame encoding a polypeptide of interest or a translation product. This bypass can further result in the initiation of polypeptide synthesis from an alternate or alternative initiation codon, and thereby promote the translation of partial, aberrant, or otherwise undesirable open reading frames within the mRNA. The negative impact caused by the failure to initiate translation of the therapeutic protein or peptide at the desired initiator codon, as a consequence of leaky scanning or other mechanisms, poses a challenge in the development of mRNA therapeutics.

Accordingly, the present disclosure provides mRNAs having novel chemical and/or structural modifications, which provide a desired translational regulatory activity, including promoting translation of only one open reading frame encoding a desired polypeptide or translation product. In some aspects, the desired translational regulatory activity reduces, inhibits or eliminates the failure to initiate translation of the therapeutic protein or peptide at the desired initiator codon, which otherwise may occur as a consequence of leaky scanning or other mechanisms. Thus, the present disclosure provides mRNA having chemical and/or structural modifications which are useful to modulate (e.g., control) translation of an mRNA to produce a desired translation product.

In one aspect, the present disclosure is based, at least in part, on the results of a screening of a large library of random 5′UTRs to identify RNA elements that reduce leaky scanning of ribosomes on mRNA. Specifically, at mRNAs containing 5′UTRs including either 50 or 18 randomized nucleotides, theoretically containing 10³⁰ or 69 billion unique sequences respectively, were screened to identify sequence elements that may impact start site fidelity and/or ribosome loading (e.g., ribosome density). It was discovered that RNA sequence elements comprising a C-rich region of at least 50% or greater cytosine nucleotides, with low to no guanosine content, located proximal to the 5′ end of the mRNA (e.g., proximal to the 5′ cap), gave rise to initiation at a first AUG codon that begins an open reading frame encoding a desired translation product. When incorporated into a 5′UTR of an mRNA, it was discovered that a C-rich RNA element of the disclosure resulted in a 37% reduction in leaky scanning relative to an mRNA lacking the C-rich element. Accordingly, the present disclosure provides mRNAs having 5′ UTRs comprising a C-rich RNA element which provides a desired translational regulatory activity to the mRNA, including a reduction in leaky scanning and/or increase in ribosomal density.

In some aspects, the present disclosure provides a messenger RNA (mRNA), wherein the mRNA comprises: a 5′cap, a 5′untranslated region (UTR), a Kozak-like sequence, an initiation codon, a full open reading frame encoding a polypeptide, and a 3′ UTR, wherein the 5′ UTR comprises a C-rich RNA element located proximal to the 5′ cap, wherein the C-rich RNA element comprises a sequence of linked nucleotides, or derivatives or analogs thereof, wherein each nucleotide comprises a nucleobase selected from the group consisting of: adenine, guanine, thymine, uracil, and cytosine, linked in any order, and wherein the C-rich RNA element provides a translational regulatory activity selected from:
a. increasing residence time of a 43S pre-initiation complex (PIC) or ribosome at, or proximal to, the initiation codon;
b. increasing initiation of polypeptide synthesis at or from the initiation codon;
c. increasing an amount of polypeptide translated from the full open reading frame;
d. increasing fidelity of initiation codon decoding by the PIC or ribosome;
e. inhibiting or reducing leaky scanning by the PIC or ribosome;
f. decreasing a rate of decoding the initiation codon by the PIC or ribosome;
g. inhibiting or reducing initiation of polypeptide synthesis at any codon within the mRNA other than the initiation codon;
h. inhibiting or reducing the amount of polypeptide translated from any open reading frame within the mRNA other than the full open reading frame;
i. inhibiting or reducing the production of aberrant translation products;
j. increasing ribosomal density on the mRNA; and
k. a combination of any two or more of (a)-(j).

In any of the foregoing aspects, the C-rich element comprises a sequence of about 100%, about 95%, about 90%, about 85%, about 80%, about 75%, about 70%, about 65%, about 60%, about 55%, about 50%, or greater than 50% cytosine nucleobases or derivatives or analogs thereof.

In any of the foregoing aspects, the C-rich element comprises a sequence of less than about 25%, less than about 20%, less than about 15%, less than about 10%, or less than about 5% guanosine nucleobases, or derivatives or analogs thereof. In some aspects, the C-rich element comprises a sequence of less than about 25% guanosine nucleobases, or derivatives or analogs thereof.

In any of the foregoing aspects, the C-rich element comprises a sequence of about 50% or greater cytosine nucleobases and about 50% or less adenosine nucleobases and/or uracil nucleobases, or derivatives or analogs thereof (e.g., pseudouridine, N1-methyl pseudouridine or 5-methoxyuridine).

In any of the foregoing aspects, the C-rich RNA element comprises a sequence of about 3-20 nucleotides, about 4-18 nucleotides, about 6-16 nucleotides, about 6-14 nucleotides, about 6-12 nucleotides, about 6-10 nucleotides, about 8-14 nucleotides, about 8-12 nucleotides, about 8-10 nucleotides, about 10-12 nucleotides, about 10-14 nucleotides, about 14 nucleotides, about 13 nucleotides, about 12 nucleotides, about 11 nucleotides, about 10 nucleotides, or about 20, 19, 18, 17, 16, 15, 14, 13, 12, 11, 10, 9, 8, 7, 6, 5, 4, or 3 nucleotides or derivatives or analogs thereof, linked in any order,

In some aspects, the C-rich RNA element comprises a sequence of about 14 nucleotides, or derivatives or analogs thereof, linked in any order, wherein the sequence is about 100%, about 95%, about 90%, about 85%, about 80%, about 75%, about 70%, about 65%, about 60%, about 55%, about 50%, or greater than 50% cytosine nucleobases or derivatives or analogs thereof. In some aspects, the C-rich RNA element comprises a sequence of about 13 nucleotides, or derivatives or analogs thereof, linked in any order, wherein the sequence is about 100%, about 95%, about 90%, about 85%, about 80%, about 75%, about 70%, about 65%, about 60%, about 55%, about 50%, or greater than 50% cytosine nucleobases or derivatives or analogs thereof. In some aspects, the C-rich RNA element comprises a sequence of about 12 nucleotides, or derivatives or analogs thereof, linked in any order, wherein the sequence is about 100%, about 95%, about 90%, about 85%, about 80%, about 75%, about 70%, about 65%, about 60%, about 55%, about 50%, or greater than 50% cytosine nucleobases or derivatives or analogs thereof. In some aspects, the C-rich RNA element comprises a sequence of about 11 nucleotides, or derivatives or analogs thereof, linked in any order, wherein the sequence is about 100%, about 95%, about 90%, about 85%, about 80%, about 75%, about 70%, about 65%, about 60%, about 55%, about 50%, or greater than 50% cytosine nucleobases or derivatives or analogs thereof. In some aspects, the C-rich RNA element comprises a sequence of about 10 nucleotides, or derivatives or analogs thereof, linked in any order, wherein the sequence is about 100%, about 95%, about 90%, about 85%, about 80%, about 75%, about 70%, about 65%, about 60%, about 55%, about 50%, or greater than 50% cytosine nucleobases or derivatives or analogs thereof.

In any of the foregoing aspects, the C-rich RNA element is located downstream of and immediately adjacent to the 5′ cap in the 5′ UTR.

In any of the foregoing aspects, the C-rich RNA element is located about 45-50, about 40-45, about 35-40, about 30-35, about 25-30, about 20-25, about 15-20, about 10-15, about 6-10 nucleotides, about 1-5 nucleotides, or about 20, 19, 18, 17, 16, 15, 14, 13, 12, 11, 10, 9, 8, 7, 6, 5, 4, 3, 2 or 1 nucleotide(s) downstream of the 5′ cap or 5′end of the mRNA in the 5′ UTR.

In any of the foregoing aspects, the mRNA comprises a sequence of nucleotides located upstream of the C-rich RNA element which comprises a modification or sequence motif that provides a transcriptional or translational regulatory activity.

In any of the foregoing aspects, the C-rich RNA element is located upstream of a Kozak-like sequence in the 5′ UTR. In some aspects, the C-rich RNA element is located upstream of and immediately adjacent to a Kozak-like sequence in the 5′ UTR. In some aspects, the C-rich RNA element is located about 45-50, about 40-45, about 35-40, about 30-35, about 25-30, about 20-25, about 15-20, about 10-15, about 6-10 nucleotides, about 1-5 nucleotides, or about 20, 19, 18, 17, 16, 15, 14, 13, 12, 11, 10, 9, 8, 7, 6, 5, 4, 3, 2 or 1 nucleotide(s) upstream of the Kozak-like sequence in the 5′ UTR. In some aspects, the C-rich RNA element is located about 20, about 15, about 10 or about 5 nucleotides upstream of a Kozak-like sequence in the 5′ UTR. In some aspects, the C-rich RNA element is located about 5, about 4, about 3, about 2, or about 1 nucleotide upstream of a Kozak-like sequence in the 5′ UTR.

In some aspects, the disclosure provides a messenger RNA (mRNA), wherein the mRNA comprises: a 5′cap, a 5′untranslated region (UTR), a Kozak-like sequence, an initiation codon, a full open reading frame encoding a polypeptide, and a 3′ UTR, wherein the 5′ UTR comprises a C-rich RNA element, wherein the C-rich RNA element comprises:

(i) a sequence of linked nucleotides, or derivatives or analogs thereof, wherein each nucleotide comprises a nucleobase selected from the group consisting of: adenine, guanine, thymine, uracil (e.g., pseudouridine, N1-methyl pseudouridine or 5-methoxyuridine), and cytosine, linked in any order, wherein the sequence of linked nucleotides, or derivatives or analogs thereof, is about 3-20 nucleotides; and

(ii) a sequence of greater than 50% cytosine nucleobases and less than 10% guanosine nucleobases,

wherein the C-rich RNA element is located about 1-20, about 2-15, about 3-10, about 4-8, or about 6 nucleotides downstream of the 5′ cap or 5′ end of the mRNA in the 5′ UTR.

In any of the foregoing aspects, the C-rich RNA element provides a translational regulatory activity selected from:
a. increasing residence time of a 43S pre-initiation complex (PIC) or ribosome at, or proximal to, the initiation codon;
b. increasing initiation of polypeptide synthesis at or from the initiation codon;
c. increasing an amount of polypeptide translated from the full open reading frame;
d. increasing fidelity of initiation codon decoding by the PIC or ribosome;
e. inhibiting or reducing leaky scanning by the PIC or ribosome;
f. decreasing a rate of decoding the initiation codon by the PIC or ribosome;
g. inhibiting or reducing initiation of polypeptide synthesis at any codon within the mRNA other than the initiation codon;
h. inhibiting or reducing the amount of polypeptide translated from any open reading frame within the mRNA other than the full open reading frame;
i. inhibiting or reducing the production of aberrant translation products;
j. increases ribosomal density on the mRNA; and
k. a combination of any two or more of (a)-(j).

In some aspects, the C-rich RNA element provides a translational regulatory activity comprising increasing an amount of polypeptide translated from the full open reading frame. In some aspects, the C-rich RNA element provides a translational regulatory activity comprising inhibiting or reducing the amount of polypeptide translated from any open reading frame within the mRNA other than the full open reading frame. In some aspects, the C-rich RNA element provides a translational regulatory activity comprising inhibiting or reducing the production of aberrant translation products. In some aspects, the C-rich RNA element provides a translational regulatory activity comprising increases ribosomal density on the mRNA.

In any of the foregoing aspects, the C-rich element comprises a sequence of about 95%, about 90%, about 85%, about 80%, about 75%, about 70%, about 65%, about 60%, or about 55% cytosine nucleobases or derivatives or analogs thereof. In some aspects, the C-rich element comprises a sequence of less than about 5% guanosine nucleobases, or derivatives or analogs thereof.

In any of the foregoing aspects, the C-rich element comprises a sequence of 50% or greater cytosine nucleobases, less than about 5% guanosine nucleobases, and about 45% or less adenosine nucleobases and/or uracil nucleobases, or derivatives or analogs thereof (e.g., pseudouridine, N1-methyl pseudouridine, 5-methoxyuridine).

In some aspects, the C-rich RNA element comprises a sequence of about 14 nucleotides, or derivatives or analogs thereof, linked in any order, wherein the sequence is about 95%, about 90%, about 85%, about 80%, about 75%, about 70%, about 65%, about 60%, about 55% cytosine nucleobases or derivatives or analogs thereof, and less than about 5% guanosine nucleobases or derivatives or analogs thereof. In some aspects, the C-rich RNA element comprises a sequence of about 13 nucleotides, or derivatives or analogs thereof, linked in any order, wherein the sequence is about 95%, about 90%, about 85%, about 80%, about 75%, about 70%, about 65%, about 60%, about 55% cytosine nucleobases or derivatives or analogs thereof, and less than about 5% guanosine nucleobases or derivatives or analogs thereof. In some aspects, the C-rich RNA element comprises a sequence of about 12 nucleotides, or derivatives or analogs thereof, linked in any order, wherein the sequence is about 95%, about 90%, about 85%, about 80%, about 75%, about 70%, about 65%, about 60%, about 55% cytosine nucleobases or derivatives or analogs thereof, and less than about 5% guanosine nucleobases or derivatives or analogs thereof. In some aspects, the C-rich RNA element comprises a sequence of about 11 nucleotides, or derivatives or analogs thereof, linked in any order, wherein the sequence is about 95%, about 90%, about 85%, about 80%, about 75%, about 70%, about 65%, about 60%, about 55% cytosine nucleobases or derivatives or analogs thereof, and less than about 5% guanosine nucleobases or derivatives or analogs thereof. In some aspects, the C-rich RNA element comprises a sequence of about 10 nucleotides, or derivatives or analogs thereof, linked in any order, wherein the sequence is about 95%, about 90%, about 85%, about 80%, about 75%, about 70%, about 65%, about 60%, about 55% cytosine nucleobases or derivatives or analogs thereof, and less than about 5% guanosine nucleobases or derivatives or analogs thereof.

In any of the foregoing aspects, the C-rich RNA element comprises a sequence of about 4-18 nucleotides, about 6-16 nucleotides, about 6-14 nucleotides, about 6-12 nucleotides, about 6-10 nucleotides, about 8-14 nucleotides, about 8-12 nucleotides, about 8-10 nucleotides, about 10-12 nucleotides, about 10-14 nucleotides, about 14 nucleotides, about 13 nucleotides, about 12 nucleotides, about 11 nucleotides, about 10 nucleotides, or about 20, 19, 18, 17, 16, 15, 14, 13, 12, 11, 10, 9, 8, 7, 6, 5, 4, or 3 nucleotides or derivatives or analogs thereof, linked in any order.

In any of the foregoing aspects, the C-rich RNA element is located downstream of and immediately adjacent to the 5′ cap in the 5′ UTR. In some aspects, the C-rich RNA element is located about 45-50, about 40-45, about 35-40, about 30-35, about 25-30, about 20-25, about 15-20, about 10-15, about 6-10 nucleotides, about 1-5 nucleotides, or about 20, 19, 18, 17, 16, 15, 14, 13, 12, 11, 10, 9, 8, 7, 6, 5, 4, 3, 2 or 1 nucleotide(s) downstream of the 5′ cap or 5′end of the mRNA in the 5′ UTR.

In any of the foregoing aspects, the mRNA comprises a sequence of nucleotides located upstream of the C-rich RNA element which comprises a modification or sequence motif that provides a transcriptional or translational regulatory activity.

In any of the foregoing aspects, the C-rich RNA element is located upstream of a Kozak-like sequence in the 5′ UTR. In some aspects, the C-rich RNA element is located upstream of and immediately adjacent to a Kozak-like sequence in the 5′ UTR. In some aspects, the C-rich RNA element is located about 45-50, about 40-45, about 35-40, about 30-35, about 25-30, about 20-25, about 15-20, about 10-15, about 6-10 nucleotides, about 1-5 nucleotides, or about 20, 19, 18, 17, 16, 15, 14, 13, 12, 11, 10, 9, 8, 7, 6, 5, 4, 3, 2 or 1 nucleotide(s) upstream of the Kozak-like sequence in the 5′ UTR. In some aspects, the C-rich RNA element is located about 20, about 15, about 10 or about 5 nucleotides upstream of a Kozak-like sequence in the 5′ UTR. In some aspects, the C-rich RNA element is located about 5, about 4, about 3, about 2, or about 1 nucleotide upstream of a Kozak-like sequence in the 5′ UTR.

In some aspects, the disclosure provides a messenger RNA (mRNA), wherein the mRNA comprises: a 5′cap, a 5′untranslated region (UTR), a Kozak-like sequence, an initiation codon, a full open reading frame encoding a polypeptide, and a 3′ UTR, wherein the 5′ UTR comprises a C-rich RNA element, wherein the C-rich RNA element comprises:
a sequence of linked nucleotides comprising the formula

5′-[C1]ᵥ-[N1]_{w}-[N2]ₓ-[N3]_{y}-[C2]_{z}-3′,

wherein C1 and C2 are nucleotides comprising cytidine, or a derivative or analogue thereof, wherein N1, and N2 and N3 if present, are each a nucleotide comprising a nucleobase selected from the group consisting of: adenine, guanine, thymine, uracil, and cytosine, and derivatives or analogues thereof (e.g., pseudouridine, N1-methyl pseudouridine, 5-methoxyuridine), wherein v, w, x, y and z are integers whose value indicates the number of nucleotides comprising the C-rich RNA element, wherein v=2-15 nucleotides, wherein w=1-5 nucleotides, wherein x=0-5 nucleotides, wherein y=0-5 nucleotides, and wherein z=2-10 nucleotides. In some aspects, v=6-8 and z=2-5. In some aspects, v=6-8, w=1 or 2, x=0, y=0 and z=2-5. In some aspects, v=6-8, w=1 or 2, x=1, 2 or 3, y=1 or 2, and z=2-5.

In some aspects, the disclosure provides a mRNA, wherein the mRNA comprises: a 5′ cap, a 5′ UTR comprising a C-rich RNA element of about 3-20 nucleotides comprising a sequence of greater than 50% cytosine nucleobases and less than 10% guanosine nucleobases, wherein the C-rich RNA element is located about 1-50 nucleotides downstream of the 5′ cap or 5′ end of the mRNA in the 5′ UTR; an ORF encoding a polypeptide; and a 3′ UTR, wherein the C-rich RNA element comprises a sequence of linked nucleotides comprising the formula: 5′-[C1]ᵥ-[N1]_{w}-[N2]ₓ-[N3]_{y}-[C2]_{z}-3′, wherein C1 and C2 are nucleotides comprising cytidine, or a derivative or analogue thereof, wherein N1, and N2 and N3 if present, are each a nucleotide comprising a nucleobase selected from the group consisting of: adenine, guanine, thymine, uracil, and cytosine, and derivatives or analogues thereof, wherein v, w, x, y and z are integers whose value indicates the number of nucleotides comprising the C-rich RNA element, wherein v=2-15 nucleotides, wherein w=1-5 nucleotides, wherein x=0-5 nucleotides, wherein y=0-5 nucleotides, and wherein z=2-10 nucleotides.

In some aspects, an mRNA of the disclosure comprises a 5′cap, a 5′UTR, a Kozak-like sequence, an ORF encoding a polypeptide, and a 3′UTR, wherein the 5′UTR comprises a C-rich RNA element comprising the nucleotide sequence set forth in SEQ ID NO: 31 inserted within a 5′ UTR comprising the nucleotide sequence selected from a group consisting of: SEQ ID NO: 45, SEQ ID NO: 71 or SEQ ID NO: 149. In some embodiments, the 5′UTR comprises a C-rich RNA element comprising the nucleotide sequence set forth in SEQ ID NO: 32 inserted within a 5′ UTR comprising the nucleotide sequence selected from a group consisting of: SEQ ID NO: 45, SEQ ID NO: 71 or SEQ ID NO: 149. In some embodiments, the 5′UTR comprises a C-rich RNA element comprising the nucleotide sequence set forth in SEQ ID NO: 31 inserted within a 5′ UTR comprising the nucleotide sequence set forth in SEQ ID NO: 46 or the nucleotide sequence selected from a group consisting of: SEQ ID NO: 42, SEQ ID NO: 72, or SEQ ID NO: 154. In some embodiments, the 5′UTR comprises a C-rich RNA element comprising the nucleotide sequence set forth in SEQ ID NO: 32 inserted within a 5′ UTR comprising the nucleotide sequence set forth in SEQ ID NO: 46 or the nucleotide sequence selected from a group consisting of: SEQ ID NO: 42, SEQ ID NO: 72, or SEQ ID NO: 154. In some embodiments, the 5′UTR comprises a C-rich RNA element comprising the nucleotide sequence set forth in SEQ ID NO: 33 inserted within a 5′ UTR comprising the nucleotide sequence set forth in SEQ ID NO: 46. In some embodiments, the 5′UTR comprises a C-rich RNA element comprising the nucleotide sequence set forth in SEQ ID NO: 33 inserted within a 5′ UTR comprising the nucleotide sequence selected from a group consisting of: SEQ ID NO: 42, SEQ ID NO: 72, or SEQ ID NO: 154.

In some aspects, v=3-12 nucleotides, 5-10 nucleotides, 6-8 nucleotides, 3, 4, 5, 6, 7, 8, 9 or 10 nucleotides. In some aspects, z=2-7 nucleotides, 3-5 nucleotides, 2, 3, 4, 5, 6, or 7 nucleotides. In some aspects, w=1-3 nucleotides, 1, 2, or 3 nucleotide(s). In some aspects, x=0-3 nucleotides, 0, 1, 2, or 3 nucleotide(s). In some aspects, y=0-3 nucleotides, 0, 1, 2, or 3 nucleotide(s).

In any of the foregoing aspects, N1 comprises adenosine, or derivative or analogue thereof; w=1 or 2; x=0, 1, 2, or 3; and y=0, 1, 2, or 3. In some aspects, N1 comprises adenosine, or derivative or analogue thereof; w=1 or 2; x=0; and y=0.

In any of the foregoing aspects, N1 comprises uracil, or derivative or analogue thereof (e.g., pseudouridine, N1-methyl pseudouridine, 5-methoxyuridine); w=1 or 2; N2 comprises adenosine, or derivative or analogue thereof; x=1, 2, or 3; N3 is guanosine, or derivative or analogue thereof; and y=1 or 2. In some aspects, N1 comprises uracil, or derivative or analogue thereof (e.g., pseudouridine, N1-methyl pseudouridine, 5-methoxyuridine); w=1; N2 comprises adenosine, or derivative or analogue thereof; x=2; N3 is guanosine, or derivative or analogue thereof; and y=1.

In any of the foregoing aspects, the C-rich RNA element comprises the formula

5′-[C1]ᵥ-[N1]_{w}-[N2]ₓ-[N3]_{y}-[C2]_{z}-3′,

wherein C1 and C2 are nucleotides comprising cytidine, or a derivative or analogue thereof, wherein N1, and N2 and N3 if present, are each a nucleotide comprising a nucleobase selected from the group consisting of: adenine, guanine, and uracil, and derivatives or analogues thereof, (e.g., pseudouridine, N1-methyl pseudouridine, 5-methoxyuridine), wherein v, w, x, y and z are integers whose value indicates the number of nucleotides comprising the C-rich RNA element, wherein v=4-10 nucleotides, wherein w=1-3 nucleotides, wherein x=0-3 nucleotides, wherein y=0-3 nucleotides, and wherein z=2-6 nucleotides.

In some aspects, v=6-8 nucleotides, 6, 7, or 8 nucleotides. In some aspects, z=2-5 nucleotides, 2, 3, 4, or 5 nucleotides. In some aspects, w=1 or 2 nucleotide(s). In some aspects, x=0, 1 or 2 nucleotide(s). In some aspects, y=0 or 1 nucleotide(s).

In any of the foregoing aspects, N1 comprises adenosine, or derivative or analogue thereof; w=1; x=0; and y=0. In some aspects, N1 comprises adenosine, or derivative or analogue thereof; w=2; x=0; and y=0.

In any of the foregoing aspects, N1 comprises uracil, or derivative or analogue thereof (e.g., pseudouridine, N1-methyl pseudouridine, 5-methoxyuridine); w=1 or 2; N2 comprises adenosine, or derivative or analogue thereof; x=1, 2, or 3; N3 is guanosine, or derivative or analogue thereof; and y=1 or 2. In some aspects, N1 comprises uracil, or derivative or analogue thereof (e.g., pseudouridine, N1-methyl pseudouridine, 5-methoxyuridine); w=1; N2 comprises adenosine, or derivative or analogue thereof; x=2; N3 is guanosine, or derivative or analogue thereof; and y=1. In some aspects, v=6-8; N1 comprises adenosine, or derivative or analogue thereof; w=1 or 2; x=0; y=0; and z=2-5. In some aspects, v=6-8; N1 comprises uracil, or derivative or analogue thereof (e.g., pseudouridine, N1-methyl pseudouridine, 5-methoxyuridine); w=1; N2 comprises adenosine, or derivative or analogue thereof; x=2; N3 is guanosine, or derivative or analogue thereof; y=1; and z=2-5.

In any of the foregoing aspects, the C-rich RNA element comprises the nucleotide sequence

[5′-CCCCCCCCAACC'-3′]
set forth in SEQ ID NO 30.

In any of the foregoing aspects, the C-rich RNA element comprises the nucleotide sequence

[5′-CCCCCCCAACCC'-3′]
set forth in SEQ ID NO: 29.

In any of the foregoing aspects, the C-rich RNA element comprises the nucleotide sequence

[5′-CCCCCCACCCCC'-3′]
set forth in SEQ ID NO: 31.

In any of the foregoing aspects, the C-rich RNA element comprises the nucleotide sequence

[5′-CCCCCCUAAGCC'-3′]
set forth in SEQ ID NO: 32.

In any of the foregoing aspects, the C-rich RNA element comprises the nucleotide sequence

[5′-CCCCACAACC-3′]
set forth in SEQ ID NO: 33.

In any of the foregoing aspects, the C-rich RNA element comprises the nucleotide sequence

[5′-CCCCCACAACC-3′]
set forth in SEQ ID NO: 34.

In any of the foregoing aspects, the C-rich RNA element is located downstream of and immediately adjacent to the 5′ cap in the 5′ UTR.

In any of the foregoing aspects, the C-rich RNA element is located about 45-50, about 40-45, about 35-40, about 30-35, about 25-30, about 20-25, about 15-20, about 10-15, about 6-10 nucleotides, about 1-5 nucleotides, or about 20, 19, 18, 17, 16, 15, 14, 13, 12, 11, 10, 9, 8, 7, 6, 5, 4, 3, 2 or 1 nucleotide(s) downstream of the 5′ cap or 5′end of the mRNA in the 5′ UTR.

In any of the foregoing aspects, the mRNA comprises a sequence of nucleotides located upstream of the C-rich RNA element which comprises a modification or sequence motif that provides a transcriptional or translational regulatory activity.

In any of the foregoing aspects, the C-rich RNA element is located upstream of a Kozak-like sequence in the 5′ UTR. In some aspects, the C-rich RNA element is located upstream of and immediately adjacent to a Kozak-like sequence in the 5′ UTR. In some aspects, the C-rich RNA element is located about 45-50, about 40-45, about 35-40, about 30-35, about 25-30, about 20-25, about 15-20, about 10-15, about 6-10 nucleotides, about 1-5 nucleotides, or about 20, 19, 18, 17, 16, 15, 14, 13, 12, 11, 10, 9, 8, 7, 6, 5, 4, 3, 2 or 1 nucleotide(s) upstream of the Kozak-like sequence in the 5′ UTR. In some aspects, the C-rich RNA element is located about 20, about 15, about 10 or about 5 nucleotides upstream of a Kozak-like sequence in the 5′ UTR. In some aspects, the C-rich RNA element is located about 5, about 4, about 3, about 2, or about 1 nucleotide upstream of a Kozak-like sequence in the 5′ UTR.

In any of the foregoing aspects, the C-rich RNA element provides a translational regulatory activity selected from:
a. increasing residence time of a 43S pre-initiation complex (PIC) or ribosome at, or proximal to, the initiation codon;
b. increasing initiation of polypeptide synthesis at or from the initiation codon;
c. increasing an amount of polypeptide translated from the full open reading frame;
d. increasing fidelity of initiation codon decoding by the PIC or ribosome;
e. inhibiting or reducing leaky scanning by the PIC or ribosome;
f. decreasing a rate of decoding the initiation codon by the PIC or ribosome;
g. inhibiting or reducing initiation of polypeptide synthesis at any codon within the mRNA other than the initiation codon;
h. inhibiting or reducing the amount of polypeptide translated from any open reading frame within the mRNA other than the full open reading frame;
i. inhibiting or reducing the production of aberrant translation products;
j. increases ribosomal density on the mRNA; and
k. a combination of any two or more of (a)-(j).

In some aspects, the C-rich RNA element provides a translational regulatory activity comprising increasing an amount of polypeptide translated from the full open reading frame. In some aspects, the C-rich RNA element provides a translational regulatory activity comprising inhibiting or reducing the amount of polypeptide translated from any open reading frame within the mRNA other than the full open reading frame. In some aspects, the C-rich RNA element provides a translational regulatory activity comprising inhibiting or reducing the production of aberrant translation products. In some aspects, the C-rich RNA element provides a translational regulatory activity comprising increases ribosomal density on the mRNA.

In any of the foregoing aspects, the mRNA comprises:
(i) a first polynucleotide, wherein the first polynucleotide is chemically synthesized, and wherein the first polynucleotide comprises a 5′ UTR comprising at least one sequence motif, and;
(ii) a second polynucleotide, wherein the second polynucleotide is synthesized by in vitro transcription, and, wherein the second polynucleotide comprises a full open reading frame encoding a polypeptide, and a 3′ UTR.

In some aspects, the first polynucleotide and the second polynucleotide are chemically cross-linked. In some aspects, the first polynucleotide and the second polynucleotide are enzymatically ligated. In some aspects, the first polynucleotide and the second polynucleotide are operably linked.

In some aspects, the disclosure provides an mRNA comprising a 5′UTR comprising a C-rich RNA element as described herein, and a GC-rich RNA element.

In some aspects, the GC-rich RNA element comprises a sequence of linked nucleotides, or derivatives or analogs thereof, located upstream of a Kozak consensus sequence in the 5′ UTR. In some aspects, the GC-rich RNA element is located about 30, about 25, about 20, about 15, about 10, or about 5 nucleotides upstream of a Kozak consensus sequence in the 5′ UTR. In some aspects, the GC-rich RNA element is located about 20, about 15, about 10 or about 5 nucleotides upstream of a Kozak consensus sequence in the 5′ UTR. In some aspects, the GC-rich RNA element is located about 5, about 4, about 3, about 2, or about 1 nucleotide upstream of a Kozak consensus sequence in the 5′ UTR. In some aspects, the GC-rich RNA element is located about 15-30, about 15-20, about 15-25, about 10-15, or about 5-10 nucleotides upstream of a Kozak consensus sequence in the 5′ UTR. In some aspects, the GC-rich RNA element is upstream of and immediately adjacent to a Kozak consensus sequence in the 5′ UTR.

In any of the foregoing aspects, the GC-rich RNA element comprises a sequence of about 30, about 20-30, about 20, about 10-20, about 15, about 10-15, about 15, 14, 13, 12, 11, 10, 9, 8, 7, 6, 5, 4, or 3 nucleotides, or derivatives or analogs thereof, linked in any order, wherein the sequence is about 70% cytosine, about 60%-70% cytosine, about 60% cytosine, about 50%-60% cytosine, about 50% cytosine, about 40%-50% cytosine, about 40% cytosine, about 30%-40% cytosine, about 30% cytosine.

In any of the foregoing aspects, the GC-rich RNA element comprises a sequence of 3 nucleotides, or derivatives or analogs thereof, linked in any order, wherein the sequence is >50% cytosine. In some aspects, GC-rich RNA element comprises a sequence of 4 nucleotides, or derivatives or analogs thereof, linked in any order, wherein the sequence is >50% cytosine. In some aspects, the GC-rich RNA element comprises a sequence of 5 nucleotides, or derivatives or analogs thereof, linked in any order, wherein the sequence is >50% cytosine. In some aspects, the GC-rich RNA element comprises a sequence of 6 nucleotides, or derivatives or analogs thereof, linked in any order, wherein the sequence is >50% cytosine. In some aspects, the GC-rich RNA element comprises a sequence of 7 nucleotides, or derivatives or analogs thereof, linked in any order, wherein the sequence is >50% cytosine. In some aspects, the GC-rich RNA element comprises a sequence of 8 nucleotides, or derivatives or analogs thereof, linked in any order, wherein the sequence is >50% cytosine. In some aspects, the GC-rich RNA element comprises a sequence of 9 nucleotides, or derivatives or analogs thereof, linked in any order, wherein the sequence is >50% cytosine. In some aspects, the GC-rich RNA element comprises a sequence of 10 nucleotides, or derivatives or analogs thereof, linked in any order, wherein the sequence is >50% cytosine. In some aspects, the GC-rich RNA element comprises a sequence of 11 nucleotides, or derivatives or analogs thereof, linked in any order, wherein the sequence is >50% cytosine. In some aspects, the GC-rich RNA element comprises a sequence of 12 nucleotides, or derivatives or analogs thereof, linked in any order, wherein the sequence is >50% cytosine. In some aspects, the GC-rich RNA element comprises a sequence of 13 nucleotides, or derivatives or analogs thereof, linked in any order, wherein the sequence is >50% cytosine. In some aspects, the GC-rich RNA element comprises a sequence of 14 nucleotides, or derivatives or analogs thereof, linked in any order, wherein the sequence is >50% cytosine. In some aspects, the GC-rich RNA element comprises a sequence of 15 nucleotides, or derivatives or analogs thereof, linked in any order, wherein the sequence is >50% cytosine. In some aspects, the GC-rich RNA element comprises a sequence of 16 nucleotides, or derivatives or analogs thereof, linked in any order, wherein the sequence is >50% cytosine. In some aspects, the GC-rich RNA element comprises a sequence of 17 nucleotides, or derivatives or analogs thereof, linked in any order, wherein the sequence is >50% cytosine. In some aspects, the GC-rich RNA element comprises a sequence of 18 nucleotides, or derivatives or analogs thereof, linked in any order, wherein the sequence is >50% cytosine. In some aspects, the GC-rich RNA element comprises a sequence of 19 nucleotides, or derivatives or analogs thereof, linked in any order, wherein the sequence is >50% cytosine. In some aspects, the GC-rich RNA element comprises a sequence of 20 nucleotides, or derivatives or analogs thereof, linked in any order, wherein the sequence is >50% cytosine.

In any of the foregoing aspects, the GC-rich RNA element comprises a sequence of about 3-30 guanine and cytosine nucleotides, or derivatives or analogues thereof, wherein the sequence comprises a repeating GC-motif. In some aspects, the repeating GC-motif is [CCG]ₙ, wherein n=1 to 10. In some aspects, the repeating GC-motif is [CCG]ₙ, where n=1 to 5. In some aspects, the repeating GC-motif is [CCG]ₙ, where n=3. In some aspects, the repeating GC-motif is [CCG]ₙ, where n=2. In some aspects, the repeating GC-motif is [CCG]ₙ, where n=1. In some aspects, the repeating GC-motif is [GCC]ₙ, where n=1 to 10. In some aspects, the repeating GC-motif is [GCC]ₙ, where n=1 to 5. In some aspects, the repeating GC-motif is [GCC]ₙ, where n=3. In some aspects, the repeating GC-motif is [GCC]ₙ, where n=2. In some aspects, the repeating

GC-motif is [GCC]ₙ, where n=1.

In any of the foregoing aspects, the sequence of the GC-rich RNA element comprises the sequence of EK1 [CCCGCC] set forth in SEQ ID NO: 3. In some aspects, the sequence of the GC-rich RNA element comprises the sequence of EK2 [GCCGCC] set forth in SEQ ID NO: 18. In some aspects, the sequence of the GC-rich RNA element comprises the sequence of EK3 [CCGCCG] set forth in SEQ ID NO: 19. In some aspects, the sequence of the GC-rich RNA element comprises the sequence of V1 [CCCCGGCGCC] set forth in SEQ ID NO: 1. In some aspects, the sequence of the GC-rich RNA element comprises the sequence of V2 [CCCCGGC] set forth in SEQ ID NO: 2. In some aspects, the sequence of the GC-rich RNA element comprises the sequence of CG1 [GCGCCCCGCGGCGCCCCGCG] set forth in SEQ ID NO: 20. In some aspects, the sequence of the GC-rich RNA element comprises the sequence of CG2 [CCCGCCCGCCCCGCCCCGCC] set forth in SEQ ID NO: 21.

In any of the foregoing aspects, the GC-rich RNA element comprises a stable RNA secondary structure. In some aspects, the GC-rich RNA element comprising a stable RNA secondary structure is located downstream of the initiation codon. In some aspects, the GC-rich RNA element comprising a stable RNA secondary structure is located about 30, about 25, about 20, about 15, about 10, or about 5 nucleotides downstream of the initiation codon. In some aspects, the GC-rich RNA element comprising a stable RNA secondary structure is located about 20, about 15, about 10 or about 5 nucleotides downstream of the initiation codon. In some aspects, the GC-rich RNA element comprising a stable RNA secondary structure is located about 5, about 4, about 3, about 2, about 1 nucleotide downstream of the initiation codon. In some aspects, the GC-rich RNA element comprising a stable RNA secondary structure is located about 15-30, about 15-20, about 15-25, about 10-15, or about 5-10 nucleotides downstream of the initiation codon. In some aspects, the GC-rich RNA element comprising a stable RNA secondary structure is located 20, 19, 18, 17, 16, 15, 14, 13, 12, 11, or 10 nucleotides downstream of the initiation codon. In some aspects, the GC-rich RNA element comprising a stable RNA secondary structure is located 15 nucleotides downstream of the initiation codon. In some aspects, the GC-rich RNA element comprising a stable RNA secondary structure is located 14 nucleotides downstream of the initiation codon. In some aspects, the GC-rich RNA element comprising a stable RNA secondary structure is located 13 nucleotides downstream of the initiation codon. In some aspects, the GC-rich RNA element comprising a stable RNA secondary structure is located 12 nucleotides downstream of the initiation codon.

In some aspects, the GC-rich RNA element comprising a stable RNA secondary structure is located upstream of the initiation codon in the 5′ UTR. In some aspects, the GC-rich RNA element comprising a stable RNA secondary structure is located about 40, about 35, about 30, about 25, about 20, about 15, about 10, or about 5 nucleotides upstream of the initiation codon. In some aspects, the GC-rich RNA element comprising a stable RNA secondary structure is located about 20, about 15, about 10 or about 5 nucleotides upstream of the initiation codon. In some aspects, the GC-rich RNA element comprising a stable RNA secondary structure is located about 5, about 4, about 3, about 2, about 1 nucleotide upstream of the initiation codon. In some aspects, the GC-rich RNA element comprising a stable RNA secondary structure is located about 15-40, about 15-30, about 15-20, about 15-25, about 10-15, or about 5-10 nucleotides upstream of the initiation codon.

In some aspects, the stable RNA secondary structure comprises the initiation codon and one or more additional nucleotides upstream, downstream, or upstream and downstream of the initiation codon.

In any of the foregoing aspects, the GC-rich RNA element comprising a stable RNA secondary structure comprises the sequence of SL1 [CCGCGGCGCCCCGCGG] as set forth in SEQ ID NO: 24. In some aspects, the GC-rich RNA element comprising a stable RNA secondary structure comprises the sequence of SL2 [GCGCGCAUAUAGCGCGC] as set forth in SEQ ID NO: 25. In some aspects, the GC-rich RNA element comprising a stable RNA secondary structure comprises the sequence of SL3 [CATGGTGGCGGCCCGCCGCCACCATG] as set forth in SEQ ID NO: 26. In some aspects, the GC-rich RNA element comprising a stable RNA secondary structure comprises the sequence of SL4 [CATGGTGGCCCGCCGCCACCATG] as set forth in SEQ ID NO: 27. In some aspects, the GC-rich RNA element comprising a stable RNA secondary structure comprises the sequence of SL5 [CATGGTGCCCGCCGCCACCATG] as set forth in SEQ ID NO: 28.

In any of the foregoing aspects, the stable RNA secondary structure is a hairpin or a stem-loop. In any of the foregoing aspects, the stable RNA secondary structure has a deltaG of about −30 kcal/mol, about −20 to −30 kcal/mol, about −20 kcal/mol, about −10 to −20 kcal/mol, about −10 kcal/mol, about −5 to −10 kcal/mol.

In some aspects, the disclosure provides methods to inhibit or reduce the initiation of polypeptide synthesis at any codon within an mRNA other than the initiation codon in a cell, the method comprising providing a C-rich RNA element described herein into a 5′UTR of the mRNA.

In some aspects, the disclosure provides methods to inhibit or reduce the amount of polypeptide translated from any open reading frame within an mRNA other than the full open reading frame, the method comprising providing a C-rich RNA element described herein into a 5′UTR of the mRNA.

In some aspects, the disclosure provides methods, to inhibit or reduce the production of aberrant translation products encoded by an mRNA, the method comprising providing a C-rich RNA element described herein into a 5′UTR of the mRNA.

In some aspects, the disclosure provides methods of identifying an RNA element having translational regulatory activity, the method comprising:

i. providing a population of polynucleotides, wherein each polynucleotide comprises a plurality of open reading frames encoding a plurality of polypeptides, each comprising a peptide epitope tag, wherein each polynucleotide comprises:
a. at least one first AUG codon upstream of, in-frame, and operably linked to, at least one first open reading frame encoding at least one first polypeptide comprising at least one first peptide epitope tag;
b. at least one second AUG codon upstream of, in-frame, and operably linked to, at least one second open reading frame encoding at least one second polypeptide comprising at least one second peptide epitope tag, wherein the second AUG codon is downstream and out-of-frame of the first AUG codon; optionally,
c. at least one third AUG codon upstream of, in-frame, and operably linked to, at least one third open reading frame encoding at least one third polypeptide comprising at least one third peptide epitope tag, wherein the third AUG codon is downstream and out-of-frame with the first and second AUG codons, and;
d. a 5′ UTR and a 3′ UTR, wherein the 5′ UTR of each polynucleotide within the population comprises a unique nucleotide sequence;
e. no stop codons (UAG, UGA, or UAA) within any frame between the first AUG and the stop codon corresponding to the first AUG;

ii. providing conditions suitable for translation of each polynucleotide in the population of polynucleotides; and

iii. isolating a complex comprising a nascent translation product comprising the first, second and, if present, third epitope tag, and the 5′ UTR corresponding to the epitope tag and encoded polynucleotide;

iv. determining the sequences of the 5′ UTRs corresponding to each polynucleotide encoding the nascent translation product;

v. determining which nucleotides are enriched at each position in the 5′UTR of the first polynucleotide compared to the second, and optionally third, polynucleotide.

In some aspects, the first polynucleotide is eGFP.

In some aspects, the first AUG is linked to and in frame with an open reading frame that encodes the first polynucleotide, wherein the first polynucleotide encodes eGFP.

In some aspects, the peptide epitope tag is selected from the group consisting of: a FLAG tag (SEQ ID NO: 133), a 3×FLAG tag (SEQ ID NO: 111), a Myc tag (SEQ ID NO: 112), a V5 tag (SEQ ID NO: 113), a hemagglutinin A (HA) tag (SEQ ID NO: 114), a histidine tag (e.g. a 6×His tag) (SEQ ID NO: 115), an HSV tag (SEQ ID NO: 116), a VSV-G tag (SEQ ID NO: 117), an NE tag (SEQ ID NO: 118), an AviTag (SEQ ID NO: 119), a Calmodulin tag (SEQ ID NO: 120), an E tag (SEQ ID NO: 121), an S tag (SEQ ID NO: 122), an SBP tag (SEQ ID NO: 123), a Softag 1 (SEQ ID NO: 124), a Softag 3 (SEQ ID NO: 125), a Strep tag (SEQ ID NO: 126), a Ty tag (SEQ ID NO: 127), or an Xpress tag (SEQ ID NO: 128).

In some aspects, the translational regulatory activity is selected from the group consisting of:
a. increasing residence time of a 43S pre-initiation complex (PIC) or ribosome at, or proximal to, the initiation codon;
b. increasing initiation of polypeptide synthesis at or from the initiation codon;
c. increasing an amount of polypeptide translated from the full open reading frame;
d. increasing fidelity of initiation codon decoding by the PIC or ribosome;
e. inhibiting or reducing leaky scanning by the PIC or ribosome;
f. decreasing a rate of decoding the initiation codon by the PIC or ribosome;
g. inhibiting or reducing initiation of polypeptide synthesis at any codon within the mRNA other than the initiation codon;
h. inhibiting or reducing the amount of polypeptide translated from any open reading frame within the mRNA other than the full open reading frame;
i. inhibiting or reducing the production of aberrant translation products;
j. increasing ribosomal density on the mRNA; and
k. a combination of any two or more of (a)-(j).

In some aspects, the translational regulatory activity is an increase in fidelity of initiation codon decoding by the PIC or ribosome, and an increase in ribosomal density on the mRNA.

In other aspects, the disclosure provides an mRNA comprising a 5′cap, a 5′UTR, a Kozak-like sequence, an open reading frame encoding a polypeptide, and a 3′ UTR, wherein the 5′UTR comprises:

(i) a C-rich RNA element comprising a nucleotide sequence selected from the group consisting of: SEQ ID NO: 29, SEQ ID NO: 30, SEQ ID NO: 31, SEQ ID NO: 32, SEQ ID NO: 33 and SEQ ID NO: 34, and

(ii) a GC-rich RNA element comprising a nucleotide sequence selected from the group consisting of: SEQ ID NO: 1, SEQ ID NO: 2, SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 and SEQ ID NO: 28.

In some aspects, the C-rich RNA element comprises a nucleotide sequence selected from the group consisting of SEQ ID NO: 31, SEQ ID NO: 32 and SEQ ID NO: 33, and the GC-rich RNA element comprises a nucleotide sequence selected from the group consisting of: SEQ ID NO: 1, SEQ ID NO: 2 and SEQ ID NO: 23.

In some aspects, the disclosure provides an mRNA comprising a 5′cap, a 5′UTR, a Kozak-like sequence, an open reading frame encoding a polypeptide, and a 3′ UTR, wherein the 5′UTR comprises a C-rich RNA element comprising the nucleotide sequence set forth in SEQ ID NO: 31 and the GC-rich RNA element comprises the nucleotide sequence set forth in SEQ ID NO: 1.

In some aspects, the disclosure provides an mRNA comprising a 5′cap, a 5′UTR, a Kozak-like sequence, an open reading frame encoding a polypeptide, and a 3′ UTR, wherein the 5′UTR comprises a C-rich RNA element comprising the nucleotide sequence set forth in SEQ ID NO: 33 and the GC-rich RNA element comprises the nucleotide sequence set forth in SEQ ID NO: 1. In some aspects, the disclosure provides an mRNA comprising a 5′cap, a 5′UTR, a Kozak-like sequence, an open reading frame encoding a polypeptide, and a 3′ UTR, wherein the 5′UTR comprises a C-rich RNA element comprising the nucleotide sequence set forth in SEQ ID NO: 32 and the GC-rich RNA element comprises the nucleotide sequence [GCC]ₙ set forth in SEQ ID NO: 23, where n=3.

In some aspects, the mRNA comprises a Kozak-like sequence comprising the nucleotide sequence [5′-GCCACC-3′] set forth in SEQ ID NO: 17 or a Kozak-like sequence comprising the nucleotide sequence [5′-GCCGCC-3′] set forth in SEQ ID NO: 17.

In other aspects, the disclosure provides an mRNA comprising a 5′cap, a 5′UTR, a Kozak-like sequence, an open reading frame encoding a polypeptide, and a 3′ UTR, wherein the 5′UTR comprises:

(i) a C-rich RNA element comprising a nucleotide sequence selected from the group consisting of: SEQ ID NO: 29, SEQ ID NO: 30, SEQ ID NO: 31, SEQ ID NO: 32, SEQ ID NO: 33 and SEQ ID NO: 34, and

(ii) a GC-rich RNA element comprising a nucleotide sequence selected from the group consisting of: SEQ ID NO: 1, SEQ ID NO: 2, SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 and SEQ ID NO: 28, wherein the C-rich RNA element is located downstream of and immediately adjacent to the 5′ cap in the 5′UTR. In some aspects, the C-rich RNA element is located about 20-25, about 15-20, about 10-15, about 6-10 nucleotides, about 1-5 nucleotides, or about 20, 19, 18, 17, 16, 15, 14, 13, 12, 11, 10, 9, 8, 7, 6, 5, 4, 3, 2 or 1 nucleotide(s) downstream of the 5′ cap or 5′ end of the mRNA in the 5′ UTR. In some aspects, the C-rich RNA element is located upstream of the GC-rich RNA element in the 5′ UTR. In some aspects, the C-rich RNA element is located about 45-50, about 40-45, about 35-40, about 30-35, about 25-30, about 20-25, about 15-20, about 10-15, about 6-10 nucleotides upstream of the GC-rich RNA element in the 5′ UTR. In some aspects, the GC-rich RNA element is located about 20, about 15, about 10 or about 5 nucleotides upstream of the Kozak like sequence in the 5′ UTR. In some aspects, the GC-rich RNA element is located about 5, about 4, about 3, about 2, or about 1 nucleotide upstream of the Kozak like sequence in the 5′ UTR. In some aspects, the GC-rich RNA element is upstream of and immediately adjacent to the Kozak like sequence in the 5′ UTR.

In any of the foregoing or related aspects, the mRNA of the disclosure comprises a 5′ UTR comprising the nucleotide sequence set forth in SEQ ID NO: 45, wherein the 5′ UTR comprises a C-rich RNA element and, optionally a GC-rich RNA element of the disclosure.

In any of the foregoing or related aspects, the mRNA of the disclosure comprises a 5′ UTR comprising the nucleotide sequence set forth in SEQ ID NO: 46 or comprising the nucleotide sequence set forth in SEQ ID NO: 42, wherein the 5′ UTR comprises a C-rich RNA element and, optionally a GC-rich RNA element of the disclosure.

In some aspects, the disclosure provides an mRNA comprising: a 5′ UTR; an open reading frame encoding a polypeptide; and a 3′ UTR, wherein the 5′ UTR comprises the nucleotide sequence set forth in SEQ ID NO: 35.

In some aspects, the disclosure provides an mRNA comprising: a 5′ UTR; an open reading frame encoding a polypeptide; and a 3′ UTR, wherein the 5′ UTR comprises the nucleotide sequence set forth in SEQ ID NO: 36.

In some aspects, the disclosure provides an mRNA comprising: a 5′ UTR; an open reading frame encoding a polypeptide; and a 3′ UTR, wherein the 5′ UTR comprises the nucleotide sequence set forth in SEQ ID NO: 40.

In some aspects, the disclosure provides an mRNA comprising: a 5′ UTR; an open reading frame encoding a polypeptide; and a 3′ UTR, wherein the 5′ UTR comprises the nucleotide sequence set forth in SEQ ID NO: 41.

In some aspects, the disclosure provides an mRNA comprising: a 5′ UTR; an open reading frame encoding a polypeptide; and a 3′ UTR, wherein the 5′ UTR comprises the nucleotide sequence set forth in SEQ ID NO: 44.

In some aspects, an mRNA of the disclosure comprises a 5′ UTR, an ORF encoding a polypeptide, and a 3′ UTR, wherein the 5′ UTR comprises a nucleotide sequence selected from the group consisting of: SEQ ID NO: 35, SEQ ID NO: 87, SEQ ID NO: 160, SEQ ID NO: 36, SEQ ID NO: 88, SEQ ID NO: 161, SEQ ID NO: 40, SEQ ID NO: 85, SEQ ID NO: 158, SEQ ID NO: 41, SEQ ID NO: 86, SEQ ID NO: 159, SEQ ID NO: 44, SEQ ID NO: 89, SEQ ID NO: 162, SEQ ID NO: 38, SEQ ID NO: 84, or ID NO: 157.

In some aspects, the disclosure provides an mRNA comprising: a 5′cap, a 5′UTR, a Kozak-like sequence, an open reading frame encoding a polypeptide, and a 3′ UTR, wherein the 5′ UTR comprises a C-rich RNA element comprising the nucleotide sequence set forth in SEQ ID NO: 31 inserted within a 5′ UTR comprising the nucleotide sequence set forth in SEQ ID NO: 45.

In some aspects, the disclosure provides an mRNA comprising: a 5′cap, a 5′UTR, a Kozak-like sequence, an open reading frame encoding a polypeptide, and a 3′ UTR, wherein the 5′ UTR comprises a C-rich RNA element comprising the nucleotide sequence set forth in SEQ ID NO: 32 inserted within a 5′ UTR comprising the nucleotide sequence set forth in SEQ ID NO: 45.

In some aspects, the disclosure provides an mRNA comprising: a 5′cap, a 5′UTR, a Kozak-like sequence, an open reading frame encoding a polypeptide, and a 3′ UTR, wherein the 5′ UTR comprises a C-rich RNA element comprising the nucleotide sequence set forth in SEQ ID NO: 33 inserted within a 5′ UTR comprising the nucleotide sequence set forth in SEQ ID NO: 45.

In some aspects, the disclosure provides an mRNA comprising: a 5′cap, a 5′UTR, a Kozak-like sequence, an open reading frame encoding a polypeptide, and a 3′ UTR, wherein the 5′ UTR comprises a C-rich RNA element comprising the nucleotide sequence set forth in SEQ ID NO: 31 inserted within a 5′ UTR comprising the nucleotide sequence set forth in SEQ ID NO: 46 or the nucleotide sequence set forth in SEQ ID NO: 42.

In some aspects, the disclosure provides an mRNA comprising: a 5′cap, a 5′UTR, a Kozak-like sequence, an open reading frame encoding a polypeptide, and a 3′ UTR, wherein the 5′ UTR comprises a C-rich RNA element comprising the nucleotide sequence set forth in SEQ ID NO: 32 inserted within a 5′ UTR comprising the nucleotide sequence set forth in SEQ ID NO: 46 or the nucleotide sequence set forth in SEQ ID NO: 42.

In some aspects, the disclosure provides an mRNA comprising: a 5′cap, a 5′UTR, a Kozak-like sequence, an open reading frame encoding a polypeptide, and a 3′ UTR, wherein the 5′ UTR comprises a C-rich RNA element comprising the nucleotide sequence set forth in SEQ ID NO: 33 inserted within a 5′ UTR comprising the nucleotide sequence set forth in SEQ ID NO: 46 or the nucleotide sequence set forth in SEQ ID NO: 42.

In any of the foregoing aspects, the disclosure provides an mRNA wherein the C-rich RNA element is located downstream of and immediately adjacent to the 5′ cap in the 5′UTR. In some aspects, the C-rich RNA element is located about 20-25, about 15-20, about 10-15, about 6-10 nucleotides, about 1-5 nucleotides, or about 20, 19, 18, 17, 16, 15, 14, 13, 12, 11, 10, 9, 8, 7, 6, 5, 4, 3, 2 or 1 nucleotide(s) downstream of the 5′ cap or 5′ end of the mRNA in the 5′ UTR.

In any of the foregoing aspects, the disclosure provides an mRNA wherein the 5′ UTR comprises a GC-rich RNA element comprising the nucleotide sequence set forth in SEQ ID NO: 1. In any of the foregoing aspects, the C-rich RNA element is located about 45-50, about 40-45, about 35-40, about 30-35, about 25-30, about 20-25, about 15-20, about 10-15, about 6-10 nucleotides upstream of the GC-rich RNA element in the 5′ UTR. In any of the foregoing aspects, the GC-rich RNA element is located about 20, about 15, about 10 or about 5 nucleotides upstream of the Kozak like sequence in the 5′ UTR. In some aspects, the GC-rich RNA element is located about 5, about 4, about 3, about 2, or about 1 nucleotide upstream of the Kozak like sequence in the 5′ UTR. In some aspects, the GC-rich RNA element is upstream of and immediately adjacent to the Kozak like sequence in the 5′ UTR.

In other aspects, the disclosure provides a method to inhibit or reduce the initiation of polypeptide synthesis at any codon within an mRNA other than the initiation codon in a cell, the method comprising administering to a subject an mRNA comprising a 5′UTR comprising a C-rich RNA element and, optionally a GC-rich RNA element of the disclosure.

In other aspects, the disclosure provides a method to inhibit or reduce the amount of polypeptide translated from any open reading frame within an mRNA other than the full open reading frame, the method comprising administering to a subject an mRNA comprising a 5′UTR comprising a C-rich RNA element and, optionally a GC-rich RNA element of the disclosure.

In other aspects, the disclosure provides method to inhibit or reduce the production of aberrant translation products encoded by an mRNA, the method comprising administering to a subject an mRNA comprising a 5′UTR comprising a C-rich RNA element and, optionally a GC-rich RNA element of the disclosure.

### BRIEF DESCRIPTION OF DRAWINGS

FIG. 1 provides a schematic of a reporter system utilizing three separate epitope tags to assess effects of random 5′ UTR sequences in mRNA constructs on leaky scanning.

FIG. 2 is a graph showing nucleotides associated with start site fidelity in an 18 nucleotide 5′ UTR screen using the reporter system provided in FIG. 1, wherein the graph shows the ratio of the abundance of each nucleotide at each position that gave rise to initiation at the first start site compared to subsequent start sites.

FIG. 3 is a graph showing nucleotides associated with start site fidelity in a 50 nucleotide 5′ UTR screen using the reporter system provided in FIG. 1, wherein the graph shows the ratio of the abundance of each nucleotide at each position that gave rise to initiation at the first start site compared to subsequent start sites.

FIG. 4A is an example of a polysome gradient, where mRNAs bearing different numbers of ribosomes are separated by size.

FIG. 4B is a graph showing the associations between nucleotide content of the 18 nucleotide 5′UTR and relative probability of an mRNA co-sedimenting with >7 ribosomes, using the reporter system provided in FIG. 1.

FIG. 5 is a graph showing the extent of leaky scanning of reporter mRNAs encoding a 3×FLAG-eGFP leaky scanning reporter polypeptide and comprising 5′ UTRs with a C-rich RNA element (combo2_S065 SEQ ID NO: 38 and combo5_S065 SEQ ID NO: 41) relative to a reference reporter mRNA comprising a 5′ UTR that does not contain a C-rich RNA element (S065 (Ref), SEQ ID NO: 42) in HeLa cells as determined by capillary immunoblot analysis of mRNA-transfected cells.

FIGS. 6A-6B is a graph showing the extent of leaky scanning of reporter mRNAs encoding a 3×FLAG-e leaky scanning reporter polypeptide and comprising 5′ UTRs with a GC-rich RNA element in combination with a C-rich RNA element (combo1_v1.1 SEQ ID NO: 35, combo2_v1.1 SEQ ID NO: 36) relative to a reference mRNA comprising a 5′ UTR that contains a CG-rich RNA element alone (v1.1(Ref) (DNA) SEQ ID NO: 9; v1.1(Ref) (RNA) SEQ ID NO: 132) in HeLa cells (FIG. 6A) and AML12 cells (FIG. 6B) as determined by capillary immunoblot analysis of mRNA-transfected cells.

FIGS. 7A-7B is a graph showing the extent of leaky scanning of a reporter mRNA encoding a 3×FLAG-eGFP leaky scanning reporter polypeptide and comprising a 5′ UTR with a GC-rich RNA element in combination with a C-rich RNA element (CrichCR4+GCC3-ExtKozak SEQ ID NO: 44) relative to a reference mRNA comprising a 5′ UTR that contains a GC-rich RNA element alone (GCC3-ExtKozak (Ref) SEQ ID NO: 43) in HeLa cells (FIG. 7A) and AML12 cells (FIG. 7B) as determined by capillary immunoblot analysis of mRNA-transfected cells.

FIG. 8A-8B provides graphs showing the rate of leaky scanning of reporter mRNAs encoding a 3×FLAG-eGFP leaky scanning reporter polypeptide plotted against the length (i.e., number of nucleotides) of the 5′ UTR in HeLa cells (FIG. 8A) and AML12 cells (FIG. 8B).

### DETAILED DESCRIPTION

### Definitions

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Base Composition: As used herein, the term “base composition” refers to the proportion of the total bases of a nucleic acid consisting of guanine+cytosine or thymine (or uracil)+adenine nucleotides.

Base Pair: As used herein, the term “base pair” refers to two nucleobases on opposite complementary nucleic acid strands that interact via the formation of specific hydrogen bonds. As used herein, the term “Watson-Crick base pairing”, used interchangeably with “complementary base pairing”, refers to a set of base pairing rules, wherein a purine always binds with a pyrimidine such that the nucleobase adenine (A) forms a complementary base pair with thymine (T) and guanine (G) forms a complementary base pair with cytosine (C) in DNA molecules. In RNA molecules, thymine is replaced by uracil (U), which, similar to thymine (T), forms a complementary base pair with adenine (A). The complementary base pairs are bound together by hydrogen bonds and the number of hydrogen bonds differs between base pairs. As in known in the art, guanine (G)-cytosine (C) base pairs are bound by three (3) hydrogen bonds and adenine (A)-thymine (T) or uracil (U) base pairs are bound by two (2) hydrogen bonds. Base pairing interactions that do not follow these rules can occur in natural, non-natural, and synthetic nucleic acids and are referred to herein as “non-Watson-Crick base pairing” or alternatively “non-complementary base pairing”.

C-rich: As used herein, the term “C-rich” refers to the nucleobase composition of a polynucleotide (e.g., mRNA), or any portion thereof (e.g., a C-rich RNA element), comprising cytosine (C) nucleobases, or derivatives or analogs thereof, wherein the C-content is at least 50% or greater and is located proximal to the 5′ end of the mRNA (e.g., proximal to the 5′ cap). In some aspects, the term C-rich (e.g., a C-rich RNA element) comprises at least 55% or greater, at least 60% or greater, at least 65% or greater, at least 70% or greater, at least 75% or greater, at least 80% or greater, at least 85% or greater, at least 90% or greater, about 90%, about 91%, about 92%, about 93%, about 94%, or about 95% cytosine nucleobases, or derivatives or analogs thereof. In some embodiments that C-rich element comprises at least 95%, 96%, 97%, 98%, 99% or 100% cytosine nucleobases, or derivatives or analogs thereof. In some embodiments, the C-rich RNA element is about 15 nucleotides and comprises at least 90% or at 100% cytosine nucleobases, or derivatives or analogs thereof. The term “C-rich” refers to all, or to a portion, of a polynucleotide, including, but not limited to, a gene, a non-coding region, a 5′ UTR, a 3′ UTR, an open reading frame, an RNA element, a sequence motif, or any discrete sequence, fragment, or segment thereof which comprises at least 50% or greater C-content. In some aspects, C-rich polynucleotides, or any portions thereof, are exclusively comprised of cytosine (C) nucleobases. In some aspects, a C-rich polynucleotide comprises a C-rich RNA element comprising a sequence of linked nucleotides, or derivatives or analogs thereof, wherein each nucleotide comprises a nucleobase selected from the group consisting of: adenine, guanine, thymine, uracil, and cytosine, linked in any order. In some aspects, the C-rich RNA element comprises about 3-20 nucleotides. In some aspects, the C-rich RNA element is located within a 5′UTR of an mRNA and is located proximal to the 5′ end of the mRNA (e.g., proximal to the 5′ cap). In some aspects, the C-rich RNA element is located within a 5′UTR of an mRNA and is located adjacent to or within about 1-6 or about 1-10 nucleotides downstream of the 5′ end of the mRNA (e.g., adjacent to or within about 1-6 or about 1-10 nucleotides downstream of the 5′ cap). In some aspects, the C-rich RNA element is located within a 5′UTR of an mRNA and is located about 1-20, about 2-15, about 3-10, about 4-8, or about 6 nucleotides downstream of the 5′ cap in the 5′ UTR.

C-content: As used herein, the term “C-content” refers to the percentage of nucleobases in a polynucleotide (e.g., mRNA), or a portion thereof (e.g., an RNA element), that are cytosine (C) nucleobases, or derivatives or analogs thereof, (from a total number of possible nucleobases, including guanine (G), adenine (A) and thymine (T) or uracil (U), and derivatives or analogs thereof, in DNA and in RNA). The term “C-content” refers to all, or to a portion, of a polynucleotide, including, but not limited to, a gene, a non-coding region, a 5′ or 3′ UTR, an open reading frame, an RNA element, a sequence motif, or any discrete sequence, fragment, or segment thereof. In some aspects, the C-content of a C-rich RNA element comprises at least 50% or greater cytosine nucleobases, or derivatives or analogs thereof, and less than 10% guanosine nucleobases, or derivatives or analogs thereof. In some aspects, the C-content of a C-rich RNA element comprises at least 50% or greater cytosine nucleobases, or derivatives or analogs thereof, and less than 5% guanosine nucleobases, or derivatives or analogs thereof. In some aspects, the C-content of a C-rich RNA element comprises at least 50% or greater cytosine nucleobases, or derivatives or analogs thereof, with the remaining content comprising adenosine nucleobases, or derivatives or analogs thereof. In some aspects, the C-content of a C-rich RNA element comprises at least 50% or greater cytosine nucleobases, or derivatives or analogs thereof, with the remaining content comprising adenosine nucleobases and uracil nucleobases, or derivatives or analogs thereof (e.g., pseudouridine, N1-methyl pseudouridine, 5-methoxyuridine) and no guanosine nucleobases. In some aspects, the C-content of a C-rich RNA element comprises at least 50% or greater cytosine nucleobases, or derivatives or analogs thereof, with the remaining content comprising preferentially adenosine>uracil>>guanosine (A>U>>G) nucleobases, or derivatives or analogs thereof (e.g., pseudouridine, N1-methyl pseudouridine, 5-methoxyuridine). In some aspects, the C-content of a C-rich RNA element comprises at least 50% or greater cytosine nucleobases, or derivatives or analogs thereof, with the remaining content comprising preferentially adenosine (15-45%), uracil (5-10%) and guanosine (5%-10%) nucleobases, or derivatives or analogs thereof (e.g., pseudouridine, N1-methyl pseudouridine, 5-methoxyuridine).

Cap structure or 5′ cap structure: As used herein, the terms “cap structure”, “5′ cap structure” and “5′cap” refer to a non-extendible dinucleotide that facilitates translation or localization, and/or prevents degradation of an RNA transcript when incorporated at the 5′ end of an RNA transcript, wherein the cap structure can be a natural cap, a derivative of a natural cap, or any chemical group that protects the 5′end of an RNA from degradation and/or is essential for translation initiation. In nature, the modified base 7-methylguanosine is joined in the opposite orientation, 5′ to 5′ rather than 5′ to 3′, to the rest of the molecule via three phosphate groups (i.e., P1-guanosine-5′-yl P3-7-methylguanosine-5′-yl triphosphate (m⁷G5′ppp5′G)). In some embodiments, the mRNA provided herein comprises a “cap analog”, which refers to a structural derivative of an RNA cap that may differ by as little as a single element. In some embodiments, the mRNA provided herein comprises a “mCAP”, which refers to a dinucleotide cap with the N7 position of the guanosine having a methyl group. The structure can be represented as m⁷G(5′)ppp(g′)G, through a triphosphate, a tetraphosphate or a pentaphosphate group can join the two nucleotides.

Codon: As used herein, the term “codon” refers to a sequence of three nucleotides that together form a unit of genetic code in a DNA or RNA molecule. A codon is operationally defined by the initial nucleotide from which translation starts and sets the frame for a run of successive nucleotide triplets, which is known as an “open reading frame” (ORF). For example, the string GGGAAACCC, if read from the first position, contains the codons GGG, AAA, and CCC; if read from the second position, it contains the codons GGA and AAC; and if read from the third position, GAA and ACC. Thus, every nucleic sequence read in its 5′→3′ direction comprises three reading frames, each producing a possibly distinct amino acid sequence (in the given example, Gly-Lys-Pro, Gly-Asn, or Glu-Thr, respectively). DNA is double-stranded defining six possible reading frames, three in the forward orientation on one strand and three reverse on the opposite strand. Open reading frames encoding polypeptides are typically defined by a start codon, usually the first AUG codon in the sequence.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., an isolated mRNA, nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle or an isolated mRNA) and a cell may be contacted, for example, by adding the composition to the culture medium of the cell and may involve or result in transfection. Moreover, more than one cell may be contacted by a nanoparticle composition.

Denaturation: As used herein, the term “denaturation” refers to the process by which the hydrogen bonding between base paired nucleotides in a nucleic acid is disrupted, resulting in the loss of secondary and/or tertiary nucleic acid structure (e.g. the separation of previously annealed strands). Denaturation can occur by the application of an external substance, energy, or biochemical process to a nucleic acid. For example, local denaturation of nucleic acid structure by enzymatic activity occurs when biologically important transactions such as DNA replication, transcription, translation, or DNA repair need to occur. Folded structures (e.g. secondary and tertiary nucleic acid structures) of an mRNA can constitute a barrier to the scanning function of the PIC or the elongation function of the ribosome, resulting in a lower translation rate. During translation initiation, helicase activity provided by eIFs (e.g. eIF4A) can denature or unwind duplexed, double-stranded RNA structure to facilitate PIC scanning.

Epitope Tag: As used herein, the term “epitope tag” refers to an artificial epitope, also known as an antigenic determinant, which is fused to a polypeptide sequence by placing the sequence encoding the epitope in-frame with the coding sequence or open reading frame of a polypeptide. An epitope-tagged polypeptides is considered a fusion protein. Epitope tags are relatively short peptide sequences ranging from about 10-30 amino acids in length. Epitope tags are usually fused to either the N- or C-terminus in order to minimize tertiary structure disruptions that may alter protein function. Epitope tags are reactive to high-affinity antibodies that can be reliably produced in many different species. Exemplary epitope tags include the V5-tag, Myc-tag, HA-tag and 3×FLAG-tag. These tags are useful for detection or purification of fusion proteins by Western blotting, immunofluorescence, or immunoprecipitation techniques.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Identity: As used herein, the term “identity” refers to the overall relatedness between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux et al., Nucleic Acids Research, 12(1): 387,1984, BLASTP, BLASTN, and FASTA, Altschul, S. F. et al., J. Molec. Biol., 215, 403, 1990.

Fragment: A “fragment,” as used herein, refers to a portion. For example, fragments of proteins may include polypeptides obtained by digesting full-length protein isolated from cultured cells or obtained through recombinant DNA techniques.

Fusion Protein: The term “fusion protein” means a polypeptide sequence that is comprised of two or more polypeptide sequences linked by a peptide bond(s). “Fusion proteins” that do not occur in nature can be generated using recombinant DNA techniques.

GC-rich: As used herein, the term “GC-rich” refers to the nucleobase composition of a polynucleotide (e.g., mRNA), or any portion thereof (e.g., an RNA element), comprising guanine (G) and/or cytosine (C) nucleobases, or derivatives or analogs thereof, wherein the GC-content is at least 50% or greater. The term “GC-rich” refers to all, or to a portion, of a polynucleotide, including, but not limited to, a gene, a non-coding region, a 5′ UTR, a 3′ UTR, an open reading frame, an RNA element, a sequence motif, or any discrete sequence, fragment, or segment thereof which comprises at least 50% or greater GC-content. In some aspects, the term GC-rich (e.g., a GC-rich RNA element) comprises at least 55% or greater, at least 60% or greater, at least 65% or greater, at least 70% or greater, at least 75% or greater, at least 80% or greater, at least 85% or greater, at least 90% or greater, or at least 95%, 96%, 97%, 98%, 99% or 100% guanosine and cytosine nucleobases, or derivatives or analogs thereof. In some embodiments of the disclosure, GC-rich polynucleotides, or any portions thereof, are exclusively comprised of guanine (G) and/or cytosine (C) nucleobases.

GC-content: As used herein, the term “GC-content” refers to the percentage of nucleobases in a polynucleotide (e.g., mRNA), or a portion thereof (e.g., an RNA element), that are either guanine (G) and cytosine (C) nucleobases, or derivatives or analogs thereof, (from a total number of possible nucleobases, including adenine (A) and thymine (T) or uracil (U), and derivatives or analogs thereof, in DNA and in RNA (e.g., pseudouridine, N1-methyl pseudouridine, 5-methoxyuridine)). The term “GC-content” refers to all, or to a portion, of a polynucleotide, including, but not limited to, a gene, a non-coding region, a 5′ or 3′ UTR, an open reading frame, an RNA element, a sequence motif, or any discrete sequence, fragment, or segment thereof.

Genetic code: As used herein, the term “genetic code” refers to the set of rules by which genetic information encoded within genetic material (DNA or RNA sequences) is translated by the ribosome into polypeptides. The code defines how sequences of nucleotide triplets, referred to as “codons”, specify which amino acid will be added next during protein synthesis. A three-nucleotide codon in a nucleic acid sequence specifies a single amino acid. The vast majority of genes are encoded with a single scheme of rules referred to as the canonical or standard genetic code, or simply the genetic code, though variant codes (such as in human mitochondria) exist.

Heterologous: As used herein, “heterologous” indicates that a sequence (e.g., an amino acid sequence or the polynucleotide that encodes an amino acid sequence) is not normally present in a given natural polypeptide or polynucleotide. For example, an amino acid sequence that corresponds to a domain or motif of one protein may be heterologous to a second protein.

Hybridization: As used herein, the term “hybridization” refers to the process of a first single-stranded nucleic acid, or a portion, fragment, or region thereof, annealing to a second single-stranded nucleic acid, or a portion, fragment, or region thereof, either from the same or separate nucleic acid molecules, mediated by Watson-Crick base pairing to form a secondary and/or tertiary structure. Complementary strands of linked nucleobases able to undergo hybridization can be from either the same or separate nucleic acids. Due to the thermodynamically favorable hydrogen bonding interaction between complementary base pairs, hybridization is a fundamental property of complementary nucleic acid sequences. Such hybridization of nucleic acids, or a portion or fragment thereof, may occur with “near” or “substantial” complementarity, as well as with exact complementarity.

Initiation Codon: As used herein, the term “initiation codon”, used interchangeably with the term “start codon”, refers to the first codon of an open reading frame that is translated by the ribosome and is comprised of a triplet of linked adenine-uracil-guanine nucleobases. The initiation codon is depicted by the first letter codes of adenine (A), uracil (U), and guanine (G) and is often written simply as “AUG”. Although natural mRNAs may use codons other than AUG as the initiation codon, which are referred to herein as “alternative initiation codons”, the initiation codons of polynucleotides described herein use the AUG codon. During the process of translation initiation, the sequence comprising the initiation codon is recognized via complementary base-pairing to the anticodon of an initiator tRNA (Met-tRNAᵢ^{Met}) bound by the ribosome. Open reading frames may contain more than one AUG initiation codon, which are referred to herein as “alternate initiation codons”.

The initiation codon plays a critical role in translation initiation. The initiation codon is the first codon of an open reading frame that is translated by the ribosome. Typically, the initiation codon comprises the nucleotide triplet AUG, however, in some instances translation initiation can occur at other codons comprised of distinct nucleotides. The initiation of translation in eukaryotes is a multistep biochemical process that involves numerous protein-protein, protein-RNA, and RNA-RNA interactions between messenger RNA molecules (mRNAs), the 40S ribosomal subunit, other components of the translation machinery (e.g., eukaryotic initiation factors; eIFs). The current model of mRNA translation initiation postulates that the pre-initiation complex (alternatively “43S pre-initiation complex”; abbreviated as “PIC”) translocates from the site of recruitment on the mRNA (typically the 5′ cap) to the initiation codon by scanning nucleotides in a 5′ to 3′ direction until the first AUG codon that resides within a specific translation-promotive nucleotide context (the Kozak sequence) is encountered (Kozak (1989) J Cell Biol 108:229-241). Scanning by the PIC ends upon complementary base-pairing between nucleotides comprising the anticodon of the initiator Met-tRNAᵢ^{Met} transfer RNA and nucleotides comprising the initiation codon of the mRNA. Productive base-pairing between the AUG codon and the Met-tRNAᵢ^{Met} anticodon elicits a series of structural and biochemical events that culminate in the joining of the large 60S ribosomal subunit to the PIC to form an active ribosome that is competent for translation elongation.

Insertion: As used herein, an “insertion” or an “addition” refers to a change in an amino acid or nucleotide sequence resulting in the addition of one or more amino acid residues or nucleotides, respectively, to a molecule as compared to a reference sequence, for example, the sequence found in a naturally-occurring molecule.

Insertion Site: As used herein, an “insertion site” is a position or region of a scaffold polypeptide that is amenable to insertion of an amino acid sequence of a heterologous polypeptide. It is to be understood that an insertion site also may refer to the position or region of the polynucleotide that encodes the polypeptide (e.g., a codon of a polynucleotide that codes for a given amino acid in the scaffold polypeptide). In some embodiments, insertion of an amino acid sequence of a heterologous polypeptide into a scaffold polypeptide has little to no effect on the stability (e.g., conformational stability), expression level, or overall secondary structure of the scaffold polypeptide.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Kozak Sequence: The term “Kozak sequence” (also referred to as “Kozak consensus sequence”) refers to a translation initiation enhancer element to enhance expression of a gene or open reading frame, and which in eukaryotes, is located in the 5′ UTR. The Kozak consensus sequence was originally defined as the sequence GCCRCC, where R=a purine, following an analysis of the effects of single mutations surrounding the initiation codon (AUG) on translation of the preproinsulin gene (Kozak (1986) Cell 44:283-292). Polynucleotides disclosed herein comprise a Kozak consensus sequence, or a derivative or modification thereof. (Examples of translational enhancer compositions and methods of use thereof, see U.S. Pat. No. 5,807,707 to Andrews et al., incorporated herein by reference in its entirety; U.S. Pat. No. 5,723,332 to Chernajovsky, incorporated herein by reference in its entirety; U.S. Pat. No. 5,891,665 to Wilson, incorporated herein by reference in its entirety.)

Kozak-like sequence: As used herein, the term “Kozak-like sequence” refers to a sequence similar to the Kozak sequence described supra, comprising an adenine or guanine three nucleotides upstream of the AUG start codon. In some embodiments, the Kozak-like sequence is gcc(X)ccAUG, wherein X is A or G, and wherein the lower case letters indicate bases that are weakly preferred.

Leaky scanning: As used herein, the term “leaky scanning” refers to a biological phenomenon whereby the pre-initiation complex (PIC) bypasses the initiation codon of an mRNA and instead continues scanning downstream until an alternate or alternative initiation codon is recognized. Depending on the frequency of occurrence, the bypass of the initiation codon by the PIC can result in a decrease in translation efficiency. Furthermore, translation from this downstream AUG codon can occur, which will result in the production of an undesired, aberrant translation product that may not be capable of eliciting the desired therapeutic response. In some cases, the aberrant translation product may in fact cause a deleterious response (Kracht et al., (2017) Nat Med 23(4):501-507).

mRNA: As used herein, an “mRNA” refers to a messenger ribonucleic acid. An mRNA may be naturally or non-naturally occurring or synthetic. For example, an mRNA may include modified and/or non-naturally occurring components such as one or more nucleobases, nucleosides, nucleotides, or linkers. An mRNA may include a cap structure, a 5′ transcript leader, a 5′ untranslated region, an initiator codon, an open reading frame, a stop codon, a chain terminating nucleoside, a stem-loop, a hairpin, a polyA sequence, a polyadenylation signal, and/or one or more cis-regulatory elements. An mRNA may have a nucleotide sequence encoding a polypeptide. Translation of an mRNA, for example, in vivo translation of an mRNA inside a mammalian cell, may produce a polypeptide. Traditionally, the basic components of a natural mRNA molecule include at least a coding region, a 5′-untranslated region (5′-UTR), a 3′UTR, a 5′ cap and a polyA sequence.

microRNA (miRNA) binding site: As used herein, a “microRNA (miRNA) binding site” refers to a miRNA target site or a miRNA recognition site, or any nucleotide sequence to which a miRNA binds or associates. In some embodiments, a miRNA binding site represents a nucleotide location or region of an mRNA to which at least the “seed” region of a miRNA binds. It should be understood that “binding” may follow traditional Watson-Crick hybridization rules or may reflect any stable association of the miRNA with the target sequence at or adjacent to the microRNA site.

miRNA seed: As used herein, a “seed” region of a miRNA refers to a sequence in the region of positions 2-8 of a mature miRNA, which typically has perfect Watson-Crick complementarity to the miRNA binding site. A miRNA seed may include positions 2-8 or 2-7 of a mature miRNA. In some embodiments, a miRNA seed may comprise 7 nucleotides (e.g., nucleotides 2-8 of a mature miRNA), wherein the seed-complementary site in the corresponding miRNA binding site is flanked by an adenine (A) opposed to miRNA position 1. In some embodiments, a miRNA seed may comprise 6 nucleotides (e.g., nucleotides 2-7 of a mature miRNA), wherein the seed-complementary site in the corresponding miRNA binding site is flanked by an adenine (A) opposed to miRNA position 1. When referring to a miRNA binding site, an miRNA seed sequence is to be understood as having complementarity (e.g., partial, substantial, or complete complementarity) with the seed sequence of the miRNA that binds to the miRNA binding site.

Modified: As used herein “modified” or “modification” refers to a changed state or a change in composition or structure of a polynucleotide (e.g., mRNA). Polynucleotides may be modified in various ways including chemically, structurally, and/or functionally. For example, polynucleotides may be structurally modified by the incorporation of one or more RNA elements, wherein the RNA element comprises a sequence and/or an RNA secondary structure(s) that provides one or more functions (e.g., translational regulatory activity). Accordingly, polynucleotides of the disclosure may be comprised of one or more modifications (e.g., may include one or more chemical, structural, or functional modifications, including any combination thereof).

Nascent translation product: As used herein, the term “nascent translation product” refers to a series of linked amino acids undergoing elongation catalyzed by the ribosome. The nascent translation product is characterized by association with the ribosome. In some embodiments, association with the ribosome is in the peptide exit channel. In some embodiments, the nascent translation product is characterized by covalent association with a tRNA. In some embodiments, the nascent translation product is characterized by association with the ribosome in the peptide exit channel and covalent association with a tRNA. In some embodiments, the nascent translation product is characterized by association with the ribosome in the peptide exit channel, covalent association with a tRNA, and non-covalent association with the mRNA.

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside/Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Nucleic acid: As used herein, the term “nucleic acid” is used in its broadest sense and encompasses any compound and/or substance that includes a polymer of nucleotides, or derivatives or analogs thereof. These polymers are often referred to as “polynucleotides”. Accordingly, as used herein the terms “nucleic acid” and “polynucleotide” are equivalent and are used interchangeably. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, mRNAs, modified mRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

Nucleic Acid Structure: As used herein, the term “nucleic acid structure” (used interchangeably with “polynucleotide structure”) refers to the arrangement or organization of atoms, chemical constituents, elements, motifs, and/or sequence of linked nucleotides, or derivatives or analogs thereof, that comprise a nucleic acid (e.g., an mRNA). The term also refers to the two-dimensional or three-dimensional state of a nucleic acid. Accordingly, the term “RNA structure” refers to the arrangement or organization of atoms, chemical constituents, elements, motifs, and/or sequence of linked nucleotides, or derivatives or analogs thereof, comprising an RNA molecule (e.g., an mRNA) and/or refers to a two-dimensional and/or three dimensional state of an RNA molecule. Nucleic acid structure can be further demarcated into four organizational categories referred to herein as “molecular structure”, “primary structure”, “secondary structure”, and “tertiary structure” based on increasing organizational complexity.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Pre-Initiation Complex: As used herein, the term “pre-initiation complex” (alternatively “43S pre-initiation complex”; abbreviated as “PIC”) refers to a ribonucleoprotein complex comprising a 40S ribosomal subunit, eukaryotic initiation factors (eIF1, eIF1A, eIF3, eIF5), and the eIF2-GTP-Met-tRNAᵢ^{Met} ternary complex, that is intrinsically capable of attachment to the 5′ cap of an mRNA molecule and, after attachment, of performing ribosome scanning of the 5′ UTR.

Polypeptide: As used herein, the term “polypeptide” or “polypeptide of interest” refers to a polymer of amino acid residues typically joined by peptide bonds that can be produced naturally (e.g., isolated or purified) or synthetically.

Increase in Potency: As used herein, the term “increase in potency” refers to an increase in functional protein from an mRNA. In some embodiments, an increase in potency occurs due to an increase in total protein output translated from an mRNA. In some embodiments, the increase in total protein output translated from an mRNA occurs due to an increase in mRNA half-life and/or an increase in number of protein molecules translated per mRNA. In some embodiments, an increase in potency occurs due to an increase in translation fidelity by (i) an inhibition or reduction in leaky scanning, (ii) an increase in codon decoding fidelity, and/or (iii) minimizing stop codon read through. In some embodiments, an increase in potency occurs due to an increase in functional protein by targeting a protein to the site of its function.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7(2):194-206), translation enhancer elements (e.g., the APP mRNA translation enhancer element, see Rogers et al., (1999) J Biol Chem 274(10):6421-6431), mRNA stability elements (e.g., AU-rich elements (AREs), see Garneau et al., (2007) Nat Rev Mol Cell Biol 8(2):113-126), translational repression element (see e.g., Blumer et al., (2002) Mech Dev 110(1-2):97-112), protein-binding RNA elements (e.g., iron-responsive element, see Selezneva et al., (2013) J Mol Biol 425(18):3301-3310), cytoplasmic polyadenylation elements (Villalba et al., (2011) Curr Opin Genet Dev 21(4):452-457), and catalytic RNA elements (e.g., ribozymes, see Scott et al., (2009) Biochim Biophys Acta 1789(9-10):634-641).

Residence time: As used herein, the term “residence time” refers to the time of occupancy of a pre-initiation complex (PIC) or a ribosome at a discrete position or location along an mRNA molecule.

Ribosomal density: As used herein, the term “ribosomal density” refers to the quantity or number of ribosomes attached to a single mRNA molecule. Ribosomal density plays an important role in translation of mRNA into protein and affects a number of intracellular phenomena. Low ribosomal density may lead to a low translation rate, and a high degradation rate of mRNA molecules. Conversely, a ribosome density that is too high may lead to ribosomal traffic jams, collisions and abortions. It may also contribute to co-translational misfolding of proteins. In some embodiments, the RNA element(s) in an mRNA as described herein increase ribosomal density on the mRNA. In some embodiments, the RNA element(s) result in an optimal ribosomal density on the mRNA to maximize the protein translation rate.

Stable RNA Secondary Structure: As used herein, the term “stable RNA secondary structure” refers to a structure, fold, or conformation adopted by an RNA molecule, or local segment or portion thereof, that is persistently maintained under physiological conditions and characterized by a low free energy state. Typical examples of stable RNA secondary structures include duplexes, hairpins, and stem-loops. Stable RNA secondary structures are known in the art to exhibit various biological activities.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect.

Transcription start site: As used herein, the term “transcription start site” refers to at least one nucleotide that initiates transcription by an RNA polymerase. In some embodiments, an mRNA described herein comprises a transcription start site. In some embodiments, the transcription start site initiates transcription by T7 RNA polymerase, and the transcription start site is referred to as a “T7 start site”. In some embodiments, the transcription start site comprises a single G. In some embodiments, the transcription start site comprises GG. In some embodiments, the mRNA comprises a transcription start site comprising the sequence GGGAAA.

Transcriptional Regulatory Activity: As used herein, the term “transcriptional regulatory activity” (used interchangeably with “transcriptional regulatory function”) refers to a biological function, mechanism, or process that modulates (e.g., regulates, influences, controls, varies) the activity of the transcriptional apparatus, including the activity of RNA polymerase. In some aspects, the desired transcriptional regulatory activity promotes and/or enhances the transcriptional fidelity of DNA transcription. In some aspects, the desired transcriptional regulatory activity reduces and/or inhibits leaky scanning.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning.

Translation of a polynucleotide comprising an open reading frame encoding a polypeptide can be controlled and regulated by a variety of mechanisms that are provided by various cis-acting nucleic acid structures. For example, naturally-occurring, cis-acting RNA elements that form hairpins or other higher-order (e.g., pseudoknot) intramolecular mRNA secondary structures can provide a translational regulatory activity to a polynucleotide, wherein the RNA element influences or modulates the initiation of polynucleotide translation, particularly when the RNA element is positioned in the 5′ UTR close to the 5′-cap structure (Pelletier and Sonenberg (1985) Cell 40(3):515-526; Kozak (1986) Proc Natl Acad Sci 83:2850-2854). Cis-acting RNA elements can also affect translation elongation, being involved in numerous frameshifting events (Namy et al., (2004) Mol Cell 13(2):157-168). Internal ribosome entry sequences (IRES) represent another type of cis-acting RNA element that are typically located in 5′ UTRs, but have also been reported to be found within the coding region of naturally-occurring mRNAs (Holcik et al. (2000) Trends Genet 16(10):469-473). In cellular mRNAs, IRES often coexist with the 5′-cap structure and provide mRNAs with the functional capacity to be translated under conditions in which cap-dependent translation is compromised (Gebauer et al., (2012) Cold Spring Harb Perspect Biol 4(7):a012245). Another type of naturally-occurring cis-acting RNA element comprises upstream open reading frames (uORFs). Naturally-occurring uORFs occur singularly or multiply within the 5′ UTRs of numerous mRNAs and influence the translation of the downstream major ORF, usually negatively (with the notable exception of GCN4 mRNA in yeast and ATF4 mRNA in mammals, where uORFs serve to promote the translation of the downstream major ORF under conditions of increased eIF2 phosphorylation (Hinnebusch (2005) Annu Rev Microbiol 59:407-450)). Additional exemplary translational regulatory activities provided by components, structures, elements, motifs, and/or specific sequences comprising polynucleotides (e.g., mRNA) include, but are not limited to, mRNA stabilization or destabilization (Baker & Parker (2004) Curr Opin Cell Biol 16(3):293-299), translational activation (Villalba et al., (2011) Curr Opin Genet Dev 21(4):452-457), and translational repression (Blumer et al., (2002) Mech Dev 110(1-2):97-112). Studies have shown that naturally-occurring, cis-acting RNA elements can confer their respective functions when used to modify, by incorporation into, heterologous polynucleotides (Goldberg-Cohen et al., (2002) J Biol Chem 277(16):13635-13640).

Transfect: As used herein, the terms “transfect”, “transfection” or “transfecting” refer to the act or method of introducing a molecule, usually a nucleic acid, into a cell.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Uridine-Modified Sequence: The terms “uridine-modified sequence” refers to a sequence optimized nucleic acid (e.g., a synthetic mRNA sequence) with a different overall or local uridine content (higher or lower uridine content) or with different uridine patterns (e.g., gradient distribution or clustering) with respect to the uridine content and/or uridine patterns of a candidate nucleic acid sequence. In the content of the present disclosure, the terms “uridine-modified sequence” and “uracil-modified sequence” are considered equivalent and interchangeable.

A “high uridine codon” is defined as a codon comprising two or three uridines, a “low uridine codon” is defined as a codon comprising one uridine, and a “no uridine codon” is a codon without any uridines. In some embodiments, a uridine-modified sequence comprises substitutions of high uridine codons with low uridine codons, substitutions of high uridine codons with no uridine codons, substitutions of low uridine codons with high uridine codons, substitutions of low uridine codons with no uridine codons, substitution of no uridine codons with low uridine codons, substitutions of no uridine codons with high uridine codons, and combinations thereof. In some embodiments, a high uridine codon can be replaced with another high uridine codon. In some embodiments, a low uridine codon can be replaced with another low uridine codon. In some embodiments, a no uridine codon can be replaced with another no uridine codon. A uridine-modified sequence can be uridine enriched or uridine rarefied.

Uridine Enriched: As used herein, the terms “uridine enriched” and grammatical variants refer to the increase in uridine content (expressed in absolute value or as a percentage value) in a sequence optimized nucleic acid (e.g., a synthetic mRNA sequence) with respect to the uridine content of the corresponding candidate nucleic acid sequence. Uridine enrichment can be implemented by substituting codons in the candidate nucleic acid sequence with synonymous codons containing less uridine nucleobases. Uridine enrichment can be global (i.e., relative to the entire length of a candidate nucleic acid sequence) or local (i.e., relative to a subsequence or region of a candidate nucleic acid sequence).

Uridine Rarefied: As used herein, the terms “uridine rarefied” and grammatical variants refer to a decrease in uridine content (expressed in absolute value or as a percentage value) in a sequence optimized nucleic acid (e.g., a synthetic mRNA sequence) with respect to the uridine content of the corresponding candidate nucleic acid sequence. Uridine rarefication can be implemented by substituting codons in the candidate nucleic acid sequence with synonymous codons containing less uridine nucleobases. Uridine rarefication can be global (i.e., relative to the entire length of a candidate nucleic acid sequence) or local (i.e., relative to a subsequence or region of a candidate nucleic acid sequence).

### Polynucleotides Comprising Functional RNA Elements

The present disclosure provides synthetic polynucleotides comprising a modification (e.g., an RNA element), wherein the modification provides a desired translational regulatory activity. In some embodiments, the disclosure provides a polynucleotide comprising a 5′ untranslated region (UTR), an initiation codon, a full open reading frame encoding a polypeptide, a 3′ UTR, and at least one modification, wherein the at least one modification provides a desired translational regulatory activity, for example, a modification that promotes and/or enhances the translational fidelity of mRNA translation. In some embodiments, the disclosure provides a polynucleotide comprising a 5′cap, a 5′ untranslated region (UTR), a Kozak-like sequence, an initiation codon, a full open reading frame encoding a polypeptide, a 3′ UTR, and at least one modification, wherein the at least one modification provides a desired translational regulatory activity, for example, a modification that promotes and/or enhances the translational fidelity of mRNA translation.

In some embodiments, the desired translational regulatory activity is a cis-acting regulatory activity. In some embodiments, the desired translational regulatory activity is an increase in the residence time of the 43S pre-initiation complex (PIC) or ribosome at, or proximal to, the initiation codon. In some embodiments, the desired translational regulatory activity is an increase in the initiation of polypeptide synthesis at or from the initiation codon. In some embodiments, the desired translational regulatory activity is an increase in the amount of polypeptide translated from the full open reading frame. In some embodiments, the desired translational regulatory activity is an increase in the fidelity of initiation codon decoding by the PIC or ribosome. In some embodiments, the desired translational regulatory activity is inhibition or reduction of leaky scanning by the PIC or ribosome. In some embodiments, the desired translational regulatory activity is a decrease in the rate of decoding the initiation codon by the PIC or ribosome. In some embodiments, the desired translational regulatory activity is inhibition or reduction in the initiation of polypeptide synthesis at any codon within the mRNA other than the initiation codon. In some embodiments, the desired translational regulatory activity is inhibition or reduction of the amount of polypeptide translated from any open reading frame within the mRNA other than the full open reading frame. In some embodiments, the desired translational regulatory activity is inhibition or reduction in the production of aberrant translation products. In some embodiments, the desired translational regulatory activity is an increase in ribosomal density on the mRNA. In some embodiments, the desired translational regulatory activity is a combination of one or more of the foregoing translational regulatory activities.

Accordingly, the present disclosure provides a polynucleotide, e.g., an mRNA, comprising an RNA element that comprises a sequence and/or an RNA secondary structure(s) that provides a desired translational regulatory activity as described herein. In some aspects, the mRNA comprises an RNA element that comprises a sequence and/or an RNA secondary structure(s) that promotes and/or enhances the translational fidelity of mRNA translation. In some aspects, the mRNA comprises an RNA element that comprises a sequence and/or an RNA secondary structure(s) that provides a desired translational regulatory activity, such as inhibiting and/or reducing leaky scanning. In some aspects, the disclosure provides an mRNA that comprises an RNA element that comprises a sequence and/or an RNA secondary structure(s) that inhibits and/or reduces leaky scanning thereby promoting the translational fidelity of the mRNA.

In some embodiments, the RNA element comprises natural and/or modified nucleotides. In some embodiments, the RNA element comprises of a sequence of linked nucleotides, or derivatives or analogs thereof, that provides a desired translational regulatory activity as described herein. In some embodiments, the RNA element comprises a sequence of linked nucleotides, or derivatives or analogs thereof, that forms or folds into a stable RNA secondary structure, wherein the RNA secondary structure provides a desired translational regulatory activity as described herein. RNA elements can be identified and/or characterized based on the primary sequence of the element (e.g., GC-rich element and/or C-rich element), by RNA secondary structure formed by the element (e.g. stem-loop), by the location of the element within the RNA molecule (e.g., located within the 5′ UTR of an mRNA), by the biological function and/or activity of the element (e.g., “translational enhancer element”), and any combination thereof.

### GC-Rich Elements

In some aspects, the disclosure provides an mRNA having one or more structural modifications that inhibits leaky scanning and/or promotes the translational fidelity of mRNA translation, wherein at least one of the structural modifications is a GC-rich RNA element. In some aspects, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In any of the foregoing or related aspects, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is 70-80% cytosine, 60-70% cytosine, 50%-60% cytosine, 40-50% cytosine, 30-40% cytosine bases. In any of the foregoing or related aspects, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is about 80% cytosine, about 70% cytosine, about 60% cytosine, about 50% cytosine, about 40% cytosine, or about 30% cytosine.

In any of the foregoing or related aspects, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is 70-80% cytosine, 60-70% cytosine, 50%-60% cytosine, 40-50% cytosine, or 30-40% cytosine. In any of the foregoing or related aspects, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is about 80% cytosine, about 70% cytosine, about 60% cytosine, about 50% cytosine, about 40% cytosine, or about 30% cytosine.

In some embodiments, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of 3, 4, 5, 6, 7, 8, 9, 10, 11, 12, 13, 14, 15, 16, 17, 18, 19, or 20 nucleotides, or derivatives or analogs thereof, linked in any order, wherein the sequence composition is >50% cytosine. In some embodiments, the sequence composition is >55% cytosine, >60% cytosine, >65% cytosine, >70% cytosine, >75% cytosine, >80% cytosine, >85% cytosine, or >90% cytosine.

In other aspects, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of about 3-30, 5-25, 10-20, 15-20 or about 20, about 15, about 12, about 10, about 6 or about 3 nucleotides, or derivatives or analogues thereof, wherein the sequence comprises a repeating GC-motif, wherein the repeating GC-motif is [CCG]n, wherein n=1 to 10, n=2 to 8, n=3 to 6, or n=4 to 5. In some embodiments, the sequence comprises a repeating GC-motif [CCG]n, wherein n=1, 2, 3, 4 or 5. In some embodiments, the sequence comprises a repeating GC-motif [CCG]n, wherein n=1, 2, or 3. In some embodiments, the sequence comprises a repeating GC-motif [CCG]n, wherein n=1. In some embodiments, the sequence comprises a repeating GC-motif [CCG]n, wherein n=2. In some embodiments, the sequence comprises a repeating GC-motif [CCG]n, wherein n=3. In some embodiments, the sequence comprises a repeating GC-motif [CCG]n, wherein n=4. In some embodiments, the sequence comprises a repeating GC-motif [CCG]n, wherein n=5.

In another aspect, the disclosure provides an mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element comprises any one of the sequences set forth in Table 1. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located about 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In other aspects, the disclosure provides an mRNA comprising at least one modification, wherein at least one modification is a GC-rich RNA element comprising the sequence V1 [CCCCGGCGCC] (SEQ ID NO: 1), or derivatives or analogs thereof, preceding a Kozak consensus sequence in the 5′ UTR of the mRNA. In some embodiments, the GC-rich element comprises the sequence V1 as set forth in Table 1 located immediately adjacent to and upstream of the Kozak consensus sequence in the 5′ UTR of the mRNA. In some embodiments, the GC-rich element comprises the sequence V1 as set forth in Table 1 located 1, 2, 3, 4, 5, 6, 7, 8, 9 or 10 bases upstream of the Kozak consensus sequence in the 5′ UTR of the mRNA. In other embodiments, the GC-rich element comprises the sequence V1 as set forth in Table 1 located 1-3, 3-5, 5-7, 7-9, 9-12, or 12-15 bases upstream of the Kozak consensus sequence in the 5′ UTR of the mRNA.

In other aspects, the disclosure provides an mRNA comprising at least one modification, wherein at least one modification is a GC-rich RNA element comprising the sequence V2 [CCCCGGC] (SEQ ID NO: 2), or derivatives or analogs thereof, preceding a Kozak consensus sequence in the 5′ UTR of the mRNA. In some embodiments, the GC-rich element comprises the sequence V2 as set forth in Table 1 located immediately adjacent to and upstream of the Kozak consensus sequence in the 5′ UTR of the mRNA. In some embodiments, the GC-rich element comprises the sequence V2 as set forth in Table 1 located 1, 2, 3, 4, 5, 6, 7, 8, 9 or 10 bases upstream of the Kozak consensus sequence in the 5′ UTR of the mRNA. In other embodiments, the GC-rich element comprises the sequence V2 as set forth in Table 1 located 1-3, 3-5, 5-7, 7-9, 9-12, or 12-15 bases upstream of the Kozak consensus sequence in the 5′ UTR of the mRNA.

In other aspects, the disclosure provides an mRNA comprising at least one modification, wherein at least one modification is a GC-rich RNA element comprising the sequence EK [GCCGCC] (SEQ ID NO: 3), or derivatives or analogs thereof, preceding a Kozak consensus sequence in the 5′ UTR of the mRNA. In some embodiments, the GC-rich element comprises the sequence EK as set forth in Table 1 located immediately adjacent to and upstream of the Kozak consensus sequence in the 5′ UTR of the mRNA. In some embodiments, the GC-rich element comprises the sequence EK as set forth in Table 1 located 1, 2, 3, 4, 5, 6, 7, 8, 9 or 10 bases upstream of the Kozak consensus sequence in the 5′ UTR of the mRNA. In other embodiments, the GC-rich element comprises the sequence EK as set forth in Table 1 located 1-3, 3-5, 5-7, 7-9, 9-12, or 12-15 bases upstream of the Kozak consensus sequence in the 5′ UTR of the mRNA.

In yet other aspects, the disclosure provides an mRNA comprising at least one modification, wherein at least one modification is a GC-rich RNA element comprising the sequence V1 [CCCCGGCGCC] (SEQ ID NO:1), or derivatives or analogs thereof, preceding a Kozak consensus sequence in the 5′ UTR of the mRNA, wherein the 5′ UTR comprises the following sequence:

(SEQ ID NO: 4)
GGGAAATAAGAGAGAAAAGAAGAGTAAGAAGAAATATAAGA.

In some embodiments, the 5′ UTR comprises SEQ ID NO: 5.

In some embodiments, the GC-rich element comprises the sequence V1 as set forth in Table 1 located immediately adjacent to and upstream of the Kozak consensus sequence in the 5′ UTR sequence shown in Table 1. In some embodiments, the GC-rich element comprises the sequence V1 as set forth in Table 1 located 1, 2, 3, 4, 5, 6, 7, 8, 9 or 10 bases upstream of the Kozak consensus sequence in the 5′ UTR of the mRNA, wherein the 5′ UTR comprises the following sequence:

(SEQ ID NO: 4)
GGGAAATAAGAGAGAAAAGAAGAGTAAGAAGAAATATAAGA.

In some embodiments, the GC-rich element comprises the sequence V1 as set forth in Table 1 located 1, 2, 3, 4, 5, 6, 7, 8, 9 or 10 bases upstream of the Kozak consensus sequence in the 5′ UTR of the mRNA, wherein the 5′ UTR comprises SEQ ID NO: 5.

In other embodiments, the GC-rich element comprises the sequence V1 as set forth in Table 1 located 1-3, 3-5, 5-7, 7-9, 9-12, or 12-15 bases upstream of the Kozak consensus sequence in the 5′ UTR of the mRNA, wherein the 5′ UTR comprises the following sequence:

(SEQ ID NO: 4)
GGGAAATAAGAGAGAAAAGAAGAGTAAGAAGAAATATAAGA.

In other embodiments, the GC-rich element comprises the sequence V1 as set forth in Table 1 located 1-3, 3-5, 5-7, 7-9, 9-12, or 12-15 bases upstream of the Kozak consensus sequence in the 5′ UTR of the mRNA, wherein the 5′ UTR comprises SEQ ID NO: 5.

In some embodiments, the 5′ UTR comprises the following sequence: GGGAAATAAGAGAGAAAAGAAGAGTAAGAAGAAATATAAGACCCCGGCGCCGCCA CC (SEQ ID NO: 7). In some embodiments, the 5′ UTR comprises SEQ ID NO: 6.

In another aspect, the disclosure provides an mRNA comprising at least one modification, wherein at least one modification is a GC-rich RNA element comprising a stable RNA secondary structure comprising a sequence of nucleotides, or derivatives or analogs thereof, linked in an order which forms a hairpin or a stem-loop. In one embodiment, the stable RNA secondary structure is upstream or downstream of the initiation codon. In another embodiment, the stable RNA secondary structure is located about 30, about 25, about 20, about 15, about 10, or about 5 nucleotides upstream or downstream of the initiation codon. In another embodiment, the stable RNA secondary structure is located about 20, about 15, about 10 or about 5 nucleotides upstream or downstream of the initiation codon. In another embodiment, the stable RNA secondary structure is located about 5, about 4, about 3, about 2, about 1 nucleotides upstream or downstream of the initiation codon. In another embodiment, the stable RNA secondary structure is located about 15-30, about 15-20, about 15-25, about 10-15, or about 5-10 nucleotides upstream or downstream of the initiation codon. In another embodiment, the stable RNA secondary structure is located 12-15 nucleotides upstream and downstream of the initiation codon. In another embodiment, the stable RNA secondary structure comprises the initiation codon. In another embodiment, the stable RNA secondary structure has a deltaG of about −30 kcal/mol, about −20 to −30 kcal/mol, about −20 kcal/mol, about −10 to −20 kcal/mol, about −10 kcal/mol, about −5 to −10 kcal/mol.

In another embodiment, the modification is operably linked to an open reading frame encoding a polypeptide and wherein the modification and the open reading frame are heterologous.

In another embodiment, the sequence of the GC-rich RNA element is comprised exclusively of guanine (G) and cytosine (C) nucleobases.

Exemplary GC-rich RNA elements useful in the mRNAs provided by the disclosure are provided in Table 1.

TABLE 1
Exemplary GC-Rich RNA Elements

| | | SEQ |
|---|---|---|
| | Sequence | ID NO |
| GC-Rich RNA | | |
| Elements | | |
| K0 (Traditional | [GCC[A/G]CC] | 17 |
| Kozak consensus) | | |
| K1 (Kozak-like) | GCCACC | 148 |
| EK1 | [CCCGCC] | 3 |
| EK2 | [GCCGCC] | 18 |
| EK3 | [CCGCCG] | 19 |
| V1 | [CCCCGGCGCC] | 1 |
| V2 | [CCCCGGC] | 2 |
| CG1 | [GCGCCCCGCGGCGCCCCGCG] | 20 |
| CG2 | [CCCGCCCGCCCCGCCCCGCC] | 21 |
| (CCG)ₙ, n = 1-10 | [CCG]ₙ | 22 |
| (GCC)ₙ, n = 1-10 | [GCC]ₙ | 23 |
| Stable RNA | | |
| Secondary | | |
| Structures | | |
| SL1 | CCGCGGCGCCCCGCGG | 24 |
| | (−9.90 kcal/mol) | |
| SL2 | GCGCGCAUAUAGCGCGC | 25 |
| | (−10.90 kcal/mol) | |
| SL3 | CATGGTGGCGGCCCGCCGCCACCATG | 26 |
| | (−22.10 kcal/mol) | |
| SL4 | CATGGTGGCCCGCCGCCACCATG | 27 |
| | (−14.90 kcal/mol) | |
| SL5 | CATGGTGCCCGCCGCCACCATG | 28 |
| | (−8.00 kcal/mol) | |

### C-Rich Elements

In some aspects, the disclosure provides an mRNA having one or more structural modifications that inhibit leaky scanning and/or promote the translational fidelity of mRNA translation, wherein at least one of the structural modifications is a C-rich RNA element. In some aspects, the disclosure provides an mRNA comprising at least one modification, wherein at least one modification is a C-rich RNA element comprising a sequence of linked nucleotides, or derivatives or analogs thereof, located proximal to the 5′ cap or 5′ end of the mRNA, wherein the C-rich element comprises a sequence of linked nucleotides, or derivatives or analogs thereof, in a 5′ UTR of the mRNA. In one embodiment, the C-rich RNA element is located about 45-50, about 40-45, about 35-40, about 30-35 about 25-30, about 20-25, about 15-20, about 10-15, about 6-10, about 1-5 nucleotides, or about 20, 19, 18, 17, 16, 15, 14, 13, 12, 11, 10, 9, 8, 7, 6, 5, 4, 3, 2 or 1 nucleotide(s) downstream of the 5′ cap or 5′ end of the mRNA. In some embodiments, the C-rich element is located about 1-20, about 2-15, about 3-10, about 4-8 or about 6 nucleotides downstream of the 5′ cap or 5′ end of the mRNA. In some embodiments, the C-rich element is located downstream of the 5′ cap or 5′ end of the mRNA with a transcription start site located between the 5′ cap or 5′end of the mRNA and the C-rich element

In some embodiments, the C-rich RNA element comprises a sequence of about 100%, about 95%, about 90%, about 85%, about 80%, about 75%, about 70%, about 65%, about 60%, about 55%, about 50%, or greater than 50% cytosine nucleobases or derivatives or analogs thereof. In some embodiments, the C-rich RNA element comprises a sequence of less than about 25%, less than about 20%, less than about 15%, less than about 10%, or less than about 5% guanosine nucleobases, or derivatives or analogs thereof. In some embodiments, the C-rich RNA element comprises a sequence of less than about 50%, less than about 45%, less than about 40%, less than about 35%, less than about 30%, less than about 25%, less than about 20%, less than about 15%, less than about 10%, or less than about 5% guanosine nucleobases, or derivatives or analogs thereof. In some embodiments, the C-rich RNA element comprises a sequence of less than about 25% guanosine nucleobases, or derivatives or analogs thereof.

In some embodiments, the C-rich RNA element is located upstream of a Kozak-like sequence in the 5′UTR. In some embodiments, the C-rich RNA element is located about 50, about 45, about 40, about 35, about 30, about 25, about 20, about 15, about 10 or about 5 nucleotides upstream of a Kozak-like sequence in the 5′UTR. In some embodiments, the C-rich RNA element is located about 5, about 4, about 3, about 2 or about 1 nucleotide upstream of a Kozak-like sequence in the 5′UTR. In some embodiments, the C-rich RNA element is located about 15-50, about 15-40, about 15-30, about 15-20, about 10-15 or about 5-10 nucleotides upstream of a Kozak-like sequence in the 5′UTR. In some embodiments, the C-rich RNA element is located upstream of and immediately adjacent to a Kozak-like sequence in the 5′UTR.

In some embodiments, the C-rich RNA element comprises a sequence of about 3-20, about 4-18, about 6-16, about 6-14, about 6-12, about 6-10, about 8-14, about 8-12, about 8-10, about 10-12, about 10-14, about 14, about 12, about 11, about 10 or about 20, 19, 18, 17, 16, 15, 14, 13, 12, 11, 10, 9, 8, 7, 6, 5, 4 or 3 nucleotides, derivatives or analogs thereof, linked in any order. In some embodiments, the C-rich RNA element comprises a sequence of about 20 nucleotides. In some embodiments, the C-rich RNA element comprises a sequence of about 19 nucleotides. In some embodiments, the C-rich RNA element comprises a sequence of about 18 nucleotides. In some embodiments, the C-rich RNA element comprises a sequence of about 17 nucleotides. In some embodiments, the C-rich RNA element comprises a sequence of about 16 nucleotides. In some embodiments, the C-rich RNA element comprises a sequence of about 15 nucleotides. In some embodiments, the C-rich RNA element comprises a sequence of about 14 nucleotides. In some embodiments, the C-rich RNA element comprises a sequence of about 13 nucleotides. In some embodiments, the C-rich RNA element comprises a sequence of about 12 nucleotides. In some embodiments, the C-rich RNA element comprises a sequence of about 11 nucleotides. In some embodiments, the C-rich RNA element comprises a sequence of about 10 nucleotides. In some embodiments, the C-rich RNA element comprises a sequence of about 9 nucleotides. In some embodiments, the C-rich RNA element comprises a sequence of about 8 nucleotides. In some embodiments, the C-rich RNA element comprises a sequence of about 7 nucleotides. In some embodiments, the C-rich RNA element comprises a sequence of about 6 nucleotides. In some embodiments, the C-rich RNA element comprises a sequence of about 5 nucleotides. In some embodiments, the C-rich RNA element comprises a sequence of about 4 nucleotides. In some embodiments, the C-rich RNA element comprises a sequence of about 3 nucleotides.

In some embodiments, the C-rich RNA element comprises a sequence of about 3-20, about 4-18, about 6-16, about 6-14, about 6-12, about 6-10, about 8-14, about 8-12, about 8-10, about 10-12, about 10-14, about 14, about 12, about 11, about 10 or about 20, 19, 18, 17, 16, 15, 14, 13, 12, 11, 10, 9, 8, 7, 6, 5, 4 or 3 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases. In some embodiments, the C-rich RNA element comprises a sequence of about 14 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases. In some embodiments, the C-rich RNA element comprises a sequence of about 14 nucleotides, derivatives or analogs thereof, linked in any order, wherein the sequence composition is greater than about 90% cytosine bases. In some embodiments, the C-rich RNA element comprises a sequence of about 13 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases. In some embodiments, the C-rich RNA element comprises a sequence of about 13 nucleotides, derivatives or analogs thereof, linked in any order, wherein the sequence composition is greater than about 90% cytosine bases. In some embodiments, the C-rich RNA element comprises a sequence of about 12 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases. In some embodiments, the C-rich RNA element comprises a sequence of about 12 nucleotides, derivatives or analogs thereof, linked in any order, wherein the sequence composition is greater than about 90% cytosine bases. In some embodiments, the C-rich RNA element comprises a sequence of about 11 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases. In some embodiments, the C-rich RNA element comprises a sequence of about 11 nucleotides, derivatives or analogs thereof, linked in any order, wherein the sequence composition is greater than about 90% cytosine bases. In some embodiments, the C-rich RNA element comprises a sequence of about 10 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases. In some embodiments, the C-rich RNA element comprises a sequence of about 10 nucleotides, derivatives or analogs thereof, linked in any order, wherein the sequence composition is greater than about 90% cytosine bases.

In some embodiments, the C-rich RNA element is depleted of guanosine. In some embodiments, the C-rich element comprises a sequence of less than about 25%, less than about 20%, less than about 15%, less than about 10% or less than about 5% guanosine bases.

In some embodiments, the C-rich RNA element comprises a sequence of about 14 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases, wherein the sequence is located upstream of a Kozak-like sequence in the 5′UTR, and wherein the sequence is located downstream of the 5′cap or 5′end of the mRNA. In some embodiments, the C-rich RNA element comprises a sequence of about 13 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases, wherein the sequence is located upstream of a Kozak-like sequence in the 5′UTR, and wherein the sequence is located downstream of the 5′cap or 5′end of the mRNA. In some embodiments, the C-rich RNA element comprises a sequence of about 12 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases, wherein the sequence is located upstream of a Kozak-like sequence in the 5′UTR, and wherein the sequence is located downstream of the 5′cap or 5′end of the mRNA. In some embodiments, the C-rich RNA element comprises a sequence of about 11 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases, wherein the sequence is located upstream of a Kozak-like sequence in the 5′UTR, and wherein the sequence is located downstream of the 5′cap or 5′end of the mRNA. In some embodiments, the C-rich RNA element comprises a sequence of about 10 nucleotides, derivatives or analogs thereof, linked in any order, wherein the sequence composition is about 100%, about 95%, about 90%, about 85%, about 80%, about 75%, about 70%, about 65%, about 60%, about 55% or about 50% cytosine bases, wherein the sequence is located upstream of a Kozak-like sequence in the 5′UTR, and wherein the sequence is located downstream of the 5′cap or 5′end of the mRNA.

In some embodiments, the C-rich RNA element comprises a sequence comprising the formula 5′-[C1]ᵥ-[N1]_{w}-[N2]ₓ-[N3]_{y}-[C2]_{z}-3′, wherein C1 and C2 are nucleotides comprising cytidine, or a derivative or analogue thereof, wherein N1, and N2 and N3 if present, are each a nucleotide comprising a nucleobase selected from the group consisting of: adenine, guanine, thymine, uracil, and cytosine, and derivatives or analogues thereof (e.g., pseudouridine, N1-methyl pseudouridine, 5-methoxyuridine), wherein v, w, x, y and z are integers whose value indicates the number of nucleotides comprising the C-rich RNA element.

In some embodiments, v=12-15 nucleotides, 3-12 nucleotides, 5-10 nucleotides, 6-8 nucleotides, 3, 4, 5, 6, 7, 8, 9 or 10 nucleotides. In some embodiments, z=2-10 nucleotides, 2-7 nucleotides, 3-5 nucleotides, 2, 3, 4, 5, 6, or 7 nucleotides. In some embodiments, w-1-5 nucleotides, 1-3 nucleotides, 1, 2, or 3 nucleotide(s). In some embodiments, x=0-5 nucleotides, 0-3 nucleotides, 0, 1, 2, or 3 nucleotide(s). In some embodiments, y=0-5 nucleotides, 0-3 nucleotides, 0, 1, 2, or 3 nucleotide(s).

In some embodiments, N1 comprises adenosine, or derivative or analogue thereof; w=1 or 2; x=0, 1, 2, or 3; and y=0, 1, 2, or 3. In some embodiments, N1 comprises adenosine, or derivative or analogue thereof; w=1 or 2; x=0; and y=0. In some embodiments, N1 comprises uracil, or derivative or analogue thereof (e.g., pseudouridine, N1-methyl pseudouridine, 5-methoxyuridine); w=1 or 2; N2 comprises adenosine, or derivative or analogue thereof; x=1, 2, or 3; N3 is guanosine, or derivative or analogue thereof; and y=1 or 2. In some embodiments, N1 comprises uracil, or derivative or analogue thereof (e.g., pseudouridine, N1-methyl pseudouridine, 5-methoxyuridine); w=1; N2 comprises adenosine, or derivative or analogue thereof; x=2; N3 is guanosine, or derivative or analogue thereof; and y=1.

In some embodiments, the C-rich RNA element comprises the formula

5′-[C1]ᵥ-[N1]_{w}-[N2]ₓ-[N3]_{y}-[C2]_{z}-3′,

wherein C1 and C2 are nucleotides comprising cytidine, or a derivative or analogue thereof, wherein N1, and N2 and N3 if present, are each a nucleotide comprising a nucleobase selected from the group consisting of: adenine, guanine, and uracil, and derivatives or analogues thereof, (e.g., pseudouridine, N1-methyl pseudouridine, 5-methoxyuridine), wherein v, w, x, y and z are integers whose value indicates the number of nucleotides comprising the C-rich RNA element. In some embodiments, v=4-10 nucleotides, 6-8 nucleotides, 6, 7, or 8 nucleotides. In some embodiments, w=1-3 nucleotides, 1 or 2 nucleotide(s). In some embodiments, x=0-3 nucleotides, 0, 1 or 2 nucleotide(s). In some embodiments, y=0-3 nucleotides, 0 or 1 nucleotide(s). In some embodiments, z=2-6 nucleotides, 2-5 nucleotides, 2, 3, 4, or 5 nucleotides. In some embodiments, N1 comprises adenosine, or derivative or analogue thereof; w=1; x=0; and y=0. In some embodiments, N1 comprises adenosine, or derivative or analogue thereof; w=2; x=0; and y=0. In some embodiments, N1 comprises uracil, or derivative or analogue thereof (e.g., pseudouridine, N1-methyl pseudouridine, 5-methoxyuridine); w=1 or 2; N2 comprises adenosine, or derivative or analogue thereof; x=1, 2, or 3; N3 is guanosine, or derivative or analogue thereof; and y=1 or 2. In some embodiments, N1 comprises uracil, or derivative or analogue thereof (e.g., pseudouridine, N1-methyl pseudouridine, 5-methoxyuridine); w=1; N2 comprises adenosine, or derivative or analogue thereof; x=2; N3 is guanosine, or derivative or analogue thereof; and y=1.

In some embodiments, the C-rich RNA element comprises a nucleotide sequence selected from the group consisting of: SEQ ID NO: 29, SEQ ID NO: 30, SEQ ID NO: 31, SEQ ID NO: 32, SEQ ID NO: 33 and SEQ ID NO: 34. In some embodiments, the C-rich RNA element comprises the nucleotide sequence 5′-CCCCCCCAACCC-3′ (SEQ ID NO: 29). In some embodiments, the C-rich RNA element comprises the nucleotide sequence 5′-CCCCCCCCAACC-3′ (SEQ ID NO: 30). In some embodiments, the C-rich RNA element comprises the nucleotide sequence 5′-CCCCCCACCCCC-3′ (SEQ ID NO: 31). In some embodiments, the C-rich RNA element comprises the nucleotide sequence 5′-CCCCCCUAAGCC-3′ (SEQ ID NO: 32). In some embodiments, the C-rich RNA element comprises the nucleotide sequence 5′-CCCCACAACC-3′ (SEQ ID NO: 33). In some embodiments, the C-rich RNA element comprises the nucleotide sequence 5′-CCCCCACAACC-3′ (SEQ ID NO: 34)

Exemplary C-rich elements provided by the disclosure are set forth in Table 2. These C-rich RNA elements and 5′ UTRs comprising these C-rich RNA elements are useful in the mRNAs of the disclosure.

TABLE 2
C-Rich RNA Elements

| C-Rich RNA Element | Sequence | SEQ ID NO |
|---|---|---|
| CR1 | CCCCCCCCAACC | 30 |
| CR2 | CCCCCCCAACCC | 29 |
| CR3 | CCCCCCACCCCC | 31 |
| CR4 | CCCCCCUAAGCC | 32 |
| CR5 | CCCCACAACC | 33 |
| CR6 | CCCCCACAACC | 34 |

### Combination of RNA Elements

In some aspects, the disclosure provides an mRNA comprising a 5′UTR comprising both a C-rich RNA element and a GC-rich RNA element, such as those described herein. In some embodiments, the amount or extent of leaky scanning from the mRNA is additively or synergistically decreased by a combination of a C-rich RNA element and the GC-rich RNA element of the disclosure. In some embodiments, leaky scanning of an mRNA comprising a 5′UTR comprising a C-rich RNA element and a GC-rich RNA element of the disclosure is reduced by about 1-fold, about 2-fold, about 3-fold, about 4-fold, about 5-fold, about 6-fold, about 7-fold, about 8-fold, about 9-fold, about 10-fold relative to the leaky scanning of an mRNA comprising a 5′UTR comprising a C-rich RNA element alone or an mRNA comprising a 5′UTR comprising a GC-rich RNA element alone. In some embodiments, leaky scanning of an mRNA comprising a 5′UTR comprising a C-rich RNA element and a GC-rich RNA element of the disclosure is reduced by about 1-fold, about 2-fold, about 3-fold, about 4-fold, about 5-fold, about 6-fold, about 7-fold, about 8-fold, about 9-fold, about 10-fold relative to the leaky scanning of an mRNA comprising a 5′UTR without a C-rich RNA element or a GC-rich RNA element. In some embodiments, the leaky scanning of an mRNA comprising a 5′UTR comprising a C-rich RNA element and a GC-rich RNA element is reduced by about 5%, about 10%, about 15%, about 20%, about 30%, about 40%, about 50%, about 60%, about 70%, about 80%, about 90% or about 100% relative to the leaky scanning of an mRNA comprising a 5′UTR comprising a C-rich RNA element alone or an mRNA comprising a 5′UTR comprising a GC-rich RNA element alone. In some embodiments, the leaky scanning of an mRNA comprising a 5′UTR comprising a C-rich RNA element and a GC-rich RNA element is reduced by about 5%, about 10%, about 15%, about 20%, about 30%, about 40%, about 50%, about 60%, about 70%, about 80%, about 90% or about 100% relative to the leaky scanning of an mRNA comprising a 5′UTR comprising without a C-rich RNA element or a GC-rich RNA element. In some embodiments, the leaky scanning of an mRNA comprising a C-rich RNA element and a GC-rich RNA element is abolished or undetectable.

In some aspects, the disclosure provides an mRNA comprising one or more C-rich RNA elements (e.g., 2, 3, 4) and one or more GC-rich RNA elements (e.g., 2, 3, 4).

In some embodiments, the disclosure provides an mRNA having a GC-rich RNA element and a C-rich RNA element as described herein, wherein the C-rich RNA element and the GC-rich RNA element precede a Kozak-like sequence or Kozak consensus sequence, in the 5′ UTR. In some embodiments, the C-rich RNA element is upstream the GC-rich RNA element in the 5′UTR. In some embodiments, the C-rich RNA element is proximal to the 5′ end or 5′ cap of the mRNA relative to the location of the GC-rich RNA element in the 5′ UTR. In some embodiments, the C-rich RNA element is located adjacent to or within about 1-6, or about 1-10 nucleotides of the 5′end or 5′ cap of the mRNA and the GC-rich RNA element is located proximal to the Kozak-like sequence or Kozak consensus sequence in the 5′ UTR. In some embodiments, the C-rich RNA element is located adjacent to or within about 1-6, or about 1-10 nucleotides of the 5′end or 5′ cap of the mRNA and the GC-rich RNA element is located adjacent to or within about 1-6 or about 1-10 nucleotides of the Kozak-like sequence or Kozak consensus sequence in the 5′ UTR.

In some embodiments, a 5′ UTR comprising both a GC-rich RNA element and a C-rich RNA element provides enhanced translational regulatory activity compared to a 5′UTR comprising a GC-rich RNA element or a C-rich RNA element.

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising a nucleotide sequence selected from the group consisting of: SEQ ID NO: 29, SEQ ID NO: 30, SEQ ID NO: 31, SEQ ID NO: 32, SEQ ID NO: 33 and SEQ ID NO: 34, and comprises a GC-rich RNA element comprising a nucleotide sequence selected from the group consisting of: SEQ ID NO: 1, SEQ ID NO: 2, SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 and SEQ ID NO: 28.

In some embodiments, the C-rich RNA element comprises a nucleotide sequence selected from the group consisting of SEQ ID NO: 31, SEQ ID NO: 32 and SEQ ID NO: 33, and the GC-rich RNA element comprises a nucleotide sequence selected from the group consisting of: SEQ ID NO: 1, SEQ ID NO: 2 and SEQ ID NO: 23.

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence set forth in SEQ ID NO: 31 and a GC-rich RNA element comprising the nucleotide sequence set forth in SEQ ID NO: 1.

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence set forth in SEQ ID NO: 33 and a GC-rich RNA element comprises the nucleotide sequence set forth in SEQ ID NO: 1.

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence set forth in SEQ ID NO: 32 and a GC-rich RNA element comprises the nucleotide sequence set forth in SEQ ID NO: 23.

In some aspects, the disclosure provides an mRNA, wherein the mRNA comprises: a 5′ cap, a 5′ untranslated region (UTR), a Kozak-like sequence, an initiation codon, a full open reading frame encoding a polypeptide, and a 3′ UTR, wherein the 5′ UTR comprises a C-rich RNA element comprising the nucleotide sequence set forth in SEQ ID NO: 32 and a GC-rich RNA element comprises the nucleotide sequence [GCC]n set forth in SEQ ID NO: 23, where n=3.

In some aspects, the disclosure provides an mRNA, wherein the mRNA comprises a 5′ UTR comprising a C-rich RNA element and a GC-rich RNA element, wherein the 5′UTR comprises the nucleotide sequence set forth in SEQ ID NO: 35.

In some aspects, the disclosure provides an mRNA, wherein the mRNA comprises a 5′ UTR comprising a C-rich RNA element and a GC-rich RNA element, wherein the 5′UTR comprises the nucleotide sequence set forth in SEQ ID NO: 36.

In some aspects, the disclosure provides an mRNA, wherein the mRNA comprises a 5′ UTR comprising a C-rich RNA element and a GC-rich RNA element, wherein the 5′UTR comprises the nucleotide sequence set forth in SEQ ID NO: 40.

In some aspects, the disclosure provides an mRNA, wherein the mRNA comprises a 5′ UTR comprising a C-rich RNA element and a GC-rich RNA element, wherein the 5′UTR comprises the nucleotide sequence set forth in SEQ ID NO: 41.

In some aspects, the disclosure provides an mRNA, wherein the mRNA comprises a 5′ UTR comprising a C-rich RNA element and a GC-rich RNA element, wherein the 5′UTR comprises the nucleotide sequence set forth in SEQ ID NO: 44.

5′ UTRs Comprising C-Rich and/or GC-Rich RNA Elements

In some aspects, the disclosure provides mRNAs having RNA elements (e.g., C-rich or GC-rich RNA elements) which provide a desired translational regulatory activity to the mRNA. In one aspect, the mRNAs of the disclosure comprise a 5′ UTR described herein to which a C-rich RNA element, a GC-rich RNA element, or a combination thereof, described herein is added or inserted, wherein the addition of the C-rich RNA element, the GC-rich RNA element, or the combination thereof, provides one or more translational regulatory activities described herein (e.g. inhibition of leaky scanning). In some embodiments, an mRNA provided by the disclosure comprises a 5′ UTR comprising a C-rich RNA element described herein, wherein the C-rich RNA element provides one or more translational regulatory activities described herein (e.g., inhibition of leaky scanning). In some embodiments, an mRNA provided by the disclosure comprises a 5′ UTR comprising a C-rich RNA element and a GC-rich RNA element of the disclosure, wherein the C-rich RNA element and GC-rich RNA element provide one or more translational regulatory activities described herein (e.g., inhibition of leaky scanning). Translational regulatory activities provided by the C-rich RNA element, GC-rich RNA element, or combination thereof, includes promoting translation of only one open reading frame encoding a desired polypeptide or translation product, or reducing, inhibiting or eliminating the failure to initiate translation of the therapeutic protein or peptide at a desired initiator codon, as a consequence of leaky scanning or other mechanisms.

In some embodiments, the mRNAs of the disclosure comprise a 5′ UTR to which a C-rich RNA element, a GC-rich RNA element, or a combination thereof, described herein, is added or inserted, thereby reducing leaky scanning of the 5′ UTR by the cellular translation machinery. In some embodiments, the mRNAs provided by the disclosure comprise a core 5′ UTR nucleotide sequence to which a C-rich RNA element, a GC-rich RNA element, or a combination thereof, described herein is added, thereby reducing leaky scanning of the 5′ UTR by the cellular translation machinery. In some embodiments, the core 5′ UTR comprises the nucleotide sequence set forth in SEQ ID NO: 45. In some embodiments, the core 5′ UTR comprises the nucleotide sequence set forth in SEQ ID NO: 46.

In one aspect, the mRNA of the disclosure comprises a 5′ UTR comprising the nucleotide set forth in SEQ ID NO: 9 in which a C-rich RNA element and a GC-rich RNA element is inserted. In one aspect, the mRNA of the disclosure comprises a 5′ UTR comprising the nucleotide set forth in SEQ ID NO: 132 in which a C-rich RNA element and a GC-rich RNA element is inserted. In some embodiments, the mRNA of the disclosure comprises a 5′ UTR comprising the nucleotide set forth in SEQ ID NO: 150 in which a C-rich RNA element and a GC-rich RNA element is inserted.

In one aspect, the mRNA of the disclosure comprises a 5′ UTR comprising the nucleotide set forth in SEQ ID NO: 10 in which a C-rich RNA element and a GC-rich RNA element is inserted. In one aspect, the mRNA of the disclosure comprises a 5′ UTR comprising the nucleotide set forth in SEQ ID NO: 130 in which a C-rich RNA element and a GC-rich RNA element is inserted. In one aspect, the mRNA of the disclosure comprises a 5′ UTR comprising the nucleotide set forth in SEQ ID NO: 163 in which a C-rich RNA element and a GC-rich RNA element is inserted.

In one aspect, the mRNA of the disclosure comprises a 5′ UTR comprising the nucleotide set forth in SEQ ID NO: 11 in which a C-rich RNA element and a GC-rich RNA element is inserted. In one aspect, the mRNA of the disclosure comprises a 5′ UTRs comprising the nucleotide set forth in SEQ ID NO: 131 in which a C-rich RNA element and a GC-rich RNA element is inserted. In one aspect, the mRNA of the disclosure comprises a 5′ UTRs comprising the nucleotide set forth in SEQ ID NO: 151 in which a C-rich RNA element and a GC-rich RNA element is inserted.

In one aspect, the mRNA of the disclosure comprises a 5′ UTR comprising the nucleotide set forth in SEQ ID NO: 12 in which a C-rich RNA element and a GC-rich RNA element is inserted, wherein SEQ ID NO: 12 is a coding DNA sequence for the 5′ UTR. In one aspect, the mRNA of the disclosure comprises a 5′ UTRs comprising the nucleotide set forth in SEQ ID NO: 70 in which a C-rich RNA element and a GC-rich RNA element is inserted. In one aspect, the mRNA of the disclosure comprises a 5′ UTRs comprising the nucleotide set forth in SEQ ID NO: 152 in which a C-rich RNA element and a GC-rich RNA element is inserted.

In one aspect, the mRNA of the disclosure comprises a 5′ UTR comprising the nucleotide selected from SEQ ID NO: 11-16 in which a C-rich RNA element and a GC-rich RNA element is inserted.

In one aspect, the mRNA of the disclosure comprises a 5′ UTR comprising the nucleotide set forth in SEQ ID NO: 43 in which a C-rich RNA element and, optionally, a GC-rich RNA element is inserted. In one aspect, the mRNA of the disclosure comprises a 5′ UTR comprising the nucleotide set forth in SEQ ID NO: 153 in which a C-rich RNA element and, optionally, a GC-rich RNA element is inserted.

In one aspect, the mRNA of the disclosure comprises a 5′ UTR comprising the nucleotide set forth in SEQ ID NO: 45 in which a C-rich RNA element and, optionally, a GC-rich RNA element is inserted. In one aspect, the mRNA of the disclosure comprises a 5′ UTR comprising the nucleotide set forth in SEQ ID NO: 149 in which a C-rich RNA element and, optionally, a GC-rich RNA element is inserted.

In one aspect, the mRNA of the disclosure comprises a 5′ UTR comprising the nucleotide set forth in SEQ ID NO: 8 in which a C-rich RNA element and, optionally, a GC-rich RNA element is inserted.

In one aspect, the mRNA of the disclosure comprises a 5′ UTR comprising the nucleotide set forth in SEQ ID NO: 46 in which a C-rich RNA element and, optionally, a GC-rich RNA element is inserted.

In one aspect, the mRNA of the disclosure comprises a 5′ UTR comprising the nucleotide set forth in SEQ ID NO: 42 in which a C-rich RNA element and, optionally, a GC-rich RNA element is inserted. In one aspect, the mRNA of the disclosure comprises a 5′ UTR comprising the nucleotide set forth in SEQ ID NO: 154 in which a C-rich RNA element and, optionally, a GC-rich RNA element is inserted.

In one aspect, the mRNA of the disclosure comprises a 5′ UTR comprising the nucleotide set forth in SEQ ID NO: 39 in which a C-rich RNA element and, optionally, a GC-rich RNA element is inserted. In one aspect, the mRNA of the disclosure comprises a 5′ UTR comprising the nucleotide set forth in SEQ ID NO: 155 in which a C-rich RNA element and, optionally, a GC-rich RNA element is inserted.

In one aspect, the mRNA of the disclosure comprises a 5′ UTR comprising the nucleotide set forth in SEQ ID NO: 48 in which a C-rich RNA element and, optionally, a GC-rich RNA element is inserted.

Exemplary 5′ UTRs comprising C-rich RNA elements, GC-rich elements, and combinations thereof provided by the disclosure are set forth in Table 3. These 5′ UTRs are useful in the mRNAs of the disclosure.

TABLE 3
Exemplary 5′UTRs and 5′UTRs with GC-Rich RNA Elements

| | | SEQ ID |
|---|---|---|
| 5′ UTRs | Sequence | NO |
| V0-UTR | GGGAAAUAAGAGAGAAAAGAAGAGUAAGAAGAAAUAUAAG | 45 |
| (v1.0 Ref) | AGCCACC | |
| V0-UTR-A | AGGAAAUAAGAGAGAAAAGAAGAGUAAGAAGAAAUAUAAG | 71 |
| (v1.0 Ref) | AGCCACC | |
| V0-UTR-0 | UAAGAGAGAAAAGAAGAGUAAGAAGAAAUAUAAGAGCCACC | 149 |
| (v1.0 Ref) | | |
| 5′UTR-001 | UAAGAGAGAAAAGAAGAGUAAGAAGAAAUAUAAGA | 8 |
| Core | | |
| F418 (V1-UTR | GGGAAATAAGAGAGAAAAGAAGAGTAAGAAGAAATATAAGA | 9 |
| (v1.1 Ref)) | CCCCGGCGCCGCCACC | |
| (DNA) | | |
| F418 (V1-UTR | GGGAAAUAAGAGAGAAAAGAAGAGUAAGAAGAAAUAUAA | 132 |
| (v1.1 Ref)) | GACCCCGGCGCCGCCACC | |
| (RNA) | | |
| F418-A (V1- | AGGAAAUAAGAGAGAAAAGAAGAGUAAGAAGAAAUAUAA | 74 |
| UTR (v1.1 | GACCCCGGCGCCGCCACC | |
| Ref)) (RNA) | | |
| F418-0 (V1- | UAAGAGAGAAAAGAAGAGUAAGAAGAAAUAUAAGACCCCG | 150 |
| UTR (v1.1 | GCGCCGCCACC | |
| Ref)) (RNA) | | |
| V2-UTR | GGGAAATAAGAGAGAAAAGAAGAGTAAGAAGAAATATAAGA | 10 |
| (DNA) | CCCCGGCGCCACC | |
| V2-UTR (RNA) | GGGAAAUAAGAGAGAAAAGAAGAGUAAGAAGAAAUAUAA | 130 |
| | GACCCCGGCGCCACC | |
| V2-UTR-A | AGGAAAUAAGAGAGAAAAGAAGAGUAAGAAGAAAUAUAA | 75 |
| (RNA) | GACCCCGGCGCCACC | |
| V2-UTR-0 | UAAGAGAGAAAAGAAGAGUAAGAAGAAAUAUAAGACCCCG | 163 |
| | GCGCCACC | |
| CG1-UTR | GGGAAATAAGAGAGAAAAGAAGAGTAAGAAGAAATATAAGA | 11 |
| (DNA) | GCGCCCCGCGGCGCCCCGCGGCCACC | |
| CG1-UTR | GGGAAAUAAGAGAGAAAAGAAGAGUAAGAAGAAAUAUAA | 131 |
| (RNA) | GAGCGCCCCGCGGCGCCCCGCGGCCACC | |
| CG1-UTR-A | AGGAAAUAAGAGAGAAAAGAAGAGUAAGAAGAAAUAUAA | 76 |
| | GAGCGCCCCGCGGCGCCCCGCGGCCACC | |
| CG1-UTR-0 | UAAGAGAGAAAAGAAGAGUAAGAAGAAAUAUAAGAGCGCC | 151 |
| | CCGCGGCGCCCCGCGGCCACC | |
| CG2-UTR | GGGAAATAAGAGAGAAAAGAAGAGTAAGAAGAAATATAAGA | 12 |
| (DNA) | CCCGCCCGCCCCGCCCCGCCGCCACC | |
| CG2-UTR | GGGAAAUAAGAGAGAAAAGAAGAGUAAGAAGAAAUAUAA | 70 |
| (RNA) | GACCCGCCCGCCCCGCCCCGCCGCCACC | |
| CG2-UTR-A | AGGAAAUAAGAGAGAAAAGAAGAGUAAGAAGAAAUAUAA | 77 |
| | GACCCGCCCGCCCCGCCCCGCCGCCACC | |
| CG2-UTR-0 | UAAGAGAGAAAAGAAGAGUAAGAAGAAAUAUAAGACCCGC | 152 |
| | CCGCCCCGCCCCGCCGCCACC | |
| KT1-UTR | GGGCCCGCCGCCAAC | 13 |
| KT1-UTR-A | AGGCCCGCCGCCAAC | 78 |
| KT2-UTR | GGGCCCGCCGCCACC | 14 |
| KT2-UTR-A | AGGCCCGCCGCCACC | 79 |
| KT3-UTR | GGGCCCGCCGCCGAC | 15 |
| KT3-UTR-A | AGGCCCGCCGCCGAC | 80 |
| KT4-UTR | GGGCCCGCCGCCGCC | 16 |
| KT4-UTR-A | AGGCCCGCCGCCGCC | 81 |
| GCC3- | GGGAAAGCCGCCGCCGCCACC | 43 |
| ExtKozak (Ref) | | |
| GCC3- | AGGAAAGCCGCCGCCGCCACC | 82 |
| ExtKozak-A | | |
| GCC3- | GCCGCCGCCGCCACC | 153 |
| ExtKozak | | |
| (Ref)-0 | | |
| S065 core | CCUCAUAUCCAGGCUCAAGAAUAGAGCUCAGUGUUUUGUU | 46 |
| | GUUUAAUCAUUCCGACGUGUUUUGCGAUAUUCGCGCAAAG | |
| | CAGCCAGUCGCGCGCUUGCUUUUAAGUAGAGUUGUUUUUC | |
| | CACCCGUUUGCCAGGCAUCUUUAAUUUAACAUAUUUUUAU | |
| | UUUUCAGGCUAACCUA | |
| S065 | GGGAGACCUCAUAUCCAGGCUCAAGAAUAGAGCUCAGUGU | 42 |
| | UUUGUUGUUUAAUCAUUCCGACGUGUUUUGCGAUAUUCGC | |
| | GCAAAGCAGCCAGUCGCGCGCUUGCUUUUAAGUAGAGUUG | |
| | UUUUUCCACCCGUUUGCCAGGCAUCUUUAAUUUAACAUAU | |
| | UUUUAUUUUUCAGGCUAACCUAAAGCAGAGAA | |
| S065-A | AGGAGACCUCAUAUCCAGGCUCAAGAAUAGAGCUCAGUGU | 72 |
| | UUUGUUGUUUAAUCAUUCCGACGUGUUUUGCGAUAUUCGC | |
| | GCAAAGCAGCCAGUCGCGCGCUUGCUUUUAAGUAGAGUUG | |
| | UUUUUCCACCCGUUUGCCAGGCAUCUUUAAUUUAACAUAU | |
| | UUUUAUUUUUCAGGCUAACCUAAAGCAGAGAA | |
| S065-0 | CCUCAUAUCCAGGCUCAAGAAUAGAGCUCAGUGUUUUGUU | 154 |
| | GUUUAAUCAUUCCGACGUGUUUUGCGAUAUUCGCGCAAAG | |
| | CAGCCAGUCGCGCGCUUGCUUUUAAGUAGAGUUGUUUUUC | |
| | CACCCGUUUGCCAGGCAUCUUUAAUUUAACAUAUUUUUAU | |
| | UUUUCAGGCUAACCUAAAGCAGAGAA | |
| combo3_S065 | GGGAGACCUCAUAUCCAGGCUCAAGAAUAGAGCUCAGUGU | 39 |
| (SO65 core | UUUGUUGUUUAAUCAUUCCGACGUGUUUUGCGAUAUUCGC | |
| extended | GCAAAGCAGCCAGUCGCGCGCUUGCUUUUAAGUAGAGUUG | |
| Kozak) | UUUUUCCACCCGUUUGCCAGGCAUCUUUAAUUUAACAUAU | |
| | UUUUAUUUUUCAGGCUAACCUACGCCGCCACC | |
| combo3_S065- | AGGAGACCUCAUAUCCAGGCUCAAGAAUAGAGCUCAGUGU | 73 |
| A | UUUGUUGUUUAAUCAUUCCGACGUGUUUUGCGAUAUUCGC | |
| (S065 | GCAAAGCAGCCAGUCGCGCGCUUGCUUUUAAGUAGAGUUG | |
| ExtKozak) | UUUUUCCACCCGUUUGCCAGGCAUCUUUAAUUUAACAUAU | |
| | UUUUAUUUUUCAGGCUAACCUACGCCGCCACC | |
| combo3_S065- | CCUCAUAUCCAGGCUCAAGAAUAGAGCUCAGUGUUUUGUU | 155 |
| 0 | GUUUAAUCAUUCCGACGUGUUUUGCGAUAUUCGCGCAAAG | |
| | CAGCCAGUCGCGCGCUUGCUUUUAAGUAGAGUUGUUUUUC | |
| (S065 | CACCCGUUUGCCAGGCAUCUUUAAUUUAACAUAUUUUUAU | |
| ExtKozak) | UUUUCAGGCUAACCUACGCCGCCACC | |
| 5′ UTR-026 | UUCCGGUUGGGUGUCACG | 48 |

(GC-Rich Elements underlined)

In other aspects, the mRNA of the disclosure comprises a 5′ UTR comprising the nucleotide set forth in SEQ ID NO: 37 in which a C-rich RNA element and, optionally, a GC-rich RNA element is inserted. In other aspects, the mRNA of the disclosure comprises a 5′ UTR comprising the nucleotide set forth in SEQ ID NO: 156 in which a C-rich RNA element and, optionally, a GC-rich RNA element is inserted.

In one aspect, the mRNA of the disclosure comprises a 5′ UTR comprising the nucleotide set forth in SEQ ID NO: 38 in which a C-rich RNA element and, optionally, a GC-rich RNA element is inserted. In one aspect, the mRNA of the disclosure comprises a 5′ UTR comprising the nucleotide set forth in SEQ ID NO: 157 in which a C-rich RNA element and, optionally, a GC-rich RNA element is inserted.

In one aspect, the mRNA of the disclosure comprises a 5′ UTR comprising the nucleotide set forth in SEQ ID NO: 40 in which a C-rich RNA element and, optionally, a GC-rich RNA element is inserted. In one aspect, the mRNA of the disclosure comprises a 5′ UTR comprising the nucleotide set forth in SEQ ID NO: 158 in which a C-rich RNA element and, optionally, a GC-rich RNA element is inserted.

In one aspect, the mRNA of the disclosure comprises a 5′ UTR comprising the nucleotide set forth in SEQ ID NO: 41 in which a C-rich RNA element and, optionally, a GC-rich RNA element is inserted. In one aspect, the mRNA of the disclosure comprises a 5′ UTR comprising the nucleotide set forth in SEQ ID NO: 159 in which a C-rich RNA element and, optionally, a GC-rich RNA element is inserted.

Exemplary 5′ UTRs comprising C-rich RNA elements, and combinations with GC-rich elements, provided by the disclosure are set forth in Table 4. These 5′ UTRs are useful in the mRNAs of the disclosure.

TABLE 4
Exemplary 5′ UTRs with C-Rich RNA Elements

| | | SEQ ID |
|---|---|---|
| 5′ UTR | Sequence | NO |
| combo1_S065 | GGGAAACCCCCCACCCCCGCCUCAUAUCCAGGCUCAAG | 37 |
| | AAUAGAGCUCAGUGUUUUGUUGUUUAAUCAUUCCGACG | |
| | UGUUUUGCGAUAUUCGCGCAAAGCAGCCAGUCGCGCGCU | |
| | UGCUUUUAAGUAGAGUUGUUUUUCCACCCGUUUGCCAG | |
| | GCAUCUUUAAUUUAACAUAUUUUUAUUUUUCAGGCUAA | |
| | CCUAAAGCAGAGAA | |
| combo1_S065-A | AGGAAACCCCCCACCCCCGCCUCAUAUCCAGGCUCAAG | 83 |
| | AAUAGAGCUCAGUGUUUUGUUGUUUAAUCAUUCCGACG | |
| | UGUUUUGCGAUAUUCGCGCAAAGCAGCCAGUCGCGCGCU | |
| | UGCUUUUAAGUAGAGUUGUUUUUCCACCCGUUUGCCAG | |
| | GCAUCUUUAAUUUAACAUAUUUUUAUUUUUCAGGCUAA | |
| | CCUAAAGCAGAGAA | |
| combo1_S065-0 | CCCCCCACCCCCGCCUCAUAUCCAGGCUCAAGAAUAGA | 156 |
| | GCUCAGUGUUUUGUUGUUUAAUCAUUCCGACGUGUUUU | |
| | GCGAUAUUCGCGCAAAGCAGCCAGUCGCGCGCUUGCUUU | |
| | UAAGUAGAGUUGUUUUUCCACCCGUUUGCCAGGCAUCU | |
| | UUAAUUUAACAUAUUUUUAUUUUUCAGGCUAACCUAAA | |
| | GCAGAGAA | |
| combo2_S065 | GGGAAAUCCCCACAACCGCCUCAUAUCCAGGCUCAAGA | 38 |
| | AUAGAGCUCAGUGUUUUGUUGUUUAAUCAUUCCGACGU | |
| | GUUUUGCGAUAUUCGCGCAAAGCAGCCAGUCGCGCGCUU | |
| | GCUUUUAAGUAGAGUUGUUUUUCCACCCGUUUGCCAGG | |
| | CAUCUUUAAUUUAACAUAUUUUUAUUUUUCAGGCUAAC | |
| | CUAAAGCAGAGAA | |
| combo2_S065-A | AGGAAAUCCCCACAACCGCCUCAUAUCCAGGCUCAAGA | 84 |
| | AUAGAGCUCAGUGUUUUGUUGUUUAAUCAUUCCGACGU | |
| | GUUUUGCGAUAUUCGCGCAAAGCAGCCAGUCGCGCGCUU | |
| | GCUUUUAAGUAGAGUUGUUUUUCCACCCGUUUGCCAGG | |
| | CAUCUUUAAUUUAACAUAUUUUUAUUUUUCAGGCUAAC | |
| | CUAAAGCAGAGAA | |
| combo2_S065-0 | UCCCCACAACCGCCUCAUAUCCAGGCUCAAGAAUAGAG | 157 |
| | CUCAGUGUUUUGUUGUUUAAUCAUUCCGACGUGUUUUG | |
| | CGAUAUUCGCGCAAAGCAGCCAGUCGCGCGCUUGCUUUU | |
| | AAGUAGAGUUGUUUUUCCACCCGUUUGCCAGGCAUCUU | |
| | UAAUUUAACAUAUUUUUAUUUUUCAGGCUAACCUAAAG | |
| | CAGAGAA | |
| combo4_S065 | GGGAAACCCCCCACCCCCGCCUCAUAUCCAGGCUCAAG | 40 |
| | AAUAGAGCUCAGUGUUUUGUUGUUUAAUCAUUCCGACG | |
| | UGUUUUGCGAUAUUCGCGCAAAGCAGCCAGUCGCGCGCU | |
| | UGCUUUUAAGUAGAGUUGUUUUUCCACCCGUUUGCCAG | |
| | GCAUCUUUAAUUUAACAUAUUUUUAUUUUUCAGGCUAA | |
| | CCUACGCCGCCACC | |
| combo4_S065-A | AGGAAACCCCCCACCCCCGCCUCAUAUCCAGGCUCAAG | 85 |
| | AAUAGAGCUCAGUGUUUUGUUGUUUAAUCAUUCCGACG | |
| | UGUUUUGCGAUAUUCGCGCAAAGCAGCCAGUCGCGCGCU | |
| | UGCUUUUAAGUAGAGUUGUUUUUCCACCCGUUUGCCAG | |
| | GCAUCUUUAAUUUAACAUAUUUUUAUUUUUCAGGCUAA | |
| | CCUACGCCGCCACC | |
| combo4_S065-0 | CCCCCCACCCCCGCCUCAUAUCCAGGCUCAAGAAUAGA | 158 |
| | GCUCAGUGUUUUGUUGUUUAAUCAUUCCGACGUGUUUU | |
| | GCGAUAUUCGCGCAAAGCAGCCAGUCGCGCGCUUGCUUU | |
| | UAAGUAGAGUUGUUUUUCCACCCGUUUGCCAGGCAUCU | |
| | UUAAUUUAACAUAUUUUUAUUUUUCAGGCUAACCUACG | |
| | CCGCCACC | |
| combo5_S065 | GGGAAAUCCCCACAACCGCCUCAUAUCCAGGCUCAAGA | 41 |
| | AUAGAGCUCAGUGUUUUGUUGUUUAAUCAUUCCGACGU | |
| | GUUUUGCGAUAUUCGCGCAAAGCAGCCAGUCGCGCGCUU | |
| | GCUUUUAAGUAGAGUUGUUUUUCCACCCGUUUGCCAGG | |
| | CAUCUUUAAUUUAACAUAUUUUUAUUUUUCAGGCUAAC | |
| | CUACGCCGCCACC | |
| combo5_S065-A | AGGAAAUCCCCACAACCGCCUCAUAUCCAGGCUCAAGA | 86 |
| | AUAGAGCUCAGUGUUUUGUUGUUUAAUCAUUCCGACGU | |
| | GUUUUGCGAUAUUCGCGCAAAGCAGCCAGUCGCGCGCUU | |
| | GCUUUUAAGUAGAGUUGUUUUUCCACCCGUUUGCCAGG | |
| | CAUCUUUAAUUUAACAUAUUUUUAUUUUUCAGGCUAAC | |
| | CUACGCCGCCACC | |
| combo5_S065-0 | UCCCCACAACCGCCUCAUAUCCAGGCUCAAGAAUAGAG | 159 |
| | CUCAGUGUUUUGUUGUUUAAUCAUUCCGACGUGUUUUG | |
| | CGAUAUUCGCGCAAAGCAGCCAGUCGCGCGCUUGCUUUU | |
| | AAGUAGAGUUGUUUUUCCACCCGUUUGCCAGGCAUCUU | |
| | UAAUUUAACAUAUUUUUAUUUUUCAGGCUAACCUACGC | |
| | CGCCACC | |

(C-rich RNA element in bold; Kozak italicized)

In other aspects, the mRNA of the disclosure comprises a 5′ UTR comprising the nucleotide set forth in SEQ ID NO: 35 in which a C-rich RNA element and a GC-rich RNA element is inserted. In other aspects, the mRNA of the disclosure comprises a 5′ UTR comprising the nucleotide set forth in SEQ ID NO: 160 in which a C-rich RNA element and a GC-rich RNA element is inserted.

In one aspect, the mRNA of the disclosure comprises a 5′ UTR comprising the nucleotide set forth in SEQ ID NO: 36 in which a C-rich RNA element and, optionally, a GC-rich RNA element is inserted. In one aspect, the mRNA of the disclosure comprises a 5′ UTR comprising the nucleotide set forth in SEQ ID NO: 161 in which a C-rich RNA element and, optionally, a GC-rich RNA element is inserted.

In one aspect, the mRNA of the disclosure comprises a 5′ UTR comprising the nucleotide set forth in SEQ ID NO: 44 in which a C-rich RNA element and, optionally, a GC-rich RNA element is inserted. In one aspect, the mRNA of the disclosure comprises a 5′ UTR comprising the nucleotide set forth in SEQ ID NO: 162 in which a C-rich RNA element and, optionally, a GC-rich RNA element is inserted.

Exemplary 5′ UTRs comprising C-rich RNA elements, and combinations with GC-rich elements, provided by the disclosure are set forth in Table 5. These 5′ UTRs are useful in the mRNAs of the disclosure.

TABLE 5
Exemplary 5′ UTRs with C-Rich RNA Elements and GC-Rich RNA Elements
(GC-Rich Elements underlined; C-rich RNA element in bold; Kozak italicized)

| 5′UTR | Sequence | SEQ ID NO |
|---|---|---|
| combol_V1.1 | GGGAAACCCCCCACCCCCGGGGAAAUAAGAGAGAAAAGAAGAGUAAGAAGAAA | 35 |
| | UAUAAGACCCCGGCGCCGCCACC | |
| combol_V1.1-A | AGGAAACCCCCCACCCCCGGGGAAAUAAGAGAGAAAAGAAGAGUAAGAAGAAA | 87 |
| | UAUAAGACCCCGGCGCCGCCACC | |
| combol_V1.1-0 | CCCCCCACCCCCGGGGAAAUAAGAGAGAAAAGAAGAGUAAGAAGAAAUAUAAG | 160 |
| | ACCCCGGCGCCGCCACC | |
| combo2_V1.1 | GGGAAAUCCCCACAACCGGGGAAAUAAGAGAGAAAAGAAGAGUAAGAAGAAAU | 36 |
| | AUAAGACCCCGGCGCCGCCACC | |
| combo2_V1.1-A | AGGAAAUCCCCACAACCGGGGAAAUAAGAGAGAAAAGAAGAGUAAGAAGAAAU | 88 |
| | AUAAGACCCCGGCGCCGCCACC | |
| combo2_V1.1-0 | UCCCCACAACCGGGGAAAUAAGAGAGAAAAGAAGAGUAAGAAGAAAUAUAAGA | 161 |
| | CCCCGGCGCCGCCACC | |
| CrichCR4 + GCC3- | | |
| ExtKozak | GGGAAACCCCCCUAAGCCGCCGCCGCCGCCACC | 44 |
| CrichCR4 + GCC3- | | |
| ExtKozak-A | AGGAAACCCCCCUAAGCCGCCGCCGCCGCCACC | 89 |
| CrichCR4 + GCC3- | CCCCCCUAAGCCGCCGCCGCCGCCACC | 162 |
| ExtKozak-0 | | |

### Methods To Identify and Characterize the Function of RNA Elements

In one aspect, the disclosure provides methods to identify and/or characterize RNA elements that provide a desired translational regulatory activity of the disclosure, including those that modulate (e.g., reduce) leaking scanning to polynucleotides (e.g., mRNA).

### Ribosome Profiling

In one aspect, RNA elements that provide a desired translational regulatory activity, including modulation of leaking scanning, to polynucleotides e.g., mRNA, are identified and/or characterized by ribosome profiling.

Ribosome profiling is a technique that allows the determination of the number and position of ribosomes bound to mRNAs (see e.g., Ingolia et al., (2009) Science 324(5924):218-23, incorporated herein by reference). The technique is based on protection by the ribosome of a region or segment of mRNA from ribonuclease digestion, which region or segment is subsequently assayed. In this approach, a cell lysate is treated with ribonucleases, leading to generation of 80S ribosomes with fragments of mRNA to which they are bound. The 80S ribosomes are then purified by techniques known in the art (e.g., density gradient centrifugation), and mRNA fragments that are protected by the ribosomes are isolated. Protection results in the generation of a 30-bp fragment of RNA termed a ‘footprint’. The number and sequence of RNA footprints can be analyzed by methods known in the art (e.g., Ribo-seq, RNA-seq). The footprint is roughly centered on the A-site of the ribosome. During translation, a ribosome may dwell at a particular position or location along an mRNA (e.g., at an initiation codon). Footprints generated at these dwell positions are more abundant than footprints generated at positions along the mRNA where the ribosome is more processive. Studies have shown that more footprints are generated at positions where the ribosome exhibits decreased processivity (dwell positions) and fewer footprints where the ribosome exhibits increased processivity (Gardin et al., (2014) eLife 3:e03735). High-throughput sequencing of these footprints provides information on the mRNA locations (sequence of footprints) of ribosomes and generates a quantitative measure of ribosome density (number of footprints comprising a particular sequence) along an mRNA. Accordingly, ribosome profiling data provides information that can be used to identify and/or characterize RNA elements that provide a desired translational regulatory activity of the disclosure, including those that reduce leaky scanning, to polynucleotides as described herein e.g., mRNA.

Ribosome profiling can also be used to determine the extent of ribosome density (aka “ribosome loading”) on an mRNA. It is known that dissociated ribosomal subunits initiate translation at the initiation codon within the 5′-terminal region of mRNA. Upon initiation, the translating ribosome moves along the mRNA chain toward the 3′-end of mRNA, thus vacating the initiation site for loading the next ribosome on the mRNA. In this way a group of ribosomes moving one after another and translating the same mRNA chain is formed. Such a group is referred to as a “polyribosome” or “polysome” (Warner et al., (1963) Proc Natl Acad Sci USA 49:122-129). The number of different mRNA fragments protected by ribosomes per mRNA, per region of an mRNA (e.g., a 5′ UTR), or per location in an mRNA (e.g., an initiation codon) indicates an extent of ribosome density. In general, an increase in the number of ribosomes bound to an mRNA (i.e. ribosome density) is associated with increased levels of protein synthesis.

Accordingly, in some embodiments, an increase in ribosome density of a polynucleotide (e.g., an mRNA) comprising one or more of the modifications or RNA elements of the disclosure, relative to a polynucleotide (e.g., an mRNA) that does not comprise the one or more modifications or RNA elements, is determined by ribosome profiling. In some embodiments, an increase in ribosome density of a polynucleotide (e.g., an mRNA) comprising a C-rich element of the disclosure, relative to a polynucleotide (e.g., an mRNA) that does not comprise the C-rich element, is determined by ribosome density.

Ribosome profiling is also used to determine the time, extent, rate and/or fidelity of ribosome decoding of a particular codon of an mRNA (and by extension the expected number of corresponding RNA-seq reads in a library of isolated footprints), which in turn is determined by the amount of time a ribosome spends at a particular codon (dwell time). The latter is referred to as a “codon elongation rate” or a “codon decoding rate”. Relative dwell time of ribosomes between two locations in an mRNA, instead of the actual or absolute dwell time at a single location, can also be determined by the comparing the number of sequencing reads of protected mRNA fragments at each location (e.g., a codon) (O'Connor et al., (2016) Nature Commun 7:12915). For example, initiation of polypeptide synthesis at or from an initiation codon can be determined from an observed increase in dwell time of ribosomes at the initiation codon relative to dwell time of ribosomes at a downstream alternate or alternative initiation codon in an mRNA. Accordingly, initiation of polypeptide synthesis at or from an initiation codon in a polynucleotide (e.g., an mRNA) comprising one or more modifications or RNA elements of the disclosure, relative to a polynucleotide (e.g., an mRNA) that does not comprise the one or more modifications or RNA elements, can be determined from an observed increase in the dwell time of ribosomes at the initiation codon relative to the dwell time of ribosomes at a downstream alternate or alternative initiation codon in each polynucleotide (e.g., mRNA).

In some embodiments, an increase in residence time or the time of occupancy (dwell time) of a ribosome at a discrete position or location (e.g., an initiation codon) along a polynucleotide (e.g., an mRNA) comprising one or more of the modifications or RNA elements of the disclosure, relative to a polynucleotide (e.g., an mRNA) that does not comprise the one or more modifications or RNA elements, is determined by ribosome profiling. In some aspects, an increase in residence time or the time of occupancy of a ribosome at an initiation codon in a polynucleotide (e.g., mRNA) comprising a C-rich element of the disclosure relative to a polynucleotide (e.g., mRNA) that does not comprise the C-rich element, is determined by ribosome profiling.

In other aspects, an increase in the initiation of polypeptide synthesis at or from the initiation codon in polynucleotide (e.g., an mRNA) comprising one or more of the modifications or RNA elements of the disclosure, relative to a polynucleotide (e.g., an mRNA) that does not comprise the one or more modifications or RNA elements, is determined by ribosome profiling. In some embodiments, an increase in the initiation of polypeptide synthesis at or from the initiation codon in a polynucleotide (e.g., mRNA) comprising a C-rich element of the disclosure relative to a polynucleotide (e.g., mRNA) that does not comprise the C-rich element, is determined by ribosome profiling.

In some embodiments, an increase in fidelity of initiation codon decoding by the ribosome of a polynucleotide (e.g., an mRNA) comprising one or more of the modifications or RNA elements of the disclosure, relative to a polynucleotide (e.g., mRNA) that does not comprise the one or more modifications or RNA elements, is determined by ribosome profiling. In some embodiments, an increase in fidelity of initiation codon decoding by the ribosome of a polynucleotide (e.g., mRNA) comprising a C-rich element of the disclosure relative to a polynucleotide (e.g., mRNA) that does not comprise the C-rich element, is determined by ribosome profiling.

In some embodiments, an increase in fidelity of initiation codon decoding by the ribosome of a polynucleotide (e.g., an mRNA) comprising one or more of the modifications or RNA elements of the disclosure, relative to a polynucleotide (e.g., an mRNA) that does not comprise the one or more modifications or RNA elements, is determined by ribosome profiling. In some embodiments, an increase in fidelity of initiation codon decoding by the ribosome in a polynucleotide (e.g., mRNA) comprising a C-rich element of the disclosure relative to a polynucleotide (e.g., mRNA) that does not comprise the C-rich element, is determined by ribosome profiling.

In some embodiments, a decrease in a rate of decoding an initiation codon by the ribosome of a polynucleotide (e.g., an mRNA) comprising one or more of the modifications or RNA elements of the disclosure, relative to a polynucleotide (e.g., an mRNA) that does not comprise the one or more modifications or RNA elements, is determined by ribosome profiling. In some embodiments, a decrease in a rate of decoding an initiation codon by the ribosome of a polynucleotide (e.g., mRNA) comprising a C-rich element of the disclosure relative to a polynucleotide (e.g., mRNA) that does not comprise the C-rich element, is determined by ribosome profiling.

### Small Ribosomal Subunit Mapping

In some aspects, RNA elements that provide a desired translational regulatory activity, including modulation of leaking scanning, to polynucleotides e.g., mRNA, are identified and/or characterized by small ribosomal subunit mapping.

Small ribosomal subunit (SSU) mapping is a technique similar to ribosome profiling that allows the determination of the number and position of small 40S ribosomal subunits or pre-initiation complexes (PICs) comprising small 40S ribosomal subunits bound to mRNAs. Similar to the technique of ribosome profiling described herein, small ribosomal subunit mapping involves analysis of a region or segment of mRNA protected by the 40S subunit from ribonuclease digestion, resulting in a ‘footprint’, the number and sequence of which can be analyzed by methods known in the art (e.g., RNA-seq). As described herein, the current model of mRNA translation initiation postulates that the pre-initiation complex (alternatively “43S pre-initiation complex”; abbreviated as “PIC”) translocates from the site of recruitment on the mRNA (typically the 5′ cap) to the initiation codon by scanning nucleotides in a 5′ to 3′ direction until the first AUG codon that resides within a specific translation-promotive nucleotide context (the Kozak sequence) is encountered (Kozak (1989) J Cell Biol 108:229-241). “Leaky scanning” by the PIC, whereby the PIC bypasses the initiation codon of an mRNA and instead continues scanning downstream until an alternate or alternative initiation codon is recognized, can occur and result in a decrease in translation efficiency and/or the production of an undesired, aberrant translation product. Thus, analysis of the number of SSUs positioned, or mapped, over AUGs downstream of the first AUG in an mRNA allows for the determination of the extent or frequency at which leaky scanning occurs. SSU mapping provides information that can be used to identify or determine a characteristic (e.g., a translational regulatory activity) of a modification or RNA element of the disclosure, that affects the activity of a small 40S ribosomal subunit (SSU or a PIC comprising the SSU.

Accordingly, an inhibition or reduction of leaky scanning by an SSU or a PIC comprising an SSU of a polynucleotide (e.g., an mRNA) comprising one or more of the modifications or RNA elements of the disclosure, relative to a polynucleotide (e.g., an mRNA) that does not comprise the one or more modifications or RNA elements, is determined by small ribosomal subunit mapping. In some aspects, an inhibition or reduction of leaky scanning by an SSU or a PIC comprising an SSU of a polynucleotide (e.g., an mRNA) comprising a C-rich element of the disclosure, relative to a polynucleotide (e.g., an mRNA) that does not comprise the C-rich element, is determined by small ribosomal subunit mapping.

In some embodiments, an increase in residence time or the time of occupancy (dwell time) of an SSU or a PIC comprising an SSU at a discrete position or location (e.g., an initiation codon) along a polynucleotide (e.g. an mRNA) comprising one or more of the modifications or RNA elements of the disclosure, relative to a polynucleotide (e.g., an mRNA) that does not comprise the one or more modifications or RNA elements, is determined by ribosome profiling. In some embodiments, an increase in residence time or the time of occupancy of an SSU or a PIC comprising an SSU at an initiation codon in a polynucleotide (e.g., an mRNA) comprising a C-rich element of the disclosure, relative to a polynucleotide (e.g., an mRNA) that does not comprise the C-rich element, is determined by ribosome profiling.

In some embodiments, an increase in the initiation of polypeptide synthesis at or from the initiation codon in polynucleotide (e.g., an mRNA) comprising one or more of the modifications or RNA elements of the disclosure, relative to a polynucleotide (e.g., an mRNA) that does not comprise the one or more modifications or RNA elements, is determined by ribosome profiling. In some embodiments, an increase in the initiation of polypeptide synthesis at or from the initiation codon in a polynucleotide (e.g., an mRNA) comprising a C-rich element of the disclosure, relative to a polynucleotide (e.g., an mRNA) that does not comprise the C-rich element, is determined by ribosome profiling.

In some embodiments, an increase in fidelity of initiation codon decoding by an SSU or a PIC comprising an SSU of a polynucleotide (e.g., an mRNA) comprising one or more of the modifications or RNA elements of the disclosure, relative to a polynucleotide that does not comprise the one or more modifications or RNA elements, is determined by ribosome profiling. In some embodiments, an increase in fidelity of initiation codon decoding by an SSU or a PIC comprising an SSU of a polynucleotide (e.g., an mRNA) comprising a C-rich element of the disclosure, relative to a polynucleotide (e.g., an mRNA) that does not comprise the C-rich element, is determined by ribosome profiling.

In some embodiments, an increase in fidelity of initiation codon decoding by an SSU or a PIC comprising an SSU of a polynucleotide (e.g., an mRNA) comprising one or more of the modifications or RNA elements of the disclosure, relative to a polynucleotide that does not comprise the one or more modifications or RNA elements, is determined by ribosome profiling. In some embodiments, an increase in fidelity of initiation codon decoding by an SSU or a PIC comprising an SSU of a polynucleotide (e.g., an mRNA) comprising a C-rich element of the disclosure, relative to a polynucleotide (e.g., an mRNA) that does not comprise the C-rich element, is determined by ribosome profiling.

In some embodiments, a decrease in a rate of decoding an initiation codon comprising a polynucleotide (e.g., an mRNA) comprising any one or more of the modifications or RNA elements of the disclosure, relative to a polynucleotide (e.g., an mRNA) that does not comprise the one or more modifications or RNA elements, is determined by ribosome profiling. In some embodiments, a decrease in a rate of decoding an initiation codon decoding by the ribosome of a polynucleotide (e.g., an mRNA) comprising a C-rich element of the disclosure, relative to a polynucleotide (e.g., an mRNA) that does not comprise the C-rich element, is determined by ribosome profiling.

### RiboFrame-Seq

In some aspects, RNA elements that provide a desired translational regulatory activity, including modulation of leaking scanning, to polynucleotides e.g., mRNA, are identified and/or characterized by RiboFrame-seq.

RiboFrame-seq is an assay that allows for the high-throughput measurement of leaky scanning for many different 5′-UTR sequences. A population of mRNAs is generated with a library of different 5′ UTR sequences, each of which contains a 5′ cap and a coding sequence that encodes a polypeptide comprising two to three different epitope tags, each in a different frame and preceded by an AUG. The mRNA population is transfected into cells and allowed to be translated. Cells are then lysed and immunoprecipitations performed against each of the encoded epitope tags. Each of these immunoprecipitations is designed to isolate a nascent polypeptide chain encoding the particular epitope, as well as the active ribosome performing its synthesis, and the mRNA that encodes it. The complement of 5′-UTRs present in each immunoprecipitate is then analyzed by methods known in the art (e.g., RNA-seq). The 5′-UTRs comprising sequences (e.g. RNA elements) that correlate with reduced, inhibited or low leaky scanning are characterized by being abundant in the immunoprecipitate corresponding to the first epitope tag relative to the other immunoprecipitates.

Accordingly, in some embodiments, a modification or RNA element having a translational regulatory activity of the disclosure is identified or characterized by RiboFrame-seq. In some aspects, a modification or RNA element having reduced, inhibited or low leaky scanning when located in a 5′ UTR of an mRNA are identified or characterized by being abundant in the immunoprecipitate corresponding to the first epitope tag relative to the other immunoprecipitates as determined by RiboFrame-seq.

### Western Blot (Immunodetection)

In some aspects, the disclosure provides a method of identifying, isolating, and/or characterizing a modification (e.g., an RNA element) that provides a translational regulatory activity by synthesizing a 1st control mRNA comprising a polynucleotide sequence comprising an open reading frame encoding a reporter polypeptide (e.g., eGFP) and a 1st AUG codon upstream of, in-frame, and operably linked to, the open reading frame encoding the reporter polypeptide. The 1st control mRNA also comprises a coding sequence for a first epitope tag (e.g. 3×FLAG) upstream of, in-frame, and operably linked to the 1st AUG codon, a 2nd AUG codon upstream of, in-frame, and operably linked to, the coding sequence for the first epitope tag. Optionally, the 1st control mRNA further comprises a coding sequence for a second epitope tag (e.g. V5) upstream of, in-frame, and operably linked to the 2nd AUG codon, and a 3rd AUG codon upstream of, in-frame, and operably linked to, the coding sequence for the second epitope tag. The 1st control mRNA also comprises a 5′ UTR and a 3′ UTR. The method further comprises synthesizing a 2nd test mRNA comprising a polynucleotide sequence comprising the 1st control mRNA and further comprising a modification (e.g. an RNA element). The method further comprises introducing the 1st control mRNA and 2nd test mRNA to conditions suitable for translation of the polynucleotide sequence encoding the reporter polypeptide. The method further comprises measuring the effect of the candidate modification on the amount of reporter polypeptide from each of the three AUG codons. Following transfection of this mRNA into cells, the cell lysate is analyzed by Western blot using antibodies that specifically bind to and detect the reporter polypeptide. This analysis generates two or three bands: a higher band that corresponds to protein generated from the first AUG and lower bands derived from protein generated from the second AUG and, optionally, third AUG.

Leaky scanning is calculated as abundance of the lower bands divided by the sum of the abundance of both bands, as determined by methods known in the art (e.g. densitometry). A test mRNA comprising one or more modifications or RNA elements of the disclosure, that correlate with reduced, inhibited or low leaky scanning is characterized by an increase in amount of polypeptide comprising the second epitope tag compared to the amount of polypeptide that does not comprise an epitope tag, optionally, the amount of polypeptide comprising the first epitope tag, translated from the test mRNA, relative to the control mRNA that does not comprise the one or more modifications or RNA elements. Accordingly, in some embodiments, a modification or RNA element having a translational regulatory activity of the disclosure, is identified by Western blot.

In some embodiments, an inhibition or reduction in leaky scanning of a polynucleotide (e.g., an mRNA) comprising one or more of the modifications or RNA elements of the disclosure, relative to a polynucleotide (e.g., an mRNA) that does not comprise the one or more modifications or RNA elements, is determined by Western blot. In some embodiments, an inhibition or reduction in leaky scanning of a polynucleotide (e.g., an mRNA) comprising a C-rich element of the disclosure, relative to a polynucleotide (e.g., an mRNA) that does not comprise the C-rich element, is determined by Western blot.

In some embodiments, an increase in the initiation of polypeptide synthesis at or from the initiation codon comprising a polynucleotide (e.g., an mRNA) comprising any one or more of the modifications or RNA elements of the disclosure, relative to a polynucleotide that does not comprise the one or more modifications or RNA elements, is determined by Western blot. In some embodiments, an increase in the initiation of polypeptide synthesis at or from the initiation codon comprising a polynucleotide (e.g., an mRNA) comprising a C-rich element of the disclosure, relative to a polynucleotide (e.g., an mRNA) that does not comprise the C-rich element, is determined by Western blot.

In some embodiments, an increase in an amount of polypeptide translated from the full open reading frame comprising a polynucleotide (e.g., an mRNA) comprising any one or more of the modifications or RNA elements of the disclosure, relative to a polynucleotide (e.g., an mRNA) that does not comprise the one or more modifications or RNA elements, is determined by Western blot. In some embodiments, an increase in an amount of polypeptide translated from the full open reading frame comprising a polynucleotide (e.g., an mRNA) comprising a C-rich element of the disclosure, relative to a polynucleotide (e.g., an mRNA) that does not comprise the C-rich element, is determined by Western blot.

In some embodiments, an inhibition or reduction in an amount of polypeptide translated from any open reading frame other than a full open reading frame comprising a polynucleotide (e.g., an mRNA) comprising one or more of the modifications or RNA elements of the disclosure, relative to a polynucleotide (e.g., an mRNA) that does not comprise the one or more modifications or RNA elements, is determined by Western blot. In some embodiments, an inhibition or reduction in an amount of polypeptide translated from any open reading frame other than a full open reading frame comprising a polynucleotide (e.g., an mRNA) comprising a C-rich element of the disclosure, relative to a polynucleotide (e.g., an mRNA) that does not comprise the C-rich element, is determined by Western blot.

In some embodiments, an inhibition or reduction in the production of aberrant translation products translated from a polynucleotide (e.g., an mRNA) comprising any one or more of the modifications or RNA elements of the disclosure, relative to a polynucleotide (e.g., an mRNA) that does not comprise the one or more modifications or RNA elements, is determined by Western blot. In some embodiments, an inhibition or reduction in the production of aberrant translation products translated from a polynucleotide (e.g., an mRNA) comprising a C-rich element of the disclosure, relative to a polynucleotide (e.g., an mRNA) that does not comprise the C-rich element, is determined by Western blot.

In some embodiments, leaky scanning by a 43S pre-initiation complex (PIC) or ribosome of a polynucleotide (e.g., an mRNA) comprising one or more of the modifications or RNA elements (e.g., C-rich element) of the disclosure is decreased by about 80%-100%, about 60%-80%, about 40%-60%, about 20%-40%, about 10%-20%, about 5%-10%, about 1%-5% relative to a polynucleotide (e.g., an mRNA) that does not comprise the one or more modifications or RNA elements, as determined by SSU mapping and/or ribosome profiling methods, as described herein.

In some embodiments, leaky scanning by a 43S pre-initiation complex (PIC) or ribosome of a polynucleotide (e.g., an mRNA) comprising any one or more of the modifications or RNA elements of the disclosure is decreased by about 80%-100%, about 60%-80%, about 40%-60%, about 20%-40%, about 10%-20%, about 5%-10%, about 1%-5% and an amount of a polypeptide translated from a full reading frame is increased by about 80%-100%, about 60%-80%, about 40%-60%, about 20%-40%, about 10%-20%, about 5%-10%, about 1%-5% relative to a polynucleotide (e.g., an mRNA) that does not comprise the one or more modification or RNA elements, as determined by SSU mapping and Western blot, respectively, as described herein.

In some embodiments, leaky scanning by the 43S pre-initiation complex (PIC) or ribosome of a polynucleotide (e.g., an mRNA) comprising any one or more of the modifications or RNA elements (e.g., C-rich element) of the disclosure is decreased by about 80%-100%, about 60%-80%, about 40%-60%, about 20%-40%, about 10%-20%, about 5%-10%, about 1%-5%, an amount of a polypeptide translated from a full open reading frame is increased by about 80%-100%, about 60%-80%, about 40%-60%, about 20%-40%, about 10%-20%, about 5%-10%, about 1%-5%, and potency of the polypeptide is increased by about 80%-100%, about 60%-80%, about 40%-60%, about 20%-40%, about 10%-20%, about 5%-10%, about 1%-5%, relative to a polynucleotide (e.g., an mRNA) that does not comprise the one or more modification or RNA elements, as determined by SSU mapping and Western blot.

In some aspects, the disclosure provides a reporter system to characterize RNA elements that provide a desired translational regulatory activity. Specifically, a method of identifying RNA elements having translational regulatory activity comprises:

(i) providing a population of polynucleotides, wherein each polynucleotide comprises a plurality of open reading frames encoding a plurality of polypeptides, each comprises a peptide epitope tag, wherein each polynucleotide comprises:
(a) at least one first AUG codon upstream of, in-frame, and operably linked to at least one first open reading frame encoding at least one first polypeptide comprising at least one first peptide epitope tag;
(b) at least one second AUG codon upstream of, in-frame, and operably linked to at least one second open reading frame encoding at least one second polypeptide comprising at least one second peptide epitope tag, wherein the second AUG codon is downstream and out-of-frame of the first AUG codon; optionally,
(c) at least one third AUG codon upstream of, in-frame, and operably linked to at least one third open reading frame encoding at least one third polypeptide comprising at least one second peptide epitope tag, wherein the third AUG codon is downstream and out-of-frame with the first and second AUG codons; and
(d) a 5′UTR and a 3′UTR, wherein the 5′UTR of each polynucleotide within the population comprises a unique nucleotide sequence;
(e) no stop codons (UAG, UGA, or UAA) within any frame between the first AUG and the stop codon corresponding to the first AUG;

(ii) providing conditions suitable for translation of each polynucleotide in the population of polynucleotides;

(iii) isolating a complex comprising a nascent translation product comprising the first, second and, if present, third epitope tag, and the 5′ UTR corresponding to the epitope tag and encoded polynucleotide;

(iv) determining the sequences of the 5′UTRs corresponding to each polynucleotide encoding the nascent translation product; and

(v) determining which nucleotides are enriched at each position in the 5′UTR of the first polynucleotide compared to the second, and optionally third, polynucleotide.

In some embodiments, the first polynucleotide encodes a reporter polypeptide, such as eGFP. In some embodiments, the first AUG is linked to and in frame with an open reading frame that encodes eGFP. Reporter polypeptides are known to those of skill in the art.

In some embodiments, the peptide epitope tag is selected from the group consisting of: a FLAG tag (DYKDDDDK; SEQ ID NO: 133); a 3×FLAG tag (DYKDHDGDYKDHDIDYKDDDK; SEQ ID NO: 111); a Myc tag (EQKLISEEDL; SEQ ID NO: 112); a V5 tag (GKPIPNPLLGLDST; SEQ ID NO: 113); a hemagglutinin A (HA) tag (YPYDVPDYA; SEQ ID NO: 114); a histidine tag (e.g., a 6×His tag; HHHHHH; SEQ ID NO: 115); an HSV tag (QPELAPEDPED; SEQ ID NO: 116); a VSV-G tag (YTDIEMNRLGK; SEQ ID NO: 117); an NE tag (TKENPRSNQEESYDDNES; SEQ ID NO: 118); an AViTag (GLNDIFEAQKIEWHE; SEQ ID NO: 119); a calmodulin tag (KRRWKKNFIAVSAANRFKKISSSGAL; SEQ ID NO: 120); an E tag (GAPVPYPDPLEPR; SEQ ID NO: 121); an S tag (KETAAAKFERQHMDS; SEQ ID NO: 122); an SBP tag (MDEKTTGWRGGHVVEGLAGELEQLRARLEHHPQGQREP; SEQ ID NO: 123); a Softag 1 (SLAELLNAGLGGS; SEQ ID NO: 124); a Softag 3 (TQDPSRVG; SEQ ID NO: 125); a Strep tag (WSHPQFEK; SEQ ID NO: 126); a Ty tag (EVHTNQDPLD; SEQ ID NO:127); and an Xpress tag (DLYDDDDK; SEQ ID NO: 128).

Another RNA element known to regulate translation of mRNA is the five-prime cap (5′ cap), which is a specially altered nucleotide the 5′ end of natural mRNA co-transcriptionally. This process, known as mRNA capping, is highly regulated and is vital in the creation of stable and mature messenger RNA able to undergo translation. In eukaryotes, the structure of the 5′ cap consists of a guanine nucleotide connected to 5′ end of an mRNA via an unusual 5′ to 5′ triphosphate linkage. This guanosine is methylated on the 7 position directly after capping in vivo by a methyltransferase, and as such, is sometimes referred to as a 7-methylguanylate cap, and abbreviated m7G. A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m27,O3′GpppG, m27,O3′GppppG, m27,O2′GppppG, m7Gpppm7G, m73′dGpppG, m27,O3′GpppG, m27,O3′GppppG, and m27,O2′GppppG. Accordingly, in some embodiments, the mRNAs disclosed herein comprise a 5′ cap, or derivative, analog, or modification thereof.

An early event in translation initiation involves the formation of the 43S pre-initiation complex (PIC) composed of the small 40S ribosomal subunit, the initiator transfer RNA (Met-tRNAiMet), and several various eIFs. Following recruitment to the mRNA, the PIC biochemically interrogates or “scans” the sequence of the mRNA molecule in search of an initiation codon. In some embodiments of the mRNAs disclosed herein, the mRNAs comprise at least one initiation codon. In some embodiments, the initiation codon is an AUG codon. In some embodiments, the initiation codon comprises one or more modified nucleotides.

Similar to polypeptides, polynucleotides, particularly RNA, can fold into a variety of complex three dimensional structures. The ability of a nucleic acid to form a complex, functional three dimensional structure is exemplified by a transfer RNA molecule (tRNA), which is a single chain of ˜70-90 nucleotides in length that folds into an L-shaped 3D structure allowing it to fit into the P and A sites of a ribosome and function as the physical link between the polypeptide coding sequence of mRNA and the amino acid sequence of the polypeptide. Since base pairing between complementary sequences of nucleobases determines the overall secondary (and ultimately tertiary) structure of nucleic acid molecules, sequences predicted to or known to be able to adopt a particular structure (e.g. a stem-loop) are vital considerations in the design and utility of some types of functional elements or motifs (e.g. RNA elements). Nucleic acid secondary structure is generally divided into duplexes (contiguous base pairs) and various kinds of loops (unpaired nucleotides flanked or surrounded by duplexes). As is known in the art, stable RNA secondary structures, or combinations of them, can be further classified and usefully described as, but not limited to, simple loops, tetraloops, pseudoknots, hairpins, helicies, and stem-loops. Secondary structure can also be usefully depicted as a list of nucleobases which are paired in a nucleic acid molecule.

The function(s) of a nucleic acid secondary structure are emergent from the thermodynamic properties of the secondary structure. For example, the thermodynamic stability of an RNA hairpin/stemloop structure is characterized by its free energy change (deltaG). For a spontaneous process, i.e. the formation of a stable RNA hairpin/stemloop, deltaG is negative. The lower the deltaG value, the more energy is required to reverse the process, i.e. the more energy is required to denature or melt (‘unfold’) the RNA hairpin/stemloop. The stability of an RNA hairpin/stemloop will contribute to its biological function: e.g. in the context of translation, a more stable RNA structure with a relatively low deltaG can act a physical barrier for the ribosome (Kozak, 1986; Babendure et al., 2006), leading to inhibition of protein synthesis. In contrast, a weaker or moderately stable RNA structure can be beneficial as translational enhancer, as the translational machinery will recognize it as signal for a temporary pause, but ultimately the structure will open up and allow translation to proceed (Kozal, 1986; Kozak, 1990; Babendure et al., 2006). To assign an absolute number to the deltaG value that defines a stable versus a weak/moderately stable RNA hairpin/stemloop is difficult and is very much driven by its context (sequence and structural context, biological context). In the context of the above-mentioned examples by Kozak, 1986, Kozak, 1990 and Babendure et al., 2006, stable hairpins/stemloops are characterized by approximate deltaG values lower than −30 kcal/mol, while weak/moderately stable hairpins are characterized by approximate deltaG values between −10 and −30 kcal/mol.

Accordingly, in some embodiments, an mRNA comprises at least one modification, wherein the at least one modification is a structural modification. In some embodiments, the structural modification is an RNA element. In some embodiments, the structural modification is a GC-rich RNA element. In some embodiments, the structural modification is a viral RNA element. In some embodiments, the structural modification is a protein-binding RNA element. In some embodiments, the structural modification is a translation initiation element. In some embodiments, the structural modification is a translation enhancer element. In some embodiments, the structural modification is a translation fidelity enhancing element. In some embodiments, the structural modification is an mRNA nuclear export element. In some embodiments, the structural modification is a stable RNA secondary structure.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, Calif.) and/or proprietary methods. In one embodiment, the mRNA sequence is optimized using optimization algorithms, e.g., to optimize expression in mammalian cells or enhance mRNA stability. Accordingly, in some embodiments, an mRNA comprises a structural modification, wherein the structural modification is a codon optimized open reading frame. In some embodiments, the structural modification is a modification of base composition.

mRNA Construct Components

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An exemplary 5′ UTR for use in the constructs is shown in SEQ ID NO: 45 (V0-UTR (v1.0 Ref)) or any 5′ UTR referred to by sequence in Table 6. An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G(5′)ppp(5′)G, commonly written as m⁷GpppG. A cap species may also be an anti-reverse cap analog. A non-limiting list of possible cap species includes m⁷GpppG, m⁷Gpppm⁷G, m⁷3′dGpppG, m2^{7,O3′}GpppG, m2^{7,O3′}GppppG, m₂^{7,O2′}GppppG, m⁷Gpppm⁷G, m⁷3′dGpppG, m2^{7,O3′}GpppG, m2^{7,O3′}GppppG, and m2^{7,O2′}GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

### 5′ UTR and Translation Initiation

In certain embodiments, the polynucleotide (e.g., mRNA) encoding a polypeptide of the present disclosure comprises a 5′ UTR and/or a translation initiation sequence. Natural 5′ UTRs comprise sequences involved in translation initiation. For example, Kozak sequences comprise natural 5′ UTRs and are commonly known to be involved in the process by which the ribosome initiates translation of many genes. 5′ UTRs also have been known to form secondary structures which are involved in elongation factor binding.

By engineering the features typically found in abundantly expressed genes of specific target organs, one can enhance the stability and protein production of the polynucleotides of the disclosure. For example, introduction of 5′ UTR of mRNA known to be upregulated in cancers, such as c-myc, could be used to enhance expression of a nucleic acid molecule, such as a polynucleotide, in cancer cells. Untranslated regions useful in the design and manufacture of polynucleotides include, but are not limited, to those disclosed in International Patent Publication No. WO 2014/164253 (see also US20160022840).

Shown in Table 6 is a listing of exemplary 5′ UTRs. Variants of 5′ UTRs can be utilized wherein one or more nucleotides are added or removed to the termini, including A, U, C or G.

TABLE 6
Exemplary 5′-UTRs

| 5′UTR | Name/ | | SEQ |
|---|---|---|---|
| Identifier | Description | Sequence | ID NO. |
| V0-UTR | Upstream | GGGAAAUAAGAGAGAAAAGAAGAGUAAGAA | 45 |
| (v1.0 Ref) | UTR | GAAAUAUAAGAGCCACC | |
| V0-UTR | Upstream | AGGAAAUAAGAGAGAAAAGAAGAGUAAGAA | 71 |
| (v1.0 Ref)-A | UTR | GAAAUAUAAGAGCCACC | |
| 5′UTR-001 Core | Upstream | UAAGAGAGAAAAGAAGAGUAAGAAGAAAUA | 8 |
| | UTR | UAAGA | |
| 5UTR-002 | Upstream | GGGAGAUCAGAGAGAAAAGAAGAGUAAGAA | 50 |
| | UTR | GAAAUAUAAGAGCCACC | |
| 5′UTR-003 | Upstream | GGAAUAAAAGUCUCAACACAACAUAUACAA | 51 |
| | UTR | AACAAACGAAUCUCAAGCAAUCAAGCAUUC | |
| | | UACUUCUAUUGCAGCAAUUUAAAUCAUUUC | |
| | | UUUUAAAGCAAAAGCAAUUUUCUGAAAAUU | |
| | | UUCACCAUUUACGAACGAUAGCAAC | |
| 5′UTR-004 | Upstream | GGGAGACAAGCUUGGCAUUCCGGUACUGUU | 52 |
| | UTR | GGUAAAGCCACC | |
| 5′UTR-005 | Upstream | GGGAGAUCAGAGAGAAAAGAAGAGUAAGAA | 53 |
| | UTR | GAAAUAUAAGAGCCACC | |
| 5′UTR-006 | Upstream | GGAAUAAAAGUCUCAACACAACAUAUACAA | 54 |
| | UTR | AACAAACGAAUCUCAAGCAAUCAAGCAUUC | |
| | | UACUUCUAUUGCAGCAAUUUAAAUCAUUUC | |
| | | UUUUAAAGCAAAAGCAAUUUUCUGAAAAUU | |
| | | UUCACCAUUUACGAACGAUAGCAAC | |
| 5′UTR-007 | Upstream | GGGAGACAAGCUUGGCAUUCCGGUACUGUU | 55 |
| | UTR | GGUAAAGCCACC | |
| 5′UTR-008 | Upstream | GGGAAUUAACAGAGAAAAGAAGAGUAAGAA | 56 |
| | UTR | GAAAUAUAAGAGCCACC | |
| 5′UTR-009 | Upstream | GGGAAAUUAGACAGAAAAGAAGAGUAAGAA | 57 |
| | UTR | GAAAUAUAAGAGCCACC | |
| 5′UTR-010 | Upstream | GGGAAAUAAGAGAGUAAAGAACAGUAAGAA | 58 |
| | UTR | GAAAUAUAAGAGCCACC | |
| 5′UTR-011 | Upstream | GGGAAAAAAGAGAGAAAAGAAGACUAAGAA | 59 |
| | UTR | GAAAUAUAAGAGCCACC | |
| 5′UTR-012 | Upstream | GGGAAAUAAGAGAGAAAAGAAGAGUAAGAA | 60 |
| | UTR | GAUAUAUAAGAGCCACC | |
| 5′UTR-013 | Upstream | GGGAAAUAAGAGACAAAACAAGAGUAAGAA | 61 |
| | UTR | GAAAUAUAAGAGCCACC | |
| 5′UTR-014 | Upstream | GGGAAAUUAGAGAGUAAAGAACAGUAAGUA | 62 |
| | UTR | GAAUUAAAAGAGCCACC | |
| 5′UTR-015 | Upstream | GGGAAAUAAGAGAGAAUAGAAGAGUAAGAA | 63 |
| | UTR | GAAAUAUAAGAGCCACC | |
| 5′UTR-016 | Upstream | GGGAAAUAAGAGAGAAAAGAAGAGUAAGAA | 64 |
| | UTR | GAAAAUUAAGAGCCACC | |
| 5′UTR-017 | Upstream | GGGAAAUAAGAGAGAAAAGAAGAGUAAGAA | 65 |
| | UTR | GAAAUUUAAGAGCCACC | |
| 5′UTR-018 | Upstream | GGGAAAUAAGAGAGAAAAGAAGAGUAAGAA | 66 |
| | UTR | GAAAUAUAAGAGCCACC | |
| 5′UTR-019 | Upstream | UCAAGCUUUUGGACCCUCGUACAGAAGCUA | 67 |
| | UTR | AUACGACUCACUAUAGGGAAAUAAGAGAGA | |
| | | AAAGAAGAGUAAGAAGAAAUAUAAGAGCCA | |
| | | CC | |
| 5′UTR-020 | Upstream | GGACAGAUCGCCUGGAGACGCCUACCACGC | 68 |
| | UTR | UGUUUUGACCUCCAUAGAAGACACCGGGAC | |
| | | CGAUCCAGCCUCCGCGGCCGGGAACGGUGC | |
| | | AUUGGAACGCGGAUUCCCCGUGCCAAGAGU | |
| | | GACUCACCGUCCUUGACACG | |
| 5′UTR-021 | Upstream | GGCGCUGCCUACGGAGGUGGCAGCCAUCUC | 69 |
| | UTR | CUUCUCGGCAUC | |
| S065 core | Upstream | CCUCAUAUCCAGGCUCAAGAAUAGAGCUCA | 46 |
| | UTR | GUGUUUUGUUGUUUAAUCAUUCCGACGUGU | |
| | | UUUGCGAUAUUCGCGCAAAGCAGCCAGUCG | |
| | | CGCGCUUGCUUUUAAGUAGAGUUGUUUUUC | |
| | | CACCCGUUUGCCAGGCAUCUUUAAUUUAAC | |
| | | AUAUUUUUAUUUUUCAGGCUAACCUA | |
| S065 | Upstream | GGGAGACCUCAUAUCCAGGCUCAAGAAUAG | 42 |
| | UTR | AGCUCAGUGUUUUGUUGUUUAAUCAUUCCG | |
| | | ACGUGUUUUGCGAUAUUCGCGCAAAGCAGC | |
| | | CAGUCGCGCGCUUGCUUUUAAGUAGAGUUG | |
| | | UUUUUCCACCCGUUUGCCAGGCAUCUUUAA | |
| | | UUUAACAUAUUUUUAUUUUUCAGGCUAACC | |
| | | UAAAGCAGAGAA | |
| S065-A | Upstream | AGGAGACCUCAUAUCCAGGCUCAAGAAUAG | 72 |
| | UTR | AGCUCAGUGUUUUGUUGUUUAAUCAUUCCG | |
| | | ACGUGUUUUGCGAUAUUCGCGCAAAGCAGC | |
| | | CAGUCGCGCGCUUGCUUUUAAGUAGAGUUG | |
| | | UUUUUCCACCCGUUUGCCAGGCAUCUUUAA | |
| | | UUUAACAUAUUUUUAUUUUUCAGGCUAACC | |
| | | UAAAGCAGAGAA | |
| combo3_S065 | Upstream | GGGAGACCUCAUAUCCAGGCUCAAGAAUAG | 39 |
| (S065 ExtKozak) | UTR | AGCUCAGUGUUUUGUUGUUUAAUCAUUCCG | |
| | | ACGUGUUUUGCGAUAUUCGCGCAAAGCAGC | |
| | | CAGUCGCGCGCUUGCUUUUAAGUAGAGUUG | |
| | | UUUUUCCACCCGUUUGCCAGGCAUCUUUAA | |
| | | UUUAACAUAUUUUUAUUUUUCAGGCUAACC | |
| | | UACGCCGCCACC | |
| combo3_S065 | Upstream | AGGAGACCUCAUAUCCAGGCUCAAGAAUAG | 73 |
| (S065 ExtKozak)-A | UTR | AGCUCAGUGUUUUGUUGUUUAAUCAUUCCG | |
| | | ACGUGUUUUGCGAUAUUCGCGCAAAGCAGC | |
| | | CAGUCGCGCGCUUGCUUUUAAGUAGAGUUG | |
| | | UUUUUCCACCCGUUUGCCAGGCAUCUUUAA | |
| | | UUUAACAUAUUUUUAUUUUUCAGGCUAACC | |
| | | UACGCCGCCACC | |

Other non-UTR sequences can also be used as regions or subregions within the polynucleotides. For example, introns or portions of introns sequences can be incorporated into regions of the polynucleotides. Incorporation of intronic sequences can increase protein production as well as polynucleotide levels.

Combinations of features can be included in flanking regions and can be contained within other features. For example, the ORF can be flanked by a 5′ UTR which can contain a strong Kozak translational initiation signal and/or a 3′ UTR which can include an oligo(dT) sequence for templated addition of a poly-A tail. A 5′ UTR can comprise a first polynucleotide fragment and a second polynucleotide fragment from the same and/or different genes such as the 5′ UTRs described in U.S. Patent Application Publication No. 2010-0293625.

These UTRs or portions thereof can be placed in the same orientation as in the transcript from which they were selected or can be altered in orientation or location. Hence a 5′ or 3′ UTR can be inverted, shortened, lengthened, made with one or more other 5′ UTRs or 3′ UTRs.

In some embodiments, the UTR sequences can be changed in some way in relation to a reference sequence. For example, a 3′ or 5′ UTR can be altered relative to a wild type or native UTR by the change in orientation or location as taught above or can be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ or 3′ UTR can be used. As used herein, a “double” UTR is one in which two copies of the same UTR are encoded either in series or substantially in series. For example, a double beta-globin 3′ UTR can be used as described in U.S. Patent Application Publication No. 2010-0129877.

In some embodiments, flanking regions can be heterologous. In some embodiments, the 5′ untranslated region can be derived from a different species than the 3′ untranslated region. The untranslated region can also include translation enhancer elements (TEE). As a non-limiting example, the TEE can include those described in U.S. Patent Application Publication No. 2009-0226470.

In some embodiments, the mRNAs provided by the disclosure comprise a 5′ UTR comprising a T7 leader sequence at the 5′ end of the 5′ UTR. In some embodiments, the mRNA of the disclosure comprises a 5′ UTR comprising a T7 leader sequence comprising the sequence GGGAGA at the 5′ end of the 5′ UTR. In some embodiments, the mRNA of the disclosure comprises a 5′ UTR comprising a T7 leader sequence comprising the sequence GGGAAA at the 5′ end of the 5′ UTR. In some embodiments, the mRNA comprises a 5′ UTR which does not comprise a T7 leader sequence at the 5′ end of the 5′ UTR. In another aspect, the disclosure provides an mRNA comprising a 5′ UTR, wherein the nucleotide sequence of the 5′ UTR comprises any one of the nucleotide sequences set forth in Table 6.

### 3′ UTR and the AU Rich Elements

In certain embodiments, the polynucleotide (e.g., mRNA) encoding a polypeptide further comprises a 3′ UTR. 3′-UTR is the section of mRNA that immediately follows the translation termination codon and often contains regulatory regions that post-transcriptionally influence gene expression. Regulatory regions within the 3′-UTR can influence polyadenylation, translation efficiency, localization, and stability of the mRNA. In one embodiment, the 3′-UTR useful for the disclosure comprises a binding site for regulatory proteins or microRNAs. In some embodiments, the 3′-UTR has a silencer region, which binds to repressor proteins and inhibits the expression of the mRNA. In other embodiments, the 3′-UTR comprises an AU-rich element. Proteins bind AREs to affect the stability or decay rate of transcripts in a localized manner or affect translation initiation. In other embodiments, the 3′-UTR comprises the sequence AAUAAA that directs addition of several hundred adenine residues called the poly(A) tail to the end of the mRNA transcript.

Table 7 shows a listing of 3′-untranslated regions useful for the mRNAs encoding a polypeptide. Variants of 3′ UTRs can be utilized wherein one or more nucleotides are added or removed to the termini, including A, U, C or G.

TABLE 7
Exemplary 3′-Untranslated Regions

| 3′UTR | | | |
|---|---|---|---|
| Identifier | Name/Description | Sequence | SEQ ID NO. |
| 3′UTR-001 | Creatine Kinase | GCGCCUGCCCACCUGCCACCGACUGC | 90 |
| | | UGGAACCCAGCCAGUGGGAGGGCCUG | |
| | | GCCCACCAGAGUCCUGCUCCCUCACU | |
| | | CCUCGCCCCGCCCCCUGUCCCAGAGU | |
| | | CCCACCUGGGGGCUCUCUCCACCCUU | |
| | | CUCAGAGUUCCAGUUUCAACCAGAGU | |
| | | UCCAACCAAUGGGCUCCAUCCUCUGG | |
| | | AUUCUGGCCAAUGAAAUAUCUCCCUG | |
| | | GCAGGGUCCUCUUCUUUUCCCAGAGC | |
| | | UCCACCCCAACCAGGAGCUCUAGUUA | |
| | | AUGGAGAGCUCCCAGCACACUCGGAG | |
| | | CUUGUGCUUUGUCUCCACGCAAAGCG | |
| | | AUAAAUAAAAGCAUUGGUGGCCUUUG | |
| | | GUCUUUGAAUAAAGCCUGAGUAGGAA | |
| | | GUCUAGA | |
| 3′UTR0002 | Myoglobin | GCCCCUGCCGCUCCCACCCCCACCCA | 91 |
| | | UCUGGGCCCCGGGUUCAAGAGAGAGC | |
| | | GGGGUCUGAUCUCGUGUAGCCAUAUA | |
| | | GAGUUUGCUUCUGAGUGUCUGCUUUG | |
| | | UUUAGUAGAGGUGGGCAGGAGGAGCU | |
| | | GAGGGGCUGGGGCUGGGGUGUUGAAG | |
| | | UUGGCUUUGCAUGCCCAGCGAUGCGC | |
| | | CUCCCUGUGGGAUGUCAUCACCCUGG | |
| | | GAACCGGGAGUGGCCCUUGGCUCACU | |
| | | GUGUUCUGCAUGGUUUGGAUCUGAAU | |
| | | UAAUUGUCCUUUCUUCUAAAUCCCAA | |
| | | CCGAACUUCUUCCAACCUCCAAACUG | |
| | | GCUGUAACCCCAAAUCCAAGCCAUUA | |
| | | ACUACACCUGACAGUAGCAAUUGUCU | |
| | | GAUUAAUCACUGGCCCCUUGAAGACA | |
| | | GCAGAAUGUCCCUUUGCAAUGAGGAG | |
| | | GAGAUCUGGGCUGGGCGGGCCAGCUG | |
| | | GGGAAGCAUUUGACUAUCUGGAACUU | |
| | | GUGUGUGCCUCCUCAGGUAUGGCAGU | |
| | | GACUCACCUGGUUUUAAUAAAACAAC | |
| | | CUGCAACAUCUCAUGGUCUUUGAAUA | |
| | | AAGCCUGAGUAGGAAGUCUAGA | |
| 3′UTR-003 | α-actin | ACACACUCCACCUCCAGCACGCGACU | 92 |
| | | UCUCAGGACGACGAAUCUUCUCAAUG | |
| | | GGGGGGCGGCUGAGCUCCAGCCACCC | |
| | | CGCAGUCACUUUCUUUGUAACAACUU | |
| | | CCGUUGCUGCCAUCGUAAACUGACAC | |
| | | AGUGUUUAUAACGUGUACAUACAUUA | |
| | | ACUUAUUACCUCAUUUUGUUAUUUUU | |
| | | CGAAACAAAGCCCUGUGGAAGAAAAU | |
| | | GGAAAACUUGAAGAAGCAUUAAAGUC | |
| | | AUUCUGUUAAGCUGCGUAAAUGGUCU | |
| | | UUGAAUAAAGCCUGAGUAGGAAGUCU | |
| | | AGA | |
| 3′UTR-004 | Albumin | CAUCACAUUUAAAAGCAUCUCAGCCU | 93 |
| | | ACCAUGAGAAUAAGAGAAAGAAAAUG | |
| | | AAGAUCAAAAGCUUAUUCAUCUGUUU | |
| | | UUCUUUUUCGUUGGUGUAAAGCCAAC | |
| | | ACCCUGUCUAAAAAACAUAAAUUUCU | |
| | | UUAAUCAUUUUGCCUCUUUUCUCUGU | |
| | | GCUUCAAUUAAUAAAAAAUGGAAAGA | |
| | | AUCUAAUAGAGUGGUACAGCACUGUU | |
| | | AUUUUUCAAAGAUGUGUUGCUAUCCU | |
| | | GAAAAUUCUGUAGGUUCUGUGGAAGU | |
| | | UCCAGUGUUCUCUCUUAUUCCACUUC | |
| | | GGUAGAGGAUUUCUAGUUUCUUGUGG | |
| | | GCUAAUUAAAUAAAUCAUUAAUACUC | |
| | | UUCUAAUGGUCUUUGAAUAAAGCCUG | |
| | | AGUAGGAAGUCUAGA | |
| 3′UTR-005 | α-globin | GCUGCCUUCUGCGGGGCUUGCCUUCU | 94 |
| | | GGCCAUGCCCUUCUUCUCUCCCUUGC | |
| | | ACCUGUACCUCUUGGUCUUUGAAUAA | |
| | | AGCCUGAGUAGGAAGGCGGCCGCUCG | |
| | | AGCAUGCAUCUAGA | |
| 3′UTR-006 | G-CSF | GCCAAGCCCUCCCCAUCCCAUGUAUU | 95 |
| | | UAUCUCUAUUUAAUAUUUAUGUCUAU | |
| | | UUAAGCCUCAUAUUUAAAGACAGGGA | |
| | | AGAGCAGAACGGAGCCCCAGGCCUCU | |
| | | GUGUCCUUCCCUGCAUUUCUGAGUUU | |
| | | CAUUCUCCUGCCUGUAGCAGUGAGAA | |
| | | AAAGCUCCUGUCCUCCCAUCCCCUGG | |
| | | ACUGGGAGGUAGAUAGGUAAAUACCA | |
| | | AGUAUUUAUUACUAUGACUGCUCCCC | |
| | | AGCCCUGGCUCUGCAAUGGGCACUGG | |
| | | GAUGAGCCGCUGUGAGCCCCUGGUCC | |
| | | UGAGGGUCCCCACCUGGGACCCUUGA | |
| | | GAGUAUCAGGUCUCCCACGUGGGAGA | |
| | | CAAGAAAUCCCUGUUUAAUAUUUAAA | |
| | | CAGCAGUGUUCCCCAUCUGGGUCCUU | |
| | | GCACCCCUCACUCUGGCCUCAGCCGA | |
| | | CUGCACAGCGGCCCCUGCAUCCCCUU | |
| | | GGCUGUGAGGCCCCUGGACAAGCAGA | |
| | | GGUGGCCAGAGCUGGGAGGCAUGGCC | |
| | | CUGGGGUCCCACGAAUUUGCUGGGGA | |
| | | AUCUCGUUUUUCUUCUUAAGACUUUU | |
| | | GGGACAUGGUUUGACUCCCGAACAUC | |
| | | ACCGACGCGUCUCCUGUUUUUCUGGG | |
| | | UGGCCUCGGGACACCUGCCCUGCCCC | |
| | | CACGAGGGUCAGGACUGUGACUCUUU | |
| | | UUAGGGCCAGGCAGGUGCCUGGACAU | |
| | | UUGCCUUGCUGGACGGGGACUGGGGA | |
| | | UGUGGGAGGGAGCAGACAGGAGGAAU | |
| | | CAUGUCAGGCCUGUGUGUGAAAGGAA | |
| | | GCUCCACUGUCACCCUCCACCUCUUC | |
| | | ACCCCCCACUCACCAGUGUCCCCUCC | |
| | | ACUGUCACAUUGUAACUGAACUUCAG | |
| | | GAUAAUAAAGUGUUUGCCUCCAUGGU | |
| | | CUUUGAAUAAAGCCUGAGUAGGAAGG | |
| | | CGGCCGCUCGAGCAUGCAUCUAGA | |
| 3′UTR-007 | Col1a2; | ACUCAAUCUAAAUUAAAAAAGAAAGA | 96 |
| | collagen, | AAUUUGAAAAAACUUUCUCUUUGCCA | |
| | | UUUCUUCUUCUUCUUUUUUAACUGAA | |
| | | AGCUGAAUCCUUCCAUUUCUUCUGCA | |
| | | CAUCUACUUGCUUAAAUUGUGGGCAA | |
| | | AAGAGAAAAAGAAGGAUUGAUCAGAG | |
| | | CAUUGUGCAAUACAGUUUCAUUAACU | |
| | | CCUUCCCCCGCUCCCCCAAAAAUUUG | |
| | | AAUUUUUUUUUCAACACUCUUACACC | |
| | | UGUUAUGGAAAAUGUCAACCUUUGUA | |
| | | AGAAAACCAAAAUAAAAAUUGAAAAA | |
| | | UAAAAACCAUAAACAUUUGCACCACU | |
| | | UGUGGCUUUUGAAUAUCUUCCACAGA | |
| | | GGGAAGUUUAAAACCCAAACUUCCAA | |
| | | AGGUUUAAACUACCUCAAAACACUUU | |
| | | CCCAUGAGUGUGAUCCACAUUGUUAG | |
| | | GUGCUGACCUAGACAGAGAUGAACUG | |
| | | AGGUCCUUGUUUUGUUUUGUUCAUAA | |
| | | UACAAAGGUGCUAAUUAAUAGUAUUU | |
| | | CAGAUACUUGAAGAAUGUUGAUGGUG | |
| | | CUAGAAGAAUUUGAGAAGAAAUACUC | |
| | | CUGUAUUGAGUUGUAUCGUGUGGUGU | |
| | | AUUUUUUAAAAAAUUUGAUUUAGCAU | |
| | | UCAUAUUUUCCAUCUUAUUCCCAAUU | |
| | | AAAAGUAUGCAGAUUAUUUGCCCAAA | |
| | | UCUUCUUCAGAUUCAGCAUUUGUUCU | |
| | | UUGCCAGUCUCAUUUUCAUCUUCUUC | |
| | | CAUGGUUCCACAGAAGCUUUGUUUCU | |
| | | UGGGCAAGCAGAAAAAUUAAAUUGUA | |
| | | CCUAUUUUGUAUAUGUGAGAUGUUUA | |
| | | AAUAAAUUGUGAAAAAAAUGAAAUAA | |
| | | AGCAUGUUUGGUUUUCCAAAAGAACA | |
| | | UAU | |
| 3′UTR-008 | Col6a2; collagen, | CGCCGCCGCCCGGGCCCCGCAGUCGA | 97 |
| | type VI, alpha 2 | GGGUCGUGAGCCCACCCCGUCCAUGG | |
| | | UGCUAAGCGGGCCCGGGUCCCACACG | |
| | | GCCAGCACCGCUGCUCACUCGGACGA | |
| | | CGCCCUGGGCCUGCACCUCUCCAGCU | |
| | | CCUCCCACGGGGUCCCCGUAGCCCCG | |
| | | GCCCCCGCCCAGCCCCAGGUCUCCCC | |
| | | AGGCCCUCCGCAGGCUGCCCGGCCUC | |
| | | CCUCCCCCUGCAGCCAUCCCAAGGCU | |
| | | CCUGACCUACCUGGCCCCUGAGCUCU | |
| | | GGAGCAAGCCCUGACCCAAUAAAGGC | |
| | | UUUGAACCCAU | |
| 3′UTR-009 | RPN1; | GGGGCUAGAGCCCUCUCCGCACAGCG | 98 |
| | ribophorin I | UGGAGACGGGGCAAGGAGGGGGGUUA | |
| | | UUAGGAUUGGUGGUUUUGUUUUGCUU | |
| | | UGUUUAAAGCCGUGGGAAAAUGGCAC | |
| | | AACUUUACCUCUGUGGGAGAUGCAAC | |
| | | ACUGAGAGCCAAGGGGUGGGAGUUGG | |
| | | GAUAAUUUUUAUAUAAAAGAAGUUUU | |
| | | UCCACUUUGAAUUGCUAAAAGUGGCA | |
| | | UUUUUCCUAUGUGCAGUCACUCCUCU | |
| | | CAUUUCUAAAAUAGGGACGUGGCCAG | |
| | | GCACGGUGGCUCAUGCCUGUAAUCCC | |
| | | AGCACUUUGGGAGGCCGAGGCAGGCG | |
| | | GCUCACGAGGUCAGGAGAUCGAGACU | |
| | | AUCCUGGCUAACACGGUAAAACCCUG | |
| | | UCUCUACUAAAAGUACAAAAAAUUAG | |
| | | CUGGGCGUGGUGGUGGGCACCUGUAG | |
| | | UCCCAGCUACUCGGGAGGCUGAGGCA | |
| | | GGAGAAAGGCAUGAAUCCAAGAGGCA | |
| | | GAGCUUGCAGUGAGCUGAGAUCACGC | |
| | | CAUUGCACUCCAGCCUGGGCAACAGU | |
| | | GUUAAGACUCUGUCUCAAAUAUAAAU | |
| | | AAAUAAAUAAAUAAAUAAAUAAAUAA | |
| | | AUAAAAAUAAAGCGAGAUGUUGCCCU | |
| | | CAAA | |
| 3′UTR-010 | LRP1; low density | GGCCCUGCCCCGUCGGACUGCCCCCA | 99 |
| | lipoprotein | GAAAGCCUCCUGCCCCCUGCCAGUGA | |
| | receptor-related | AGUCCUUCAGUGAGCCCCUCCCCAGC | |
| | protein 1 | CAGCCCUUCCCUGGCCCCGCCGGAUG | |
| | | UAUAAAUGUAAAAAUGAAGGAAUUAC | |
| | | AUUUUAUAUGUGAGCGAGCAAGCCGG | |
| | | CAAGCGAGCACAGUAUUAUUUCUCCA | |
| | | UCCCCUCCCUGCCUGCUCCUUGGCAC | |
| | | CCCCAUGCUGCCUUCAGGGAGACAGG | |
| | | CAGGGAGGGCUUGGGGCUGCACCUCC | |
| | | UACCCUCCCACCAGAACGCACCCCAC | |
| | | UGGGAGAGCUGGUGGUGCAGCCUUCC | |
| | | CCUCCCUGUAUAAGACACUUUGCCAA | |
| | | GGCUCUCCCCUCUCGCCCCAUCCCUG | |
| | | CUUGCCCGCUCCCACAGCUUCCUGAG | |
| | | GGCUAAUUCUGGGAAGGGAGAGUUCU | |
| | | UUGCUGCCCCUGUCUGGAAGACGUGG | |
| | | CUCUGGGUGAGGUAGGCGGGAAAGGA | |
| | | UGGAGUGUUUUAGUUCUUGGGGGAGG | |
| | | CCACCCCAAACCCCAGCCCCAACUCC | |
| | | AGGGGCACCUAUGAGAUGGCCAUGCU | |
| | | CAACCCCCCUCCCAGACAGGCCCUCC | |
| | | CUGUCUCCAGGGCCCCCACCGAGGUU | |
| | | CCCAGGGCUGGAGACUUCCUCUGGUA | |
| | | AACAUUCCUCCAGCCUCCCCUCCCCU | |
| | | GGGGACGCCAAGGAGGUGGGCCACAC | |
| | | CCAGGAAGGGAAAGCGGGCAGCCCCG | |
| | | UUUUGGGGACGUGAACGUUUUAAUAA | |
| | | UUUUUGCUGAAUUCCUUUACAACUAA | |
| | | AUAACACAGAUAUUGUUAUAAAUAAA | |
| | | AUUGU | |
| 3′UTR-011 | Nnt1; | AUAUUAAGGAUCAAGCUGUUAGCUAA | 100 |
| | cardiothrophin-like | UAAUGCCACCUCUGCAGUUUUGGGAA | |
| | cytokine factor 1 | CAGGCAAAUAAAGUAUCAGUAUACAU | |
| | | GGUGAUGUACAUCUGUAGCAAAGCUC | |
| | | UUGGAGAAAAUGAAGACUGAAGAAAG | |
| | | CAAAGCAAAAACUGUAUAGAGAGAUU | |
| | | UUUCAAAAGCAGUAAUCCCUCAAUUU | |
| | | UAAAAAAGGAUUGAAAAUUCUAAAUG | |
| | | UCUUUCUGUGCAUAUUUUUUGUGUUA | |
| | | GGAAUCAAAAGUAUUUUAUAAAAGGA | |
| | | GAAAGAACAGCCUCAUUUUAGAUGUA | |
| | | GUCCUGUUGGAUUUUUUAUGCCUCCU | |
| | | CAGUAACCAGAAAUGUUUUAAAAAAC | |
| | | UAAGUGUUUAGGAUUUCAAGACAACA | |
| | | UUAUACAUGGCUCUGAAAUAUCUGAC | |
| | | ACAAUGUAAACAUUGCAGGCACCUGC | |
| | | AUUUUAUGUUUUUUUUUUCAACAAAU | |
| | | GUGACUAAUUUGAAACUUUUAUGAAC | |
| | | UUCUGAGCUGUCCCCUUGCAAUUCAA | |
| | | CCGCAGUUUGAAUUAAUCAUAUCAAA | |
| | | UCAGUUUUAAUUUUUUAAAUUGUACU | |
| | | UCAGAGUCUAUAUUUCAAGGGCACAU | |
| | | UUUCUCACUACUAUUUUAAUACAUUA | |
| | | AAGGACUAAAUAAUCUUUCAGAGAUG | |
| | | CUGGAAACAAAUCAUUUGCUUUAUAU | |
| | | GUUUCAUUAGAAUACCAAUGAAACAU | |
| | | ACAACUUGAAAAUUAGUAAUAGUAUU | |
| | | UUUGAAGAUCCCAUUUCUAAUUGGAG | |
| | | AUCUCUUUAAUUUCGAUCAACUUAUA | |
| | | AUGUGUAGUACUAUAUUAAGUGCACU | |
| | | UGAGUGGAAUUCAACAUUUGACUAAU | |
| | | AAAAUGAGUUCAUCAUGUUGGCAAGU | |
| | | GAUGUGGCAAUUAUCUCUGGUGACAA | |
| | | AAGAGUAAAAUCAAAUAUUUCUGCCU | |
| | | GUUACAAAUAUCAAGGAAGACCUGCU | |
| | | ACUAUGAAAUAGAUGACAUUAAUCUG | |
| | | UCUUCACUGUUUAUAAUACGGAUGGA | |
| | | UUUUUUUUCAAAUCAGUGUGUGUUUU | |
| | | GAGGUCUUAUGUAAUUGAUGACAUUU | |
| | | GAGAGAAAUGGUGGCUUUUUUUAGCU | |
| | | ACCUCUUUGUUCAUUUAAGCACCAGU | |
| | | AAAGAUCAUGUCUUUUUAUAGAAGUG | |
| | | UAGAUUUUCUUUGUGACUUUGCUAUC | |
| | | GUGCCUAAAGCUCUAAAUAUAGGUGA | |
| | | AUGUGUGAUGAAUACUCAGAUUAUUU | |
| | | GUCUCUCUAUAUAAUUAGUUUGGUAC | |
| | | UAAGUUUCUCAAAAAAUUAUUAACAC | |
| | | AUGAAAGACAAUCUCUAAACCAGAAA | |
| | | AAGAAGUAGUACAAAUUUUGUUACUG | |
| | | UAAUGCUCGCGUUUAGUGAGUUUAAA | |
| | | ACACACAGUAUCUUUUGGUUUUAUAA | |
| | | UCAGUUUCUAUUUUGCUGUGCCUGAG | |
| | | AUUAAGAUCUGUGUAUGUGUGUGUGU | |
| | | GUGUGUGUGCGUUUGUGUGUUAAAGC | |
| | | AGAAAAGACUUUUUUAAAAGUUUUAA | |
| | | GUGAUAAAUGCAAUUUGUUAAUUGAU | |
| | | CUUAGAUCACUAGUAAACUCAGGGCU | |
| | | GAAUUAUACCAUGUAUAUUCUAUUAG | |
| | | AAGAAAGUAAACACCAUCUUUAUUCC | |
| | | UGCCCUUUUUCUUCUCUCAAAGUAGU | |
| | | UGUAGUUAUAUCUAGAAAGAAGCAAU | |
| | | UUUGAUUUCUUGAAAAGGUAGUUCCU | |
| | | GCACUCAGUUUAAACUAAAAAUAAUC | |
| | | AUACUUGGAUUUUAUUUAUUUUUGUC | |
| | | AUAGUAAAAAUUUUAAUUUAUAUAUA | |
| | | UUUUUAUUUAGUAUUAUCUUAUUCUU | |
| | | UGCUAUUUGCCAAUCCUUUGUCAUCA | |
| | | AUUGUGUUAAAUGAAUUGAAAAUUCA | |
| | | UGCCCUGUUCAUUUUAUUUUACUUUA | |
| | | UUGGUUAGGAUAUUUAAAGGAUUUUU | |
| | | GUAUAUAUAAUUUCUUAAAUUAAUAU | |
| | | UCCAAAAGGUUAGUGGACUUAGAUUA | |
| | | UAAAUUAUGGCAAAAAUCUAAAAACA | |
| | | ACAAAAAUGAUUUUUAUACAUUCUAU | |
| | | UUCAUUAUUCCUCUUUUUCCAAUAAG | |
| | | UCAUACAAUUGGUAGAUAUGACUUAU | |
| | | UUUAUUUUUGUAUUAUUCACUAUAUC | |
| | | UUUAUGAUAUUUAAGUAUAAAUAAUU | |
| | | AAAAAAAUUUAUUGUACCUUAUAGUC | |
| | | UGUCACCAAAAAAAAAAAAUUAUCUG | |
| | | UAGGUAGUGAAAUGCUAAUGUUGAUU | |
| | | UGUCUUUAAGGGCUUGUUAACUAUCC | |
| | | UUUAUUUUCUCAUUUGUCUUAAAUUA | |
| | | GGAGUUUGUGUUUAAAUUACUCAUCU | |
| | | AAGCAAAAAAUGUAUAUAAAUCCCAU | |
| | | UACUGGGUAUAUACCCAAAGGAUUAU | |
| | | AAAUCAUGCUGCUAUAAAGACACAUG | |
| | | CACACGUAUGUUUAUUGCAGCACUAU | |
| | | UCACAAUAGCAAAGACUUGGAACCAA | |
| | | CCCAAAUGUCCAUCAAUGAUAGACUU | |
| | | GAUUAAGAAAAUGUGCACAUAUACAC | |
| | | CAUGGAAUACUAUGCAGCCAUAAAAA | |
| | | AGGAUGAGUUCAUGUCCUUUGUAGGG | |
| | | ACAUGGAUAAAGCUGGAAACCAUCAU | |
| | | UCUGAGCAAACUAUUGCAAGGACAGA | |
| | | AAACCAAACACUGCAUGUUCUCACUC | |
| | | AUAGGUGGGAAUUGAACAAUGAGAAC | |
| | | ACUUGGACACAAGGUGGGGAACACCA | |
| | | CACACCAGGGCCUGUCAUGGGGUGGG | |
| | | GGGAGUGGGGAGGGAUAGCAUUAGGA | |
| | | GAUAUACCUAAUGUAAAUGAUGAGUU | |
| | | AAUGGGUGCAGCACACCAACAUGGCA | |
| | | CAUGUAUACAUAUGUAGCAAACCUGC | |
| | | ACGUUGUGCACAUGUACCCUAGAACU | |
| | | UAAAGUAUAAUUAAAAAAAAAAAGAA | |
| | | AACAGAAGCUAUUUAUAAAGAAGUUA | |
| | | UUUGCUGAAAUAAAUGUGAUCUUUCC | |
| | | CAUUAAAAAAAUAAAGAAAUUUUGGG | |
| | | GUAAAAAAACACAAUAUAUUGUAUUC | |
| | | UUGAAAAAUUCUAAGAGAGUGGAUGU | |
| | | GAAGUGUUCUCACCACAAAAGUGAUA | |
| | | ACUAAUUGAGGUAAUGCACAUAUUAA | |
| | | UUAGAAAGAUUUUGUCAUUCCACAAU | |
| | | GUAUAUAUACUUAAAAAUAUGUUAUA | |
| | | CACAAUAAAUACAUACAUUAAAAAAU | |
| | | AAGUAAAUGUA | |
| 3′UTR-012 | Col6a1; collagen, | CCCACCCUGCACGCCGGCACCAAACC | 101 |
| | type VI, alpha 1 | CUGUCCUCCCACCCCUCCCCACUCAU | |
| | | CACUAAACAGAGUAAAAUGUGAUGCG | |
| | | AAUUUUCCCGACCAACCUGAUUCGCU | |
| | | AGAUUUUUUUUAAGGAAAAGCUUGGA | |
| | | AAGCCAGGACACAACGCUGCUGCCUG | |
| | | CUUUGUGCAGGGUCCUCCGGGGCUCA | |
| | | GCCCUGAGUUGGCAUCACCUGCGCAG | |
| | | GGCCCUCUGGGGCUCAGCCCUGAGCU | |
| | | AGUGUCACCUGCACAGGGCCCUCUGA | |
| | | GGCUCAGCCCUGAGCUGGCGUCACCU | |
| | | GUGCAGGGCCCUCUGGGGCUCAGCCC | |
| | | UGAGCUGGCCUCACCUGGGUUCCCCA | |
| | | CCCCGGGCUCUCCUGCCCUGCCCUCC | |
| | | UGCCCGCCCUCCCUCCUGCCUGCGCA | |
| | | GCUCCUUCCCUAGGCACCUCUGUGCU | |
| | | GCAUCCCACCAGCCUGAGCAAGACGC | |
| | | CCUCUCGGGGCCUGUGCCGCACUAGC | |
| | | CUCCCUCUCCUCUGUCCCCAUAGCUG | |
| | | GUUUUUCCCACCAAUCCUCACCUAAC | |
| | | AGUUACUUUACAAUUAAACUCAAAGC | |
| | | AAGCUCUUCUCCUCAGCUUGGGGCAG | |
| | | CCAUUGGCCUCUGUCUCGUUUUGGGA | |
| | | AACCAAGGUCAGGAGGCCGUUGCAGA | |
| | | CAUAAAUCUCGGCGACUCGGCCCCGU | |
| | | CUCCUGAGGGUCCUGCUGGUGACCGG | |
| | | CCUGGACCUUGGCCCUACAGCCCUGG | |
| | | AGGCCGCUGCUGACCAGCACUGACCC | |
| | | CGACCUCAGAGAGUACUCGCAGGGGC | |
| | | GCUGGCUGCACUCAAGACCCUCGAGA | |
| | | UUAACGGUGCUAACCCCGUCUGCUCC | |
| | | UCCCUCCCGCAGAGACUGGGGCCUGG | |
| | | ACUGGACAUGAGAGCCCCUUGGUGCC | |
| | | ACAGAGGGCUGUGUCUUACUAGAAAC | |
| | | AACGCAAACCUCUCCUUCCUCAGAAU | |
| | | AGUGAUGUGUUCGACGUUUUAUCAAA | |
| | | GGCCCCCUUUCUAUGUUCAUGUUAGU | |
| | | UUUGCUCCUUCUGUGUUUUUUUCUGA | |
| | | ACCAUAUCCAUGUUGCUGACUUUUCC | |
| | | AAAUAAAGGUUUUCACUCCUCUC | |
| 3′UTR-013 | Calr; | AGAGGCCUGCCUCCAGGGCUGGACUG | 102 |
| | calreticulin | AGGCCUGAGCGCUCCUGCCGCAGAGC | |
| | | UGGCCGCGCCAAAUAAUGUCUCUGUG | |
| | | AGACUCGAGAACUUUCAUUUUUUUCC | |
| | | AGGCUGGUUCGGAUUUGGGGUGGAUU | |
| | | UUGGUUUUGUUCCCCUCCUCCACUCU | |
| | | CCCCCACCCCCUCCCCGCCCUUUUUU | |
| | | UUUUUUUUUUUUAAACUGGUAUUUUA | |
| | | UCUUUGAUUCUCCUUCAGCCCUCACC | |
| | | CCUGGUUCUCAUCUUUCUUGAUCAAC | |
| | | AUCUUUUCUUGCCUCUGUCCCCUUCU | |
| | | CUCAUCUCUUAGCUCCCCUCCAACCU | |
| | | GGGGGGCAGUGGUGUGGAGAAGCCAC | |
| | | AGGCCUGAGAUUUCAUCUGCUCUCCU | |
| | | UCCUGGAGCCCAGAGGAGGGCAGCAG | |
| | | AAGGGGGUGGUGUCUCCAACCCCCCA | |
| | | GCACUGAGGAAGAACGGGGCUCUUCU | |
| | | CAUUUCACCCCUCCCUUUCUCCCCUG | |
| | | CCCCCAGGACUGGGCCACUUCUGGGU | |
| | | GGGGCAGUGGGUCCCAGAUUGGCUCA | |
| | | CACUGAGAAUGUAAGAACUACAAACA | |
| | | AAAUUUCUAUUAAAUUAAAUUUUGUG | |
| | | UCUCC | |
| 3′UTR-014 | Colla1; collagen, | CUCCCUCCAUCCCAACCUGGCUCCCU | 103 |
| | type I, alpha 1 | CCCACCCAACCAACUUUCCCCCCAAC | |
| | | CCGGAAACAGACAAGCAACCCAAACU | |
| | | GAACCCCCUCAAAAGCCAAAAAAUGG | |
| | | GAGACAAUUUCACAUGGACUUUGGAA | |
| | | AAUAUUUUUUUCCUUUGCAUUCAUCU | |
| | | CUCAAACUUAGUUUUUAUCUUUGACC | |
| | | AACCGAACAUGACCAAAAACCAAAAG | |
| | | UGCAUUCAACCUUACCAAAAAAAAAA | |
| | | AAAAAAAAAGAAUAAAUAAAUAACUU | |
| | | UUUAAAAAAGGAAGCUUGGUCCACUU | |
| | | GCUUGAAGACCCAUGCGGGGGUAAGU | |
| | | CCCUUUCUGCCCGUUGGGCUUAUGAA | |
| | | ACCCCAAUGCUGCCCUUUCUGCUCCU | |
| | | UUCUCCACACCCCCCUUGGGGCCUCC | |
| | | CCUCCACUCCUUCCCAAAUCUGUCUC | |
| | | CCCAGAAGACACAGGAAACAAUGUAU | |
| | | UGUCUGCCCAGCAAUCAAAGGCAAUG | |
| | | CUCAAACACCCAAGUGGCCCCCACCC | |
| | | UCAGCCCGCUCCUGCCCGCCCAGCAC | |
| | | CCCCAGGCCCUGGGGGACCUGGGGUU | |
| | | CUCAGACUGCCAAAGAAGCCUUGCCA | |
| | | UCUGGCGCUCCCAUGGCUCUUGCAAC | |
| | | AUCUCCCCUUCGUUUUUGAGGGGGUC | |
| | | AUGCCGGGGGAGCCACCAGCCCCUCA | |
| | | CUGGGUUCGGAGGAGAGUCAGGAAGG | |
| | | GCCACGACAAAGCAGAAACAUCGGAU | |
| | | UUGGGGAACGCGUGUCAAUCCCUUGU | |
| | | GCCGCAGGGCUGGGCGGGAGAGACUG | |
| | | UUCUGUUCCUUGUGUAACUGUGUUGC | |
| | | UGAAAGACUACCUCGUUCUUGUCUUG | |
| | | AUGUGUCACCGGGGCAACUGCCUGGG | |
| | | GGCGGGGAUGGGGGCAGGGUGGAAGC | |
| | | GGCUCCCCAUUUUAUACCAAAGGUGC | |
| | | UACAUCUAUGUGAUGGGUGGGGUGGG | |
| | | GAGGGAAUCACUGGUGCUAUAGAAAU | |
| | | UGAGAUGCCCCCCCAGGCCAGCAAAU | |
| | | GUUCCUUUUUGUUCAAAGUCUAUUUU | |
| | | UAUUCCUUGAUAUUUUUCUUUUUUUU | |
| | | UUUUUUUUUUUGUGGAUGGGGACUUG | |
| | | UGAAUUUUUCUAAAGGUGCUAUUUAA | |
| | | CAUGGGAGGAGAGCGUGUGCGGCUCC | |
| | | AGCCCAGCCCGCUGCUCACUUUCCAC | |
| | | CCUCUCUCCACCUGCCUCUGGCUUCU | |
| | | CAGGCCUCUGCUCUCCGACCUCUCUC | |
| | | CUCUGAAACCCUCCUCCACAGCUGCA | |
| | | GCCCAUCCUCCCGGCUCCCUCCUAGU | |
| | | CUGUCCUGCGUCCUCUGUCCCCGGGU | |
| | | UUCAGAGACAACUUCCCAAAGCACAA | |
| | | AGCAGUUUUUCCCCCUAGGGGUGGGA | |
| | | GGAAGCAAAAGACUCUGUACCUAUUU | |
| | | UGUAUGUGUAUAAUAAUUUGAGAUGU | |
| | | UUUUAAUUAUUUUGAUUGCUGGAAUA | |
| | | AAGCAUGUGGAAAUGACCCAAACAUA | |
| | | AUCCGCAGUGGCCUCCUAAUUUCCUU | |
| | | CUUUGGAGUUGGGGGAGGGGUAGACA | |
| | | UGGGGAAGGGGCUUUGGGGUGAUGGG | |
| | | CUUGCCUUCCAUUCCUGCCCUUUCCC | |
| | | UCCCCACUAUUCUCUUCUAGAUCCCU | |
| | | CCAUAACCCCACUCCCCUUUCUCUCA | |
| | | CCCUUCUUAUACCGCAAACCUUUCUA | |
| | | CUUCCUCUUUCAUUUUCUAUUCUUGC | |
| | | AAUUUCCUUGCACCUUUUCCAAAUCC | |
| | | UCUUCUCCCCUGCAAUACCAUACAGG | |
| | | CAAUCCACGUGCACAACACACACACA | |
| | | CACUCUUCACAUCUGGGGUUGUCCAA | |
| | | ACCUCAUACCCACUCCCCUUCAAGCC | |
| | | CAUCCACUCUCCACCCCCUGGAUGCC | |
| | | CUGCACUUGGUGGCGGUGGGAUGCUC | |
| | | AUGGAUACUGGGAGGGUGAGGGGAGU | |
| | | GGAACCCGUGAGGAGGACCUGGGGGC | |
| | | CUCUCCUUGAACUGACAUGAAGGGUC | |
| | | AUCUGGCCUCUGCUCCCUUCUCACCC | |
| | | ACGCUGACCUCCUGCCGAAGGAGCAA | |
| | | CGCAACAGGAGAGGGGUCUGCUGAGC | |
| | | CUGGCGAGGGUCUGGGAGGGACCAGG | |
| | | AGGAAGGCGUGCUCCCUGCUCGCUGU | |
| | | CCUGGCCCUGGGGGAGUGAGGGAGAC | |
| | | AGACACCUGGGAGAGCUGUGGGGAAG | |
| | | GCACUCGCACCGUGCUCUUGGGAAGG | |
| | | AAGGAGACCUGGCCCUGCUCACCACG | |
| | | GACUGGGUGCCUCGACCUCCUGAAUC | |
| | | CCCAGAACACAACCCCCCUGGGCUGG | |
| | | GGUGGUCUGGGGAACCAUCGUGCCCC | |
| | | CGCCUCCCGCCUACUCCUUUUUAAGC | |
| | | UU | |
| 3′UTR-015 | Plod1; | UUGGCCAGGCCUGACCCUCUUGGACC | 104 |
| | procollagen- | UUUCUUCUUUGCCGACAACCACUGCC | |
| | lysine, 2- | CAGCAGCCUCUGGGACCUCGGGGUCC | |
| | oxoglutarate | CAGGGAACCCAGUCCAGCCUCCUGGC | |
| | 5-dioxygenase 1 | UGUUGACUUCCCAUUGCUCUUGGAGC | |
| | | CACCAAUCAAAGAGAUUCAAAGAGAU | |
| | | UCCUGCAGGCCAGAGGCGGAACACAC | |
| | | CUUUAUGGCUGGGGCUCUCCGUGGUG | |
| | | UUCUGGACCCAGCCCCUGGAGACACC | |
| | | AUUCACUUUUACUGCUUUGUAGUGAC | |
| | | UCGUGCUCUCCAACCUGUCUUCCUGA | |
| | | AAAACCAAGGCCCCCUUCCCCCACCU | |
| | | CUUCCAUGGGGUGAGACUUGAGCAGA | |
| | | ACAGGGGCUUCCCCAAGUUGCCCAGA | |
| | | AAGACUGUCUGGGUGAGAAGCCAUGG | |
| | | CCAGAGCUUCUCCCAGGCACAGGUGU | |
| | | UGCACCAGGGACUUCUGCUUCAAGUU | |
| | | UUGGGGUAAAGACACCUGGAUCAGAC | |
| | | UCCAAGGGCUGCCCUGAGUCUGGGAC | |
| | | UUCUGCCUCCAUGGCUGGUCAUGAGA | |
| | | GCAAACCGUAGUCCCCUGGAGACAGC | |
| | | GACUCCAGAGAACCUCUUGGGAGACA | |
| | | GAAGAGGCAUCUGUGCACAGCUCGAU | |
| | | CUUCUACUUGCCUGUGGGGAGGGGAG | |
| | | UGACAGGUCCACACACCACACUGGGU | |
| | | CACCCUGUCCUGGAUGCCUCUGAAGA | |
| | | GAGGGACAGACCGUCAGAAACUGGAG | |
| | | AGUUUCUAUUAAAGGUCAUUUAAACC | |
| | | A | |
| 3′UTR-016 | Nucb1; | UCCUCCGGGACCCCAGCCCUCAGGAU | 105 |
| | nucleobindin 1 | UCCUGAUGCUCCAAGGCGACUGAUGG | |
| | | GCGCUGGAUGAAGUGGCACAGUCAGC | |
| | | UUCCCUGGGGGCUGGUGUCAUGUUGG | |
| | | GCUCCUGGGGCGGGGGCACGGCCUGG | |
| | | CAUUUCACGCAUUGCUGCCACCCCAG | |
| | | GUCCACCUGUCUCCACUUUCACAGCC | |
| | | UCCAAGUCUGUGGCUCUUCCCUUCUG | |
| | | UCCUCCGAGGGGCUUGCCUUCUCUCG | |
| | | UGUCCAGUGAGGUGCUCAGUGAUCGG | |
| | | CUUAACUUAGAGAAGCCCGCCCCCUC | |
| | | CCCUUCUCCGUCUGUCCCAAGAGGGU | |
| | | CUGCUCUGAGCCUGCGUUCCUAGGUG | |
| | | GCUCGGCCUCAGCUGCCUGGGUUGUG | |
| | | GCCGCCCUAGCAUCCUGUAUGCCCAC | |
| | | AGCUACUGGAAUCCCCGCUGCUGCUC | |
| | | CGGGCCAAGCUUCUGGUUGAUUAAUG | |
| | | AGGGCAUGGGGUGGUCCCUCAAGACC | |
| | | UUCCCCUACCUUUUGUGGAACCAGUG | |
| | | AUGCCUCAAAGACAGUGUCCCCUCCA | |
| | | CAGCUGGGUGCCAGGGGCAGGGGAUC | |
| | | CUCAGUAUAGCCGGUGAACCCUGAUA | |
| | | CCAGGAGCCUGGGCCUCCCUGAACCC | |
| | | CUGGCUUCCAGCCAUCUCAUCGCCAG | |
| | | CCUCCUCCUGGACCUCUUGGCCCCCA | |
| | | GCCCCUUCCCCACACAGCCCCAGAAG | |
| | | GGUCCCAGAGCUGACCCCACUCCAGG | |
| | | ACCUAGGCCCAGCCCCUCAGCCUCAU | |
| | | CUGGAGCCCCUGAAGACCAGUCCCAC | |
| | | CCACCUUUCUGGCCUCAUCUGACACU | |
| | | GCUCCGCAUCCUGCUGUGUGUCCUGU | |
| | | UCCAUGUUCCGGUUCCAUCCAAAUAC | |
| | | ACUUUCUGGAACAAA | |
| 3′UTR-017 | α-globin | GCUGGAGCCUCGGUGGCCAUGCUUCU | 106 |
| | | UGCCCCUUGGGCCUCCCCCCAGCCCC | |
| | | UCCUCCCCUUCCUGCACCCGUACCCC | |
| | | CGUGGUCUUUGAAUAAAGUCUGAGUG | |
| | | GGCGGC | |
| 3′UTR-018 | Downstream UTR | UAAUAGGCUGGAGCCUCGGUGGCCAU | 107 |
| | | GCUUCUUGCCCCUUGGGCCUCCCCCC | |
| | | AGCCCCUCCUCCCCUUCCUGCACCCG | |
| | | UACCCCCGUGGUCUUUGAAUAAAGUC | |
| | | UGAGUGGGCGGC | |
| 3′UTR-019 | Downstream UTR | UGAUAAUAGGCUGGAGCCUCGGUGGC | 108 |
| | | CAUGCUUCUUGCCCCUUGGGCCUCCC | |
| | | CCCAGCCCCUCCUCCCCUUCCUGCAC | |
| | | CCGUACCCCCUGGUCUUUGAAUAAAG | |
| | | UCUGAGUGGGCGGC | |
| v1.1 | Downstream UTR | UGAUAAUAGGCUGGAGCCUCGGUGGC | 109 |
| 3′UTR | | CUAGCUUCUUGCCCCUUGGGCCUCCC | |
| | | CCCAGCCCCUCCUCCCCUUCCUGCAC | |
| | | CCGUACCCCCGUGGUCUUUGAAUAAA | |
| | | GUCUGAGUGGGCGGC | |
| 3′UTR-020 | Downstream UTR | UGAUAAUAGGCUGGAGCCUCGGUGGC | 110 |
| | | CAUGCUUCUUGCCCCUUGGGCCUCCC | |
| | | CCCAGCCCCUCCUCCCCUUCCUGCAC | |
| | | CCGUACCCCCGUGGUCUUUGAAUAAA | |
| | | GUCUGAGUGGGCGGC | |

In certain embodiments, the 3′ UTR sequence useful for the disclosure comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of SEQ ID NOs: 90-110 and any combination thereof. In a particular embodiment, the 3′ UTR sequence further comprises a miRNA binding site, e.g., miR-122 binding site. In other embodiments, a 3′UTR sequence useful for the disclosure comprises 3′ UTR-018 (SEQ ID NO: 107). In other embodiments, a 3′ UTR sequence useful for the disclosure comprises 3′ UTR comprised of nucleotide sequence set forth in SEQ ID NO: 109. In other embodiments, a 3′ UTR sequence useful for the disclosure comprises 3′ UTR comprised of nucleotide sequence set forth in SEQ ID NO: 110.

In certain embodiments, the 3′ UTR sequence comprises one or more miRNA binding sites, e.g., miR-122 binding sites, or any other heterologous nucleotide sequences therein, without disrupting the function of the 3′ UTR. Some examples of 3′ UTR sequences comprising a miRNA binding site are listed in Table 8.

TABLE 8
Exemplary 3′ UTR with miRNA Binding Sites

| 3′UTR Identifier/ | | | |
|---|---|---|---|
| miRNA binding site | Name/Description | Sequence | SEQ ID NO. |
| 3′UTR-018 + | Downstream UTR | UAAUAGGCUGGAGCCUCGGUGGC | 134 |
| miR-122-5p | | CAUGCUUCUUGCCCCUUGGGCCU | |
| binding site | | CCCCCCAGCCCCUCCUCCCCUUC | |
| | | CUGCACCCGUACCCCCCAAACAC | |
| | | CAUUGUCACACUCCAGUGGUCUU | |
| | | UGAAUAAAGUCUGAGUGGGCGGC | |
| 3′UTR-018 + | Downstream UTR | UAAUAGGCUGGAGCCUCGGUGGC | 135 |
| miR-122-3p | | CAUGCUUCUUGCCCCUUGGGCCU | |
| binding site | | CCCCCCAGCCCCUCCUCCCCUUC | |
| | | CUGCACCCGUACCCCCUAUUUAG | |
| | | UGUGAUAAUGGCGUUGUGGUCUU | |
| | | UGAAUAAAGUCUGAGUGGGCGGC | |
| 3′UTR-019 + | Downstream UTR | UGAUAAUAGGCUGGAGCCUCGGU | 136 |
| miR-122 | | GGCCAUGCUUCUUGCCCCUUGGG | |
| binding site | | CCUCCCCCCAGCCCCUCCUCCCC | |
| | | UUCCUGCACCCGUACCCCCCAAA | |
| | | CACCAUUGUCACACUCCAGUGGU | |
| | | CUUUGAAUAAAGUCUGAGUGGGC | |
| | | GGC | |
| 3′UTR + miR- | Downstream UTR | GCUGGAGCCUCGGUGGCCAUGCU | 137 |
| 142-3p | | UCUUGCCCCUUGGGCCUCCCCCC | |
| binding site | | AGCCCCUCCUCCCCUUCCUGCAC | |
| | | CCGUACCCCCUCCAUAAAGUAGG | |
| | | AAACACUACAGUGGUCUUUGAAU | |
| | | AAAGUCUGAGUGGGCGGC | |

*miRNA binding site is bolded.

In certain embodiments, the 3′ UTR sequence useful for the disclosure comprises a nucleotide sequence at least about 60%, at least about 70%, at least about 80%, at least about t90%, at least about 95%, at least about 96%, at least about 97%, at least about 98%, at least about 99%, or about 100% identical to the sequence set forth as SEQ ID NO: 107 or 108.

### Regions Having a 5′ Cap

The polynucleotide comprising an mRNA encoding a polypeptide of the present disclosure can further comprise a 5′ cap. The 5′ cap useful for polypeptide encoding mRNA can bind the mRNA Cap Binding Protein (CBP), thereby increasing mRNA stability. The cap can further assist the removal of 5′ proximal introns removal during mRNA splicing.

In some embodiments, the polynucleotide comprising an mRNA encoding a polypeptide of the present disclosure comprises a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides.

In certain embodiments, the 5′ cap comprises 2′-O-methylation of the ribose sugars of 5′-terminal and/or 5′-anteterminal nucleotides on the 2′-hydroxyl group of the sugar ring. In other embodiments, the caps for the polypeptide-encoding mRNA include cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs can be chemically (i.e. non-enzymatically) or enzymatically synthesized and/or linked to the polynucleotides of the disclosure.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which can equivalently be designated 3′ O-Me-m7G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

In some embodiments, the cap is a dinucleotide cap analog. As a non-limiting example, the dinucleotide cap analog can be modified at different phosphate positions with a boranophosphate group or a phophoroselenoate group such as the dinucleotide cap analogs described in U.S. Pat. No. 8,519,110.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m^{3′—O} G(5′)ppp(5′)G cap analog. See, e.g., the various cap analogs and the methods of synthesizing cap analogs described in Kore et al. (2013) Bioorganic & Medicinal Chemistry 21:4570-4574. In another embodiment, a cap analog of the present disclosure is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

An mRNA of the present disclosure can also be capped post-manufacture (whether IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects.

Non-limiting examples of more authentic 5′ cap structures of the present disclosure are those which, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)NlmpNp (cap 1), and 7mG(5′)-ppp(5′)NlmpN2mp (cap 2).

According to the present disclosure, 5′ terminal caps can include endogenous caps or cap analogs. According to the present disclosure, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### 5′ Capping and 5′ Trinucleotide Cap

It is desirable to manufacture therapeutic RNAs enzymatically using in vitro transcription (IVT). In general, a DNA-dependent RNA polymerase transcribes a DNA template containing an appropriate promoter into an RNA transcript. The poly(A) tail can be generated co-transcriptionally by incorporating a poly(T) tract in the template DNA or separately by using a poly(A) polymerase. Eukaryotic mRNAs start with a 5′ cap (e.g., a 5′ m7GpppX cap). Typically, the 5′ cap begins with an inverted G with N⁷Me (required for eIF4E binding). A preferred cap, Cap1 contains 2′OMe at the +1 position) followed by any nucleoside at +2 position. This cap can be installed post-transcriptionally, e.g., enzymatically (after transcription) or co-transcriptionally (during transcription).

Post-transcriptional capping can be carried out using the vaccinia capping enzyme and allows for complete capping of the RNA, generating a cap 0 structure on RNA carrying a 5′ terminal triphosphate or diphosphate group, the cap 0 structure being required for efficient translation of the mRNA in vivo. The cap 0 structure can then be further modified into cap 1 using a cap-specific 2′O methyltransferase. Vaccinia capping enzyme and 2′O methyltransferase have been used to generate cap 0 and cap 1 structures on in vitro transcripts, for example, for use in transfecting eukaryotic cells or in mRNA therapeutic applications to drive protein synthesis. While post-transcriptional capping by vaccinia capping enzymes can yield either Cap 0 or Cap 1 structures, it is an expensive process when utilized for large-scale mRNA production, for example, vaccinia is costly and in limited supply and there can be difficulties in purifying an IVT mRNA (e.g., removing S-adenosylmethionine (SAM) and 2′O-methyltransferase). Moreover, capping can be incomplete due to inaccessibility of structured 5′ ends.

Co-transcriptional capping using a cap analog has certain advantages over vaccinia capping, for example, the process requires a simpler workflow (e.g., no need for a purification step between transcription and capping). Traditional co-transcriptional capping methods utilize the dinucleotide ARCA (anti-reverse cap analog) and yield Cap 0 structures. ARCA capping has drawbacks, however, for example, the resulting Cap 0 structures can be immunogenic and the process often results in low yields and/or poorly capped material. Another potential drawback of this approach is a theoretical capping efficiency of <100%, due to competition from the GTP for the starting nucleotide. For example, co-transcriptonal capping using ARCA typically requires a 10:1 ratio of ARCA:GTP to achieve >90% capping (needed to outcompete GTP for initiation).

In some embodiments, mRNAs of the disclosure are comprised of trinucleotide mRNA cap analogs, prepared using co-transcriptional capping methods (e.g., featuring T7 RNA polymerase) for the in vitro synthesis of mRNA. Use of a trinucleotide cap analog may provide a solution to several of the above-described problems associated with vaccinia or ARCA capping. In addition, the methods of co-transcriptional capping described provide flexibility in modifying the penultimate nucleobase which may alter binding behavior, or affect the affinity of these caps towards decapping enzymes, or both, thus potentially improving stability of the respective mRNA. An exemplary trinucleotide for use in the herein-described co-transcriptional capping methods is the m7GpppAG (GAG) trinucleotide. Use of this trinucleotide results in the nucleotide at the +1 position being A instead of G. Both +1G and +1A are caps that can be found in naturally-occurring mRNAs.

T7 RNA polymerase prefers to initiate with 5′ GTP. Accordingly, Most conventional mRNA transcripts start with 5′-GGG (based on transcription from a T7 promoter sequence such as 5′TAATACGACTCACTATAGGGNNNNNNNNN . . . 3′ (TATA being referred to as the “TATA box”). T7 RNA polymerase typically transcribes DNA downstream of a T7 promoter (5′ TAATACGACTCACTATAG 3′, referencing the coding strand). T7 polymerase starts transcription at the underlined G in the promoter sequence. The polymerase then transcribes using the opposite strand as a template from 5′→3′. The first base in the transcript will be a G.

The herein-described processes capitalize on the fact that the T7 enzyme has limited initiation activity with the single nucleotide ATP, driving T7 to initiate with the trinucleotide rather than ATP. The process thus generates an mRNA product with >90% functional cap post-transcription. The process is an efficient “one-pot” mRNA production method that includes, for example, the GAG trinucleotide (GpppAG; ^{m}GpppAₘG) in equimolar concentration with the NTPs, GTP, ATP, CTP and UTP. The process features an “A-start” DNA template that initiates transcription with 5′ adenosine (A). As defined herein, “A-start” and “G-start” DNA templates are double-stranded DNA having requisite nucleosides in the template strand, such that the coding strand (and corresponding mRNA) begin with A or G, respectively. For example, a G-start DNA template features a template strand having the nucleobases CC complementary to GG immediately downstream of the TATA box in the T7 promoter (referencing the coding strand), and an A-start DNA template features a template strand having the nucleobases TC complementary to the AG immediately downstream of the TATA box in the T7 promoter (referencing the coding strand).

An exemplary T7 promoter sequence featured in an A-start DNA template of the present disclosure is depicted here:

5′ TAATACGACTCACTATAAGNNNNNNNNNN . . . 3′
3′ ATTATGCTGAGTGATATTCNNNNNNNNNN . . . 3′

The trinucleotide-based capping methods described herein provide flexibility in dictating the penultimate nucleobase. The trinucleotide capping methods of the present disclosure provide efficient production of capped mRNA, for example, 95-98% capped mRNA with a natural cap 1 structure.

### Trinucleotide Caps

Provided herein are co-transcriptional capping methods for ribonucleic acid (RNA) synthesis. That is, RNA is produced in a “one-pot” reaction, without the need for a separate capping reaction. Thus, the methods, in some embodiments, comprise reacting a DNA template with a T7 RNA polymerase variant, nucleoside triphosphates, and a cap analog under in vitro transcription reaction conditions to produce RNA transcript.

A cap analog may be, for example, a dinucleotide cap, a trinucleotide cap, or a tetranucleotide cap. In some embodiments, a cap analog is a dinucleotide cap. In some embodiments, a cap analog is a trinucleotide cap. In some embodiments, a cap analog is a tetranucleotide cap.

A trinucleotide cap, in some embodiments, comprises a compound of formula (I)

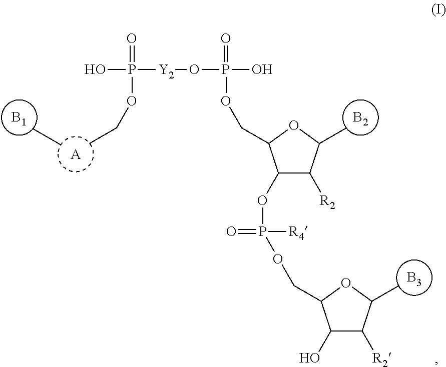

or a stereoisomer, tautomer or salt thereof, wherein

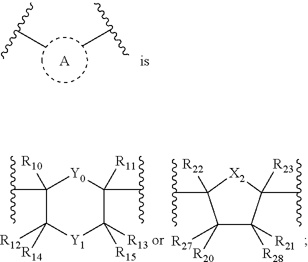

ring B₁ is a modified or unmodified Guanine;

ring B₂ and ring B₃ each independently is a nucleobase or a modified nucleobase;

X₂ is O, S(O)ₚ, NR₂₄, or CR₂₅R₂₆ in which p is 0, 1, or 2;

Y₀ is O or CR₆R₇;

Y1 is O, S(O)ₙ, CR₆R₇, or NR₈, in which n is 0, 1, or 2;

each [symbol] is a single bond or absent, wherein when each [symbol] is a single bond, Yi is O, S(O)ₙ, CR₆R₇, or NR₈; and when each [symbol] is absent, Y₁ is void;

Y₂ is (OP(O)R₄)ₘ in which m is 0, 1, or 2, or —O—(CR₄₀R₄₁)u-Q₀-(CR₄₂R₄₃)v-, in which Q₀ is a bond, O, S(O)ᵣ, NR₄₄, or CR₄₅R₄₆, r is 0, 1, or 2, and each of u and v independently is 1, 2, 3 or 4;

each R₂ and R₂′ independently is halo, LNA, or OR₃;

each R₃ independently is H, C₁-C₆ alkyl, C₂-C₆ alkenyl, or C₂-C₆ alkynyl and R₃, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;

each R₄ and R₄′ independently is H, halo, C₁-C₆ alkyl, OH, SH, SeH, or BH₃⁻;

each of R₆, R₇, and R₈, independently, is -Q₁-T₁, in which Q₁ is a bond or C₁-C₃ alkyl linker optionally substituted with one or more of halo, cyano, OH and C₁-C₆ alkoxy, and T₁ is H, halo, OH, COOH, cyano, or Rₛ₁, in which Rₛ₁ is C₁-C₃ alkyl, C₂-C₆ alkenyl, C₂-C₆ alkynyl, C₁-C₆ alkoxyl, C(O)O—C₁-C₆ alkyl, C₃-C₈ cycloalkyl, C₆-C₁₀ aryl, NR₃₁R₃₂, (NR₃₁R₃₂R₃₃)⁺, 4 to 12-membered heterocycloalkyl, or 5- or 6-membered heteroaryl, and Rₛ₁ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₈ cycloalkyl, C₆-C₁₀ aryl, 4 to 12-membered heterocycloalkyl, and 5- or 6-membered heteroaryl;

each of R₁₀, R₁₁, R₁₂, R₁₃ R₁₄, and R₁₅, independently, is -Q₂-T₂, in which Q₂ is a bond or C₁-C₃ alkyl linker optionally substituted with one or more of halo, cyano, OH and C₁-C₆ alkoxy, and T₂ is H, halo, OH, NH₂, cyano, NO₂, N₃, Rₛ₂, or ORₛ₂, in which Rₛ₂ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, NR₃₁R₃₂, (NR₃₁R₃₂R₃₃)⁺, 4 to 12-membered heterocycloalkyl, or 5- or 6-membered heteroaryl, and Rₛ₂ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₈ cycloalkyl, C₆-C₁₀ aryl, 4 to 12-membered heterocycloalkyl, and 5- or 6-membered heteroaryl; or alternatively Ru together with R₁₄ is oxo, or R₁₃ together with R₁₅ is oxo,

each of R₂₀, R₂₁, R₂₂, and R₂₃ independently is -Q₃-T₃, in which Q₃ is a bond or C₁-C₃ alkyl linker optionally substituted with one or more of halo, cyano, OH and C₁-C₆ alkoxy, and T₃ is H, halo, OH, NH₂, cyano, NO₂, N₃, Rₛ₃, or ORₛ₃, in which Rₛ₃ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, mono-C₁-C₆ alkylamino, di-C₁-C₆ alkylamino, 4 to 12-membered heterocycloalkyl, or 5- or 6-membered heteroaryl, and Rₛ₃ is optionally substituted with one or more substituents selected from the group consisting of halo, OH, oxo, C₁-C₆ alkyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl;

each of R₂₄, R₂₅, and R₂₆ independently is H or C₁-C₆ alkyl;

each of R₂₇ and R₂₈ independently is H or OR₂₉; or R₂₇ and R₂₈ together form O—R₃₀—O; each R₂₉ independently is H, C₁-C₆ alkyl, C₂-C₆ alkenyl, or C₂-C₆ alkynyl and R₂₉, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;

R₃₀ is C₁-C₆ alkylene optionally substituted with one or more of halo, OH and C₁-C₆ alkoxyl;

each of R₃₁, R₃₂, and R₃₃, independently is H, C₁-C₆ alkyl, C₃-C₈ cycloalkyl, C₆-C₁₀ aryl, 4 to 12-membered heterocycloalkyl, or 5- or 6-membered heteroaryl;

each of R₄₀, R₄₁, R₄₂, and R₄₃ independently is H, halo, OH, cyano, N₃, OP(O)R₄₇R₄₈, or C₁-C₆ alkyl optionally substituted with one or more OP(O)R₄₇R₄₈, or one R₄₁ and one R₄₃, together with the carbon atoms to which they are attached and Q₀, form C₄-C₁₀ cycloalkyl, 4- to 14-membered heterocycloalkyl, C₆-C₁₀ aryl, or 5- to 14-membered heteroaryl, and each of the cycloalkyl, heterocycloalkyl, phenyl, or 5- to 6-membered heteroaryl is optionally substituted with one or more of OH, halo, cyano, N₃, oxo, OP(O)R₄₇R₄₈, C₁-C₆ alkyl, C₁-C₆ haloalkyl, COOH, C(O)O—C₁-C₆ alkyl, C₁-C₆ alkoxyl, C₁-C₆ haloalkoxyl, amino, mono-C₁-C₆ alkylamino, and di-C₁-C₆ alkylamino;

R₄₄ is H, C₁-C₆ alkyl, or an amine protecting group;

each of R₄₅ and R₄₆ independently is H, OP(O)R₄₇R₄₈, or C₁-C₆ alkyl optionally substituted with one or more OP(O)R₄₇R₄₈, and

each of R₄₇ and R₄₈, independently is H, halo, C₁-C₆ alkyl, OH, SH, SeH, or BH₃⁻.

It should be understood that a cap analog, as provided herein, may include any of the cap analogs described in International Publication No. WO 2017/066797, published on 20 Apr. 2017, incorporated by reference herein in its entirety.

In some embodiments, the B₂ middle position can be a non-ribose molecule, such as arabinose.

In some embodiments R₂ is ethyl-based.

Thus, in some embodiments, a trinucleotide cap comprises the following structure:

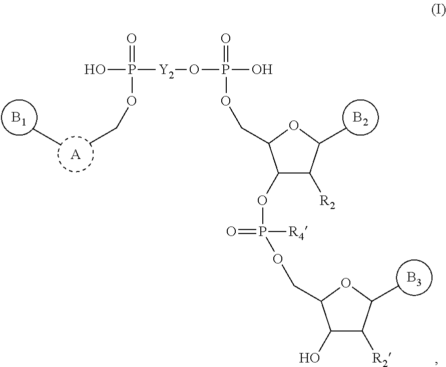

In other embodiments, a trinucleotide cap comprises the following structure:

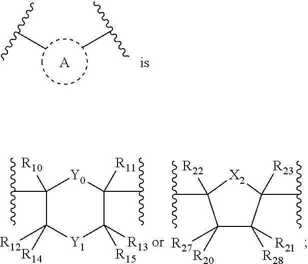

In yet other embodiments, a trinucleotide cap comprises the following structure:

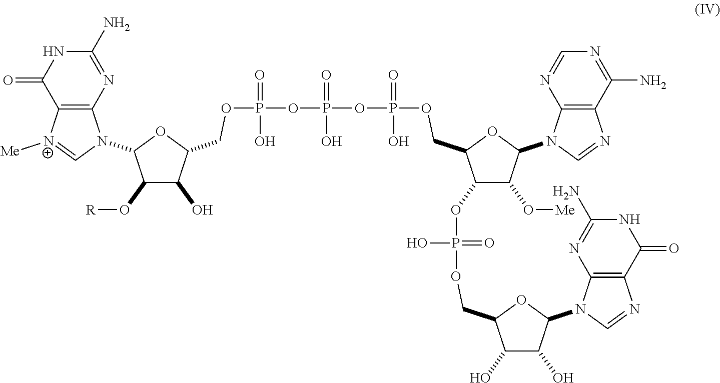

In still other embodiments, a trinucleotide cap comprises the following structure:

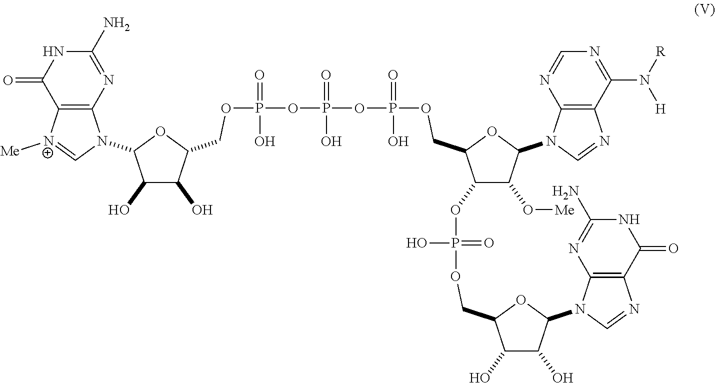

A trinucleotide cap, in some embodiments, comprises a sequence selected from the following sequences: GAA, GAC, GAG, GAU, GCA, GCC, GCG, GCU, GGA, GGC, GGG, GGU, GUA, GUC, GUG, and GUU.

In some embodiments, a trinucleotide cap comprises a sequence selected from the following sequences: m⁷GpppApA, m⁷GpppApC, m⁷GpppApG, m⁷GpppApU, m⁷GpppCpA, m⁷GpppCpC, m⁷GpppCpG, m⁷GpppCpU, m⁷GpppGpA, m⁷GpppGpC, m⁷GpppGpG, m⁷GpppGpU, m⁷GpppUpA, m⁷GpppUpC, m⁷GpppUpG, and m⁷GpppUpU.

A trinucleotide cap, in some embodiments, comprises a sequence selected from the following sequences: m⁷G_{3′OMe}pppApA, m⁷G_{3′OMe}pppApC, m⁷G_{3′OMe}pppApG, m⁷G_{3′OMe}pppApU, m⁷G_{3′OMe}pppCpA, m⁷G_{3′OMe}pppCpC, m⁷G_{3′OMe}pppCpG, m⁷G_{3′OMe}pppCpU, m⁷G_{3′OMe}pppGpA, m⁷G_{3′OMe}pppGpC, m⁷G_{3′OMe}pppGpG, m⁷G_{3′OMe}pppGpU, m⁷G_{3′OMe}pppUpA, m⁷G_{3′OMe}pppUpC, m⁷G_{3′OMe}pppUpG, and m⁷G_{3′OMe}pppUpU.

A trinucleotide cap, in other embodiments, comprises a sequence selected from the following sequences: m⁷G_{3′OMe}pppA_{2′OMe}pA, m⁷G_{3′OMe}pppA_{2′OMe}pC, m⁷G_{3′OMe}pppA_{2′OMe}pG, m⁷G_{3′OMe}pppA_{2′OMe}pU, m⁷G_{3′OMe}pppC_{2′OMe}pA, m⁷G_{3′OMe}pppC_{2′OMe}pC, m⁷G_{3′OMe}pppC_{2′OMe}pG, m⁷G_{3′OMe}pppC_{2′OMe}pU, m⁷G_{3′OMe}pppG_{2′OMe}pA, m⁷G_{3′OMe}pppG_{2′OMe}pC, m⁷G_{3′OMe}pppG_{2′OMe}pG, m⁷G_{3′OMe}pppG_{2′OMe}pU, m⁷G_{3′OMe}pppU_{2′OMe}pA, m⁷G_{3′OMe}pppU_{2′OMe}pC, m⁷G_{3′OMe}pppU_{2′OMe}pG, and m⁷G_{3′OMe}pppU_{2′OMe}pU.

A trinucleotide cap, in still other embodiments, comprises a sequence selected from the following sequences: m⁷GpppA_{2′OMe}pA, m⁷GpppA_{2′OMe}pC, m⁷GpppA_{2′OMe}pG, m⁷GpppA_{2′OMe}pU, m⁷GpppC_{2′OMe}pA, m⁷GpppC_{2′OMe}pC, m⁷GpppC_{2′OMe}pG, m⁷GpppC_{2′OMe}pU, m⁷GpppG_{2′OMe}pA, m⁷GpppG_{2′OMe}pC, m⁷GpppG_{2′OMe}pG, m⁷GpppG_{2′OMe}pU, m⁷GpppU_{2′OMe}pA, m⁷GpppU_{2′OMe}pC, m⁷GpppU_{2′OMe}pG, and m⁷GpppU_{2′OMe}pU.

A trinucleotide cap, in further embodiments, comprises a sequence selected from the following sequences: m⁷Gpppm⁶A_{2′OMe}pA, m⁷Gpppm⁶A_{2′OMe}pC, and m⁷Gpppm⁶A_{2′OMe}pG, m⁷Gpppm⁶A_{2′OMe}pU

A trinucleotide cap, in yet other embodiments, comprises a sequence selected from the following sequences: m⁷Gpppe⁶A_{2′OMe}pA, m⁷Gpppe⁶A_{2′OMe}pC, and m⁷Gpppe⁶A_{2′OMe}pG, In some embodiments, a trinucleotide cap comprises GAG. In some embodiments, a trinucleotide cap comprises GCG. In some embodiments, a trinucleotide cap comprises GUG. In some embodiments, a trinucleotide cap comprises GGG.

### Transcription

Some aspects of the present disclosure provide co-transcriptional capping methods that comprise reacting a DNA template with a RNA polymerase (e.g., T7 RNA polymerase), nucleoside triphosphates, and a trinucleotide cap analog under in vitro transcription reaction conditions to produce RNA transcript. A RNA transcript, in some embodiments, is a messenger RNA (mRNA) that includes a nucleotide sequence encoding a polypeptide (e.g., protein or peptide) of interest (e.g., biologics, antibodies, antigens (vaccines), and therapeutic proteins) linked to a polyA tail. In some embodiments, the mRNA is modified mRNA (mmRNA), which includes at least one modified nucleotide. In some embodiments, a modified mRNA is comprised of one or more RNA elements.

IVT conditions typically require a purified linear DNA template containing a promoter, nucleoside triphosphates, a buffer system that includes dithiothreitol (DTT) and magnesium ions, and a RNA polymerase. The exact conditions used in the transcription reaction depend on the amount of RNA needed for a specific application. Typical IVT reactions are performed by incubating a DNA template with a RNA polymerase and nucleoside triphosphates, including GTP, ATP, CTP, and UTP (or nucleotide analogs) in a transcription buffer. A RNA transcript having a 5′ terminal guanosine triphosphate is produced from this reaction.

A DNA template may encode a polypeptide of interest. A DNA template, in some embodiments, includes a RNA polymerase promoter (e.g., a T7 RNA polymerase promoter) located 5′ from and operably linked to a polynucleotide encoding a polypeptide of interest. A DNA template may also include a nucleotide sequence encoding a polyadenylation (polyA) tail located at the 3′ end of the gene of interest.

In some embodiments, the DNA template includes a 2′-deoxythymidine residue at template position +1. In some embodiments, the DNA template includes a 2′-deoxycytidine residue at template position +1. In some embodiments, the DNA template includes a 2′-deoxyadenosine residue at template position +1. In some embodiments, the DNA template includes a 2′-deoxyguanosine residue at template position +1.

In some embodiments, use of a DNA template that includes a 2′-deoxythymidine residue or 2′-deoxycytidine residue at template position +1 results in the production of RNA transcript, wherein greater than 80% (e.g., greater than 85%, greater than 90%, or greater than 95%) of the RNA transcript produced includes a functional cap. Thus, in some embodiments, a DNA template used, for example, in an IVT reaction, includes a 2′-deoxythymidine residue at template position +1. In other embodiments, a DNA template used, for example, in an IVT reaction, includes a 2′-deoxycytidine residue at template position +1.

The addition of nucleoside triphosphates (NTPs) to the 3′ end of a growing RNA strand is catalyzed by a RNA polymerase, such as T7 RNA polymerase. In some embodiments, the RNA polymerase is present in a reaction (e.g., an IVT reaction) at a concentration of 0.01 mg/ml to 1 mg/ml. For example, the RNA polymerase may be present in a reaction at a concentration of 0.01 mg/mL, 0.05 mg/ml, 0.1 mg/ml, 0.5 mg/ml or 1.0 mg/ml.

In some embodiments, a co-transcriptional capping method for RNA synthesis comprises reacting a DNA template with a RNA polymerase, nucleoside triphosphates, and a trinucleotide cap (e.g., comprising sequence GpppA_{3′OMe}pG), under in vitro transcription reaction conditions to produce RNA transcript, wherein the DNA template includes a 2′-deoxythymidine residue or a 2′-deoxycytidine residue at template position +1.

The combination of a RNA polymerase with a trinucleotide cap analog (e.g., GpppA_{3′OMe}pG), in an in vitro transcription reaction, for example, results in the production of RNA transcript, wherein greater than 80% of the RNA transcript produced includes a functional cap. In some embodiments, greater than 85% of the RNA transcript produced includes a functional cap. In some embodiments, greater than 90% of the RNA transcript produced includes a functional cap. In some embodiments, greater than 95% of the RNA transcript produced includes a functional cap. In some embodiments, greater than 96% of the RNA transcript produced includes a functional cap. In some embodiments, greater than 97% of the RNA transcript produced includes a functional cap. In some embodiments, greater than 98% of the RNA transcript produced includes a functional cap. In some embodiments, greater than 99% of the RNA transcript produced includes a functional cap.

In some embodiments, the disclosure provides an mRNA, wherein the 5′ UTR is comprised of a 5′ trinucleotide cap and a GC-rich RNA element comprising a nucleotide sequence selected from a group consisting of: SEQ ID NO: 1, SEQ ID NO: 2, SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 and SEQ ID NO: 28. In some embodiments, the disclosure provides an mRNA, wherein the 5′ UTR is comprised of a 5′ trinucleotide cap and a GC-rich RNA element, wherein the 5′ UTR sequence is selected from a group consisting of: SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, and SEQ ID NO: 82. In some embodiments, the disclosure provides an mRNA, wherein the 5′ UTR is comprised of a 5′ trinucleotide cap and a GC-rich RNA element, wherein the 5′ UTR sequence is set for by SEQ ID NO: 74. In some embodiments, the disclosure provides an mRNA, wherein the 5′ UTR is comprised of a 5′ trinucleotide cap and a GC-rich RNA element, wherein the 5′ UTR sequence is set for by SEQ ID NO: 73.

In some embodiments, the disclosure provides an mRNA, wherein the 5′ UTR is comprised of a 5′ trinucleotide cap and a C-rich RNA element comprising a nucleotide sequence selected from a group consisting of: SEQ ID NO: 29, SEQ ID NO: 30, SEQ ID NO: 31, SEQ ID NO: 32, SEQ ID NO: 33 and SEQ ID NO: 34. In some embodiments, the disclosure provides an mRNA, wherein the 5′ UTR is comprised of a 5′ trinucleotide cap and a C-rich RNA element, wherein the 5′ UTR sequence is selected from a group consisting of: SEQ ID NO: 83, SEQ ID NO: 84, SEQ ID NO: 85, and SEQ ID NO: 86. In some embodiments, the disclosure provides an mRNA, wherein the 5′ UTR is comprised of a 5′ trinucleotide cap and a C-rich RNA element, wherein the 5′ UTR sequence is set for by SEQ ID NO: 84. In some embodiments, the disclosure provides an mRNA, wherein the 5′ UTR is comprised of a 5′ trinucleotide cap and a C-rich RNA element, wherein the 5′ UTR sequence is set for by SEQ ID NO: 86.

In some embodiments, the disclosure provides an mRNA, wherein the 5′ UTR is comprised of a 5′ trinucleotide cap, a C-rich RNA element and a GC-rich RNA element comprising a nucleotide sequence selected from a group consisting of: SEQ ID NO: 1, SEQ ID NO: 2, SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 and SEQ ID NO: 28. In some embodiments, the disclosure provides an mRNA, wherein the 5′ UTR is comprised of a 5′ trinucleotide cap, a GC-rich RNA element and a C-rich RNA element comprising a nucleotide sequence selected from a group consisting of: SEQ ID NO: 29, SEQ ID NO: 30, SEQ ID NO: 31, SEQ ID NO: 32, SEQ ID NO: 33 and SEQ ID NO: 34. In some embodiments, the disclosure provides an mRNA, wherein the 5′ UTR is comprised of a 5′ trinucleotide cap, a GC-rich RNA element and a C-rich RNA element, wherein the 5′ UTR sequence is selected from a group consisting of: SEQ ID NO: 87, SEQ ID NO: 88, and SEQ ID NO: 89. In some embodiments, the disclosure provides an mRNA, wherein the 5′ UTR is comprised of a 5′ trinucleotide cap, a GC-rich RNA element and a C-rich RNA element, wherein the 5′ UTR sequence is set forth by SEQ ID NO: 87. In some embodiments, the disclosure provides an mRNA, wherein the 5′ UTR is comprised of a 5′ trinucleotide cap, a GC-rich RNA element and a C-rich RNA element, wherein the 5′ UTR sequence is set forth by SEQ ID NO: 88. In some embodiments, the disclosure provides an mRNA, wherein the 5′ UTR is comprised of a 5′ trinucleotide cap, a GC-rich RNA element and a C-rich RNA element, wherein the 5′ UTR sequence is set forth by SEQ ID NO: 89.

### Poly-A Tails

In some embodiments, a polynucleotide comprising an mRNA encoding a polypeptide of the present disclosure further comprises a poly A tail. In further embodiments, terminal groups on the poly-A tail can be incorporated for stabilization. In other embodiments, a poly-A tail comprises des-3′ hydroxyl tails. The useful poly-A tails can also include structural moieties or 2′-Omethyl modifications as taught by Li et al. (2005) Current Biology 15:1501-1507.

In one embodiment, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present disclosure are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone.

### Start Codon Region

In some embodiments, an mRNA of the present disclosure further comprises regions that are analogous to or function like a start codon region.

In some embodiments, the translation of a polynucleotide initiates on a codon which is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG. See Touriol et al. (2003) Biology of the Cell 95:169-178 and Matsuda and Mauro (2010) PLoS ONE 5:11. As a non-limiting example, the translation of a polynucleotide begins on the alternative start codon ACG. As another non-limiting example, polynucleotide translation begins on the alternative start codon CUG. As yet another non-limiting example, the translation of a polynucleotide begins on the alternative start codon GUG.

Nucleotides flanking a codon that initiates translation such as, but not limited to, a start codon or an alternative start codon, are known to affect the translation efficiency, the length and/or the structure of the polynucleotide. See, e.g., Matsuda and Mauro (2010) PLoS ONE 5:11. Masking any of the nucleotides flanking a codon that initiates translation can be used to alter the position of translation initiation, translation efficiency, length and/or structure of a polynucleotide.

In some embodiments, a masking agent is used near the start codon or alternative start codon in order to mask or hide the codon to reduce the probability of translation initiation at the masked start codon or alternative start codon. Non-limiting examples of masking agents include antisense locked nucleic acids (LNA) polynucleotides and exon-junction complexes (EJCs). See, e.g., Matsuda and Mauro (2010) PLoS ONE 5:11, describing masking agents LNA polynucleotides and EJCs.

In another embodiment, a masking agent is used to mask a start codon of a polynucleotide in order to increase the likelihood that translation will initiate on an alternative start codon. In some embodiments, a masking agent is used to mask a first start codon or alternative start codon in order to increase the chance that translation will initiate on a start codon or alternative start codon downstream to the masked start codon or alternative start codon.

In some embodiments, a start codon or alternative start codon is located within a perfect complement for a miR binding site. The perfect complement of a miR binding site can help control the translation, length and/or structure of the polynucleotide similar to a masking agent. As a non-limiting example, the start codon or alternative start codon is located in the middle of a perfect complement for a miR-122 binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide is removed from the polynucleotide sequence in order to have the translation of the polynucleotide begin on a codon which is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence in order to have translation initiate on a downstream start codon or alternative start codon. The polynucleotide sequence where the start codon was removed can further comprise at least one masking agent for the downstream start codon and/or alternative start codons in order to control or attempt to control the initiation of translation, the length of the polynucleotide and/or the structure of the polynucleotide.

### Stop Codon Region

In some embodiments, mRNA of the present disclosure can further comprise at least one stop codon or at least two stop codons before the 3′ untranslated region (UTR). The stop codon can be selected from UGA, UAA, and UAG. In some embodiments, the polynucleotides of the present disclosure include the stop codon UGA and one additional stop codon. In a further embodiment the addition stop codon can be UAA. In another embodiment, the polynucleotides of the present disclosure include three stop codons, four stop codons, or more.

### RNA Chemical Modifications

Numerous approaches for the chemical modification of mRNA to improve translation efficiency and reduce immunogenicity are known, including modifications at the 5′ cap, 5′ and 3′-UTRs, the open reading frame, and the poly(A) tail (Sahin et al., (2014) Nat Rev Drug Discovery 13:759-780). For example, pseudouridine (ψ) modified mRNA was shown to increased expression of encoded erythropoietin (Kariko et al., (2012) Mol Ther 20:948-953). A combination of 2-thiouridine (s2U) and 5-methylcytidine (5meC) in modified mRNAs was shown to extend the expression of encoded protein (Kormann et al., (2011) Nat Biotechnol 29:154-157). A recent study demonstrated the induction of vascular regeneration using modified (5meC and w) mRNA encoding human vascular endothelial growth factor (Zangi et al., (2013) Nat Biotechnol 31:898-907). These studies demonstrate the utility of incorporating chemically modified nucleotides to achieve mRNA structural and functional optimization.

Accordingly, in some embodiments, an mRNA described herein comprises a modification, wherein the modification is the incorporation of one or more chemically modified nucleotides. In some embodiments, one or more chemically modified nucleotides is incorporated into the initiation codon of the mRNA and functions to increases binding affinity between the initiation codon and the anticodon of the initiator Met-tRNAiMet. In some embodiments, the one or more chemically modified nucleotides is 2-thiouridine. In some embodiments, the one or more chemically modified nucleotides is 2′-O-methyl-2-thiouridine. In some embodiments, the one or more chemically modified nucleotides is 2-selenouridine. In some embodiments, the one or more chemically modified nucleotides is 2′-O-methyl ribose. In some embodiments, the one or more chemically modified nucleotides is selected from a locked nucleic acid, inosine, 2-methylguanosine, or 6-methyl-adenosine. In some embodiments, deoxyribonucleotides are incorporated into mRNA. An mRNA of the disclosure may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

Modified mRNAs

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (™⁵U), 1-taurinomethyl-pseudouridine, 5-taurinomethyl-2-thio-uridine(™⁵s²U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s²U), 1-methyl-4-thio-pseudouridine (m¹ s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxyprop yl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Urn), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine. In some aspects, the modified uridine is N1-methyl-pseudouridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k2C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include α-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms² m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶₂A), N6-hydroxynoryalylcarbamoyl-adenosine (hn⁶A), 2-methylthio-N6-hydroxynoryalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶₂Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include α-thio-guanosine, inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²₂G), N2,7-dimethyl-guanosine (m^{2,7}G), N2, N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²2 Gm), 1-methyl-2′-O-methyl-guanosine (m¹Gm), N2,7-dimethyl-2′-O-methyl-guanosine (m²′⁷Gm), 2′-O-methyl-inosine (Im), 1,2′-O-dimethyl-inosine (m¹Im), 2′-O-ribosylguanosine (phosphate) (Gr(p)), 1-thio-guanosine, 06-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is pseudouridine (ψ), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified cytosine. Exemplary nucleobases and nucleosides having a modified cytosine include N4-acetyl-cytidine (ac⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, 2-thio-cytidine (s²C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified adenine. Exemplary nucleobases and nucleosides having a modified adenine include 7-deaza-adenine, 1-methyl-adenosine (m¹A), 2-methyl-adenine (m²A), N6-methyl-adenosine (m⁶A). In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified guanine. Exemplary nucleobases and nucleosides having a modified guanine include inosine (I), 1-methyl-inosine (m¹I), wyosine (imG), methylwyosine (mimG), 7-deaza-guanosine, 7-cyano-7-deaza-guanosine (preQ₀), 7-aminomethyl-7-deaza-guanosine (preQ₁), 7-methyl-guanosine (m⁷G), 1-methyl-guanosine (m¹G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is 1-methyl-pseudouridine (m¹ψ), 5-methoxy-uridine (mo⁵U), 5-methyl-cytidine (m⁵C), pseudouridine (ψ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases.)

In some embodiments, the mRNA comprises pseudouridine (ψ). In some embodiments, the mRNA comprises pseudouridine (ψ) and 5-methyl-cytidine (m⁵C). In some embodiments, the mRNA comprises 1-methyl-pseudouridine (m¹ψ). In some embodiments, the mRNA comprises 1-methyl-pseudouridine (m¹ψ) and 5-methyl-cytidine (m⁵C). In some embodiments, the mRNA comprises 2-thiouridine (s²U). In some embodiments, the mRNA comprises 2-thiouridine and 5-methyl-cytidine (m⁵C). In some embodiments, the mRNA comprises 5-methoxy-uridine (mo⁵U). In some embodiments, the mRNA comprises 5-methoxy-uridine (mo⁵U) and 5-methyl-cytidine (m⁵C). In some embodiments, the mRNA comprises 2′-O-methyl uridine. In some embodiments, the mRNA comprises 2′-O-methyl uridine and 5-methyl-cytidine (m⁵C). In some embodiments, the mRNA comprises N6-methyl-adenosine (m⁶A). In some embodiments, the mRNA comprises N6-methyl-adenosine (m⁶A) and 5-methyl-cytidine (m⁵C).

In certain embodiments, an mRNA of the disclosure is uniformly modified (i.e., fully modified, modified through-out the entire sequence) for a particular modification. For example, an mRNA can be uniformly modified with 5-methyl-cytidine (m⁵C), meaning that all cytosine residues in the mRNA sequence are replaced with 5-methyl-cytidine (m⁵C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mmRNAs of the present disclosure include, but are not limited to, those described in PCT Patent Application Publications: WO2012045075, WO2014081507, WO2014093924, WO2014164253, and WO2014159813.

The mmRNAs of the disclosure can include a combination of modifications to the sugar, the nucleobase, and/or the internucleoside linkage. These combinations can include any one or more modifications described herein.

Examples of modified nucleosides and modified nucleoside combinations are provided below in Table 9 and Table 10 These combinations of modified nucleotides can be used to form the mmRNAs of the disclosure. In certain embodiments, the modified nucleosides may be partially or completely substituted for the natural nucleotides of the mRNAs of the disclosure. As a non-limiting example, the natural nucleotide uridine may be substituted with a modified nucleoside described herein. In another non-limiting example, the natural nucleoside uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9% of the natural uridines) with at least one of the modified nucleoside disclosed herein.

TABLE 9
Combinations of Nucleoside Modifications

| Modified Nucleotide | Modified Nucleotide Combination |
|---|---|
| α-thio-cytidine | α-thio-cytidine/5-iodo-uridine |
| | α-thio-cytidine/N1-methyl-pseudouridine |
| | α-thio-cytidine/α-thio-uridine |
| | α-thio-cytidine/5-methyl-uridine |
| | α-thio-cytidine/pseudo-uridine |
| | about 50% of the cytosines are α-thio-cytidine |
| pseudoisocytidine | pseudoisocytidine/5-iodo-uridine |
| | pseudoisocytidine/N1-methyl-pseudouridine |
| | pseudoisocytidine/α-thio-uridine |
| | pseudoisocytidine/5-methyl-uridine |
| | pseudoisocytidine/pseudouridine |
| | about 25% of cytosines are pseudoisocytidine |
| | pseudoisocytidine/about 50% of uridines are N1- |
| | methyl-pseudouridine and about 50% of uridines |
| | are pseudouridine |
| | pseudoisocytidine/about 25% of uridines are N1- |
| | methyl-pseudouridine and about 25% of uridines |
| | are pseudouridine |
| pyrrolo-cytidine | pyrrolo-cytidine/5-iodo-uridine |
| | pyrrolo-cytidine/N1-methyl-pseudouridine |
| | pyrrolo-cytidine/α-thio-uridine |
| | pyrrolo-cytidine/5-methyl-uridine |
| | pyrrolo-cytidine/pseudouridine |
| | about 50% of the cytosines are pyrrolo-cytidine |
| 5-methyl-cytidine | 5-methyl-cytidine/5-iodo-uridine |
| | 5-methyl-cytidine/N1-methyl-pseudouridine |
| | 5-methyl-cytidine/α-thio-uridine |
| | 5-methyl-cytidine/5-methyl-uridine |
| | 5-methyl-cytidine/pseudouridine |
| | about 25% of cytosines are 5-methyl-cytidine |
| | about 50% of cytosines are 5-methyl-cytidine |
| | 5-methyl-cytidine/5-methoxy-uridine |
| | 5-methyl-cytidine/5-bromo-uridine |
| | 5-methyl-cytidine/2-thio-uridine |
| | 5-methyl-cytidine/about 50% of uridines are 2- |
| | thio-uridine |
| | about 50% of uridines are 5-methyl-cytidine/about |
| | 50% of uridines are 2-thio-uridine |
| N4-acetyl-cytidine | N4-acetyl-cytidine/5-iodo-uridine |
| | N4-acetyl-cytidine/N1-methyl-pseudouridine |
| | N4-acetyl-cytidine/α-thio-uridine |
| | N4-acetyl-cytidine/5-methyl-uridine |
| | N4-acetyl-cytidine/pseudouridine |
| | about 50% of cytosines are N4-acetyl-cytidine |
| | about 25% of cytosines are N4-acetyl-cytidine |
| | N4-acetyl-cytidine/5-methoxy-uridine |
| | N4-acetyl-cytidine/5-bromo-uridine |
| | N4-acetyl-cytidine/2-thio-uridine |
| | about 50% of cytosines are N4-acetyl-cytidine/ |
| | about 50% of uridines are 2-thio-uridine |

TABLE 10
Modified Nucleosides and Combinations Thereof

1-(2,2,2-Trifluoroethyl)pseudo-UTP
1-Ethyl-pseudo-UTP
1-Methyl-pseudo-U-alph-thio-TP
1-methyl-pseudouridine TP, ATP, GTP, CTP
1-methyl-pseudo-UTP/5-methyl-CTP/ATP/GTP
1-methyl-pseudo-UTP/CTP/ATP/GTP
1-Propyl-pseudo-UTP
25% 5-Aminoallyl-CTP + 75% CTP/25% 5-Methoxy-UTP + 75% UTP
25% 5-Aminoallyl-CTP + 75% CTP/75% 5-Methoxy-UTP + 25% UTP
25% 5-Bromo-CTP + 75% CTP/25% 5-Methoxy-UTP + 75% UTP
25% 5-Bromo-CTP + 75% CTP/75% 5-Methoxy-UTP + 25% UTP
25% 5-Bromo-CTP + 75% CTP/1-Methyl-pseudo-UTP
25% 5-Carboxy-CTP + 75% CTP/25% 5-Methoxy-UTP + 75% UTP
25% 5-Carboxy-CTP + 75% CTP/75% 5-Methoxy-UTP + 25% UTP
25% 5-Ethyl-CTP + 75% CTP/25% 5-Methoxy-UTP + 75% UTP
25% 5-Ethyl-CTP + 75% CTP/75% 5-Methoxy-UTP + 25% UTP
25% 5-Ethynyl-CTP + 75% CTP/25% 5-Methoxy-UTP + 75% UTP
25% 5-Ethynyl-CTP + 75% CTP/75% 5-Methoxy-UTP + 25% UTP
25% 5-Fluoro-CTP + 75% CTP/25% 5-Methoxy-UTP + 75% UTP
25% 5-Fluoro-CTP + 75% CTP/75% 5-Methoxy-UTP + 25% UTP
25% 5-Formyl-CTP + 75% CTP/25% 5-Methoxy-UTP + 75% UTP
25% 5-Formyl-CTP + 75% CTP/75% 5-Methoxy-UTP + 25% UTP
25% 5-Hydroxymethyl-CTP + 75% CTP/25% 5-Methoxy-UTP + 75%
UTP
25% 5-Hydroxymethyl-CTP + 75% CTP/75% 5-Methoxy-UTP + 25%
UTP
25% 5-Iodo-CTP + 75% CTP/25% 5-Methoxy-UTP + 75% UTP
25% 5-Iodo-CTP + 75% CTP/75% 5-Methoxy-UTP + 25% UTP
25% 5-Methoxy-CTP + 75% CTP/25% 5-Methoxy-UTP + 75% UTP
25% 5-Methoxy-CTP + 75% CTP/75% 5-Methoxy-UTP + 25% UTP
25% 5-Methyl-CTP + 75% CTP/25% 5-Methoxy-UTP + 75% 1-Methyl-
pseudo-UTP
25% 5-Methyl-CTP + 75% CTP/25% 5-Methoxy-UTP + 75% UTP
25% 5-Methyl-CTP + 75% CTP/50% 5-Methoxy-UTP + 50% 1-Methyl-
pseudo-UTP
25% 5-Methyl-CTP + 75% CTP/50% 5-Methoxy-UTP + 50% UTP
25% 5-Methyl-CTP + 75% CTP/5-Methoxy-UTP
25% 5-Methyl-CTP + 75% CTP/75% 5-Methoxy-UTP + 25% 1-Methyl-
pseudo-UTP
25% 5-Methyl-CTP + 75% CTP/75% 5-Methoxy-UTP + 25% UTP
25% 5-Phenyl-CTP + 75% CTP/25% 5-Methoxy-UTP + 75% UTP
25% 5-Phenyl-CTP + 75% CTP/75% 5-Methoxy-UTP + 25% UTP
25% 5-Trifluoromethyl-CTP + 75% CTP/25% 5-Methoxy-UTP + 75%
UTP
25% 5-Trifluoromethyl-CTP + 75% CTP/75% 5-Methoxy-UTP + 25%
UTP
25% 5-Trifluoromethyl-CTP + 75% CTP/1-Methyl-pseudo-UTP
25% N4-Ac-CTP + 75% CTP/25% 5-Methoxy-UTP + 75% UTP
25% N4-Ac-CTP + 75% CTP/75% 5-Methoxy-UTP + 25% UTP
25% N4-Bz-CTP + 75% CTP/25% 5-Methoxy-UTP + 75% UTP
25% N4-Bz-CTP + 75% CTP/75% 5-Methoxy-UTP + 25% UTP
25% N4-Methyl-CTP + 75% CTP/25% 5-Methoxy-UTP + 75% UTP
25% N4-Methyl-CTP + 75% CTP/75% 5-Methoxy-UTP + 25% UTP
25% Pseudo-iso-CTP + 75% CTP/25% 5-Methoxy-UTP + 75% UTP
25% Pseudo-iso-CTP + 75% CTP/75% 5-Methoxy-UTP + 25% UTP
25% 5-Bromo-CTP/75% CTP/Pseudo-UTP
25% 5-methoxy-UTP/25% 5-methyl-CTP/ATP/GTP
25% 5-methoxy-UTP/5-methyl-CTP/ATP/GTP
25% 5-methoxy-UTP/75% 5-methyl-CTP/ATP/GTP
25% 5-methoxy-UTP/CTP/ATP/GTP
25% 5-metoxy-UTP/50% 5-methyl-CTP/ATP/GTP
2-Amino-ATP
2-Thio-CTP
2-thio-pseudouridine TP, ATP, GTP, CTP
2-Thio-pseudo-UTP
2-Thio-UTP
3-Methyl-CTP
3-Methyl-pseudo-UTP
4-Thio-UTP
50% 5-Bromo-CTP + 50% CTP/1-Methyl-pseudo-UTP
50% 5-Hydroxymethyl-CTP + 50% CTP/1-Methyl-pseudo-UTP
50% 5-methoxy-UTP/5-methyl-CTP/ATP/GTP
50% 5-Methyl-CTP + 50% CTP/25% 5-Methoxy-UTP + 75% 1-Methyl-
pseudo-UTP
50% 5-Methyl-CTP + 50% CTP/25% 5-Methoxy-UTP + 75% UTP
50% 5-Methyl-CTP + 50% CTP/50% 5-Methoxy-UTP + 50% 1-Methyl-
pseudo-UTP
50% 5-Methyl-CTP + 50% CTP/50% 5-Methoxy-UTP + 50% UTP
50% 5-Methyl-CTP + 50% CTP/5-Methoxy-UTP
50% 5-Methyl-CTP + 50% CTP/75% 5-Methoxy-UTP + 25% 1-Methyl-
pseudo-UTP
50% 5-Methyl-CTP + 50% CTP/75% 5-Methoxy-UTP + 25% UTP
50% 5-Trifluoromethyl-CTP + 50% CTP/1-Methyl-pseudo-UTP
50% 5-Bromo-CTP/50% CTP/Pseudo-UTP
50% 5-methoxy-UTP/25% 5-methyl-CTP/ATP/GTP
50% 5-methoxy-UTP/50% 5-methyl-CTP/ATP/GTP
50% 5-methoxy-UTP/75% 5-methyl-CTP/ATP/GTP
50% 5-methoxy-UTP/CTP/ATP/GTP
5-Aminoallyl-CTP
5-Aminoallyl-CTP/5-Methoxy-UTP
5-Aminoallyl-UTP
5-Bromo-CTP
5-Bromo-CTP/5-Methoxy-UTP
5-Bromo-CTP/1-Methyl-pseudo-UTP
5-Bromo-CTP/Pseudo-UTP
5-bromocytidine TP, ATP, GTP, UTP
5-Bromo-UTP
5-Carboxy-CTP/5-Methoxy-UTP
5-Ethyl-CTP/5-Methoxy-UTP
5-Ethynyl-CTP/5-Methoxy-UTP
5-Fluoro-CTP/5-Methoxy-UTP
5-Formyl-CTP/5-Methoxy-UTP
5-Hydroxy- methyl-CTP/5-Methoxy-UTP
5-Hydroxymethyl-CTP
5-Hydroxymethyl-CTP/1-Methyl-pseudo-UTP
5-Hydroxymethyl-CTP/5-Methoxy-UTP
5-hydroxymethyl-cytidine TP, ATP, GTP, UTP
5-Iodo-CTP/5-Methoxy-UTP
5-Me-CTP/5-Methoxy-UTP
5-Methoxy carbonyl methyl-UTP
5-Methoxy-CTP/5-Methoxy-UTP
5-methoxy-uridine TP, ATP, GTP, UTP
5-methoxy-UTP
5-Methoxy-UTP
5-Methoxy-UTP/N6-Isopentenyl-ATP
5-methoxy-UTP/25%5-methyl-CTP/ATP/GTP
5-methoxy-UTP/5-methyl-CTP/ATP/GTP
5-methoxy-UTP/75% 5-methyl-CTP/ATP/GTP
5-methoxy-UTP/CTP/ATP/GTP
5-Methyl-2-thio-UTP
5-Methylaminomethyl-UTP
5-Methyl-CTP/5-Methoxy-UTP
5-Methyl-CTP/5-Methoxy-UTP(cap 0)
5-Methyl-CTP/5-Methoxy-UTP(No cap)
5-Methyl-CTP/25% 5-Methoxy-UTP + 75% 1-Methyl-pseudo-UTP
5-Methyl-CTP/25% 5-Methoxy-UTP + 75% UTP
5-Methyl-CTP/50% 5-Methoxy-UTP + 50% 1-Methyl-pseudo-UTP
5-Methyl-CTP/50% 5-Methoxy-UTP + 50% UTP
5-Methyl-CTP/5-Methoxy-UTP/N6-Me-ATP
5-Methyl-CTP/75% 5-Methoxy-UTP + 25% 1-Methyl-pseudo-UTP
5-Methyl-CTP/75% 5-Methoxy-UTP + 25% UTP
5-Phenyl-CTP/5-Methoxy-UTP
5-Trifluoro- methyl-CTP/5-Methoxy-UTP
5-Trifluoromethyl-CTP
5-Trifluoromethyl-CTP/5-Methoxy-UTP
5-Trifluoromethyl-CTP/1-Methyl-pseudo-UTP
5-Trifluoromethyl-CTP/Pseudo-UTP
5-Trifluoromethyl-UTP
5-trifluromethylcytidine TP, ATP, GTP, UTP
75% 5-Aminoallyl-CTP + 25% CTP/25% 5-Methoxy-UTP + 75% UTP
75% 5-Aminoallyl-CTP + 25% CTP/75% 5-Methoxy-UTP + 25% UTP
75% 5-Bromo-CTP + 25% CTP/25% 5-Methoxy-UTP + 75% UTP
75% 5-Bromo-CTP + 25% CTP/75% 5-Methoxy-UTP + 25% UTP
75% 5-Carboxy-CTP + 25% CTP/25% 5-Methoxy-UTP + 75% UTP
75% 5-Carboxy-CTP + 25% CTP/75% 5-Methoxy-UTP + 25% UTP
75% 5-Ethyl-CTP + 25% CTP/25% 5-Methoxy-UTP + 75% UTP
75% 5-Ethyl-CTP + 25% CTP/75% 5-Methoxy-UTP + 25% UTP
75% 5-Ethynyl-CTP + 25% CTP/25% 5-Methoxy-UTP + 75% UTP
75% 5-Ethynyl-CTP + 25% CTP/75% 5-Methoxy-UTP + 25% UTP
75% 5-Fluoro-CTP + 25% CTP/25% 5-Methoxy-UTP + 75% UTP
75% 5-Fluoro-CTP + 25% CTP/75% 5-Methoxy-UTP + 25% UTP
75% 5-Formyl-CTP + 25% CTP/25% 5-Methoxy-UTP + 75% UTP
75% 5-Formyl-CTP + 25% CTP/75% 5-Methoxy-UTP + 25% UTP
75% 5-Hydroxymethyl-CTP + 25% CTP/25% 5-Methoxy-UTP + 75%
UTP
75% 5-Hydroxymethyl-CTP + 25% CTP/75% 5-Methoxy-UTP + 25%
UTP
75% 5-Iodo-CTP + 25% CTP/25% 5-Methoxy-UTP + 75% UTP
75% 5-Iodo-CTP + 25% CTP/75% 5-Methoxy-UTP + 25% UTP
75% 5-Methoxy-CTP + 25% CTP/25% 5-Methoxy-UTP + 75% UTP
75% 5-Methoxy-CTP + 25% CTP/75% 5-Methoxy-UTP + 25% UTP
75% 5-methoxy-UTP/5-methyl-CTP/ATP/GTP
75% 5-Methyl-CTP + 25% CTP/25% 5-Methoxy-UTP + 75% 1-Methyl-
pseudo-UTP
75% 5-Methyl-CTP + 25% CTP/25% 5-Methoxy-UTP + 75% UTP
75% 5-Methyl-CTP + 25% CTP/50% 5-Methoxy-UTP + 50% 1-Methyl-
pseudo-UTP
75% 5-Methyl-CTP + 25% CTP/50% 5-Methoxy-UTP + 50% UTP
75% 5-Methyl-CTP + 25% CTP/5-Methoxy-UTP
75% 5-Methyl-CTP + 25% CTP/75% 5-Methoxy-UTP + 25% 1-Methyl-
pseudo-UTP
75% 5-Methyl-CTP + 25% CTP/75% 5-Methoxy-UTP + 25% UTP
75% 5-Phenyl-CTP + 25% CTP/25% 5-Methoxy-UTP + 75% UTP
75% 5-Phenyl-CTP + 25% CTP/75% 5-Methoxy-UTP + 25% UTP
75% 5-Trifluoromethyl-CTP + 25% CTP/25% 5-Methoxy-UTP + 75%
UTP
75% 5-Trifluoromethyl-CTP + 25% CTP/75% 5-Methoxy-UTP + 25%
UTP
75% 5-Trifluoromethyl-CTP + 25% CTP/1-Methyl-pseudo-UTP
75% N4-Ac-CTP + 25% CTP/25% 5-Methoxy-UTP + 75% UTP
75% N4-Ac-CTP + 25% CTP/75% 5-Methoxy-UTP + 25% UTP
75% N4-Bz-CTP + 25% CTP/25% 5-Methoxy-UTP + 75% UTP
75% N4-Bz-CTP + 25% CTP/75% 5-Methoxy-UTP + 25% UTP
75% N4-Methyl-CTP + 25% CTP/25% 5-Methoxy-UTP + 75% UTP
75% N4-Methyl-CTP + 25% CTP/75% 5-Methoxy-UTP + 25% UTP
75% Pseudo-iso-CTP + 25% CTP/25% 5-Methoxy-UTP + 75% UTP
75% Pseudo-iso-CTP + 25% CTP/75% 5-Methoxy-UTP + 25% UTP
75% 5-Bromo-CTP/25% CTP/1-Methyl-pseudo-UTP
75% 5-Bromo-CTP/25% CTP/Pseudo-UTP
75% 5-methoxy-UTP/25% 5-methyl-CTP/ATP/GTP
75% 5-methoxy-UTP/50% 5-methyl-CTP/ATP/GTP
75% 5-methoxy-UTP/75% 5-methyl-CTP/ATP/GTP
75% 5-methoxy-UTP/CTP/ATP/GTP
8-Aza-ATP
Alpha-thio-CTP
CTP/25% 5-Methoxy-UTP + 75% 1-Methyl-pseudo-UTP
CTP/25% 5-Methoxy-UTP + 75% UTP
CTP/50% 5-Methoxy-UTP + 50% 1-Methyl-pseudo-UTP
CTP/50% 5-Methoxy-UTP + 50% UTP
CTP/5-Methoxy-UTP
CTP/5-Methoxy-UTP (cap 0)
CTP/5-Methoxy-UTP(No cap)
CTP/75% 5-Methoxy-UTP + 25% 1-Methyl-pseudo-UTP
CTP/75% 5-Methoxy-UTP + 25% UTP
CTP/UTP(No cap)
N1-Me-GTP
N4-Ac-CTP
N4Ac-CTP/1-Methyl-pseudo-UTP
N4Ac-CTP/5-Methoxy-UTP
N4-acetyl-cytidine TP, ATP, GTP, UTP
N4-Bz-CTP/5-Methoxy-UTP
N4-methyl CTP
N4-Methyl-CTP/5-Methoxy-UTP
Pseudo-iso-CTP/5-Methoxy-UTP
PseudoU-alpha-thio-TP
pseudouridine TP, ATP, GTP, CTP
pseudo-UTP/5-methyl-CTP/ATP/GTP
UTP-5-oxyacetic acid Me ester
Xanthosine

According to the disclosure, polynucleotides of the disclosure may be synthesized to comprise the combinations or single modifications of Table 3 or Table 4.

Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP+75% CTP/25% 5-Methoxy-UTP+75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the polynucleotide. In this example all of the GTP and ATP nucleotides are left unmodified.

In certain embodiments, the present disclosure includes polynucleotides having at least 80%, at least 85%, at least 90%, at least 95%, at least 98%, or at least 99% sequence identity to any of the polynucleotide sequences described herein.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, mRNAs are made using IVT enzymatic synthesis methods. Methods of making polynucleotides by IVT are known in the art and are described in International Application PCT/US2013/30062, the contents of which are incorporated herein by reference in their entirety. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

MicroRNA (miRNA) Binding Sites

Nucleic acid molecules (e.g., RNA, e.g., mRNA) of the disclosure can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, nucleic acid molecules (e.g., RNA, e.g., mRNA) including such regulatory elements are referred to as including “sensor sequences.” Non-limiting examples of sensor sequences are described in U.S. Publication 2014/0200261, the contents of which are incorporated herein by reference in their entirety.

In some embodiments, a nucleic acid molecule (e.g., RNA, e.g., mRNA) of the disclosure comprises an open reading frame (ORF) encoding a polypeptide of interest and further comprises one or more miRNA binding site(s). Inclusion or incorporation of miRNA binding site(s) provides for regulation of nucleic acid molecules (e.g., RNA, e.g., mRNA) of the disclosure, and in turn, of the polypeptides encoded therefrom, based on tissue-specific and/or cell-type specific expression of naturally-occurring miRNAs.

A miRNA, e.g., a natural-occurring miRNA, is a 19-25 nucleotide long noncoding RNA that binds to a nucleic acid molecule (e.g., RNA, e.g., mRNA) and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27(1):91-105. miRNA profiling of the target cells or tissues can be conducted to determine the presence or absence of miRNA in the cells or tissues. In some embodiments, a nucleic acid molecule (e.g., RNA, e.g., mRNA) of the disclosure comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

As used herein, the term “microRNA (miRNA or miR) binding site” refers to a sequence within a nucleic acid molecule, e.g., within a DNA or within an RNA transcript, including in the 5′UTR and/or 3′UTR, that has sufficient complementarity to all or a region of a miRNA to interact with, associate with or bind to the miRNA. In some embodiments, a nucleic acid molecule (e.g., RNA, e.g., mRNA) of the disclosure comprising an ORF encoding a polypeptide of interest and further comprises one or more miRNA binding site(s). In exemplary embodiments, a 5′UTR and/or 3′UTR of the nucleic acid molecule (e.g., RNA, e.g., mRNA) comprises the one or more miRNA binding site(s).

A miRNA binding site having sufficient complementarity to a miRNA refers to a degree of complementarity sufficient to facilitate miRNA-mediated regulation of a nucleic acid molecule (e.g., RNA, e.g., mRNA), e.g., miRNA-mediated translational repression or degradation of the nucleic acid molecule (e.g., RNA, e.g., mRNA). In exemplary aspects of the disclosure, a miRNA binding site having sufficient complementarity to the miRNA refers to a degree of complementarity sufficient to facilitate miRNA-mediated degradation of the nucleic acid molecule (e.g., RNA, e.g., mRNA), e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the nucleic acid molecule (e.g., RNA, e.g., mRNA) comprising the miRNA binding site. In other embodiments, the miRNA binding site has imperfect complementarity so that a RISC complex comprising the miRNA induces instability in the nucleic acid molecule (e.g., RNA, e.g., mRNA) comprising the miRNA binding site. In another embodiment, the miRNA binding site has imperfect complementarity so that a RISC complex comprising the miRNA represses transcription of the nucleic acid molecule (e.g., RNA, e.g., mRNA) comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a nucleic acid molecule (e.g., RNA, e.g., mRNA) of the disclosure, the nucleic acid molecule (e.g., RNA, e.g., mRNA) can be targeted for degradation or reduced translation, provided the miRNA in question is available. This can reduce off-target effects upon delivery of the nucleic acid molecule (e.g., RNA, e.g., mRNA). For example, if a nucleic acid molecule (e.g., RNA, e.g., mRNA) of the disclosure is not intended to be delivered to a tissue or cell but ends up is said tissue or cell, then a miRNA abundant in the tissue or cell can inhibit the expression of the gene of interest if one or multiple binding sites of the miRNA are engineered into the 5′UTR and/or 3′UTR of the nucleic acid molecule (e.g., RNA, e.g., mRNA).

For example, one of skill in the art would understand that one or more miR can be included in a nucleic acid molecule (e.g., an RNA, e.g., mRNA) to minimize expression in cell types other than lymphoid cells. In one embodiment, miR122 can be used. In another embodiment, miR126 can be used. In still another embodiment, multiple copies of these miRs or combinations may be used.

Conversely, miRNA binding sites can be removed from nucleic acid molecule (e.g., RNA, e.g., mRNA) sequences in which they naturally occur in order to increase protein expression in specific tissues. For example, a binding site for a specific miRNA can be removed from a nucleic acid molecule (e.g., RNA, e.g., mRNA) to improve protein expression in tissues or cells containing the miRNA.

In one embodiment, a nucleic acid molecule (e.g., RNA, e.g., mRNA) of the disclosure can include at least one miRNA-binding site in the 5′UTR and/or 3′UTR in order to regulate cytotoxic or cytoprotective mRNA therapeutics to specific cells such as, but not limited to, normal and/or cancerous cells. In another embodiment, a nucleic acid molecule (e.g., RNA, e.g., mRNA) of the disclosure can include two, three, four, five, six, seven, eight, nine, ten, or more miRNA-binding sites in the 5′-UTR and/or 3′-UTR in order to regulate cytotoxic or cytoprotective mRNA therapeutics to specific cells such as, but not limited to, normal and/or cancerous cells.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profilings in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/1eu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA binding sites can correspond to any known sequence, including non-limiting examples described in U.S. Publication Nos. 2014/0200261, 2005/0261218, and 2005/0059005, each of which are incorporated herein by reference in their entirety. Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126). Specifically, miRNAs are known to be differentially expressed in immune cells (also called hematopoietic cells), such as antigen presenting cells (APCs) (e.g., dendritic cells and monocytes), monocytes, monocytes, B lymphocytes, T lymphocytes, granulocytes, natural killer cells, etc. Immune cell specific miRNAs are involved in immunogenicity, autoimmunity, the immune response to infection, inflammation, as well as unwanted immune response after gene therapy and tissue/organ transplantation. Immune cell specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a nucleic acid molecule (e.g., RNA, e.g., mRNA) can be shut-off by adding miR-142 binding sites to the 3′-UTR of the polynucleotide, enabling more stable gene transfer in tissues and cells. miR-142 efficiently degrades exogenous nucleic acid molecules (e.g., RNA, e.g., mRNA) in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′UTR and/or 3′UTR of a nucleic acid molecule of the disclosure can selectively repress gene expression in antigen presenting cells through miR-142 mediated degradation, limiting antigen presentation in antigen presenting cells (e.g., dendritic cells) and thereby preventing antigen-mediated immune response after the delivery of the nucleic acid molecule (e.g., RNA, e.g., mRNA). The nucleic acid molecule (e.g., RNA, e.g., mRNA) is then stably expressed in target tissues or cells without triggering cytotoxic elimination.

In one embodiment, binding sites for miRNAs that are known to be expressed in immune cells, in particular, antigen presenting cells, can be engineered into a nucleic acid molecule (e.g., RNA, e.g., mRNA) of the disclosure to suppress the expression of the nucleic acid molecule (e.g., RNA, e.g., mRNA) in antigen presenting cells through miRNA mediated RNA degradation, subduing the antigen-mediated immune response. Expression of the nucleic acid molecule (e.g., RNA, e.g., mRNA) is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′UTR and/or 3′UTR of a nucleic acid molecule of the disclosure.

To further drive the selective degradation and suppression in APCs and macrophage, a nucleic acid molecule (e.g., RNA, e.g., mRNA) of the disclosure can include a further negative regulatory element in the 5′UTR and/or 3′UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p,miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p,miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11,288, the content of each of which is incorporated herein by reference in its entirety.)

miRNAs that are known to be expressed in the liver include, but are not limited to, miR-107, miR-122-3p, miR-122-5p, miR-1228-3p, miR-1228-5p, miR-1249, miR-129-5p, miR-1303, miR-151a-3p, miR-151a-5p, miR-152, miR-194-3p, miR-194-5p, miR-199a-3p, miR-199a-5p, miR-199b-3p, miR-199b-5p, miR-296-5p, miR-557, miR-581, miR-939-3p, and miR-939-5p. miRNA binding sites from any liver specific miRNA can be introduced to or removed from a nucleic acid molecule (e.g., RNA, e.g., mRNA) of the disclosure to regulate expression of the nucleic acid molecule (e.g., RNA, e.g., mRNA) in the liver. Liver specific miRNA binding sites can be engineered alone or further in combination with immune cell (e.g., APC) miRNA binding sites in a nucleic acid molecule (e.g., RNA, e.g., mRNA) of the disclosure. In one embodiment, miRNA binding sites that promote degradation of mRNAs by hepatocytes are present in an mRNA molecule agent.

miRNAs that are known to be expressed in the lung include, but are not limited to, let-7a-2-3p, let-7a-3p, let-7a-5p, miR-126-3p, miR-126-5p, miR-12′7-3p, miR-127-5p, miR-130a-3p, miR-130a-5p, miR-130b-3p, miR-130b-5p, miR-133a, miR-133b, miR-134, miR-18a-3p, miR-18a-5p, miR-18b-3p, miR-18b-5p, miR-24-1-5p, miR-24-2-5p, miR-24-3p, miR-296-3p, miR-296-5p, miR-32-3p, miR-337-3p, miR-337-5p, miR-381-3p, and miR-381-5p. miRNA binding sites from any lung specific miRNA can be introduced to or removed from a nucleic acid molecule (e.g., RNA, e.g., mRNA) of the disclosure to regulate expression of the nucleic acid molecule (e.g., RNA, e.g., mRNA) in the lung. Lung specific miRNA binding sites can be engineered alone or further in combination with immune cell (e.g., APC) miRNA binding sites in a nucleic acid molecule (e.g., RNA, e.g., mRNA) of the disclosure.

miRNAs that are known to be expressed in the heart include, but are not limited to, miR-1, miR-133a, miR-133b, miR-149-3p, miR-149-5p, miR-186-3p, miR-186-5p, miR-208a, miR-208b, miR-210, miR-296-3p, miR-320, miR-451a, miR-451b, miR-499a-3p, miR-499a-5p, miR-499b-3p, miR-499b-5p, miR-744-3p, miR-744-5p, miR-92b-3p, and miR-92b-5p. miRNA binding sites from any heart specific microRNA can be introduced to or removed from a nucleic acid molecule (e.g., RNA, e.g., mRNA) of the disclosure to regulate expression of the nucleic acid molecule (e.g., RNA, e.g., mRNA) in the heart. Heart specific miRNA binding sites can be engineered alone or further in combination with immune cell (e.g., APC) miRNA binding sites in a nucleic acid molecule (e.g., RNA, e.g., mRNA) of the disclosure.

miRNAs that are known to be expressed in the nervous system include, but are not limited to, miR-124-5p, miR-125a-3p, miR-125a-5p, miR-125b-1-3p, miR-125b-2-3p, miR-125b-5p,miR-1271-3p, miR-1271-5p, miR-128, miR-132-5p, miR-135a-3p, miR-135a-5p, miR-135b-3p, miR-135b-5p, miR-137, miR-139-5p, miR-139-3p, miR-149-3p, miR-149-5p, miR-153, miR-181c-3p, miR-181c-5p, miR-183-3p, miR-183-5p, miR-190a, miR-190b, miR-212-3p, miR-212-5p, miR-219-1-3p, miR-219-2-3p, miR-23a-3p, miR-23a-5p,miR-30a-5p, miR-30b-3p, miR-30b-5p, miR-30c-1-3p, miR-30c-2-3p, miR-30c-5p, miR-30d-3p, miR-30d-5p, miR-329, miR-342-3p, miR-3665, miR-3666, miR-380-3p, miR-380-5p, miR-383, miR-410, miR-425-3p, miR-425-5p, miR-454-3p, miR-454-5p, miR-483, miR-510, miR-516a-3p, miR-548b-5p, miR-548c-5p, miR-571, miR-7-1-3p, miR-7-2-3p, miR-7-5p, miR-802, miR-922, miR-9-3p, and miR-9-5p. miRNAs enriched in the nervous system further include those specifically expressed in neurons, including, but not limited to, miR-132-3p, miR-132-3p, miR-148b-3p, miR-148b-5p, miR-151a-3p, miR-151a-5p, miR-212-3p, miR-212-5p, miR-320b, miR-320e, miR-323a-3p, miR-323a-5p, miR-324-5p, miR-325, miR-326, miR-328, miR-922 and those specifically expressed in glial cells, including, but not limited to, miR-1250, miR-219-1-3p, miR-219-2-3p, miR-219-5p, miR-23a-3p, miR-23a-5p, miR-3065-3p, miR-3065-5p, miR-30e-3p, miR-30e-5p, miR-32-5p, miR-338-5p, and miR-657. miRNA binding sites from any CNS specific miRNA can be introduced to or removed from a nucleic acid molecule (e.g., RNA, e.g., mRNA) of the disclosure to regulate expression of the nucleic acid molecule (e.g., RNA, e.g., mRNA) in the nervous system. Nervous system specific miRNA binding sites can be engineered alone or further in combination with immune cell (e.g., APC) miRNA binding sites in a nucleic acid molecule (e.g., RNA, e.g., mRNA) of the disclosure.

miRNAs that are known to be expressed in the pancreas include, but are not limited to, miR-105-3p, miR-105-5p, miR-184, miR-195-3p, miR-195-5p, miR-196a-3p, miR-196a-5p, miR-214-3p, miR-214-5p, miR-216a-3p, miR-216a-5p, miR-30a-3p, miR-33a-3p, miR-33a-5p, miR-375, miR-7-1-3p, miR-7-2-3p, miR-493-3p, miR-493-5p, and miR-944. miRNA binding sites from any pancreas specific miRNA can be introduced to or removed from a nucleic acid molecule (e.g., RNA, e.g., mRNA) of the disclosure to regulate expression of the nucleic acid molecule (e.g., RNA, e.g., mRNA) in the pancreas. Pancreas specific miRNA binding sites can be engineered alone or further in combination with immune cell (e.g. APC) miRNA binding sites in a nucleic acid molecule (e.g., RNA, e.g., mRNA) of the disclosure.

miRNAs that are known to be expressed in the kidney include, but are not limited to, miR-122-3p, miR-145-5p, miR-17-5p, miR-192-3p, miR-192-5p, miR-194-3p, miR-194-5p, miR-20a-3p, miR-20a-5p, miR-204-3p, miR-204-5p, miR-210, miR-216a-3p, miR-216a-5p, miR-296-3p, miR-30a-3p, miR-30a-5p, miR-30b-3p, miR-30b-5p, miR-30c-1-3p, miR-30c-2-3p, miR30c-5p, miR-324-3p, miR-335-3p, miR-335-5p, miR-363-3p, miR-363-5p, and miR-562. miRNA binding sites from any kidney specific miRNA can be introduced to or removed from a nucleic acid molecule (e.g., RNA, e.g., mRNA) of the disclosure to regulate expression of the nucleic acid molecule (e.g., RNA, e.g., mRNA) in the kidney. Kidney specific miRNA binding sites can be engineered alone or further in combination with immune cell (e.g., APC) miRNA binding sites in a nucleic acid molecule (e.g., RNA, e.g., mRNA) of the disclosure.

miRNAs that are known to be expressed in the muscle include, but are not limited to, let-7g-3p, let-7g-5p, miR-1, miR-1286, miR-133a, miR-133b, miR-140-3p, miR-143-3p, miR-143-5p, miR-145-3p, miR-145-5p, miR-188-3p, miR-188-5p, miR-206, miR-208a, miR-208b, miR-25-3p, and miR-25-5p. miRNA binding sites from any muscle specific miRNA can be introduced to or removed from a nucleic acid molecule (e.g., RNA, e.g., mRNA) of the disclosure to regulate expression of the nucleic acid molecule (e.g., RNA, e.g., mRNA) in the muscle. Muscle specific miRNA binding sites can be engineered alone or further in combination with immune cell (e.g., APC) miRNA binding sites in a nucleic acid molecule (e.g., RNA, e.g., mRNA) of the disclosure.

miRNAs are also differentially expressed in different types of cells, such as, but not limited to, endothelial cells, epithelial cells, and adipocytes.

miRNAs that are known to be expressed in endothelial cells include, but are not limited to, let-7b-3p, let-7b-5p, miR-100-3p, miR-100-5p, miR-101-3p, miR-101-5p, miR-126-3p, miR-126-5p, miR-1236-3p, miR-1236-5p, miR-130a-3p, miR-130a-5p, miR-17-5p, miR-17-3p, miR-18a-3p, miR-18a-5p, miR-19a-3p, miR-19a-5p, miR-19b-1-5p, miR-19b-2-5p, miR-19b-3p, miR-20a-3p, miR-20a-5p, miR-217, miR-210, miR-21-3p, miR-21-5p, miR-221-3p, miR-221-5p, miR-222-3p, miR-222-5p, miR-23a-3p, miR-23a-5p, miR-296-5p, miR-361-3p, miR-361-5p, miR-421, miR-424-3p, miR-424-5p, miR-513a-5p, miR-92a-1-5p, miR-92a-2-5p, miR-92a-3p, miR-92b-3p, and miR-92b-5p. Many novel miRNAs are discovered in endothelial cells from deep-sequencing analysis (e.g., Voellenkle C et al., RNA, 2012, 18, 472-484, herein incorporated by reference in its entirety). miRNA binding sites from any endothelial cell specific miRNA can be introduced to or removed from a nucleic acid molecule (e.g., RNA, e.g., mRNA) of the disclosure to regulate expression of the nucleic acid molecule (e.g., RNA, e.g., mRNA) in the endothelial cells.

miRNAs that are known to be expressed in epithelial cells include, but are not limited to, let-7b-3p, let-7b-5p, miR-1246, miR-200a-3p, miR-200a-5p, miR-200b-3p, miR-200b-5p, miR-200c-3p, miR-200c-5p, miR-338-3p, miR-429, miR-451a, miR-451b, miR-494, miR-802 and miR-34a, miR-34b-5p, miR-34c-5p, miR-449a, miR-449b-3p, miR-449b-5p specific in respiratory ciliated epithelial cells, let-7 family, miR-133a, miR-133b, miR-126 specific in lung epithelial cells, miR-382-3p, miR-382-5p specific in renal epithelial cells, and miR-762 specific in corneal epithelial cells. miRNA binding sites from any epithelial cell specific miRNA can be introduced to or removed from a nucleic acid molecule (e.g., RNA, e.g., mRNA) of the disclosure to regulate expression of the nucleic acid molecule (e.g., RNA, e.g., mRNA) in the epithelial cells.

In addition, a large group of miRNAs are enriched in embryonic stem cells, controlling stem cell self-renewal as well as the development and/or differentiation of various cell lineages, such as neural cells, cardiac, hematopoietic cells, skin cells, osteogenic cells and muscle cells (e.g., Kuppusamy K T et al., Curr. Mol Med, 2013, 13(5), 757-764; Vidigal J A and Ventura A, Semin Cancer Biol. 2012, 22(5-6), 428-436; Goff L A et al., PLoS One, 2009, 4:e7192; Morin R D et al., Genome Res, 2008,18, 610-621; Yoo J K et al., Stem Cells Dev. 2012, 21(11), 2049-2057, each of which is herein incorporated by reference in its entirety). miRNAs abundant in embryonic stem cells include, but are not limited to, let-7a-2-3p, let-α-3p, let-7a-5p, let7d-3p, let-7d-5p, miR-103a-2-3p, miR-103a-5p, miR-106b-3p, miR-106b-5p, miR-1246, miR-1275, miR-138-1-3p, miR-138-2-3p, miR-138-5p, miR-154-3p, miR-154-5p, miR-200c-3p, miR-200c-5p, miR-290, miR-301a-3p, miR-301a-5p, miR-302a-3p, miR-302a-5p, miR-302b-3p, miR-302b-5p, miR-302c-3p, miR-302c-5p, miR-302d-3p, miR-302d-5p, miR-302e, miR-367-3p, miR-367-5p, miR-369-3p, miR-369-5p, miR-370, miR-371, miR-373, miR-380-5p, miR-423-3p, miR-423-5p, miR-486-5p, miR-520c-3p, miR-548e, miR-548f, miR-548g-3p, miR-548g-5p, miR-548i, miR-548k, miR-5481, miR-548m, miR-548n, miR-548o-3p, miR-548o-5p, miR-548p, miR-664a-3p, miR-664a-5p, miR-664b-3p, miR-664b-5p, miR-766-3p, miR-766-5p, miR-885-3p, miR-885-5p,miR-93-3p, miR-93-5p, miR-941, miR-96-3p, miR-96-5p, miR-99b-3p and miR-99b-5p. Many predicted novel miRNAs are discovered by deep sequencing in human embryonic stem cells (e.g., Morin R D et al., Genome Res, 2008,18, 610-621; Goff L A et al., PLoS One, 2009, 4:e7192; Bar Metal., Stem cells, 2008, 26, 2496-2505, the content of each of which is incorporated herein by reference in its entirety).

In some embodiments, the binding sites of embryonic stem cell specific miRNAs can be included in or removed from the 3′UTR of a nucleic acid molecule (e.g., RNA, e.g., mRNA) of the disclosure to modulate the development and/or differentiation of embryonic stem cells, to inhibit the senescence of stem cells in a degenerative condition (e.g. degenerative diseases), or to stimulate the senescence and apoptosis of stem cells in a disease condition (e.g. cancer stem cells).

Many miRNA expression studies are conducted to profile the differential expression of miRNAs in various cancer cells/tissues and other diseases. Some miRNAs are abnormally over-expressed in certain cancer cells and others are under-expressed. For example, miRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (US2013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, US2010/0323357); cutaneous T cell lymphoma (WO2013/011378); colorectal cancer cells (WO2011/0281756, WO2011/076142); cancer positive lymph nodes (WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells (US2012/0309645, WO2011/095623); breast cancer cells (WO2008/154098, WO2007/081740, US2012/0214699), leukemia and lymphoma (WO2008/073915, US2009/0092974, US2012/0316081, US2012/0283310, WO2010/018563), the content of each of which is incorporated herein by reference in its entirety.

As a non-limiting example, miRNA binding sites for miRNAs that are over-expressed in certain cancer and/or tumor cells can be removed from the 3′UTR of a nucleic acid molecule (e.g., RNA, e.g., mRNA) of the disclosure, restoring the expression suppressed by the over-expressed miRNAs in cancer cells, thus ameliorating the corresponsive biological function, for instance, transcription stimulation and/or repression, cell cycle arrest, apoptosis and cell death. Normal cells and tissues, wherein miRNAs expression is not up-regulated, will remain unaffected.

miRNA can also regulate complex biological processes such as angiogenesis (e.g., miR-132) (Anand and Cheresh Curr Opin Hematol 2011 18:171-176). In the nucleic acid molecules (e.g., RNA, e.g., mRNA) of the disclosure, miRNA binding sites that are involved in such processes can be removed or introduced, in order to tailor the expression of the nucleic acid molecules (e.g., RNA, e.g., mRNA) to biologically relevant cell types or relevant biological processes. In this context, the nucleic acid molecules (e.g., RNA, e.g., mRNA) of the disclosure are defined as auxotrophic polynucleotides.

In some embodiments, the therapeutic window and/or differential expression (e.g., tissue-specific expression) of a polypeptide of the disclosure may be altered by incorporation of a miRNA binding site into a nucleic acid molecule (e.g., RNA, e.g., mRNA) encoding the polypeptide. In one example, a nucleic acid molecule (e.g., RNA, e.g., mRNA) may include one or more miRNA binding sites that are bound by miRNAs that have higher expression in one tissue type as compared to another. In another example, a nucleic acid molecule (e.g., RNA, e.g., mRNA) may include one or more miRNA binding sites that are bound by miRNAs that have lower expression in a cancer cell as compared to a non-cancerous cell of the same tissue of origin. When present in a cancer cell that expresses low levels of such an miRNA, the polypeptide encoded by the nucleic acid molecule (e.g., RNA, e.g., mRNA) typically will show increased expression.

Liver cancer cells (e.g., hepatocellular carcinoma cells) typically express low levels of miR-122 as compared to normal liver cells. Therefore, a nucleic acid molecule (e.g., RNA, e.g., mRNA) encoding a polypeptide that includes at least one miR-122 binding site (e.g., in the 3′-UTR of the mRNA) will typically express comparatively low levels of the polypeptide in normal liver cells and comparatively high levels of the polypeptide in liver cancer cells. If the polypeptide is able to induce immunogenic cell death, this can cause preferential immunogenic cell killing of liver cancer cells (e.g., hepatocellular carcinoma cells) as compared to normal liver cells.

In some embodiments, the nucleic acid molecule (e.g., RNA, e.g., mRNA) includes at least one miR-122 binding site, at least two miR-122 binding sites, at least three miR-122 binding sites, at least four miR-122 binding sites, or at least five miR-122 binding sites. In one aspect, the miRNA binding site binds miR-122 or is complementary to miR-122. In another aspect, the miRNA binding site binds to miR-122-3p or miR-122-5p. In a particular aspect, the miRNA binding site comprises a nucleotide sequence at least 80%, at least 85%, at least 90%, at least 95%, or 100% identical to SEQ ID NO: 75, wherein the miRNA binding site binds to miR-122. In another particular aspect, the miRNA binding site comprises a nucleotide sequence at least 80%, at least 85%, at least 90%, at least 95%, or 100% identical to SEQ ID NO: 73, wherein the miRNA binding site binds to miR-122. These sequences are shown below in Table 11.

In some embodiments, a nucleic acid molecule (e.g., RNA, e.g., mRNA) of the disclosure comprises a miRNA binding site, wherein the miRNA binding site comprises one or more nucleotide sequences selected from Table 11, including one or more copies of any one or more of the miRNA binding site sequences. In some embodiments, a nucleic acid molecule (e.g., RNA, e.g., mRNA) of the disclosure further comprises at least one, two, three, four, five, six, seven, eight, nine, ten, or more of the same or different miRNA binding sites selected from Table 11, including any combination thereof. In some embodiments, the miRNA binding site binds to miR-142 or is complementary to miR-142. In some embodiments, the miR-142 comprises SEQ ID NO: 66. In some embodiments, the miRNA binding site binds to miR-142-3p or miR-142-5p. In some embodiments, the miR-142-3p binding site comprises SEQ ID NO: 68. In some embodiments, the miR-142-5p binding site comprises SEQ ID NO: 70. In some embodiments, the miRNA binding site comprises a nucleotide sequence at least 80%, at least 85%, at least 90%, at least 95%, or 100% identical to SEQ ID NO: 68 or SEQ ID NO: 70.

TABLE 11
Representative microRNAs and
microRNA binding sites

| SEQ | | |
|---|---|---|
| ID NO. | Description | Sequence |
| 138 | mmiR-142 | GACAGUGCAGUCACCCAUAAAGUAGAAAGC |
| | | ACUACUAACAGCACUGGAGGGUGUAGUGUU |
| | | UCCUACUUUAUGGAUGAGUGUACUGUG |
| 139 | mmiR-142-3p | UGUAGUGUUUCCUACUUUAUGGA |
| 140 | mmiR-142-3p | UCCAUAAAGUAGGAAACACUACA |
| | binding | |
| | site | |
| 141 | mmiR-142-5p | CAUAAAGUAGAAAGCACACU |
| 142 | mmiR-142-5p | AGUAGUGCUUUCUACUUUAUG |
| | binding | |
| | site | |
| 143 | miR-122 | CCUUAGCAGAGCUGUGGAGUGUGACAAUGG |
| | | UGUUUGUGUCUAAACUAUCAAACGCCAUUA |
| | | UCACACUAAAUAGCUACUGCUAGGC |
| 144 | miR-122-3p | AACGCCAUUAUCACACUAAAUA |
| 145 | miR-122-3p | UAUUUAGUGUGAUAAUGGCGUU |
| | binding | |
| | site | |
| 146 | miR-122-5p | UGGAGUGUGACAAUGGUGUUUG |
| 147 | miR-122-5p | CAAACACCAUUGUCACACUCCA |
| | binding | |
| | site | |

In some embodiments, a miRNA binding site is inserted in the nucleic acid molecule (e.g., RNA, e.g., mRNA) of the disclosure in any position of the nucleic acid molecule (e.g., RNA, e.g., mRNA) (e.g., the 5′UTR and/or 3′UTR). In some embodiments, the 5′UTR comprises a miRNA binding site. In some embodiments, the 3′UTR comprises a miRNA binding site. In some embodiments, the 5′UTR and the 3′UTR comprise a miRNA binding site. The insertion site in the nucleic acid molecule (e.g., RNA, e.g., mRNA) can be anywhere in the nucleic acid molecule (e.g., RNA, e.g., mRNA) as long as the insertion of the miRNA binding site in the nucleic acid molecule (e.g., RNA, e.g., mRNA) does not interfere with the translation of a functional polypeptide in the absence of the corresponding miRNA; and in the presence of the miRNA, the insertion of the miRNA binding site in the nucleic acid molecule (e.g., RNA, e.g., mRNA) and the binding of the miRNA binding site to the corresponding miRNA are capable of degrading the polynucleotide or preventing the translation of the nucleic acid molecule (e.g., RNA, e.g., mRNA).

In some embodiments, a miRNA binding site is inserted in at least about 30 nucleotides downstream from the stop codon of an ORF in a nucleic acid molecule (e.g., RNA, e.g., mRNA) of the disclosure comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the disclosure. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a nucleic acid molecule (e.g., RNA, e.g., mRNA) of the disclosure. miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′UTR of the same sequence type.

In one embodiment, other regulatory elements and/or structural elements of the 5′UTR can influence miRNA mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The nucleic acid molecules (e.g., RNA, e.g., mRNA) of the disclosure can further include this structured 5′UTR in order to enhance microRNA mediated gene regulation.

At least one miRNA binding site can be engineered into the 3′UTR of a polynucleotide of the disclosure. In this context, at least two, at least three, at least four, at least five, at least six, at least seven, at least eight, at least nine, at least ten, or more miRNA binding sites can be engineered into a 3′UTR of a nucleic acid molecule (e.g., RNA, e.g., mRNA) of the disclosure. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a nucleic acid molecule (e.g., RNA, e.g., mRNA) of the disclosure. In one embodiment, miRNA binding sites incorporated into a nucleic acid molecule (e.g., RNA, e.g., mRNA) of the disclosure can be the same or can be different miRNA sites. A combination of different miRNA binding sites incorporated into a nucleic acid molecule (e.g., RNA, e.g., mRNA) of the disclosure can include combinations in which more than one copy of any of the different miRNA sites are incorporated. In another embodiment, miRNA binding sites incorporated into a nucleic acid molecule (e.g., RNA, e.g., mRNA) of the disclosure can target the same or different tissues in the body. As a non-limiting example, through the introduction of tissue-, cell-type-, or disease-specific miRNA binding sites in the 3′-UTR of a nucleic acid molecule (e.g., RNA, e.g., mRNA) of the disclosure, the degree of expression in specific cell types (e.g., hepatocytes, myeloid cells, endothelial cells, cancer cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′UTR in a nucleic acid molecule (e.g., RNA, e.g., mRNA) of the disclosure. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′UTR. As yet another non-limiting example, a miRNA binding site can be engineered near the 5′ terminus of the 3′UTR and near the 3′ terminus of the 3′UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

In one embodiment, a nucleic acid molecule (e.g., RNA, e.g., mRNA) of the disclosure can be engineered to include more than one miRNA site expressed in different tissues or different cell types of a subject. As a non-limiting example, a nucleic acid molecule (e.g., RNA, e.g., mRNA) of the disclosure can be engineered to include miR-192 and miR-122 to regulate expression of the nucleic acid molecule (e.g., RNA, e.g., mRNA) in the liver and kidneys of a subject. In another embodiment, a nucleic acid molecule (e.g., RNA, e.g., mRNA) of the disclosure can be engineered to include more than one miRNA site for the same tissue. In some embodiments, the therapeutic window and or differential expression associated with the polypeptide encoded by a nucleic acid molecule (e.g., RNA, e.g., mRNA) of the disclosure can be altered with a miRNA binding site. For example, a nucleic acid molecule (e.g., RNA, e.g., mRNA) encoding a polypeptide that provides a death signal can be designed to be more highly expressed in cancer cells by virtue of the miRNA signature of those cells. Where a cancer cell expresses a lower level of a particular miRNA, the nucleic acid molecule (e.g., RNA, e.g., mRNA) encoding the binding site for that miRNA (or miRNAs) would be more highly expressed. Hence, the polypeptide that provides a death signal triggers or induces cell death in the cancer cell. Neighboring noncancer cells, harboring a higher expression of the same miRNA would be less affected by the encoded death signal as the polynucleotide would be expressed at a lower level due to the effects of the miRNA binding to the binding site or “sensor” encoded in the 3′UTR. Conversely, cell survival or cytoprotective signals can be delivered to tissues containing cancer and non-cancerous cells where a miRNA has a higher expression in the cancer cells—the result being a lower survival signal to the cancer cell and a larger survival signal to the normal cell. Multiple nucleic acid molecule (e.g., RNA, e.g., mRNA) can be designed and administered having different signals based on the use of miRNA binding sites as described herein.

In some embodiments, the expression of a nucleic acid molecule (e.g., RNA, e.g., mRNA) of the disclosure can be controlled by incorporating at least one sensor sequence in the polynucleotide and formulating the nucleic acid molecule (e.g., RNA, e.g., mRNA) for administration. As a non-limiting example, a nucleic acid molecule (e.g., RNA, e.g., mRNA) of the disclosure can be targeted to a tissue or cell by incorporating a miRNA binding site and formulating the nucleic acid molecule (e.g., RNA, e.g., mRNA) in a lipid nanoparticle comprising a cationic lipid, including any of the lipids described herein.

A nucleic acid molecule (e.g., RNA, e.g., mRNA) of the disclosure can be engineered for more targeted expression in specific tissues, cell types, or biological conditions based on the expression patterns of miRNAs in the different tissues, cell types, or biological conditions. Through introduction of tissue-specific miRNA binding sites, a nucleic acid molecule (e.g., RNA, e.g., mRNA) of the disclosure can be designed for optimal protein expression in a tissue or cell, or in the context of a biological condition.

In some embodiments, a nucleic acid molecule (e.g., RNA, e.g., mRNA) of the disclosure can be designed to incorporate miRNA binding sites that either have 100% identity to known miRNA seed sequences or have less than 100% identity to miRNA seed sequences. In some embodiments, a nucleic acid molecule (e.g., RNA, e.g., mRNA) of the disclosure can be designed to incorporate miRNA binding sites that have at least: 60%, 65%, 70%, 75%, 80%, 85%, 90%, 95%, 96%, 97%, 98%, or 99% identity to known miRNA seed sequences. The miRNA seed sequence can be partially mutated to decrease miRNA binding affinity and as such result in reduced downmodulation of the nucleic acid molecule (e.g., RNA, e.g., mRNA). In essence, the degree of match or mis-match between the miRNA binding site and the miRNA seed can act as a rheostat to more finely tune the ability of the miRNA to modulate protein expression. In addition, mutation in the non-seed region of a miRNA binding site can also impact the ability of a miRNA to modulate protein expression.

In one embodiment, a miRNA sequence can be incorporated into the loop of a stem loop. In another embodiment, a miRNA seed sequence can be incorporated in the loop of a stem loop and a miRNA binding site can be incorporated into the 5′ or 3′ stem of the stem loop. In one embodiment, a translation enhancer element (TEE) can be incorporated on the 5′end of the stem of a stem loop and a miRNA seed can be incorporated into the stem of the stem loop. In another embodiment, a TEE can be incorporated on the 5′ end of the stem of a stem loop, a miRNA seed can be incorporated into the stem of the stem loop and a miRNA binding site can be incorporated into the 3′ end of the stem or the sequence after the stem loop. The miRNA seed and the miRNA binding site can be for the same and/or different miRNA sequences.

In one embodiment, the incorporation of a miRNA sequence and/or a TEE sequence changes the shape of the stem loop region which can increase and/or decrease translation. (see e.g, Kedde et al., “A Pumilio-induced RNA structure switch in p27-3′UTR controls miR-221 and miR-22 accessibility.” Nature Cell Biology. 2010, incorporated herein by reference in its entirety).

In one embodiment, the 5′-UTR of a nucleic acid molecule (e.g., RNA, e.g., mRNA) of the disclosure can comprise at least one miRNA sequence. The miRNA sequence can be, but is not limited to, a 19 or 22 nucleotide sequence and/or a miRNA sequence without the seed. In one embodiment the miRNA sequence in the 5′UTR can be used to stabilize a nucleic acid molecule (e.g., RNA, e.g., mRNA) of the disclosure described herein.

In another embodiment, a miRNA sequence in the 5′UTR of a nucleic acid molecule (e.g., RNA, e.g., mRNA) of the disclosure can be used to decrease the accessibility of the site of translation initiation such as, but not limited to a start codon. See, e.g., Matsuda et al., PLoS One. 2010 11(5):e15057; incorporated herein by reference in its entirety, which used antisense locked nucleic acid (LNA) oligonucleotides and exon-junction complexes (EJCs) around a start codon (−4 to +37 where the A of the AUG codons is +1) in order to decrease the accessibility to the first start codon (AUG). Matsuda showed that altering the sequence around the start codon with an LNA or EJC affected the efficiency, length and structural stability of a polynucleotide. A nucleic acid molecule (e.g., RNA, e.g., mRNA) of the disclosure can comprise a miRNA sequence, instead of the LNA or EJC sequence described by Matsuda et al, near the site of translation initiation in order to decrease the accessibility to the site of translation initiation. The site of translation initiation can be prior to, after or within the miRNA sequence. As a non-limiting example, the site of translation initiation can be located within a miRNA sequence such as a seed sequence or binding site. As another non-limiting example, the site of translation initiation can be located within a miR-122 sequence such as the seed sequence or the mir-122 binding site. In some embodiments, a nucleic acid molecule (e.g., RNA, e.g., mRNA) of the disclosure can include at least one miRNA in order to dampen the antigen presentation by antigen presenting cells. The miRNA can be the complete miRNA sequence, the miRNA seed sequence, the miRNA sequence without the seed, or a combination thereof. As a non-limiting example, a miRNA incorporated into a nucleic acid molecule (e.g., RNA, e.g., mRNA) of the disclosure can be specific to the hematopoietic system. As another non-limiting example, a miRNA incorporated into a nucleic acid molecule (e.g., RNA, e.g., mRNA) of the disclosure to dampen antigen presentation is miR-142-3p.

In some embodiments, a nucleic acid molecule (e.g., RNA, e.g., mRNA) of the disclosure can include at least one miRNA in order to dampen expression of the encoded polypeptide in a tissue or cell of interest. As a non-limiting example, a nucleic acid molecule (e.g., RNA, e.g., mRNA) of the disclosure can include at least one miR-122 binding site in order to dampen expression of an encoded polypeptide of interest in the liver. As another non-limiting example a nucleic acid molecule (e.g., RNA, e.g., mRNA) of the disclosure can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, a nucleic acid molecule (e.g., RNA, e.g., mRNA) of the disclosure can comprise at least one miRNA binding site in the 3′UTR in order to selectively degrade mRNA therapeutics in the immune cells to subdue unwanted immunogenic reactions caused by therapeutic delivery. As a non-limiting example, the miRNA binding site can make a nucleic acid molecule (e.g., RNA, e.g., mRNA) of the disclosure more unstable in antigen presenting cells. Non-limiting examples of these miRNAs include mir-142-5p, mir-142-3p, mir-146a-5p, and mir-146-3p.

In one embodiment, a nucleic acid molecule (e.g., RNA, e.g., mRNA) of the disclosure comprises at least one miRNA sequence in a region of the nucleic acid molecule (e.g., RNA, e.g., mRNA) that can interact with a RNA binding protein.

In some embodiments, the nucleic acid molecule (e.g., RNA, e.g., mRNA) of the disclosure comprising (i) a sequence-optimized nucleotide sequence (e.g., an ORF) and (ii) a miRNA binding site (e.g., a miRNA binding site that binds to miR-142).

In some embodiments, the nucleic acid molecule (e.g., RNA, e.g., mRNA) of the disclosure comprises a uracil-modified sequence encoding a polypeptide disclosed herein and a miRNA binding site disclosed herein, e.g., a miRNA binding site that binds to miR-142. In some embodiments, the uracil-modified sequence encoding a polypeptide comprises at least one chemically modified nucleobase, e.g., 5-methoxyuracil. In some embodiments, at least 95% of a type of nucleobase (e.g., uracil) in a uracil-modified sequence encoding a polypeptide of the disclosure are modified nucleobases. In some embodiments, at least 95% of uricil in a uracil-modified sequence encoding a polypeptide is 5-methoxyuridine. In some embodiments, the nucleic acid molecule (e.g., RNA, e.g., mRNA) comprising a nucleotide sequence encoding a polypeptide disclosed herein and a miRNA binding site is formulated with a delivery agent.

### 3′-Stabilizing Region

In some embodiments, the mRNAs of the disclosure comprise a 3′-stabilizing region including one or more nucleosides (e.g., 1 to 500 nucleosides such as 1 to 200, 1 to 400, 1 to 10, 5 to 15, 10 to 20, 15 to 25, 20 to 30, 25 to 35, 30 to 40, 35 to 45, 40 to 50, 45 to 65, 50 to 70, 65 to 85, 70 to 90, 85 to 105, 90 to 110, 105 to 135, 120 to 150, 130 to 170, 150 to 200 or 1, 2, 3, 4, 5, 6, 7, 8, 9, 10, 20, 30, 40, 50, 60, 70, 80, 90, 100, 110, 120, 130, 140, 150, 160, 170, 180, 190, or 200 nucleosides). In some embodiments, the 3′-stabilizing region contains one or more alternative nucleosides having an alternative nucleobase, sugar, or backbone (e.g., a 2′-deoxynucleoside, a 3′-deoxynucleoside, a 2′,3′-dideoxynucleoside, a 2′-O-methylnucleoside, a 3′-O-methylnucleoside, a 3′-O-ethyl-nucleoside, 3′-arabinoside, an L-nucleoside, alpha-thio-2′-O-methyl-adenosine, 2′-fluoro-adenosine, arabino-adenosine, hexitol-adenosine, LNA-adenosine, PNA-adenosine, inverted thymidine, or 3′-azido-2′,3′-dideoxyadenosine). In some embodiments, the 3′-stabilizing region includes a plurality of alternative nucleosides. In some embodiments, the 3′-stabilizing region includes at least one non-nucleoside (e.g., an abasic ribose) at the 5′-terminus, the 3′-terminus, or at an internal position of the 3′-stabilizing region.

In some embodiments, the 3′-stabilizing region consists of one nucleoside (e.g., a 2′-deoxynucleoside, a 3′-deoxynucleoside, a 2′,3′-dideoxynucleoside, a 2′-O-methylnucleoside, a 3′-O-methylnucleoside, a 3′-O-ethyl-nucleoside, 3′-arabinoside, an L-nucleoside, alpha-thio-2′-O-methyl-adenosine, 2′-fluoro-adenosine, arabino-adenosine, hexitol-adenosine, LNA-adenosine, PNA-adenosine, inverted thymidine, or 3′-azido-2′,3′-dideoxyadenosine). In some embodiments, one or more nucleosides in the 3′-stabilizing region include the structure:

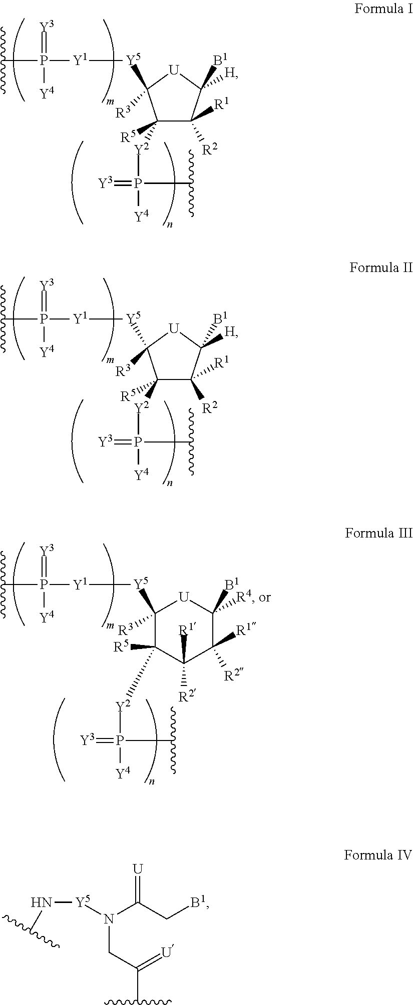

wherein B¹ is a nucleobase;

each U and U′ is, independently, O, S, N(R^{U})ₙᵤ, or C(R^{U})nu, wherein nu is 1 or 2 (e.g., 1 for N(R^{U})nu and 2 for C(R^{U})nu) and each R^{U} is, independently, H, halo, or optionally substituted C₁-C₆ alkyl;

each of R¹, R^{1′}, R^{1″}, R², R^{2′}, R^{2″}, R³, R⁴, and R⁵ is, independently, H, halo, hydroxy, thiol, optionally substituted C₁-C₆ alkyl, optionally substituted C₂-C₆ alkynyl, optionally substituted C₁-C₆heteroalkyl, optionally substituted C₂-C₆heteroalkenyl, optionally substituted C₂-C₆ heteroalkynyl, optionally substituted amino, azido, optionally substituted C₆-C₁₀ aryl; or R³ and/or R⁵ can join together with one of R¹, R^{1′}, R^{1″}, R², R^{2′}, or R^{2″} to form together with the carbons to which they are attached an optionally substituted C₃-C₁₀ carbocycle or an optionally substituted C₃-C₉heterocyclyl;

each of m and n is independently, 0, 1, 2, 3, 4, or 5;

each of Y¹, Y², and Y³, is, independently, O, S, Se, —NR^{N1}—, optionally substituted C₁-C₆ alkylene, or optionally substituted C₁-C₆heteroalkylene, wherein R^{N1} is H, optionally substituted C₁-C₆ alkyl, optionally substituted C₂-C₆ alkenyl, optionally substituted C₂-C₆ alkynyl, or optionally substituted C₆-C₁₀ aryl; and

each Y⁴ is, independently, H, hydroxy, protected hydroxy, halo, thiol, boranyl, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆heteroalkynyl, or optionally substituted amino; and

Y⁵ is 0, S, Se, optionally substituted C₁-C₆ alkylene, or optionally substituted C₁-C₆ heteroalkylene;

or is a salt thereof.

In some embodiments, the 3′-stabilizing region includes a plurality of adenosines. In some embodiments, all of the nucleosides of the 3′-stabilizing region are adenosines. In some embodiments, the 3′-stabilizing region includes at least one (e.g., at least two, at least three, at least four, at least five, at least six, at least seven, at least eight, at least nine, or at least ten) alternative nucleosides (e.g., an L-nucleoside such as L-adenosine, 2′-O-methyl-adenosine, alpha-thio-2′-O-methyl-adenosine, 2′-fluoro-adenosine, arabino-adenosine, hexitol-adenosine, LNA-adenosine, PNA-adenosine, or inverted thymidine). In some embodiments, the alternative nucleoside is an L-adenosine, a 2′-O-methyl-adenosine, or an inverted thymidine. In some embodiments, the 3′-stabilizing region includes a plurality of alternative nucleosides. In some embodiments, all of the nucleotides in the 3′-stabilizing region are alternative nucleosides. In some embodiments, the 3′-stabilizing region includes at least two different alternative nucleosides. In some embodiments, at least one alternative nucleoside is 2′-O-methyl-adenosine. In some embodiments, at least one alternative nucleoside is inverted thymidine. In some embodiments, at least one alternative nucleoside is 2′-O-methyl-adenosine, and at least one alternative nucleoside is inverted thymidine.

In some embodiments, the stabilizing region includes the structure:

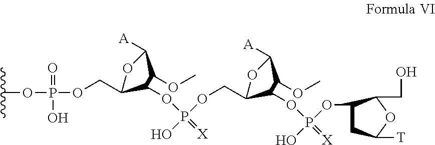

or a salt thereof;

wherein each X is, independently O or S; and

A represents adenine and T represents thymine.

In some embodiments, each X is O. In some embodiments, each X is S.

In some embodiments, all of the plurality of alternative nucleosides are the same (e.g., all of the alternative nucleosides are L-adenosine). In some embodiments, the 3′-stabilizing region includes ten nucleosides. In some embodiments, the 3′-stabilizing region includes eleven nucleosides. In some embodiments, the 3′-stabilizing region comprises at least five L-adenosines (e.g., at least ten L-adenosines, or at least twenty L-adenosines). In some embodiments, the 3′-stabilizing region consists of five L-adenosines. In some embodiments, the 3′-stabilizing region consists of ten L-adenosines. In some embodiments, the 3′-stabilizing region consists of twenty L-adenosines.

Further examples of 3′-stabilized regions are known in the art, e.g., as described in International Patent Publication Nos. WO2013/103659, WO2017/049275, and WO2017/049286, the 3′-stabilized regions of which are herein incorporated by references.

In some embodiments, the 5′-terminus of the 3′-stabilizing region is conjugated to the 3′-terminus of the 3′-UTR. In some embodiments, the 5′-terminus of the 3′-stabilizing region is conjugated to the 3′-terminus of the poly-A region. In some embodiments, the 5′-terminus of the 3′-stabilizing region is conjugated to the 3′-terminus of the poly-C region. In some embodiments of any of the foregoing polynucleotides, the 3′-stabilizing region includes the 3′-terminus of the polynucleotide.

In some embodiments, the 3′-stabilizing tail is conjugated to the remainder of the polynucleotide, e.g., at the 3′-terminus of the 3′-UTR or poly-A region via a phosphate linkage. In some embodiments, the phosphate linkage is a natural phosphate linkage. In some embodiments, the conjugation of the 3′-stabilizing tail and the remainder of the polynucleotide is produced via enzymatic or splint ligation.

In some embodiments, the 3′-stabilizing tail is conjugated to the remainder of the polynucleotide, e.g., at the 3′-terminus of the 3′-UTR or poly-A region via a chemical linkage. In some embodiments, the chemical linkage includes the structure of Formula V:

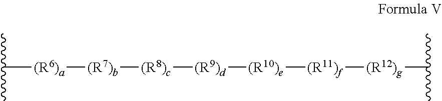

wherein a, b, c, e, f, and g are each, independently, 0 or 1;

d is 0, 1, 2, or 3;

each of R⁶, R⁸, R¹⁰, and R¹², is, independently, optionally substituted C₁-C₆ alkylene, optionally substituted C₁-C₆ heteroalkylene, optionally substituted C₂-C₆ alkenylene, optionally substituted C₂-C₆ alkynylene, or optionally substituted C₆-C₁₀ arylene, O, S, Se, and NR¹³;

R⁷ and R¹¹ are each, independently, carbonyl, thiocarbonyl, sulfonyl, or phosphoryl, wherein, if R⁷ is phosphoryl, —(R⁹)_{d}— is a bond, and e, f, and gare 0, then at least one of R⁶ or R⁸ is not O; and if R¹¹ is phosphoryl, —(R⁹)_{d}— is a bond, and a, b, and c are 0, then at least one of R¹⁰ or R¹² is not O;

each R⁹ is optionally substituted C₁-C₁₀ alkylene, optionally substituted C₂-C₁₀ alkenylene, optionally substituted C₂-C₁₀ alkynylene, optionally substituted C₂-C₁₀ heterocyclylene, optionally substituted C₆-C₁₂ arylene, optionally substituted C₂-C₁₀₀ polyethylene glycolene, or optionally substituted C₁-C₁₀ heteroalkylene, or a bond linking (R⁶)ₐ—(R⁷)_{b}—(R⁸)_{c} to (R¹⁰)ₑ—(R¹¹)_{f}—(R¹²)_{g}, wherein if —(R⁹)_{d}— is a bond, then at least one of a, b, c, e, f, or g is 1; and

R¹³ is hydrogen, optionally substituted C₁-C₄ alkyl, optionally substituted C₂-C₄ alkenyl, optionally substituted C₂-C₄ alkynyl, optionally substituted C₂-C₆ heterocyclyl, optionally substituted C₆-C₁₂ aryl, or optionally substituted C₁-C₇ heteroalkyl.

In some embodiments, the chemical linkage comprises the structure of Formula VI:

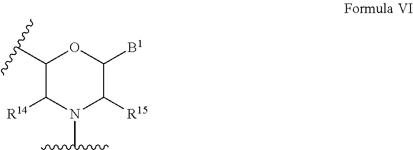

wherein B¹ is a nucleobase, hydrogen, halo, hydroxy, thiol, optionally substituted C₁-C₆ alkyl, optionally substituted C₂-C₆ alkenyl, optionally substituted C₂-C₆ alkynyl, optionally substituted C₁-C₆heteroalkyl, optionally substituted C₂-C₆heteroalkenyl, optionally substituted C₂-C₆heteroalkynyl, optionally substituted amino, azido, optionally substituted C₃-C₁₀ cycloalkyl, optionally substituted C₆-C₁₀ aryl, optionally substituted C₂-C₉ heterocycle; and

R¹⁴ and R¹⁵ are each, independently, hydrogen or hydroxy.

In some embodiments, the chemical linkage includes the structure:

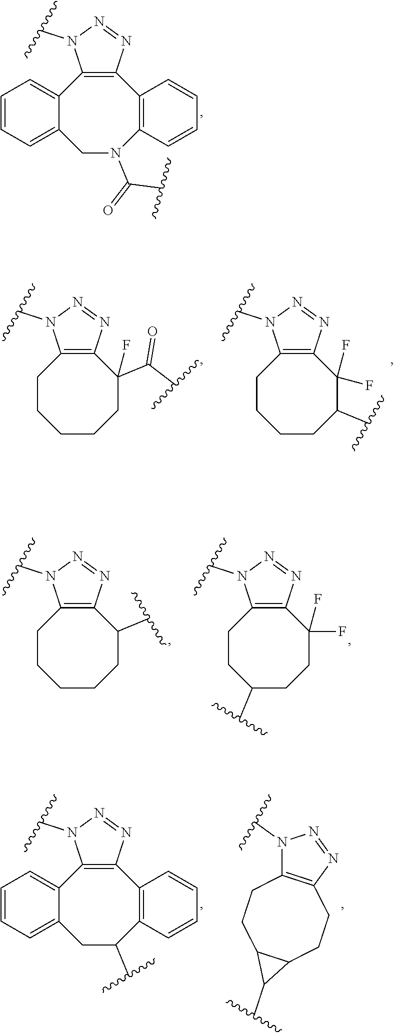

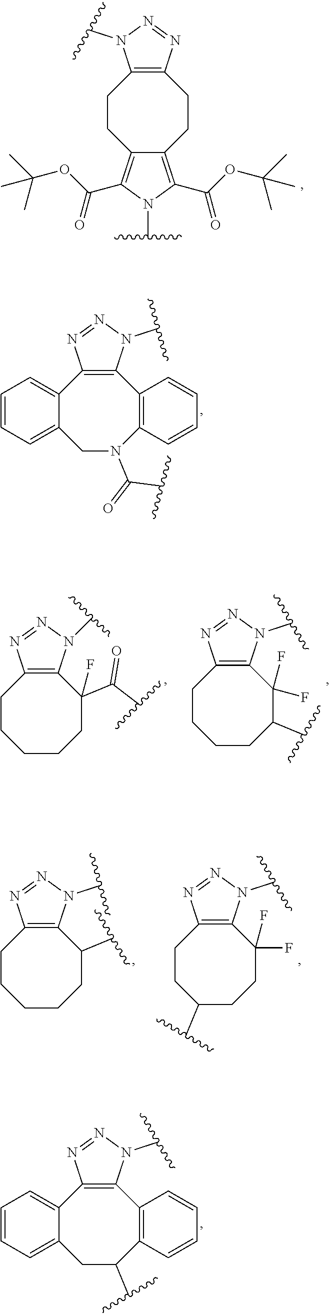

or an amide bond.
Further examples of chemical linkages to conjugate 3′-stabilized regions to the remainder of the polynucleotide are known in the art, e.g., as described in International Patent Publication Nos. WO2017/049275 and WO2017/049286, the chemical linkers of which are herein incorporated by reference.

### Delivery Agents

a. Lipid Compound

The present disclosure provides pharmaceutical compositions with advantageous properties. The lipid compositions described herein may be advantageously used in lipid nanoparticle compositions for the delivery of therapeutic and/or prophylactic agents, e.g., mRNAs, to mammalian cells or organs. For example, the lipids described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and a therapeutic or prophylactic agent, e.g., mRNA, has an increased therapeutic index as compared to a corresponding formulation which comprises a reference lipid (e.g., MC3, KC2, or DLinDMA) and the same therapeutic or prophylactic agent.

In certain embodiments, the present application provides pharmaceutical compositions comprising:

(a) an mRNA comprising a nucleotide sequence encoding a polypeptide; and

(b) a delivery agent.

Lipid Nanoparticle Formulations

In some embodiments, nucleic acids of the invention (e.g. mRNA) are formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the invention can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Nucleic acids of the present disclosure (e.g. mRNA) are typically formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

Ionizable Lipids

In some aspects, the ionizable lipids of the present disclosure may be one or more of compounds of Formula (I):

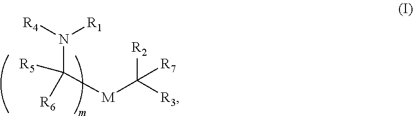

or their N-oxides, or salts or isomers thereof, wherein:

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of hydrogen, a C₃₋₆ carbocycle, —(CH₂)ₙQ, —(CH₂)ₙCHQR, —CHQR,—CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —OC(O)-M″-C(O)O—, —C(O)N(R′)—,

—N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and

H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13; and wherein when R₄ is —(CH₂)ₙQ, —(CH₂)ₙCHQR,—CHQR, or -CQ(R)₂, then (i) Q is not —N(R)₂ when n is 1, 2, 3, 4 or 5, or (ii) Q is not 5, 6, or 7-membered heterocycloalkyl when n is 1 or 2.

In certain embodiments, a subset of compounds of Formula (I) includes those of Formula (IA):

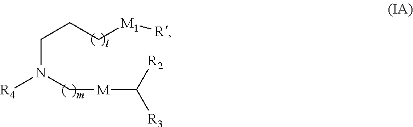

or its N-oxide, or a salt or isomer thereof, wherein 1 is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (IB):

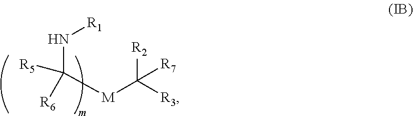

or its N-oxide, or a salt or isomer thereof in which all variables are as defined herein. For example, m is selected from 5, 6, 7, 8, and 9; R₄ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (II):

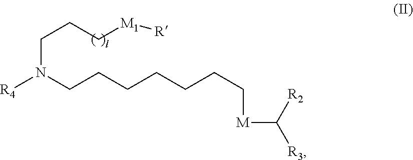

or its N-oxide, or a salt or isomer thereof, wherein 1 is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In one embodiment, the compounds of Formula (I) are of Formula (IIa),

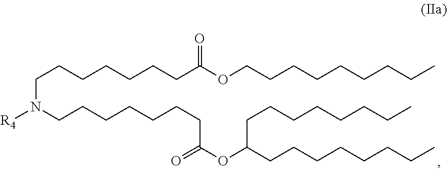

or their N-oxides, or salts or isomers thereof, wherein R₄ is as described herein.

In another embodiment, the compounds of Formula (I) are of Formula (IIb),

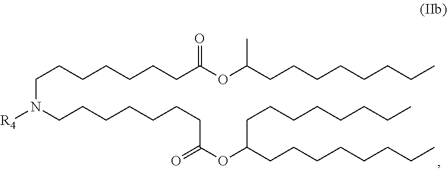

or their N-oxides, or salts or isomers thereof, wherein R₄ is as described herein.

In another embodiment, the compounds of Formula (I) are of Formula (IIc) or (IIe):

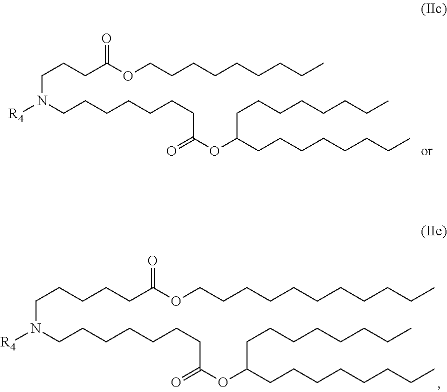

or their N-oxides, or salts or isomers thereof, wherein R₄ is as described herein.

In another embodiment, the compounds of Formula (I) are of Formula (IIf):

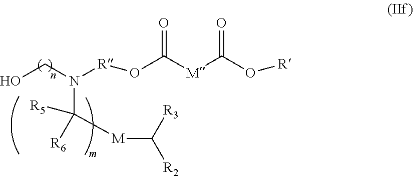

or their N-oxides, or salts or isomers thereof,

wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (I) are of Formula (IId),

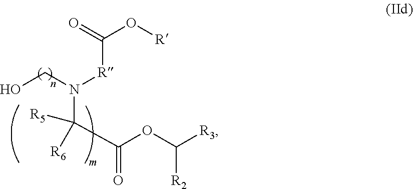

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In a further embodiment, the compounds of Formula (I) are of Formula (IIg),

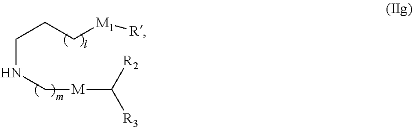

or their N-oxides, or salts or isomers thereof, wherein l is selected from 1, 2, 3, 4, and 5; m is selected from 5, 6, 7, 8, and 9; M₁ is a bond or M′; M and M′ are independently selected from

—C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

In some embodiments, the ionizable lipids are selected from Compounds 1-280 described in U.S. Application No. 62/475,166.

In some embodiments, the ionizable lipid is

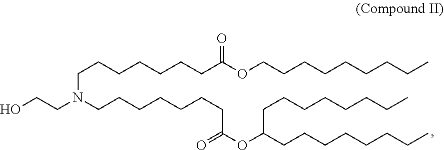 [SMILES: CCCCCCCCCOC(=O)CCCCCCCN(CCO)CCCCCCCC(=O)OC(CCCCCCCC)CCCCCCCC]

or a salt thereof.

In some embodiments, the ionizable lipid is

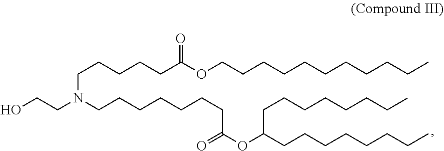 [SMILES: CCCCCCCCCCCOC(=O)CCCCCN(CCO)CCCCCCCC(=O)OC(CCCCCCCC)CCCCCCCC]

or a salt thereof.

In some embodiments, the ionizable lipid is

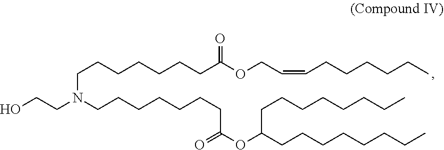 [SMILES: CCCCCC/C=C\COC(=O)CCCCCCCN(CCO)CCCCCCCC(=O)OC(CCCCCCCC)CCCCCCCC]

or a salt thereof.

In some embodiments, the ionizable lipid is

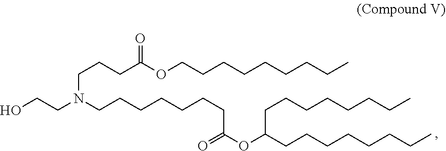 [SMILES: CCCCCCCCCOC(=O)CCCN(CCO)CCCCCCCC(=O)OC(CCCCCCCC)CCCCCCCC]

or a salt thereof.

The central amine moiety of a lipid according to Formula (I), (IA), (IB), (II), (IIa), (IIb), (IIc), (IId), (IIe), (IIf), or (IIg) may be protonated at a physiological pH. Thus, a lipid may have a positive or partial positive charge at physiological pH. Such lipids may be referred to as cationic or ionizable (amino) lipids. Lipids may also be zwitterionic, i.e., neutral molecules having both a positive and a negative charge.

In some aspects, the ionizable lipids of the present disclosure may be one or more of compounds of formula (III),

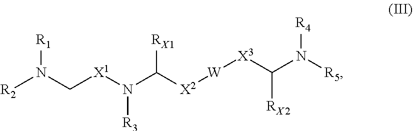

or salts or isomers thereof, wherein

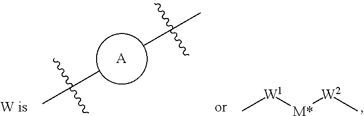

ring A is

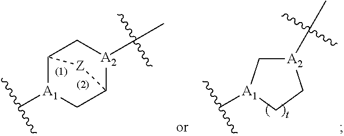

t is 1 or 2;

A₁ and A₂ are each independently selected from CH or N;

Z is CH₂ or absent wherein when Z is CH₂, the dashed lines (1) and (2) each represent a single bond; and when Z is absent, the dashed lines (1) and (2) are both absent;

R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;

R_{X1} and R_{X2} are each independently H or C₁₋₃ alkyl;

each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;

M* is C₁-C₆ alkyl,

W¹ and W² are each independently selected from the group consisting of —O— and —N(R₆)—;

each R₆ is independently selected from the group consisting of H and C₁₋₅ alkyl;

X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H;

each R″ is independently selected from the group consisting of C₃₋₁₂ alkyl, C₃₋₁₂ alkenyl and -R*MR′; and

n is an integer from 1-6;

when ring A is

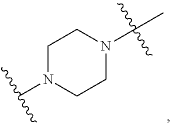 [SMILES: *N1CCN(*)CC1]

then

i) at least one of X¹, X², and X³ is not —CH₂—; and/or

ii) at least one of R₁, R₂, R₃, R₄, and R₅ is —R″MR′.

In some embodiments, the compound is of any of formulae (IIIa1)-(IIIa8):

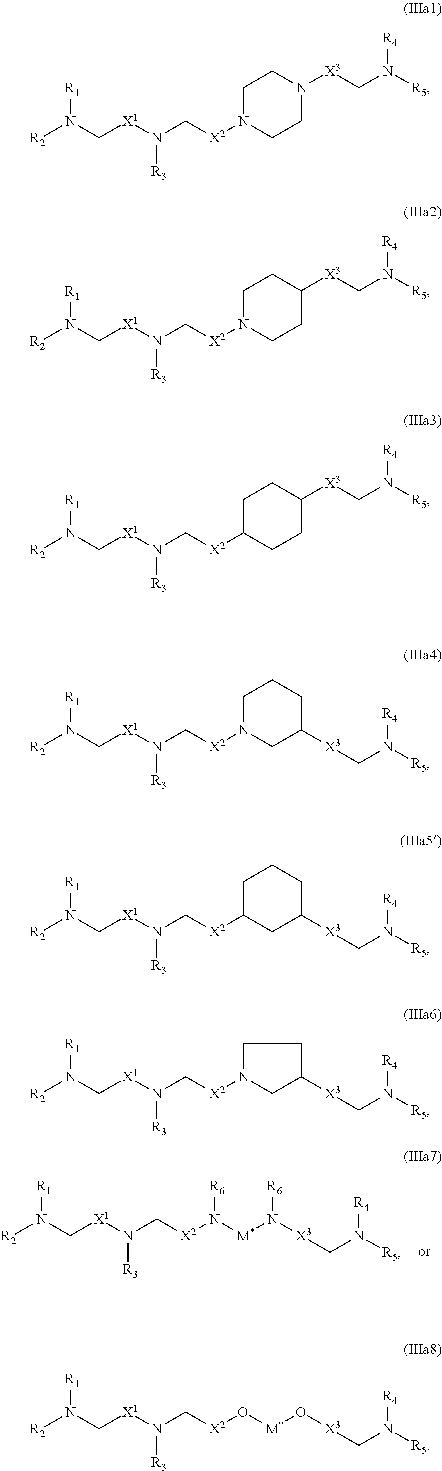

In some embodiments, the ionizable lipids are one or more of the compounds described in U.S. Application Nos. 62/271,146, 62/338,474, 62/413,345, and 62/519,826, and PCT Application No. PCT/US2016/068300.

In some embodiments, the ionizable lipids are selected from Compounds 1-156 described in U.S. Application No. 62/519,826.

In some embodiments, the ionizable lipids are selected from Compounds 1-16, 42-66, 68-76, and 78-156 described in U.S. Application No. 62/519,826.

In some embodiments, the ionizable lipid is

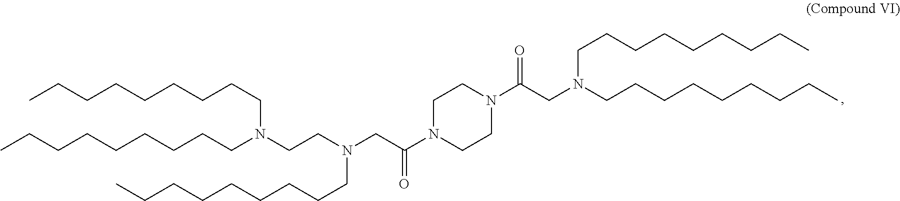 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

In some embodiments, the ionizable lipid is (Compound VII), or a salt thereof.

The central amine moiety of a lipid according to Formula (III), (IIIa1), (IIIa2), (IIIa3), (IIIa4), (IIIa5), (IIIa6), (IIIa7), or (IIIa8) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present invention is a compound of Formula (IV):

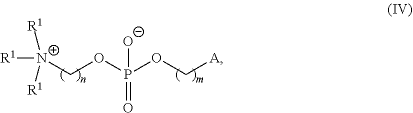

or a salt thereof, wherein:

each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;

n is 1, 2, 3, 4, 5, 6, 7, 8, 9, or 10;

m is 0, 1, 2, 3, 4, 5, 6, 7, 8, 9, or 10;

A is of the formula:

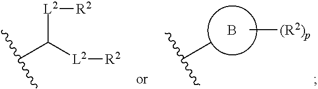

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2;

provided that the compound is not of the formula:

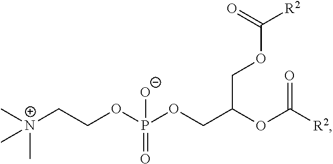

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530.

(i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IV), at least one of R¹ is not methyl. In certain embodiments, at least one of R¹ is not hydrogen or methyl. In certain embodiments, the compound of Formula (IV) is of one of the following formulae:

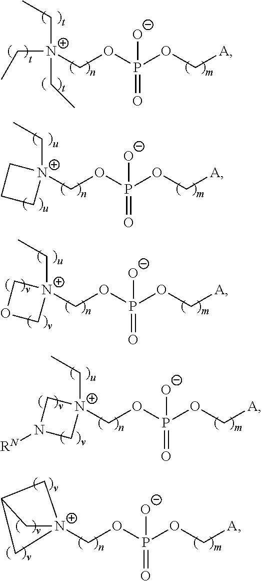

or a salt thereof, wherein:

each t is independently 1, 2, 3, 4, 5, 6, 7, 8, 9, or 10;

each u is independently 0, 1, 2, 3, 4, 5, 6, 7, 8, 9, or 10; and

each v is independently 1, 2, or 3.

In certain embodiments, a compound of Formula (IV) is of Formula (IV-a):

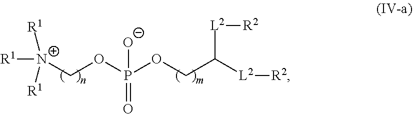

or a salt thereof.

In certain embodiments, a phospholipid useful or potentially useful in the present invention comprises a cyclic moiety in place of the glyceride moiety. In certain embodiments, a phospholipid useful in the present invention is DSPC, or analog thereof, with a cyclic moiety in place of the glyceride moiety. In certain embodiments, the compound of Formula (IV) is of Formula (IV-b):

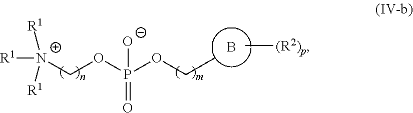

or a salt thereof.

(ii) Phospholipid Tail Modifications

In certain embodiments, a phospholipid useful or potentially useful in the present invention comprises a modified tail. In certain embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IV) is of Formula (IV-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), S(O), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O.

In certain embodiments, the compound of Formula (IV) is of Formula (IV-c):

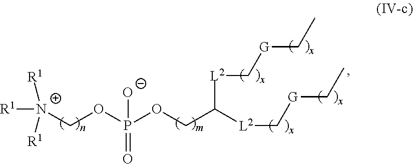

or a salt thereof, wherein:

each x is independently an integer between 0-30, inclusive; and

each instance is G is independently selected from the group consisting of optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), —OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O. Each possibility represents a separate embodiment of the present invention.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (IV), wherein n is 1, 3, 4, 5, 6, 7, 8, 9, or 10. For example, in certain embodiments, a compound of Formula (IV) is of one of the following formulae:

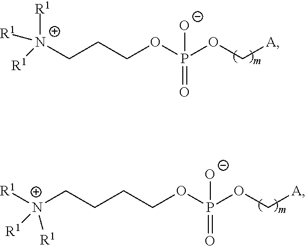

or a salt thereof.

Alternative Lipids

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful.

In certain embodiments, an alternative lipid is used in place of a phospholipid of the present disclosure.

In certain embodiments, an alternative lipid of the invention is oleic acid.

In certain embodiments, the alternative lipid is one of the following:

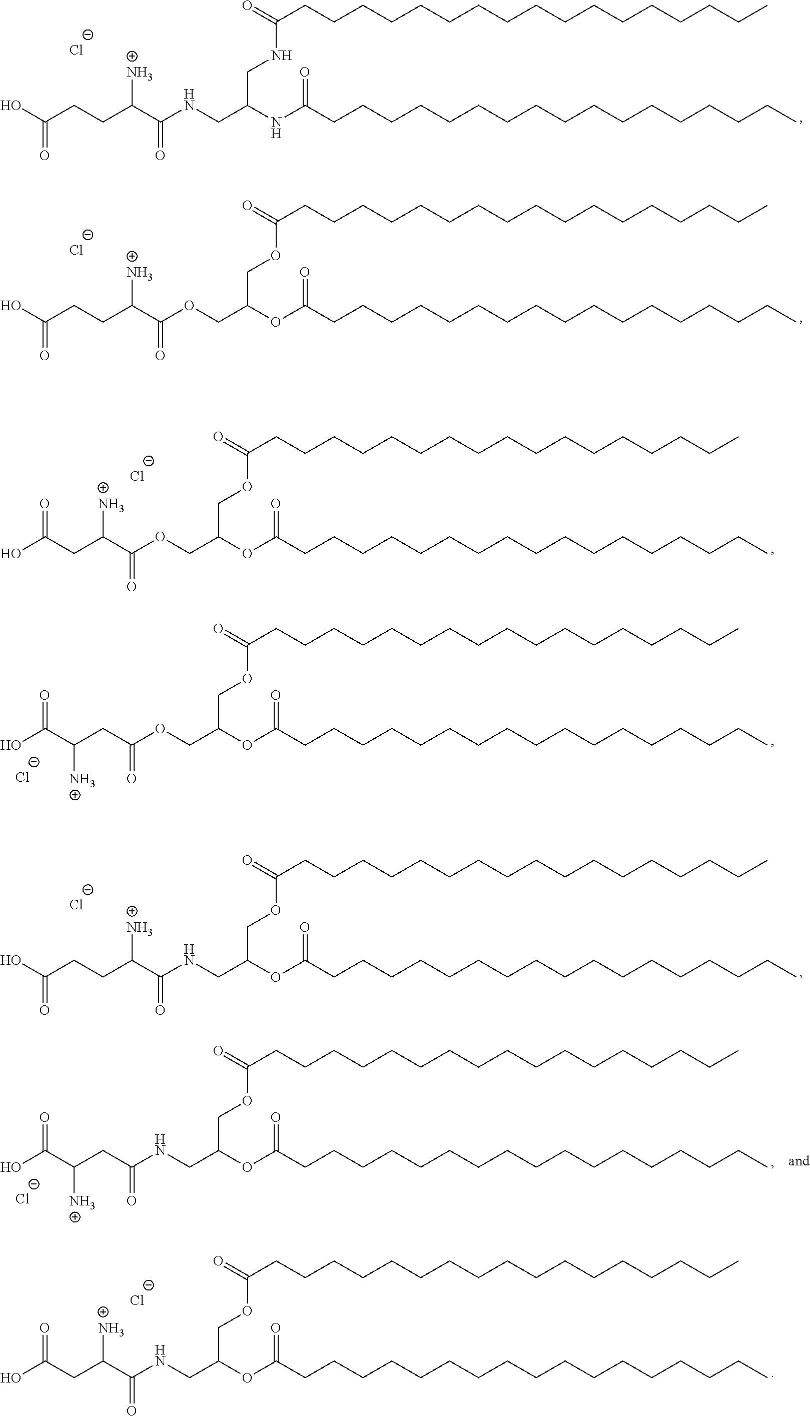

Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. Application No. 62/520,530.

Polyethylene Glycol (PEG)-Lipids

The lipid composition of a pharmaceutical composition disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DS G), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

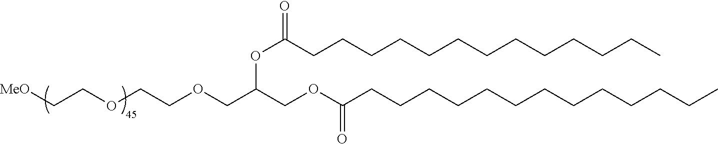

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

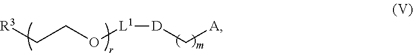

or salts thereof, wherein:

R³ is —OR^{O};

R^{O} is hydrogen, optionally substituted alkyl, or an oxygen protecting group;

r is an integer between 1 and 100, inclusive;

L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

D is a moiety obtained by click chemistry or a moiety cleavable under physiological conditions;

m is 0, 1, 2, 3, 4, 5, 6, 7, 8, 9, or 10;

A is of the formula:

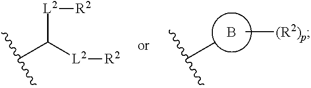

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2.

In certain embodiments, the compound of Formula (V) is a PEG-OH lipid (i.e., R³ is —OR^{O}, and R^{O} is hydrogen). In certain embodiments, the compound of Formula (V) is of Formula (V-OH):

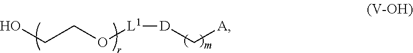

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI):

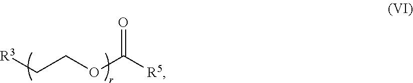

or a salts thereof, wherein:

R³ is —OR^{O};

R^{O} is hydrogen, optionally substituted alkyl or an oxygen protecting group;

r is an integer between 1 and 100, inclusive;

R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), —NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), —NR^{N}C(S)N(R^{N}), 5(0), OS(O), S(O)O, OS(O)O, OS(O)₂, S(O)₂O, OS(O)₂O, N(R^{N})S(O), —S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O; and

each instance of R^{N} is independently hydrogen, optionally substituted alkyl, or a nitrogen protecting group.

In certain embodiments, the compound of Formula (VI) is of Formula (VI-OH):

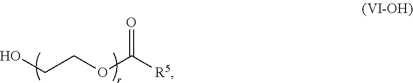

or a salt thereof. In some embodiments, r is 45.

In yet other embodiments the compound of Formula (VI) is:

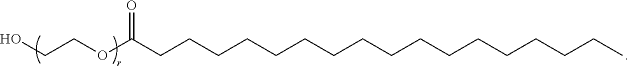

or a salt thereof.

In one embodiment, the compound of Formula (VI) is

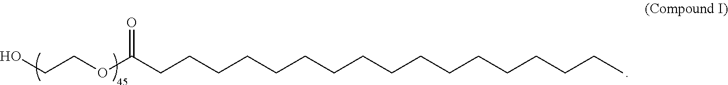

In some aspects, the lipid composition of the pharmaceutical compositions disclosed herein does not comprise a PEG-lipid.

In some embodiments, the PEG-lipids may be one or more of the PEG lipids described in U.S. Application No. 62/520,530.

In some embodiments, a PEG lipid of the invention comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a LNP of the invention comprises an ionizable cationic lipid of any of Formula I, II or III, a phospholipid comprising DSPC, a structural lipid, and a PEG lipid comprising PEG-DMG.

In some embodiments, a LNP of the invention comprises an ionizable cationic lipid of any of Formula I, II or III, a phospholipid comprising DSPC, a structural lipid, and a PEG lipid comprising a compound having Formula VI.

In some embodiments, a LNP of the invention comprises an ionizable cationic lipid of Formula I, II or III, a phospholipid comprising a compound having Formula IV, a structural lipid, and the PEG lipid comprising a compound having Formula V or VI.

In some embodiments, a LNP of the invention comprises an ionizable cationic lipid of Formula I, II or III, a phospholipid comprising a compound having Formula IV, a structural lipid, and the PEG lipid comprising a compound having Formula V or VI.

In some embodiments, a LNP of the invention comprises an ionizable cationic lipid of Formula I, II or III, a phospholipid having Formula IV, a structural lipid, and a PEG lipid comprising a compound having Formula VI.

In some embodiments, a LNP of the invention comprises an ionizable cationic lipid of

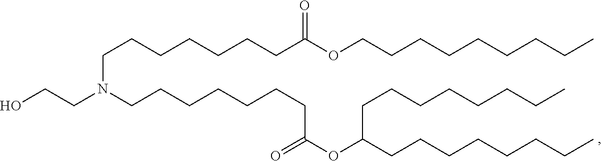 [SMILES: CCCCCCCCCOC(=O)CCCCCCCN(CCO)CCCCCCCC(=O)OC(CCCCCCCC)CCCCCCCC]

and a PEG lipid comprising Formula VI.

In some embodiments, a LNP of the invention comprises an ionizable cationic lipid of

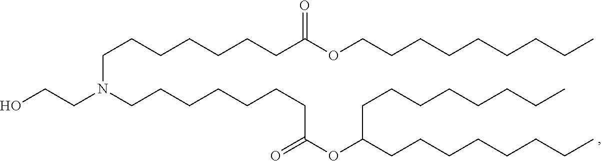 [SMILES: CCCCCCCCCOC(=O)CCCCCCCN(CCO)CCCCCCCC(=O)OC(CCCCCCCC)CCCCCCCC]

and an alternative lipid comprising oleic acid.

In some embodiments, a LNP of the invention comprises an ionizable cationic lipid of

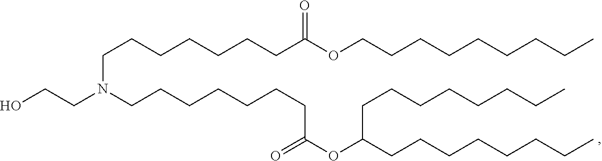 [SMILES: CCCCCCCCCOC(=O)CCCCCCCN(CCO)CCCCCCCC(=O)OC(CCCCCCCC)CCCCCCCC]

an alternative lipid comprising oleic acid, a structural lipid comprising cholesterol, and a PEG lipid comprising a compound having Formula VI.

In some embodiments, a LNP of the invention comprises an ionizable cationic lipid of

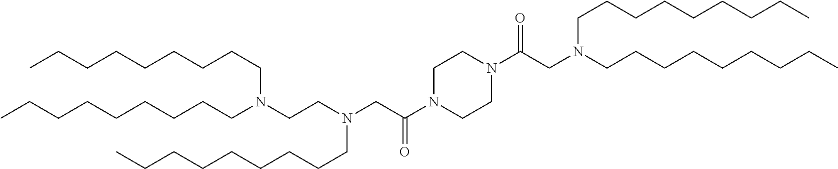 [SMILES: CCCCCCCCCN(CCCCCCCCC)CCN(CCCCCCCCC)CC(=O)N1CCN(C(=O)CN(CCCCCCCCC)CCCCCCCCC)CC1]

a phospholipid comprising DOPE, a structural lipid comprising cholesterol, and a PEG lipid comprising a compound having Formula VI.

In some embodiments, a LNP of the invention comprises an ionizable cationic lipid of

a phospholipid comprising DOPE, a structural lipid comprising cholesterol, and a PEG lipid comprising a compound having Formula VII.

In some embodiments, a LNP of the invention comprises an N:P ratio of from about 2:1 to about 30:1.

In some embodiments, a LNP of the invention comprises an N:P ratio of about 6:1.

In some embodiments, a LNP of the invention comprises an N:P ratio of about 3:1.

In some embodiments, a LNP of the invention comprises a wt/wt ratio of the ionizable cationic lipid component to the RNA of from about 10:1 to about 100:1.

In some embodiments, a LNP of the invention comprises a wt/wt ratio of the ionizable cationic lipid component to the RNA of about 20:1.

In some embodiments, a LNP of the invention comprises a wt/wt ratio of the ionizable cationic lipid component to the RNA of about 10:1.

In some embodiments, a LNP of the invention has a mean diameter from about 50 nm to about 150 nm.

In some embodiments, a LNP of the invention has a mean diameter from about 70 nm to about 120 nm.

As used herein, the term “alkyl”, “alkyl group”, or “alkylene” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C₁₋₁₄ alkyl” means an optionally substituted linear or branched, saturated hydrocarbon including 1 14 carbon atoms. Unless otherwise specified, an alkyl group described herein refers to both unsubstituted and substituted alkyl groups.

As used herein, the term “alkenyl”, “alkenyl group”, or “alkenylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C2-14 alkenyl” means an optionally substituted linear or branched hydrocarbon including 2 14 carbon atoms and at least one carbon-carbon double bond. An alkenyl group may include one, two, three, four, or more carbon-carbon double bonds. For example, C18 alkenyl may include one or more double bonds. A C18 alkenyl group including two double bonds may be a linoleyl group. Unless otherwise specified, an alkenyl group described herein refers to both unsubstituted and substituted alkenyl groups.

As used herein, the term “alkynyl”, “alkynyl group”, or “alkynylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one carbon-carbon triple bond, which is optionally substituted. The notation “C2-14 alkynyl” means an optionally substituted linear or branched hydrocarbon including 2 14 carbon atoms and at least one carbon-carbon triple bond. An alkynyl group may include one, two, three, four, or more carbon-carbon triple bonds. For example, C18 alkynyl may include one or more carbon-carbon triple bonds. Unless otherwise specified, an alkynyl group described herein refers to both unsubstituted and substituted alkynyl groups.

As used herein, the term “carbocycle” or “carbocyclic group” means an optionally substituted mono- or multi-cyclic system including one or more rings of carbon atoms. Rings may be three, four, five, six, seven, eight, nine, ten, eleven, twelve, thirteen, fourteen, fifteen, sixteen, seventeen, eighteen, nineteen, or twenty membered rings. The notation “C3-6 carbocycle” means a carbocycle including a single ring having 3-6 carbon atoms. Carbocycles may include one or more carbon-carbon double or triple bonds and may be non-aromatic or aromatic (e.g., cycloalkyl or aryl groups). Examples of carbocycles include cyclopropyl, cyclopentyl, cyclohexyl, phenyl, naphthyl, and 1,2 dihydronaphthyl groups. The term “cycloalkyl” as used herein means a non-aromatic carbocycle and may or may not include any double or triple bond. Unless otherwise specified, carbocycles described herein refers to both unsubstituted and substituted carbocycle groups, i.e., optionally substituted carbocycles.

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. The term “heterocycloalkyl” as used herein means a non-aromatic heterocycle and may or may not include any double or triple bond. Unless otherwise specified, heterocycles described herein refers to both unsubstituted and substituted heterocycle groups, i.e., optionally substituted heterocycles.

As used herein, the term “heteroalkyl”, “heteroalkenyl”, or “heteroalkynyl”, refers respectively to an alkyl, alkenyl, alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. Unless otherwise specified, heteroalkyls, heteroalkenyls, or heteroalkynyls described herein refers to both unsubstituted and substituted heteroalkyls, heteroalkenyls, or heteroalkynyls, i.e., optionally substituted heteroalkyls, heteroalkenyls, or heteroalkynyls.

As used herein, a “biodegradable group” is a group that may facilitate faster metabolism of a lipid in a mammalian entity. A biodegradable group may be selected from the group consisting of, but is not limited to, —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group. As used herein, an “aryl group” is an optionally substituted carbocyclic group including one or more aromatic rings. Examples of aryl groups include phenyl and naphthyl groups. As used herein, a “heteroaryl group” is an optionally substituted heterocyclic group including one or more aromatic rings. Examples of heteroaryl groups include pyrrolyl, furyl, thiophenyl, imidazolyl, oxazolyl, and thiazolyl. Both aryl and heteroaryl groups may be optionally substituted. For example, M and M′ can be selected from the non-limiting group consisting of optionally substituted phenyl, oxazole, and thiazole. In the formulas herein, M and M′ can be independently selected from the list of biodegradable groups above. Unless otherwise specified, aryl or heteroaryl groups described herein refers to both unsubstituted and substituted groups, i.e., optionally substituted aryl or heteroaryl groups.

Alkyl, alkenyl, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., C(O)OH), an alcohol (e.g., a hydroxyl, OH), an ester (e.g., C(O)OR OC(O)R), an aldehyde (e.g., C(O)H), a carbonyl (e.g., C(O)R, alternatively represented by C═O), an acyl halide (e.g., C(O)X, in which X is a halide selected from bromide, fluoride, chloride, and iodide), a carbonate (e.g., OC(O)OR), an alkoxy (e.g., OR), an acetal (e.g., C(OR)2R″″, in which each OR are alkoxy groups that can be the same or different and R″″ is an alkyl or alkenyl group), a phosphate (e.g., P(O)43-), a thiol (e.g., SH), a sulfoxide (e.g., S(O)R), a sulfinic acid (e.g., S(O)OH), a sulfonic acid (e.g., S(O)2OH), a thial (e.g., C(S)H), a sulfate (e.g., S(O)42-), a sulfonyl (e.g., S(O)2), an amide (e.g., C(O)NR2, or N(R)C(O)R), an azido (e.g., N3), a nitro (e.g., NO2), a cyano (e.g., CN), an isocyano (e.g., NC), an acyloxy (e.g., OC(O)R), an amino (e.g., NR2, NRH, or NH2), a carbamoyl (e.g., OC(O)NR2, OC(O)NRH, or OC(O)NH2), a sulfonamide (e.g., S(O)2NR2, S(O)2NRH, S(O)2NH2, N(R)S(O)2R, N(H)S(O)2R, N(R)S(O)2H, or N(H)S(O)2H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. For example, a C1 6 alkyl group may be further substituted with one, two, three, four, five, or six substituents as described herein.

Compounds of the disclosure that contain nitrogens can be converted to N-oxides by treatment with an oxidizing agent (e.g., 3-chloroperoxybenzoic acid (mCPBA) and/or hydrogen peroxides) to afford other compounds of the disclosure. Thus, all shown and claimed nitrogen-containing compounds are considered, when allowed by valency and structure, to include both the compound as shown and its N-oxide derivative (which can be designated as N□O or N+—O—). Furthermore, in other instances, the nitrogens in the compounds of the disclosure can be converted to N-hydroxy or N-alkoxy compounds. For example, N-hydroxy compounds can be prepared by oxidation of the parent amine by an oxidizing agent such as m CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C1-C 6 alkyl, C1-C6 alkenyl, C1-C6 alkynyl, 3-14-membered carbocycle or 3-14-membered heterocycle) derivatives.

### Other Lipid Composition Components

The lipid composition of a pharmaceutical composition disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

The ratio between the lipid composition and the polynucleotide range can be from about 10:1 to about 60:1 (wt/wt).

In some embodiments, the ratio between the lipid composition and the polynucleotide can be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the polynucleotide encoding a therapeutic agent is about 20:1 or about 15:1.

In some embodiments, the pharmaceutical composition disclosed herein can contain more than one polypeptides. For example, a pharmaceutical composition disclosed herein can contain two or more polynucleotides (e.g., RNA, e.g., mRNA).

In one embodiment, the lipid nanoparticles described herein can comprise polynucleotides (e.g., mRNA) in a lipid:polynucleotide weight ratio of 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1, from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In one embodiment, the lipid nanoparticles described herein can comprise the polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

### Nanoparticle Compositions

In some embodiments, the pharmaceutical compositions disclosed herein are formulated as lipid nanoparticles (LNP). Accordingly, the present disclosure also provides nanoparticle compositions comprising (i) a lipid composition comprising a delivery agent such as compound as described herein, and (ii) at least one mRNA encoding a polypeptide. In such nanoparticle composition, the lipid composition disclosed herein can encapsulate the at least one mRNA encoding a polypeptide.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, a lipid nanoparticle comprises an ionizable lipid, a structural lipid, a phospholipid, and mRNA. In some embodiments, the LNP comprises an ionizable lipid, a PEG-modified lipid, a sterol and a structural lipid. In some embodiments, the LNP has a molar ratio of about 20-60% ionizable lipid: about 5-25% structural lipid: about 25-55% sterol; and about 0.5-15% PEG-modified lipid.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids leads them to form liposomes, vesicles, or membranes in aqueous media.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In one embodiment, the ionizable amino lipid may have a positively charged hydrophilic head and a hydrophobic tail that are connected via a linker structure.

In addition to these, an ionizable lipid may also be a lipid including a cyclic amine group. In one embodiment, the ionizable lipid may be selected from, but not limited to, a ionizable lipid described in International Publication Nos. WO2013086354 and WO2013116126; the contents of each of which are herein incorporated by reference in their entirety.

In yet another embodiment, the ionizable lipid may be selected from, but not limited to, formula CLI-CLXXXXII of U.S. Pat. No. 7,404,969; each of which is herein incorporated by reference in their entirety.

In one embodiment, the lipid may be a cleavable lipid such as those described in International Publication No. WO2012170889, herein incorporated by reference in its entirety. In one embodiment, the lipid may be synthesized by methods known in the art and/or as described in International Publication Nos. WO2013086354; the contents of each of which are herein incorporated by reference in their entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide.

As used herein, “size” or “mean size” in the context of nanoparticle compositions refers to the mean diameter of a nanoparticle composition.

In one embodiment, the polynucleotide encoding a polypeptide is formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the nanoparticles have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

The term “encapsulation efficiency” of a polynucleotide describes the amount of the polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of the polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a polynucleotide present in a pharmaceutical composition disclosed herein can depend on multiple factors such as the size of the polynucleotide, desired target and/or application, or other properties of the nanoparticle composition as well as on the properties of the polynucleotide.

For example, the amount of an mRNA useful in a nanoparticle composition can depend on the size (expressed as length, or molecular mass), sequence, and other characteristics of the mRNA. The relative amounts of a polynucleotide in a nanoparticle composition can also vary. The relative amounts of the lipid composition and the polynucleotide present in a lipid nanoparticle composition of the present disclosure can be optimized according to considerations of efficacy and tolerability. For compositions including an mRNA as a polynucleotide, the N:P ratio can serve as a useful metric.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition.

In general, a lower N:P ratio is preferred. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et al. (2015) “Delivery of oligonucleotides with lipid nanoparticles” Adv. Drug Deliv. Rev. 87:68-80; Silva et al. (2015) “Delivery Systems for Biopharmaceuticals. Part I: Nanoparticles and Microparticles” Curr. Pharm. Technol. 16: 940-954; Naseri et al. (2015) “Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

Other Delivery Agents

a. Liposomes, Lipoplexes, and Lipid Nanoparticles

In some embodiments, the compositions or formulations of the present disclosure comprise a delivery agent, e.g., a liposome, a lioplexes, a lipid nanoparticle, or any combination thereof. The polynucleotides described herein (e.g., a polynucleotide comprising a nucleotide sequence encoding a polypeptide) can be formulated using one or more liposomes, lipoplexes, or lipid nanoparticles. Liposomes, lipoplexes, or lipid nanoparticles can be used to improve the efficacy of the mRNAs directed protein production as these formulations can increase cell transfection by the mRNA; and/or increase the translation of encoded protein. The liposomes, lipoplexes, or lipid nanoparticles can also be used to increase the stability of the mRNAs.

Liposomes are artificially-prepared vesicles that can primarily be composed of a lipid bilayer and can be used as a delivery vehicle for the administration of pharmaceutical formulations. Liposomes can be of different sizes. A multilamellar vesicle (MLV) can be hundreds of nanometers in diameter, and can contain a series of concentric bilayers separated by narrow aqueous compartments. A small unicellular vesicle (SUV) can be smaller than 50 nm in diameter, and a large unilamellar vesicle (LUV) can be between 50 and 500 nm in diameter. Liposome design can include, but is not limited to, opsonins or ligands to improve the attachment of liposomes to unhealthy tissue or to activate events such as, but not limited to, endocytosis. Liposomes can contain a low or a high pH value in order to improve the delivery of the pharmaceutical formulations.

The formation of liposomes can depend on the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimal size, polydispersity and the shelf-life of the vesicles for the intended application, and the batch-to-batch reproducibility and scale up production of safe and efficient liposomal products, etc.

As a non-limiting example, liposomes such as synthetic membrane vesicles can be prepared by the methods, apparatus and devices described in U.S. Pub. Nos. US20130177638, US20130177637, US20130177636, US20130177635, US20130177634, US20130177633, US20130183375, US20130183373, and US20130183372. In some embodiments, the mRNAs described herein can be encapsulated by the liposome and/or it can be contained in an aqueous core that can then be encapsulated by the liposome as described in, e.g., Intl. Pub. Nos. WO2012031046, WO2012031043, WO2012030901, WO2012006378, and WO2013086526; and U.S. Pub. Nos. US20130189351, US20130195969 and US20130202684. Each of the references in herein incorporated by reference in its entirety.

In some embodiments, the mRNAs described herein can be formulated in a cationic oil-in-water emulsion where the emulsion particle comprises an oil core and a cationic lipid that can interact with the mRNA anchoring the molecule to the emulsion particle. In some embodiments, the mRNAs described herein can be formulated in a water-in-oil emulsion comprising a continuous hydrophobic phase in which the hydrophilic phase is dispersed. Exemplary emulsions can be made by the methods described in Intl. Pub. Nos. WO2012006380 and WO201087791, each of which is herein incorporated by reference in its entirety.

In some embodiments, the mRNAs described herein can be formulated in a lipid-polycation complex. The formation of the lipid-polycation complex can be accomplished by methods as described in, e.g., U.S. Pub. No. US20120178702. As a non-limiting example, the polycation can include a cationic peptide or a polypeptide such as, but not limited to, polylysine, polyornithine and/or polyarginine and the cationic peptides described in Intl. Pub. No. WO2012013326 or U.S. Pub. No. US20130142818. Each of the references is herein incorporated by reference in its entirety.

In some embodiments, the mRNAs described herein can be formulated in a lipid nanoparticle (LNP) such as those described in Intl. Pub. Nos. WO2013123523, WO2012170930, WO2011127255 and WO2008103276; and U.S. Pub. No. US20130171646, each of which is herein incorporated by reference in its entirety.

Lipid nanoparticle formulations typically comprise one or more lipids. In some embodiments, the lipid is an ionizable lipid (e.g., an ionizable amino lipid), sometimes referred to in the art as an “ionizable cationic lipid”. In some embodiments, lipid nanoparticle formulations further comprise other components, including a phospholipid, a structural lipid, and a molecule capable of reducing particle aggregation, for example a PEG or PEG-modified lipid.

Exemplary ionizable lipids include, but not limited to, any one of Compounds 1-342 disclosed herein, DLin-MC3-DMA (MC3), DLin-DMA, DLenDMA, DLin-D-DMA, DLin-K-DMA, DLin-M-C₂-DMA, DLin-K-DMA, DLin-KC2-DMA, DLin-KC3-DMA, DLin-KC4-DMA, DLin-C₂K-DMA, DLin-MP-DMA, DODMA, 98N12-5, C₁₂-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), and any combination thereof. Other exemplary ionizable lipids include, (13Z,16Z)-N,N-dimethyl-3-nonyldocosa-13,16-dien-1-amine (L608), (20Z,23Z)-N,N-dimethylnonacosa-20,23-dien-10-amine, (17Z,20Z)-N,N-dimemylhexacosa-17,20-dien-9-amine, (16Z,19Z)-N5N-dimethylpentacosa-16,19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z,17Z)-N,N-dimethyltricosa-14,17-dien-4-amine, (19Z,22Z)-N,N-dimeihyloctacosa-19,22-dien-9-amine, (18Z,21Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl]pyrrolidine, (20Z)-N,N-dimethylheptacos-20-en-10-amine, (15Z)-N,N-dimethyl eptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-1-amine, N,N-dimethyl-1-[(1S,2R)-2-octylcyclopropyl] eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecylcyclopropyl] tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R-N,N-dimethyl-1-[(9Z,12Z)-octadeca-9,12-dien-1-yloxy]-3-(octyloxy)propan-2-amine, S-N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl] ethyl}pyrrolidine, (2S)-N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl] ethyl} azetidine, (25)-1-(hexyloxy)-N,N-dimethyl-3-[(9Z,12Z)-octadeca-9,12-dien-1-yloxylpropan-2-amine, (2S)-1-(heptyloxy)-N,N-dimethyl-3-[(9Z,12Z)-octadec α-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)-N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z, 16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-do cos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)-N,N-dimethyl-H(1-metoyloctyl)oxyl-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-1 [8-(2-oclylcyclopropyl)octyl]oxyl-3-(octyloxy)propan-2-amine, and (11E,20Z,23Z)-N,N-dimethylnonacosa-11,20,2-trien-10-amine, and any combination thereof.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin. In some embodiments, the phospholipids are DLPC, DMPC, DOPC, DPPC, DSPC, DUPC, 18:0 Diether PC, DLnPC, DAPC, DHAPC, DOPE, 4ME 16:0 PE, DSPE, DLPE, DLnPE, DAPE, DHAPE, DOPG, and any combination thereof. In some embodiments, the phospholipids are MPPC, MSPC, PMPC, PSPC, SMPC, SPPC, DHAPE, DOPG, and any combination thereof. In some embodiments, the amount of phospholipids (e.g., DSPC) in the lipid composition ranges from about 1 mol % to about 20 mol %.

The structural lipids include sterols and lipids containing sterol moieties. In some embodiments, the structural lipids include cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the amount of the structural lipids (e.g., cholesterol) in the lipid composition ranges from about 20 mol % to about 60 mol %.

The PEG-modified lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG DMPE, PEG-DPPC, or a PEG-DSPE lipid. In some embodiments, the PEG-lipid are 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG moiety has a size of about 1000, 2000, 5000, 10,000, 15,000 or 20,000 daltons. In some embodiments, the amount of PEG-lipid in the lipid composition ranges from about 0 mol % to about 5 mol %.

In some embodiments, the LNP formulations described herein can additionally comprise a permeability enhancer molecule. Non-limiting permeability enhancer molecules are described in U.S. Pub. No. US20050222064, herein incorporated by reference in its entirety.

The LNP formulations can further contain a phosphate conjugate. The phosphate conjugate can increase in vivo circulation times and/or increase the targeted delivery of the nanoparticle. Phosphate conjugates can be made by the methods described in, e.g., Intl. Pub. No. WO2013033438 or U.S. Pub. No. US20130196948. The LNP formulation can also contain a polymer conjugate (e.g., a water soluble conjugate) as described in, e.g., U.S. Pub. Nos. US20130059360, US20130196948, and US20130072709. Each of the references is herein incorporated by reference in its entirety.

The LNP formulations can comprise a conjugate to enhance the delivery of nanoparticles of the present invention in a subject. Further, the conjugate can inhibit phagocytic clearance of the nanoparticles in a subject. In some embodiments, the conjugate can be a “self” peptide designed from the human membrane protein CD47 (e.g., the “self” particles described by Rodriguez et al, Science 2013 339, 971-975, herein incorporated by reference in its entirety). As shown by Rodriguez et al. the self peptides delayed macrophage-mediated clearance of nanoparticles which enhanced delivery of the nanoparticles.

The LNP formulations can comprise a carbohydrate carrier. As a non-limiting example, the carbohydrate carrier can include, but is not limited to, an anhydride-modified phytoglycogen or glycogen-type material, phytoglycogen octenyl succinate, phytoglycogen beta-dextrin, anhydride-modified phytoglycogen beta-dextrin (e.g., Intl. Pub. No. WO2012109121, herein incorporated by reference in its entirety).

The LNP formulations can be coated with a surfactant or polymer to improve the delivery of the particle. In some embodiments, the LNP can be coated with a hydrophilic coating such as, but not limited to, PEG coatings and/or coatings that have a neutral surface charge as described in U.S. Pub. No. US20130183244, herein incorporated by reference in its entirety.

The LNP formulations can be engineered to alter the surface properties of particles so that the lipid nanoparticles can penetrate the mucosal barrier as described in U.S. Pat. No. 8,241,670 or Intl. Pub. No. WO2013110028, each of which is herein incorporated by reference in its entirety.

The LNP engineered to penetrate mucus can comprise a polymeric material (i.e., a polymeric core) and/or a polymer-vitamin conjugate and/or a tri-block co-polymer. The polymeric material can include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates.

LNP engineered to penetrate mucus can also include surface altering agents such as, but not limited to, mRNAs, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β 4 dornase alfa, neltenexine, erdosteine) and various DNases including rhDNase.

In some embodiments, the mucus penetrating LNP can be a hypotonic formulation comprising a mucosal penetration enhancing coating. The formulation can be hypotonic for the epithelium to which it is being delivered. Non-limiting examples of hypotonic formulations can be found in, e.g., Intl. Pub. No. WO2013110028, herein incorporated by reference in its entirety.

In some embodiments, the mRNA described herein is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, Mass.), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

In some embodiments, the mRNAs described herein are formulated as a solid lipid nanoparticle (SLN), which can be spherical with an average diameter between 10 to 1000 nm. SLN possess a solid lipid core matrix that can solubilize lipophilic molecules and can be stabilized with surfactants and/or emulsifiers. Exemplary SLN can be those as described in Intl. Pub. No. WO2013105101, herein incorporated by reference in its entirety.

In some embodiments, the mRNAs described herein can be formulated for controlled release and/or targeted delivery. As used herein, “controlled release” refers to a pharmaceutical composition or compound release profile that conforms to a particular pattern of release to effect a therapeutic outcome. In one embodiment, the mRNAs can be encapsulated into a delivery agent described herein and/or known in the art for controlled release and/or targeted delivery. As used herein, the term “encapsulate” means to enclose, surround or encase. As it relates to the formulation of the compounds of the invention, encapsulation can be substantial, complete or partial. The term “substantially encapsulated” means that at least greater than 50, 60, 70, 80, 85, 90, 95, 96, 97, 98, 99, or greater than 99% of the pharmaceutical composition or compound of the invention can be enclosed, surrounded or encased within the delivery agent. “Partially encapsulation” means that less than 10, 10, 20, 30, 40 50 or less of the pharmaceutical composition or compound of the invention can be enclosed, surrounded or encased within the delivery agent.

Advantageously, encapsulation can be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, or greater than 99% of the pharmaceutical composition or compound of the invention are encapsulated in the delivery agent.

In some embodiments, the mRNAs described herein can be encapsulated in a therapeutic nanoparticle, referred to herein as “therapeutic nanoparticle mRNAs.” Therapeutic nanoparticles can be formulated by methods described in, e.g., Intl. Pub. Nos. WO2010005740, WO2010030763, WO2010005721, WO2010005723, and WO2012054923; and U.S. Pub. Nos. US20110262491, US20100104645, US20100087337, US20100068285, US20110274759, US20100068286, US20120288541, US20120140790, US20130123351 and US20130230567; and U.S. Pat. Nos. 8,206,747, 8,293,276, 8,318,208 and 8,318,211, each of which is herein incorporated by reference in its entirety.

In some embodiments, the therapeutic nanoparticle mRNA can be formulated for sustained release. As used herein, “sustained release” refers to a pharmaceutical composition or compound that conforms to a release rate over a specific period of time. The period of time can include, but is not limited to, hours, days, weeks, months and years. As a non-limiting example, the sustained release nanoparticle of the mRNAs described herein can be formulated as disclosed in Intl. Pub. No. WO2010075072 and U.S. Pub. Nos. US20100216804, US20110217377, US20120201859 and US20130150295, each of which is herein incorporated by reference in their entirety.

In some embodiments, the therapeutic nanoparticle mRNA can be formulated to be target specific, such as those described in Intl. Pub. Nos. WO2008121949, WO2010005726, WO2010005725, WO2011084521 and WO2011084518; and U.S. Pub. Nos. US20100069426, US20120004293 and US20100104655, each of which is herein incorporated by reference in its entirety.

The LNPs can be prepared using microfluidic mixers or micromixers. Exemplary microfluidic mixers can include, but are not limited to, a slit interdigital micromixer including, but not limited to those manufactured by Microinnova (Allerheiligen bei Wildon, Austria) and/or a staggered herringbone micromixer (SHM) (see Zhigaltsev et al., “Bottom-up design and synthesis of limit size lipid nanoparticle systems with aqueous and triglyceride cores using millisecond microfluidic mixing,” Langmuir 28:3633-40 (2012); Belliveau et al., “Microfluidic synthesis of highly potent limit-size lipid nanoparticles for in vivo delivery of siRNA,” Molecular Therapy-Nucleic Acids. 1:e37 (2012); Chen et al., “Rapid discovery of potent siRNA-containing lipid nanoparticles enabled by controlled microfluidic formulation,” J. Am. Chem. Soc. 134(16):6948-51 (2012); each of which is herein incorporated by reference in its entirety). Exemplary micromixers include Slit Interdigital Microstructured Mixer (SIMM-V2) or a Standard Slit Interdigital Micro Mixer (SSIMM) or Caterpillar (CPMM) or Impinging-jet (IJMM) from the Institut für Mikrotechnik Mainz GmbH, Mainz Germany. In some embodiments, methods of making LNP using SHM further comprise mixing at least two input streams wherein mixing occurs by microstructure-induced chaotic advection (MICA). According to this method, fluid streams flow through channels present in a herringbone pattern causing rotational flow and folding the fluids around each other. This method can also comprise a surface for fluid mixing wherein the surface changes orientations during fluid cycling. Methods of generating LNPs using SHM include those disclosed in U.S. Pub. Nos. US20040262223 and US20120276209, each of which is incorporated herein by reference in their entirety.

In some embodiments, the mRNAs described herein can be formulated in lipid nanoparticles using microfluidic technology (see Whitesides, George M., “The Origins and the Future of Microfluidics,” Nature 442: 368-373 (2006); and Abraham et al., “Chaotic Mixer for Microchannels,” Science 295: 647-651 (2002); each of which is herein incorporated by reference in its entirety). In some embodiments, the mRNAs can be formulated in lipid nanoparticles using a micromixer chip such as, but not limited to, those from Harvard Apparatus (Holliston, Mass.) or Dolomite Microfluidics (Royston, UK). A micromixer chip can be used for rapid mixing of two or more fluid streams with a split and recombine mechanism.

In some embodiments, the mRNAs described herein can be formulated in lipid nanoparticles having a diameter from about 1 nm to about 100 nm such as, but not limited to, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles can have a diameter from about 10 to 500 nm. In one embodiment, the lipid nanoparticle can have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the mRNAs can be delivered using smaller LNPs. Such particles can comprise a diameter from below 0.1 μm up to 100 nm such as, but not limited to, less than 0.1 μm, less than 1.0 μm, less than 5 μm, less than 10 μm, less than 15 um, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 um, less than 250 um, less than 275 um, less than 300 um, less than 325 um, less than 350 um, less than 375 um, less than 400 um, less than 425 um, less than 450 um, less than 475 um, less than 500 um, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, or less than 975 um.

The nanoparticles and microparticles described herein can be geometrically engineered to modulate macrophage and/or the immune response. The geometrically engineered particles can have varied shapes, sizes and/or surface charges to incorporate the mRNAs described herein for targeted delivery such as, but not limited to, pulmonary delivery (see, e.g., Intl. Pub. No. WO2013082111, herein incorporated by reference in its entirety). Other physical features the geometrically engineering particles can include, but are not limited to, fenestrations, angled arms, asymmetry and surface roughness, charge that can alter the interactions with cells and tissues.

In some embodiment, the nanoparticles described herein are stealth nanoparticles or target-specific stealth nanoparticles such as, but not limited to, those described in U.S. Pub. No. US20130172406, herein incorporated by reference in its entirety. The stealth or target-specific stealth nanoparticles can comprise a polymeric matrix, which can comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates, or combinations thereof.

b. Lipidoids

In some embodiments, the compositions or formulations of the present disclosure comprise a delivery agent, e.g., a lipidoid. The mRNAs described herein (e.g., an mRNA comprising a nucleotide sequence encoding a polypeptide) can be formulated with lipidoids. Complexes, micelles, liposomes or particles can be prepared containing these lipidoids and therefore to achieve an effective delivery of the mRNA, as judged by the production of an encoded protein, following the injection of a lipidoid formulation via localized and/or systemic routes of administration. Lipidoid complexes of mRNAs can be administered by various means including, but not limited to, intravenous, intramuscular, or subcutaneous routes.

The synthesis of lipidoids is described in literature (see Mahon et al., Bioconjug. Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001; all of which are incorporated herein in their entireties).

Formulations with the different lipidoids, including, but not limited to penta[3-(1-laurylaminopropionyl)]-triethylenetetramine hydrochloride (TETA-5LAP; also known as 98N12-5, see Murugaiah et al., Analytical Biochemistry, 401:61 (2010)), C₁₂-200 (including derivatives and variants), and MD1, can be tested for in vivo activity. The lipidoid “98N12-5” is disclosed by Akinc et al., Mol Ther. 2009 17:872-879. The lipidoid “C₁₂-200” is disclosed by Love et al., Proc Natl Acad Sci USA. 2010 107:1864-1869 and Liu and Huang, Molecular Therapy. 2010 669-670. Each of the references is herein incorporated by reference in its entirety.

In one embodiment, the mRNAs described herein can be formulated in an aminoalcohol lipidoid. Aminoalcohol lipidoids can be prepared by the methods described in U.S. Pat. No. 8,450,298 (herein incorporated by reference in its entirety).

The lipidoid formulations can include particles comprising either 3 or 4 or more components in addition to mRNAs. Lipidoids and mRNA formulations comprising lipidoids are described in Intl. Pub. No. WO 2015051214 (herein incorporated by reference in its entirety.

### Polypeptides of Interest

In some embodiments, an mRNA of the disclosure encodes a polypeptide of interest that is a therapeutic polypeptide. In some embodiments, an mRNA of the disclosure encodes a polypeptide of interest that is a full-length protein. In some embodiments, an mRNA of the disclosure encodes a polypeptide of interest that is a functional fragment of a full-length protein (e.g., a fragment of the full-length protein that includes one or more functional domains such that the functional activity of the full-length protein is retained). In some embodiments, an mRNA of the disclosure encodes a polypeptide of interest that is not naturally occurring. In some embodiments, an mRNA of the disclosure encodes a polypeptide of interest that is a modified protein comprised of one or more heterologous domains (e.g., a protein that is a fusion protein comprised of one or more domains that do not naturally occur in the protein such that the function of the protein is altered).

Exemplary types of proteins (e.g., infectious disease antigens, tumor cell antigens, soluble effector molecules, antibodies, enzymes, recruitment factors, transcription factors, membrane bound receptors or ligands) that are encoded by an mRNA of the disclosure are described in detail in the following subsections.

Naturally Occurring Targets

In some embodiments, an mRNA of the disclosure encodes a polypeptide of interest that is a naturally occurring target. In some embodiments, an mRNA encodes a polypeptide of interest that when expressed, modulates a naturally occurring target (e.g., up- or down-regulates the activity of a naturally occurring target). In some embodiments, a naturally occurring target is a soluble protein that is secreted by a cell. In some embodiments, a naturally occurring target is a protein that is retained within a cell (e.g., an intracellular protein). In some embodiments, a naturally occurring target is a membrane-bound or transmembrane protein. Non-limiting examples of naturally occurring targets include soluble proteins (e.g., chemokines, cytokines, growth factors, antibodies, enzymes), intracellular proteins (e.g., intracellular signaling proteins, transcription factors, enzymes, structural proteins) and membrane-bound or transmembrane proteins (e.g., receptors, adhesion molecules, enzymes).

In some embodiments, an mRNA encodes a polypeptide of interest that when expressed is a full-length naturally occurring target (i.e., a full-length protein). In some embodiments, an mRNA encodes a polypeptide of interest that when expressed is a fragment or portion of a naturally occurring target (i.e., a fragment or portion of a full-length protein). For example, in one embodiment, the protein or fragment thereof can be an immunogenic polypeptide that can be used as a vaccine.

In some embodiments, an mRNA encodes a polypeptide that when expressed, modulates a naturally occurring target (e.g., by encoding the target itself or by functioning to modulate the activity of the target). In some embodiments, a polypeptide of interest acts in an autocrine fashion, i.e., the polypeptide exerts an effect directly on the cell into which the mRNA is delivered. In some embodiments, an encoded polypeptide of interest acts in a paracrine fashion, i.e., the encoded polypeptide exerts an indirect effect on a cell that is not the cell into which the mRNA is delivered (e.g., delivery of the mRNA into one type of cell results in secretion of a molecule that exerts an effects on another type of cell, such as a bystander cell). In some embodiments, an encoded polypeptide of interest acts in both an autocrine fashion and a paracrine fashion.

### Naturally Occurring Soluble Targets

In some embodiments, an mRNA encodes a polypeptide of interest that modulates the activity of a naturally occurring soluble target, for example by encoding the soluble target itself or by modulating the expression (e.g., transcription or translation) of the soluble target. Non-limiting examples of naturally occurring soluble targets include cytokines, chemokines, growth factors, enzymes, and antibodies.

In some embodiments, an mRNA encoding a polypeptide of interest stimulates (e.g., upregulates, enhances) the activation or activity of a cell type, for example in situations where stimulation of an immune response is desirable, such as in cancer therapy or treatment of an infectious disease (e.g., a viral, bacterial, fungal, protozoal or parasitic infection). In another embodiment, an mRNA encoding a polypeptide of interest inhibits (e.g., downregulates, reduces) the activation or activity of a cell, for example in situations where inhibition of an immune response is desirable, such as in autoimmune diseases, allergies and transplantation.

In some embodiments, an mRNA of the disclosure encodes a soluble target that is a cytokine or chemokine with desirable uses for stimulating or inhibiting immune responses, e.g., that is useful in treating cancer as described further below.

In some embodiments, an mRNA of the disclosure encodes a soluble target that is a cytokine that stimulates the activation or activity of a cell such as an immune cell.

In some embodiments, an mRNA of the disclosure encodes a chemokine or a chemokine receptor which is useful for stimulating the activation or activity of an immune cell. Chemokines have been demonstrated to control the trafficking of inflammatory cells (including granulocytes and monocytes/monocytes), as well as regulating the movement of a wide variety of immune cells (including lymphocytes, natural killer cells and dendritic cells). Thus, chemokines are involved both in regulating inflammatory responses and immune responses. Moreover, chemokines have been shown to have effects on the proliferative and invasive properties of cancer cells (fora review of chemokines, see e.g., Mukaida, N. et al. (2014) Mediators of Inflammation, Article ID 170381, pg. 1-15).

In some embodiments, an mRNA of the disclosure encodes a recruitment factor which is useful to stimulate the homing, activation or activity of a cell. In one embodiment, the cell is an immune cell and the “recruitment factor” refers to a protein that promotes recruitment of an immune cell to a desired location (e.g., to a tumor site or an inflammatory site). For example, certain chemokines, chemokine receptors and cytokines have been shown to be involved in the recruitment of lymphocytes (see e.g., Oelkrug, C. and Ramage, J. M. (2014) Clin. Exp. Immunol. 178:1-8).

In some embodiments, an mRNA of the disclosure encodes an inhibitory cytokine or an antagonist of a stimulatory cytokine which is useful for inhibiting immune responses.

In some embodiments, an mRNA of the disclosure encodes a soluble target that is an antibody. As used herein, the term “antibody” refers to a whole antibody comprising two light chain polypeptides and two heavy chain polypeptides, or an antigen-binding fragment thereof. In some embodiments, a soluble target is a monoclonal antibody (e.g., full length monoclonal antibody) that displays a single binding specificity and affinity for a particular epitope. In some embodiments, a soluble target is an antigen binding fragment of a monoclonal antibody that retains the ability to bind a target antigen. Such fragments include, e.g., a single chain antibody, a single chain Fv fragment (scFv), an Fd fragment, an Fab fragment, an Fab′ fragment, or an F(ab′)₂ fragment.

In some embodiments, an mRNA of the disclosure encodes an antibody that recognizes a tumor antigen, against which a protective or a therapeutic immune response is desired, e.g., antigens expressed by a tumor cell. In some embodiments, a suitable antigen includes tumor associated antigens for the prevention or treatment of cancers.

In some embodiments, an mRNA of the disclosure encodes an antibody that recognizes an infectious disease antigen, against which protective or therapeutic immune responses are desired, e.g., an antigen present on a pathogen or infectious agent. In some embodiments, a suitable antigen includes an infectious disease associated antigen for the prevention or treatment of an infectious disease. Methods for identification of antigens on infectious disease agents that comprise protective epitopes (e.g., epitopes that when recognized by an antibody enable neutralization or blocking of infection caused by an infectious disease agent) are described in the art as detailed by Sharon, J. et al. (2013) Immunology 142:1-23. In some embodiments, an infectious disease antigen is present on a virus or on a bacterial cell.

In some embodiments, an mRNA of the disclosure encodes a soluble target that is a growth factor with desirable uses for modulating tissue healing and repair. A growth factor is a protein that stimulates the survival, growth, proliferation, migration or differentiation of cells, often for the purposes of promoting growth of lost tissue or enhancing the body's innate healing and repair mechanisms. In some embodiments, a growth factor is used to manipulate cells that include, but are not limited to, stromal cells (e.g., fibroblasts), immune cells, vascular cells (e.g., epithelial cells, platelets, pericytes), neural cells (e.g., astrocytes, neural stem cells, microglial cells), or bone cells (e.g., osteocyte, osteoblast, osteoclast, osteogenic cells).

In some embodiments, an mRNA of the disclosure encodes a soluble target that is an enzyme with desirable uses for modulating metabolism or growth in a subject. In some embodiments, an enzyme is administered to replace an endogenous enzyme that is absent or dysfunctional as described in Brady, R. et al, (2004) Lancet Neurol. 3:752. In some embodiments, an enzyme is used to treat a metabolic storage disease. A metabolic storage disease results from the systemic accumulation of metabolites due to the absence or dysfunction of an endogenous enzyme. Such metabolites include lipids, glycoproteins, and mucopolysaccharides. In some embodiments, an enzyme is used to reduce or eliminate the accumulation of monosaccharides, polysaccharides, glycoproteins, glycopeptides, glycolipids or lipids due to a metabolic storage disease.

Naturally Occurring Intracellular Targets

In some embodiments, an mRNA of the disclosure encodes a polypeptide of interest that modulates the activity of a naturally occurring intracellular target, for example by encoding the intracellular target itself or by modulating the expression (e.g., transcription or translation) of the intracellular target in a cell. Non-limiting examples of naturally-occurring intracellular targets include transcription factors and cell signaling cascade molecules, including enzymes, that modulate cell growth, differentiation and communication. Additional examples include intracellular targets that regulate cell metabolism.

Suitable transcription factors and intracellular signaling cascade molecules for particular uses in stimulating or inhibiting cellular activity or responses are described in the art. In some embodiments, an mRNA of the disclosure encodes a transcription factor useful for stimulating the activation or activity of an immune cell. As used herein, a “transcription factor” refers to a DNA-binding protein that regulates the transcription of a gene. In some embodiments, an mRNA of the disclosure encodes a transcription factor that increases or polarizes an immune response.

In some embodiments, an mRNA of the disclosure encodes an intracellular adaptor protein (e.g., in a signal transduction pathway) useful for stimulating the activation or activity of a cell.

In some embodiments, an mRNA of the disclosure encodes an intracellular signaling protein useful for stimulating the activation or activity of a cell. In some embodiments, an mRNA of the disclosure encodes a tolerogenic transcription factor useful for inhibiting the activation or activity of an immune cell.

In some embodiments, an mRNA of the disclosure encodes an intracellular target that is a protein that is used to treat a metabolic disease or disorder.

In some embodiments, an mRNA of the disclosure encodes a polypeptide of interest that is a fully-functional mitochondrial protein (e.g., wild-type). In some embodiments, an mRNA of the disclosure encodes a mitochondrial protein encoded by mitochondrial DNA (e.g., a mitochondrial-encoded mitochondrial protein). In some embodiments, an mRNA of the disclosure encodes a mitochondrial protein encoded by nuclear DNA (e.g., a nuclear-encoded mitochondrial protein). In some embodiments, an mRNA of the disclosure is used to treat a mitochondrial disease resulting from a mutation in a mitochondrial protein. In some embodiments, translation of an mRNA encoding a mitochondrial protein provides sufficient quantity and/or activity of the protein to ameliorate a mitochondrial disease. In some embodiments, an mRNA encodes a polypeptide of interest that is a mitochondrial protein described in the MitoCarta2.0 mitochondrial protein inventory.

Naturally Occurring Membrane Bound/Transmembrane Targets

In some embodiments, an mRNA of the disclosure encodes a polypeptide of interest that modulates the activity of a naturally-occurring membrane-bound/transmembrane target, for example by encoding the membrane-bound/transmembrane target itself or by modulating the expression (e.g., transcription or translation) of the membrane-bound/transmembrane target. Non-limiting examples of naturally-occurring membrane-bound/transmembrane targets include Cell surface receptors, growth factor receptors, costimulatory molecules, immune checkpoint molecules, homing receptors and HLA molecules.

In one embodiment, the membrane-bound/transmembrane targets are useful in stimulating or inhibiting immune responses are described herein. In some embodiments, an mRNA of the disclosure encodes a costimulatory factor that upregulates an immune response or is an antagonist of a costimulatory factor that downregulates an immune response. I n some embodiments, an mRNA of the disclosure encodes an immune checkpoint protein that down-regulates immune cells (e.g., T cells). In some embodiments, an mRNA of the disclosure encodes a membrane-bound/transmembrane protein target that serves as a homing signal.

In some embodiments, an mRNA of the disclosure encodes a membrane-bound/transmembrane protein target that is an immune receptor, e.g., on a lymphocyte or monocyte.

Modified Targets

In some embodiments, an mRNA of the disclosure encodes a polypeptide of interest that is a modified polypeptide. In some embodiments, an mRNA of the disclosure encodes a polypeptide of interest that modulates a modified target (e.g., up- or down-regulates the activity of a non-naturally-occurring target). Typically, an mRNA of the disclosure encodes a modified target. Alternatively, if a cell expresses a modified target, an mRNA-encoded polypeptide functions to modulate the activity of the modified target in the cell. In some embodiments, a non-naturally occurring target is a full-length target, such as a full-length modified protein. In some embodiments, a non-naturally occurring target is a fragment or portion of a non-naturally-occurring target, such as a fragment or portion of a modified protein. In some embodiments, an mRNA-encoded polypeptide when expressed acts in an autocrine fashion to modulate a modified target, i.e., exerts an effect directly on the cell into which the mRNA is delivered. Additionally or alternatively, an mRNA-encoded polypeptide when expressed acts in a paracrine fashion to modulates a modified target, i.e., exerts an effect indirectly on a cell other than the cell into which the mRNA is delivered (e.g., delivery of the mRNA into one type of cell results in secretion of a molecule that exerts effects on another type of cell, such as bystander cells). Non-limiting examples of modified proteins include modified soluble proteins (e.g., secreted proteins), modified intracellular proteins (e.g., intracellular signaling proteins, transcription factors) and modified membrane-bound or transmembrane proteins (e.g., receptors).

Modified Soluble Targets

In some embodiments, an mRNA of the disclosure encodes a polypeptide of interest that modulates a modified soluble target (e.g., up- or down-regulates the activity of a non-naturally-occurring soluble target). In some embodiments, an mRNA of the disclosure encodes a polypeptide of interest that is a modified soluble target. In some embodiments, a modified soluble target is a soluble protein that has been modified to alter (e.g., increase or decrease) the half-life (e.g., serum half-life) of the protein. Modified soluble proteins with altered half-life include modified cytokines and chemokines. In some embodiments, a modified soluble target is a soluble protein that has been modified to incorporate a tether such that the soluble protein becomes tethered to a cell surface. Modified soluble proteins incorporating a tether include tethered cytokines and chemokines.

Modified Intracellular Targets

In some embodiments, an mRNA of the disclosure encodes a polypeptide of interest that modulates a modified intracellular target (e.g., up- or down-regulates the activity of a non-naturally-occurring intracellular target). In some embodiments, an mRNA of the disclosure encodes polypeptide of interest that is a modified intracellular target. In some embodiments, a modified intracellular target is a constitutively active mutant of an intracellular protein, such as a constitutively active transcription factor or intracellular signaling molecule. In some embodiments, a modified intracellular target is a dominant negative mutant of an intracellular protein, such as a dominant negative mutant of a transcription factor or intracellular signaling molecule. In some embodiments, a modified intracellular target is an altered (e.g., mutated) enzyme, such as a mutant enzyme with increased or decreased activity within an intracellular signaling cascade.

Modified Membrane bound/Transmembrane Targets

In some embodiments, an mRNA of the disclosure encodes a polypeptide of interest that modulates a modified membrane-bound/transmembrane target (e.g., up- or down-regulates the activity of a non-naturally-occurring membrane-bound/transmembrane target). In some embodiments, an mRNA of the disclosure encodes a polypeptide of interest that is a modified membrane-bound/transmembrane target. In some embodiments, a modified membrane-bound/transmembrane target is a constitutively active mutant of a membrane-bound/transmembrane protein, such as a constitutively active cell surface receptor (i.e., activates intracellular signaling through the receptor without the need for ligand binding). In some embodiments, a modified membrane-bound/transmembrane target is a dominant negative mutant of a membrane-bound/transmembrane protein, such as a dominant negative mutant of a cell surface receptor. In some embodiments, a modified membrane-bound/transmembrane target is a molecule that inverts signaling of a cellular synapse (e.g., agonizes or antagonizes signaling of a receptor). In some embodiments, a modified membrane-bound/transmembrane target is a chimeric membrane-bound/transmembrane protein, such as a chimeric cell surface receptor.

As used herein, the term “chimeric antigen receptor (CAR)” refers to an artificial transmembrane protein receptor comprising an extracellular domain capable of binding to a predetermined CAR ligand or antigen, an intracellular segment comprising one or more cytoplasmic domains derived from signal transducing proteins different from the polypeptide from which the extracellular domain is derived, and a transmembrane domain.

### Pharmaceutical Compositions

The present disclosure includes pharmaceutical compositions comprising an mRNA or a nanoparticle (e.g., a lipid nanoparticle) described herein, in combination with one or more pharmaceutically acceptable excipient, carrier or diluent. In particular embodiments, the mRNA is present in a nanoparticle, e.g., a lipid nanoparticle. In particular embodiments, the mRNA or nanoparticle is present in a pharmaceutical composition.

Pharmaceutical compositions may optionally include one or more additional active substances, for example, therapeutically and/or prophylactically active substances. Pharmaceutical compositions of the present disclosure may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety). In particular embodiments, a pharmaceutical composition comprises an mRNA and a lipid nanoparticle, or complexes thereof.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may include between 0.1% and 100%, e.g., between 0.5% and 70%, between 1% and 30%, between 5% and 80%, or at least 80% (w/w) active ingredient.

The mRNAs of the disclosure can be formulated using one or more excipients to: (1) increase stability; (2) increase cell transfection; (3) permit the sustained or delayed release (e.g., from a depot formulation of the mRNA); (4) alter the biodistribution (e.g., target the mRNA to specific tissues or cell types); (5) increase the translation of a polypeptide encoded by the mRNA in vivo; and/or (6) alter the release profile of a polypeptide encoded by the mRNA in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles (e.g., liposomes and micelles), polymers, lipoplexes, core-shell nanoparticles, peptides, proteins, carbohydrates, cells transfected with mRNAs (e.g., for transplantation into a subject), hyaluronidase, nanoparticle mimics and combinations thereof. Accordingly, the formulations of the disclosure can include one or more excipients, each in an amount that together increases the stability of the mRNA, increases cell transfection by the mRNA, increases the expression of a polypeptide encoded by the mRNA, and/or alters the release profile of an mRNA-encoded polypeptide. Further, the mRNAs of the present disclosure may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition. Excipients may include, for example: antiadherents, antioxidants, binders, coatings, compression aids, disintegrants, dyes (colors), emollients, emulsifiers, fillers (diluents), film formers or coatings, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

In some embodiments, the formulations described herein may include at least one pharmaceutically acceptable salt. Examples of pharmaceutically acceptable salts that may be included in a formulation of the disclosure include, but are not limited to, acid addition salts, alkali or alkaline earth metal salts, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

In some embodiments, the formulations described herein may contain at least one type of mRNA. As a non-limiting example, the formulations may contain 1, 2, 3, 4, 5 or more than 5 mRNAs described herein. In some embodiments, the formulations described herein may contain at least one mRNA encoding a polypeptide and at least one nucleic acid sequence such as, but not limited to, an siRNA, an shRNA, a snoRNA, and an miRNA.

Liquid dosage forms for e.g., parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and/or suspending agents. In certain embodiments for parenteral administration, compositions are mixed with solubilizing agents such as CREMAPHOR®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, pharmaceutical compositions including at least one mRNA described herein are administered to mammals (e.g., humans). Although the descriptions of pharmaceutical compositions provided herein are principally directed to pharmaceutical compositions which are suitable for administration to humans, it will be understood by the skilled artisan that such compositions are generally suitable for administration to any other animal, e.g., to a non-human mammal. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys. In particular embodiments, a subject is provided with two or more mRNAs described herein. In particular embodiments, the first and second mRNAs are provided to the subject at the same time or at different times, e.g., sequentially. In particular embodiments, the first and second mRNAs are provided to the subject in the same pharmaceutical composition or formulation, e.g., to facilitate uptake of both mRNAs by the same cells.

The present disclosure also includes kits comprising a container comprising a mRNA encoding a polypeptide that enhances an immune response. In another embodiment, the kit comprises a container comprising a mRNA encoding a polypeptide that enhances an immune response, as well as one or more additional mRNAs encoding one or more antigens or interest. In other embodiments, the kit comprises a first container comprising the mRNA encoding a polypeptide that enhances an immune response and a second container comprising one or more mRNAs encoding one or more antigens of interest. In particular embodiments, the mRNAs for enhancing an immune response and the mRNA(s) encoding an antigen(s) are present in the same or different nanoparticles and/or pharmaceutical compositions. In particular embodiments, the mRNAs are lyophilized, dried, or freeze-dried.

### Methods And Use

The disclosure provides methods using the mRNAs, compositions, lipid nanoparticles, or pharmaceutical compositions disclosed herein. In some aspects, the mRNAs described herein are used to increase the amount and/or quality of a polypeptide (e.g., a therapeutic polypeptide) encoded by and translated from the mRNA. In some embodiments, the mRNAs described herein are used to reduce the translation of partial, aberrant, or otherwise undesirable open reading frames within the mRNA. In some embodiments, the mRNA described herein are used to initiate translation of a polypeptide (e.g., a therapeutic polypeptide) at a desired initiator codon.

In some embodiments, the methods described herein are useful for increasing the potency of an mRNA encoding a polypeptide. In one embodiment, the disclosure provides a method of inhibiting or reducing leaky scanning of an mRNA by a PIC or ribosome, the method comprising contacting a cell with an mRNA, a composition, a lipid nanoparticle, or a pharmaceutical composition according to the disclosure.

In some embodiments, the disclosure provides a method of increasing an amount of a polypeptide translated from a full open reading frame comprising an mRNA, the method comprising contacting a cell with an mRNA, a composition, a lipid nanoparticle, or a pharmaceutical composition according to the disclosure.

In some embodiments, the disclosure provides a method of increasing potency of a polypeptide translated from an mRNA, the method comprising contacting a cell with an mRNA, a composition, a lipid nanoparticle, or a pharmaceutical composition according to the disclosure.

In some embodiments, the disclosure provides a method of increasing initiation of polypeptide synthesis at or from an initiation codon comprising an mRNA, the method comprising contacting a cell with an mRNA, a composition, a lipid nanoparticle, or a pharmaceutical composition according to the disclosure.

In some embodiments, the disclosure provides a method of inhibiting or reducing initiation of polypeptide synthesis at any codon within an mRNA other than an initiation codon, the method comprising contacting a cell with an mRNA, a composition, a lipid nanoparticle, or a pharmaceutical composition according to the disclosure.

In some embodiments, the disclosure provides a method of inhibiting or reducing an amount of polypeptide translated from any open reading frame within an mRNA other than a full open reading frame, the method comprising contacting a cell with an mRNA, a composition, a lipid nanoparticle, or a pharmaceutical composition according to the disclosure.

In some embodiments, the disclosure provides method of inhibiting or reducing translation of truncated or aberrant translation products from an mRNA, the method comprising contacting a cell with an mRNA, a composition, a lipid nanoparticle, or a pharmaceutical composition according to the disclosure.

In one embodiment, the method comprises administering to the subject a composition of the disclosure (or lipid nanoparticle thereof, or pharmaceutical composition thereof) comprising at least one mRNA construct encoding a polypeptide (e.g., a therapeutic polypeptide)

Compositions of the disclosure are administered to the subject at an effective amount or effective dose. In general, an effective amount of the composition will allow for efficient production of the encoded polypeptide in the cell. Metrics for efficiency may include polypeptide translation (indicated by polypeptide expression), level of mRNA degradation, and immune response indicators.

### Kits

The disclosure provides a variety of kits for conveniently and/or effectively using the claimed nucleotides of the present disclosure. Typically kits will comprise sufficient amounts and/or numbers of components to allow a user to perform multiple treatments of a subject(s) and/or to perform multiple experiments.

In one aspect, the present disclosure provides kits comprising the molecules (polynucleotides) of the disclosure.

Said kits are for protein production, comprising a first polynucleotides comprising a translatable region. The kit can further comprise packaging and instructions and/or a delivery agent to form a formulation composition. The delivery agent can comprise a saline, a buffered solution, a lipidoid or any delivery agent disclosed herein.

In some embodiments, the buffer solution can include sodium chloride, calcium chloride, phosphate and/or EDTA. In another embodiment, the buffer solution include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose (See, e.g., U.S. Pub. No. 20120258046; herein incorporated by reference in its entirety). In a further embodiment, the buffer solutions are precipitated or it can be lyophilized. The amount of each component is varied to enable consistent, reproducible higher concentration saline or simple buffer formulations. The components is varied in order to increase the stability of modified RNA in the buffer solution over a period of time and/or under a variety of conditions. In one aspect, the present disclosure provides kits for protein production, comprising: a polynucleotide comprising a translatable region, provided in an amount effective to produce a desired amount of a protein encoded by the translatable region when introduced into a target cell; a second polynucleotide comprising an inhibitory nucleic acid, provided in an amount effective to substantially inhibit the innate immune response of the cell; and packaging and instructions.

In one aspect, the present disclosure provides kits for protein production, comprising a polynucleotide comprising a translatable region, wherein the polynucleotide exhibits reduced degradation by a cellular nuclease, and packaging and instructions.

In one aspect, the present disclosure provides kits for protein production, comprising a polynucleotide comprising a translatable region, wherein the polynucleotide exhibits reduced degradation by a cellular nuclease, and a mammalian cell suitable for translation of the translatable region of the first nucleic acid.

### Devices

The present disclosure provides for devices that incorporate polynucleotides that encode polypeptides of interest. These devices contain in a stable formulation the reagents to synthesize a polynucleotide in a formulation available to be immediately delivered to a subject in need thereof, such as a human patient.

Devices for administration are employed to deliver the polynucleotides of the present disclosure according to single, multi- or split-dosing regimens taught herein. Such devices are taught in, for example, International Application PCT/US2013/30062 filed Mar. 9, 2013, the contents of which are incorporated herein by reference in their entirety.

Method and devices known in the art for multi-administration to cells, organs and tissues are contemplated for use in conjunction with the methods and compositions disclosed herein as embodiments of the present disclosure. These include, for example, those methods and devices having multiple needles, hybrid devices employing for example lumens or catheters as well as devices utilizing heat, electric current or radiation driven mechanisms.

According to the present disclosure, these multi-administration devices are utilized to deliver the single, multi- or split doses contemplated herein. Such devices are taught for example in, International Application PCT/US2013/30062 filed Mar. 9, 2013, the contents of which are incorporated herein by reference in their entirety.

In some embodiments, the polynucleotide is administered subcutaneously or intramuscularly via at least 3 needles to three different, optionally adjacent, sites simultaneously, or within a 60 minutes period (e.g., administration to 4, 5, 6, 7, 8, 9, or 10 sites simultaneously or within a 60 minute period).

Methods and Devices Utilizing Catheters and/or Lumens

Methods and devices using catheters and lumens are employed to administer the polynucleotides of the present disclosure on a single, multi- or split dosing schedule. Such methods and devices are described in International Application PCT/US2013/30062 filed Mar. 9, 2013 (Attorney Docket Number M300), the contents of which are incorporated herein by reference in their entirety.

Methods and Devices Utilizing Electrical Current

Methods and devices utilizing electric current are employed to deliver the polynucleotides of the present disclosure according to the single, multi- or split dosing regimens taught herein. Such methods and devices are described in International Application PCT/US2013/30062 filed Mar. 9, 2013 (Attorney Docket Number M300), the contents of which are incorporated herein by reference in their entirety.

### EXAMPLES

### Materials & Methods

Synthesis of mRNA. mRNAs were synthesized in vitro from linearized DNA templates which include the 5′ UTR, 3′UTR and polyA tail, followed by addition of a 5′ CAP.
Cell culture and transfection. HeLa (ATCC), AML12 (ATCC), primary human hepatocytes (BioReclamation IVT), and MEF cells (Oriental Bioservice Inc., Minamiyayamashiro Laboratory) were cultured under standard conditions. Cells were transfected with reporter mRNA using Lipofectamine 2000 or MC3 following standard protocols.
Capillary Immunoblot. Six hours following cell transfection, cells lysates were prepared in a denaturing lysis buffer. Lysates were analyzed using a WES ProteinSimple instrument, with antibodies reactive against GFP used to detect the abundance of truncated protein, which lacks a 3×FLAG tag, relative to full-length protein, which includes a 3×FLAG tag. Leaky scanning percentages were calculated as the peak height corresponding to the truncated protein divided by the sum of peak heights for the truncated and full-length protein. When indicated, these values were further normalized to a reference standard.

### Example 1: Reporter System to Measure Start Site Fidelity and Ribosome Loading on mRNA

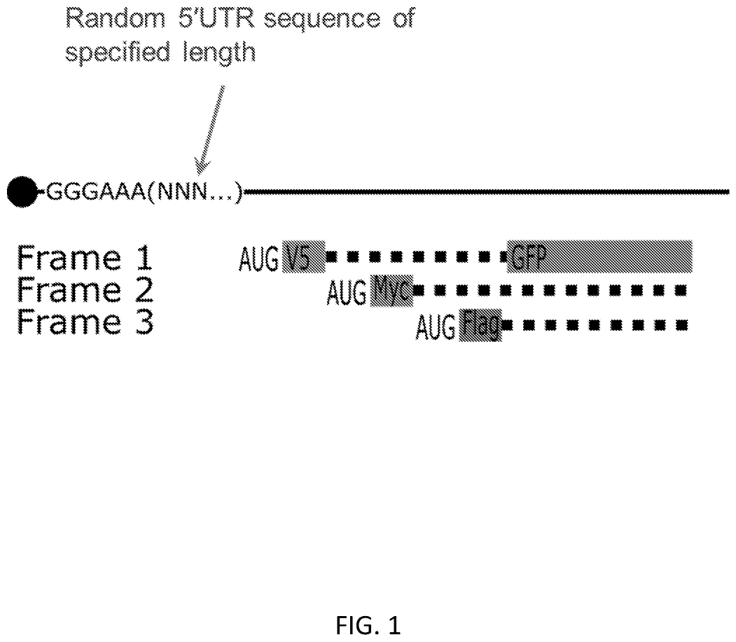

To screen large numbers of 5′ untranslated region (UTR) sequences for association with start site fidelity and ribosome loading, a reporter system was designed. Reporter mRNAs were prepared that encoded three AUG initiation codons separated by epitope tags (a first AUG followed by a V5 tag, a second AUG followed by a Myc tag, and a third AUG followed by a Flag tags) and followed by eGFP. The mRNA encoded a 3′ UTR set forth by SEQ ID NO: 110. V5 epitope tags were generated when initiation occurred at the first AUG. A Myc or FLAG epitope tag, rather than a V5 tag, were generated when initiation occurred at the second or third AUGs in alternative frames. A schematic of the reporter system is provided in FIG. 1. In the segment of coding sequence following the epitope tags, stop codons were omitted in all three frames in order to allow for retention of elongating ribosomes from all three frames. Stop codons were included in all three frames in the 3′ UTR of the mRNA.

The lengths and contents of 5′ UTRs that minimize leaky scanning were determined by analyzing the production of various epitope tags. Specifically, two 5′ UTR lengths were investigated (i.e., 50 nucleotides (10³⁰ possible unique sequences)) and 18 nucleotides (69 billion possible unique sequences)) for sequence requirements for start site fidelity and ribosome loading. An mRNA 5′UTR library, which was generated by PCR using degenerate primers followed by in vitro transcription, was transfected into cells using Lipofectamine 2000. Cells were treated with cycloheximide to halt translation elongation, then lysed. The lysate was split into three samples, each of which received a different antibody to target one of the three epitope tags (i.e., V5, Myc, FLAG). After 30 minutes of incubation, the antibody was precipitated using Protein A/G magnetic beads to bring down the whole nascent chain/ribosome/mRNA complex. RNA was purified from the beads. Deep sequencing of the RNA was used to determine a consensus sequence in the 5′UTR that gave rise to initiation at the first AUG as opposed to initiation at a later AUG.

### Example 2: C-Rich RNA Elements Decrease Leaky Scanning and Increase the Fidelity of Translation Initiation

Using the reporter system described in Example 1, 5′ UTR sequences that correlate with reduced leaky scanning were determined by comparing sequences in the immunoprecipitate isolated with an anti-V5 antibody (first start) to sequences in the immunoprecipitate isolated with either an anti-Myc antibody or an anti-FLAG antibody (leaky scanning starts). RNA elements associated with reduced leaky scanning were identified by determining the nucleotides enriched at each position in the 5′ UTR in sequences from the V5 (first start) immunoprecipitation compared to the Myc and FLAG (leaky scanning starts) immunoprecipitation. The 5′ UTR sequences that correlated with reduced leaky scanning (e.g., initiation fidelity) were determined using the following formula: (frequency of nucleotide at position with first start)/(frequency of nucleotide at position with subsequent starts).

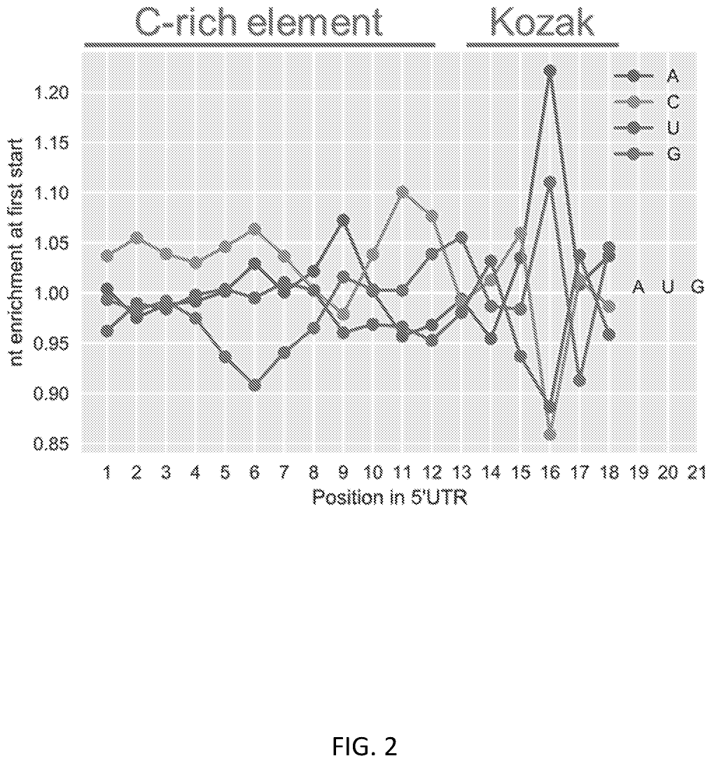

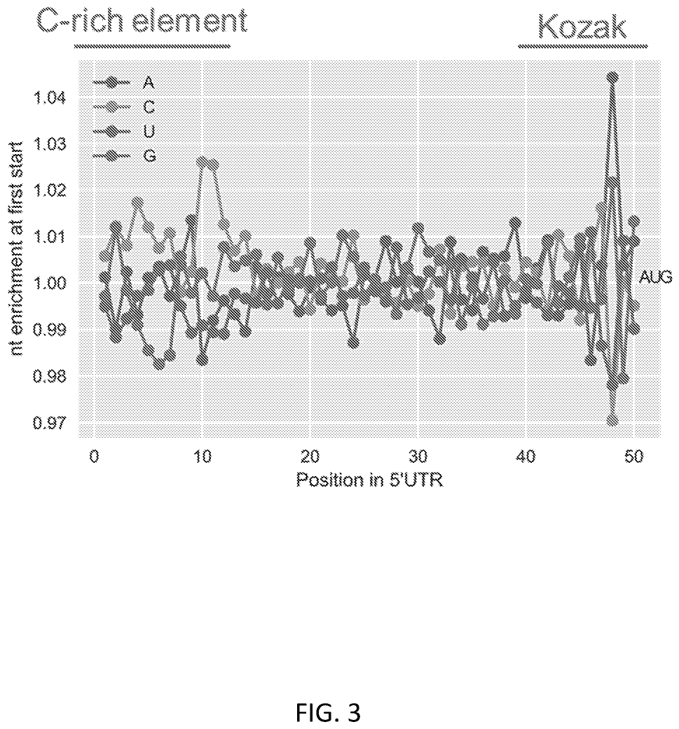

This gave rise to two apparent elements for 18 nucleotide 5′UTRs, the well characterized Kozak sequence (SEQ ID NO: 17) proximal to the AUG and an upstream C-rich element (SEQ ID NO: 29). Results are shown in FIG. 2. For 50 nucleotide 5′UTRs, the same two elements were found. With the longer UTRs, it became apparent the C-rich element was positioned relative to the 5′ end of the mRNA rather than the AUG. Results are shown in FIG. 3.

### Example 3: Enhancement of Ribosomal Density by Kozak-Like Sequence

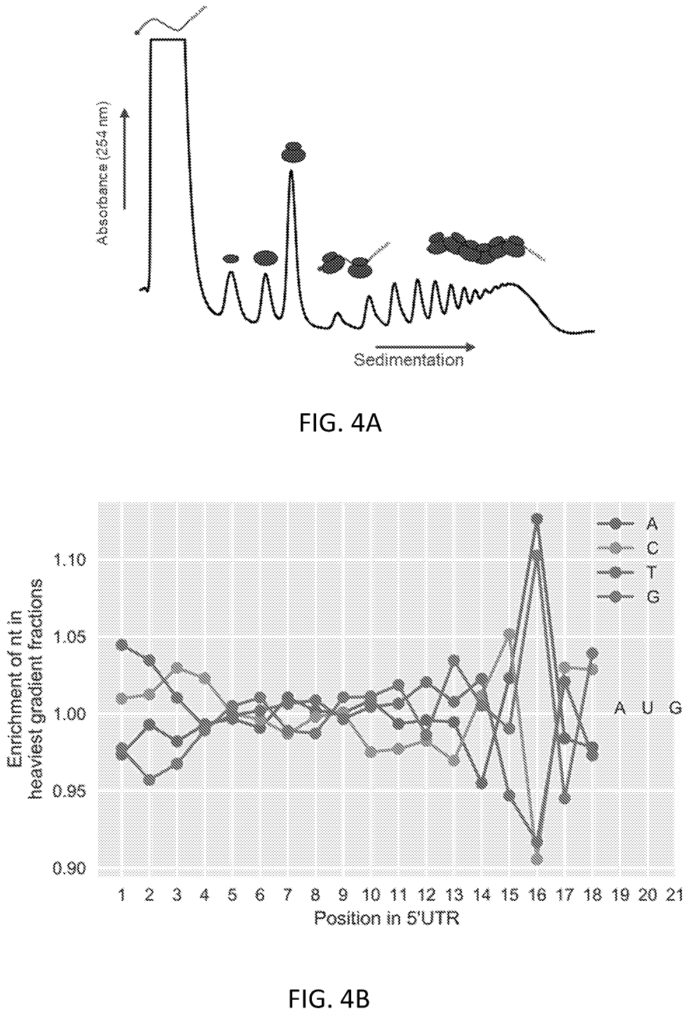

Using the reporter system described in Example 1, it was calculated which nucleotides were associated with heavy ribosome loading. The mRNAs described in Example 1 were transfected into cells using Lipofectamine 2000, then cell were lysed. Lysates were loaded over sucrose gradients from 20% w/v sucrose to 55% w/v sucrose, then centrifuged for 3 hours at 35,000 rpm using an SW-41 rotor, thus separating mRNA bearing many ribosomes from those bearing few ribosomes. Fractions from the sucrose gradient were collected and analyzed for 5′UTR content of the mRNA library using deep sequencing. FIG. 4A provides a schematic showing the relationship between ribosome loading on mRNA and sedimentation, with mRNA bearing many ribosomes (i.e., heavy polyribosomes) sedimenting more deeply than mRNA bearing few ribosomes. Results for a library of mRNAs comprising a 5′ UTR that was 18 nucleotides in length are shown in FIG. 4B. The graph shows the nucleotides enriched at each position for sequences associated with mRNA that co-sedimented with more than 7 ribosomes. The most apparent sequence associated with heavy ribosome loading was a Kozak-like sequence.

Based on the data presented in FIG. 4B, a 5′ UTR sequence can be deduced based on the nucleotides associated with heavy ribosome loading (DNA sequence from 5′ to 3′: TTCCGGTTGGGTGTCACG (SEQ ID NO: 47) and corresponding mRNA sequence with a Kozak-like sequence (canonically GCCACC) indicated by italics: UUCCGGUUGGGUGUCACG (SEQ ID NO: 48). Underlined nucleotides represent deviations from the canonical Kozak sequence.

The expression level was determined for an mRNA with a 5′ UTR comprised of a Kozak-like sequence identified as described above (SEQ ID NO: 48). To assess the amount of protein derived, an mRNA construct was generated with this 5′UTR sequence preceding an open reading frame encoding eGFP fused with a C-terminal degron sequence. A degron is a short amino acid sequence that facilitates the degradation of eGFP and prevents intracellular accumulation. The GFP fluorescence derived from this mRNA, as determined by IncuCyte S3 Live Cell Analysis System, was compared to an mRNA that was identical with the exception of its 5′UTR, which was based on a 5′UTR v1.1 (SEQ ID NO: 9). By measuring the total fluorescent intensity over a 72 h time course, it was shown that the ribosome density-derived sequence was associated with a 17% increase in overall GFP fluorescence in HeLa cells.

### Example 4: Initiation Fidelity from mRNAs Comprising C-Rich Elements

To determine the effect of C-rich RNA elements on initiation fidelity, a 3×FLAG reporter system was utilized to detect the percentage of protein that is derived from leaky scanning. Specifically, reporter mRNAs were designed such that (i) translation initiation from the initial start site downstream of the 5′ UTR would produce an eGFP polypeptide fused to a 3×FLAG epitope tag at the N-terminus; (ii) translation initiation from a second AUG codon downstream of the 5′UTR would produce only an eGFP polypeptide containing no epitope tags. The reporter mRNAs comprised a 3′UTR as set forth by SEQ ID NO: 109. Reporter mRNAs were transfected into cells, then harvested 6 hours after transfection. The lysates were analyzed by capillary immunoblot using an anti-GFP antibody. Assessed in the immunoblot are a GFP-only band corresponding to initiation at the second AUG (i.e., short band) and a 3×FLAG tag-GFP full length band corresponding to initiation at the first AUG (i.e., long band). The leaky scanning rate was calculated as the peak height for the GFP-only band relative to the combined peak height of the GFP-only band and the full length band (leaky scanning rate=short band/(short band+long band)).

An mRNA with a 5′ UTR comprising a C-rich element and a Kozak-like sequence corresponding to SEQ ID NO: 49 was compared to an mRNA with a 5′ UTR lacking a C-rich element but that is otherwise identical that corresponds to SEQ ID NO: 129. For the mRNA with a 5′ UTR comprising a C-rich element and a Kozak-like sequence, the leaky scanning rate was assigned a value of 1.0. The mRNA that lacked a C-rich element in the 5′ UTR had a leaky scanning rate of 1.59, indicating that the inclusion of a C-rich element resulted in a 37% reduction in leaky scanning.

### Example 5: C-Rich RNA Elements Alone and in Combination with GC-Rich RNA Elements Decrease Leaky Scanning

To further determine the effect of C-rich RNA elements on leaky scanning, the 3×FLAG reporter expression system described in Example 4 was used. Briefly, reporter mRNAs with 5′ UTRs with or without a C-rich RNA element were tested. The 5′ UTR denoted as combo2_S065 (SEQ ID NO: 38) contains the C-rich RNA element CR₅ (SEQ ID NO: 33). The 5′ UTR denoted as combo3_S065 (SEQ ID NO: 39) contains a Kozak sequence (GCCACC; SEQ ID NO: 17). The 5′ UTR denoted combo5_S065 (SEQ ID NO: 41) contains both the C-rich RNA element CR₅ (SEQ ID NO: 33) and a Kozak sequence (GCCACC; SEQ ID NO: 17). The 5′ UTR denoted S065 Ref (SEQ ID NO: 42) does not contain a C-rich RNA element or a Kozak sequence and was used as a comparator.

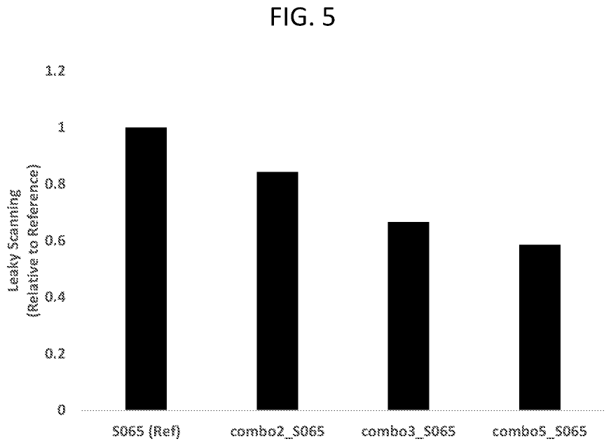

As shown in FIG. 5, the amount of leaky scanning from a reporter mRNAs comprising a 5′ UTR with a C-rich RNA element (combo2_S065, SEQ ID NO: 38) was decreased relative to a reporter mRNA comprising a 5′ UTR lacking the C-rich RNA element (S065 (Ref), SEQ ID NO: 42). These data demonstrate that presence of a C-rich RNA element in the 5′ UTR of an mRNA decreases leaky scanning of the mRNA relative to an mRNA that does not comprise the C-rich RNA element. Additionally, the amount of leaky scanning from reporter mRNAs comprising a 5′ UTR with a Kozak-like sequence (combo3_S065 SEQ ID NO: 39) was decreased relative to reporter mRNAs comprising a 5′UTR that lacked a Kozak-like sequence (S065 (Ref) SEQ ID NO: 42 and combo2_S065 SEQ ID NO: 38). These data demonstrate that presence of a Kozak-like sequence in the 5′ UTR of an mRNA decreases leaky scanning of the mRNA relative to an mRNA that does not comprise the Kozak-like sequence. The combination of a C-rich RNA element and a Kozak-like sequence (combo5_S065, SEQ ID NO: 41) resulted in the greatest overall reduction in leaky scanning. These data further demonstrate that the inclusion of a Kozak-like sequence in combination with a C-rich RNA element have an additive effect in decreasing leaky scanning of an mRNA.

To determine the effect of combining C-rich RNA elements with GC-rich RNA elements on leaky scanning, the 3×FLAG reporter expression system described in Example 4 was used. Briefly, reporter mRNAs with 5′ UTRs comprising a GC-rich RNA element alone or in combination with a C-rich RNA element were tested. Reporter mRNAs with 5′ UTRs lacking C-rich RNA elements were used as comparators.

The 5′ UTR denoted V1-UTR (v1.1 Ref) (SEQ ID NO: 9) contains the GC-rich RNA element V1 (SEQ ID NO: 1). The 5′ UTR denoted combo1_V1.1 5′ UTR (SEQ ID NO: 35) contains both the GC-rich RNA element V1 (SEQ ID NO: 1) and the C-rich RNA element CR₃ (SEQ ID NO: 31). The 5′ UTR denoted combo2_V1.1 5′ UTR (SEQ ID NO: 36) contains both the GC-rich RNA element V1 (SEQ ID NO: 1) and the C-rich RNA element CR₅ (SEQ ID NO: 33).

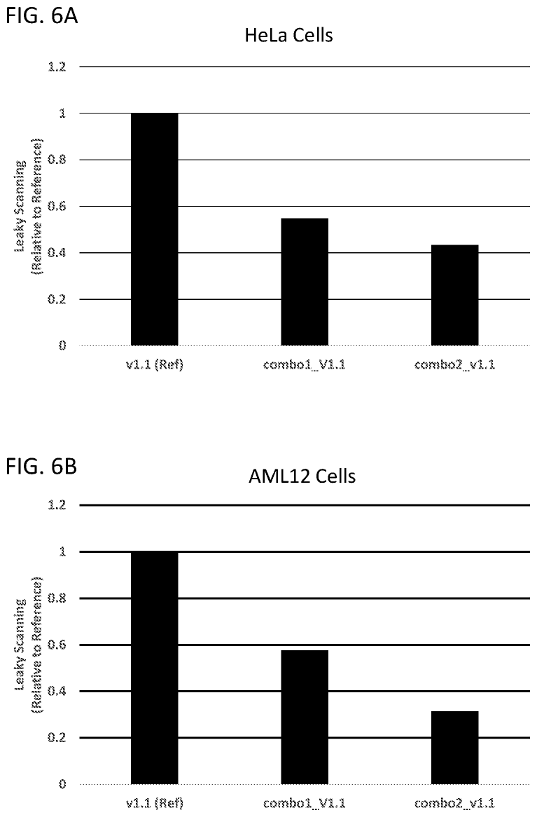

As shown in FIGS. 6A-6B, the amount of leaky scanning from reporter mRNAs comprising 5′ UTRs (combo1_V1.1 and combo2_V1.1) with a GC-rich RNA element (V1) in combination with a C-rich RNA element (CR₃ or CR₅) was decreased relative to a reporter mRNA comprising a 5′ UTR (V1-UTR (v1.1 Ref)) with the V1 GC-rich RNA element alone in both HeLa cells (FIG. 6A) and AML12 cells (FIG. 6B). These data demonstrate that presence of a GC-rich RNA element in combination with a C-rich RNA element in the 5′ UTR of an mRNA decreases leaky scanning of the mRNA relative to an mRNA that does not comprise the C-rich RNA element, indicating an additive effect on leaky scanning.

Further studies were performed to determine the effect of combining C-rich RNA elements with GC-rich RNA elements on leaky scanning. Briefly, reporter mRNAs were prepared with either a 5′ UTR comprising a GC-rich RNA element (GCC3-ExtKozak (Ref); SEQ ID NO: 43) or a GC-rich RNA element and a C-rich RNA element (CrichCR4+GCC3-ExtKozak; SEQ ID NO: 44). The GCC3-ExtKozak (Ref) 5′ UTR incorporates the GC-rich RNA element (GCC)₃ (GCCGCCGCC; SEQ ID NO: 23), while the CrichCR4+GCC3-ExtKozak 5′ UTR incorporates both the GC-rich RNA element (GCC)₃ (SEQ ID NO: 23) and the C-rich RNA element CR₄ (SEQ ID NO: 32). The effect on leaky scanning of a GC-rich RNA element alone or a combination of a GC-rich RNA element and a C-rich RNA element was evaluated using the 3×FLAG reporter expression system described in Example 4.

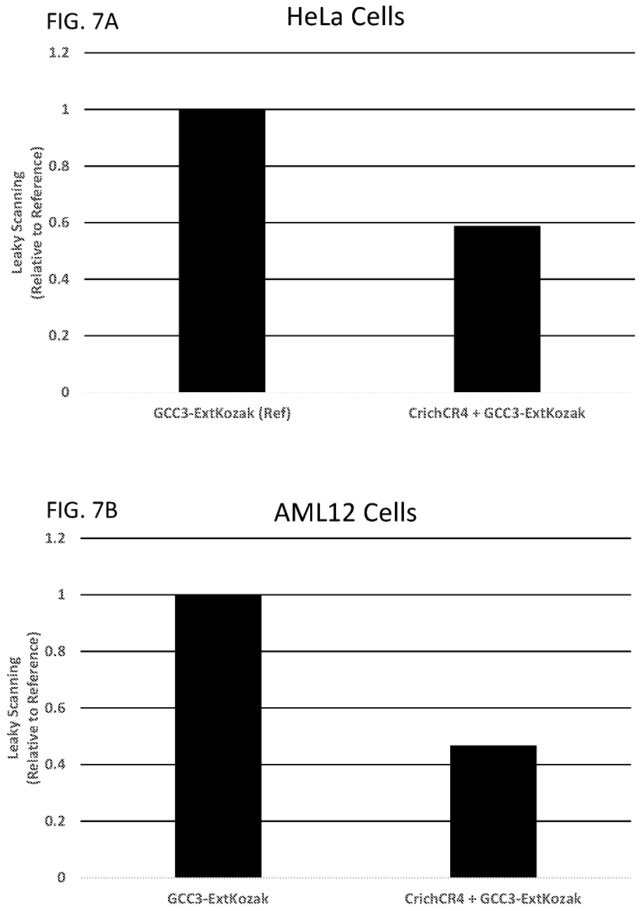

As shown in FIGS. 7A-7B, the amount of leaky scanning from a reporter mRNA comprising 5′ UTRs (CrichCR4+GCC3-ExtKozak, SEQ ID NO: 44) with a GC-rich RNA element (GCC)₃ in combination with a C-rich RNA element (CR₄) was decreased relative to a reporter mRNA comprising a 5′ UTR (GCC3-ExtKozak (Ref), SEQ ID NO: 43) with the (GCC)₃ GC-rich RNA element alone in both HeLa cells (FIG. 7A) and AML12 cells (FIG. 7B). These data further demonstrate that presence of a GC-rich RNA element in combination with a C-rich RNA element in the 5′ UTR of an mRNA decreases leaky scanning of the mRNA relative to an mRNA that does not comprise the C-rich RNA element. Thus, the combination of a C-rich RNA element and a GC-rich RNA element has an additive effect on improving initiation fidelity (i.e., decreasing leaky scanning).

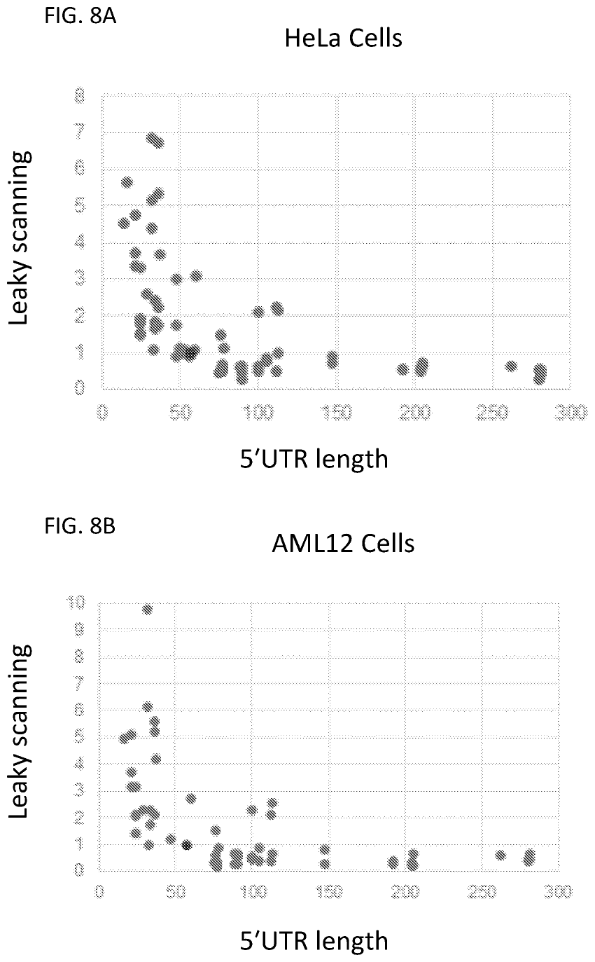

Additionally, the effect of the 5′ UTR length on leaky scanning was assessed. The length of the 5′ UTR was varied and the effect on the rate of leaky scanning of a 3×FLAG reporter mRNA was evaluated in both HeLa cells (FIG. 8A) and AML12 cells (FIG. 8B). As shown in FIG. 8A and FIG. 8B, the rate of leaky scanning is plotted against the length of the 5′ UTR (i.e., length referring to the number of nucleotides in the 5′ UTR sequence). The rate of leaky scanning is shown normalized to the rate of leaky scanning for the v1.1 Ref 5′ UTR (SEQ ID NO: 9). For both HeLa cells and AM12 cells, reporter mRNAs with a short 5′ UTR demonstrated high levels of leaky scanning relative to the v1.1 Ref 5′ UTR (SEQ ID NO: 9), while reporter mRNAs with a long 5′ UTR demonstrated low levels of leaky scanning relative to the v1.1 Ref 5′ UTR (SEQ ID NO: 9). These results demonstrate that the length of the 5′ UTR is inversely correlated with the rate of leaky scanning. Longer UTRs (>80 nt) often correlated with lower leaky scanning while shorter UTRs (<50 nt) often correlated with higher leaky scanning.

### EQUIVALENTS AND SCOPE

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### Summary of Sequence Listing

| SEQ ID NO: | Name/description | Identifier | Sequence |
|---|---|---|---|
| 1 | GC-rich RNA | V1 | [CCCCGGCGCC] |
| | element | | |
| 2 | GC-rich RNA | V2 | [CCCGGC] |
| | element | | |
| 3 | GC-rich RNA | EK1 | [CCCGCC] |
| | element | | |
| 4 | 5′UTR | 5′UTR-022 | GGGAAATAAGAGAGAAAAGAAGAGTAA |
| | | (DNA) | GAAGAAATATAAGA |
| 5 | 5′UTR | 5′UTR-022 | GGGAAAUAAGAGAGAAAAGAAGAGUAA |
| | | (RNA) | GAAGAAAUAUAAGA |
| 6 | 5′UTR | 5′UTR-023 | GGGAAAUAAGAGAGAAAAGAAGAGUAA |
| | | (RNA) | GAAGAAAUAUAAGACCCCGGCGCCGCC |
| | | | ACC |
| 7 | 5′UTR | 5′UTR-023 | GGGAAATAAGAGAGAAAAGAAGAGTAA |
| | | (DNA) | GAAGAAATATAAGACCCCGGCGCCGCC |
| | | | ACC |
| 8 | 5′UTR | 5′UTR-001 | UAAGAGAGAAAAGAAGAGUAAGAAGAA |
| | | Core (RNA) | AUAUAAGA |
| 9 | 5′UTR | F418 (V1-UTR | GGGAAATAAGAGAGAAAAGAAGAGTAA |
| | | (v1.1 Ref)) | GAAGAAATATAAGACCCCGGCGCCGCC |
| | | (DNA) | ACC |
| 10 | 5′UTR | V2-UTR (DNA) | GGGAAATAAGAGAGAAAAGAAGAGTAA |
| | | | GAAGAAATATAGACCCCGGCGCCACC |
| 11 | 5′UTR | CG1-UTR (DNA) | GGGAAATAAGAGAGAAAAGAAGAGTAA |
| | | | GAAGAAATATAAGAGCGCCCCGCGGCG |
| | | | CCCCGCGGCCACC |
| 12 | 5′UTR | CG2-UTR (DNA) | GGGAAATAAGAGAGAAAAGAAGAGTAA |
| | | | GAAGAAATATAAGACCCGCCCGCCCCG |
| | | | CCCCGCCGCCACC |
| 13 | 5′UTR | KT1-UTR | GGGCCCGCCGCCAAC |
| 14 | 5′UTR | KT2-UTR | GGGCCCGCCGCCACC |
| 15 | 5′UTR | KT3-UTR | GGGCCCGCCGCCGAC |
| 16 | 5′UTR | KT4-UT4 | GGGCCCGCCGCCGCC |
| 17 | Traditional | K0 | [GCC[A/G]CC] |
| | Kozak consensus | | |
| 18 | GC-rich RNA | EK2 | [GCCGCC] |
| | element | | |
| 19 | GC-rich RNA | EK3 | [CCGCCG] |
| | element | | |
| 20 | GC-rich RNA | CG1 | [GCGCCCCGCGGCGCCCCGCG] |
| | element | | |
| 21 | GC-rich RNA | CG2 | [CCCGCCCGCCCCGCCCCGCC] |
| | element | | |
| 22 | GC-rich RNA | (CCG)ₙ, | [CCG]ₙ |
| | element | n = 1-10 | |
| 23 | GC-rich RNA | (GCC)ₙ, | [GCC]ₙ |
| | element | n = 1-10 | |
| 24 | Stable RNA | SL1 | CCGCGGCGCCCCGCGG |
| | structures | | (−9.90 kcal/mol) |
| 25 | Stable RNA | SL2 | GCGCGCAUAUAGCGCGC |
| | structures | | (−10.90 kcal/mol) |
| 26 | Stable RNA | SL3 | CATGGTGGCGGCCCGCCGCCACCATG |
| | structures | | (−22.10 kcal/mol) |
| 27 | Stable RNA | SL4 | CATGGTGGCCCGCCGCCACCATG |
| | structures | | (−14.90 kcal/mol) |
| 28 | Stable RNA | SL5 | CATGGTGCCCGCCGCCACCATG |
| | structures | | (−8.00 kcal/mol) |
| 29 | C-Rich RNA | CR2 | CCCCCCCAACCC |
| | element | | |
| 30 | C-Rich RNA | CR1 | CCCCCCCCAACC |
| | element | | |
| 31 | C-Rich RNA | CR3 | CCCCCCACCCCC |
| | element | | |
| 32 | C-Rich RNA | CR4 | CCCCCCUAAGCC |
| | element | | |
| 33 | C-Rich RNA | CR5 | CCCCACAACC |
| | element | | |
| 34 | C-Rich RNA | CR6 | CCCCCACAACC |
| | element | | |
| 35 | 5′UTR | combo1_V1.1 | GGGAAACCCCCCACCCCCGGGGAAAUA |
| | | (RNA) | AGAGAGAAAAGAAGAGUAAGAAGAAAU |
| | | | AUAAGACCCCGGCGCCGCCACC |
| 36 | 5′UTR | combo2_V1.1 | GGGAAAUCCCCACAACCGGGGAAAUAA |
| | | (RNA) | GAGAGAAAAGAAGAGUAAGAAGAAAUA |
| | | | UAAGACCCCGGCGCCGCCACC |
| 37 | 5′UTR | combo1_S065 | GGGAAACCCCCCACCCCCGCCUCAUAU |
| | | (RNA) | CCAGGCUCAAGAAUAGAGCUCAGUGUU |
| | | | UUGUUGUUUAAUCAUUCCGACGUGUUU |
| | | | UGCGAUAUUCGCGCAAAGCAGCCAGUC |
| | | | GCGCGCUUGCUUUUAAGUAGAGUUGUU |
| | | | UUUCCACCCGUUUGCCAGGCAUCUUUA |
| | | | AUUUAACAUAUUUUUAUUUUUCAGGCU |
| | | | AACCUAAAGCAGAGAA |
| 38 | 5′UTR | combo2_S065 | GGGAAAUCCCCACAACCGCCUCAUAUC |
| | | (RNA) | CAGGCUCAAGAAUAGAGCUCAGUGUUU |
| | | | UGUUGUUUAAUCAUUCCGACGUGUUUU |
| | | | GCGAUAUUCGCGCAAAGCAGCCAGUCG |
| | | | CGCGCUUGCUUUUAAGUAGAGUUGUUU |
| | | | UUCCACCCGUUUGCCAGGCAUCUUUAA |
| | | | UUUAACAUAUUUUUAUUUUUCAGGCUA |
| | | | ACCUAAAGCAGAGAA |
| 39 | 5′UTR | combo3_S065 | GGGAGACCUCAUAUCCAGGCUCAAGAA |
| | | (S065 ExtKozak) | UAGAGCUCAGUGUUUUGUUGUUUAAUC |
| | | (RNA) | AUUCCGACGUGUUUUGCGAUAUUCGCG |
| | | | CAAAGCAGCCAGUCGCGCGCUUGCUUU |
| | | | UAAGUAGAGUUGUUUUUCCACCCGUUU |
| | | | GCCAGGCAUCUUUAAUUUAACAUAUUU |
| | | | UUAUUUUUCAGGCUAACCUACGCCGCC |
| | | | ACC |
| 40 | 5′UTR | combo4_S065 | GGGAAACCCCCCACCCCCGCCUCAUAU |
| | | (RNA) | CCAGGCUCAAGAAUAGAGCUCAGUGUU |
| | | | UUGUUGUUUAAUCAUUCCGACGUGUUU |
| | | | UGCGAUAUUCGCGCAAAGCAGCCAGUC |
| | | | GCGCGCUUGCUUUUAAGUAGAGUUGUU |
| | | | UUUCCACCCGUUUGCCAGGCAUCUUUA |
| | | | AUUUAACAUAUUUUUAUUUUUCAGGCU |
| | | | AACCUACGCCGCCACC |
| 41 | 5′UTR | F153 | GGGAAAUCCCCACAACCGCCUCAUAUC |
| | | combo5_S065 | CAGGCUCAAGAAUAGAGCUCAGUGUUU |
| | | (RNA) | UGUUGUUUAAUCAUUCCGACGUGUUUU |
| | | | GCGAUAUUCGCGCAAAGCAGCCAGUCG |
| | | | CGCGCUUGCUUUUAAGUAGAGUUGUUU |
| | | | UUCCACCCGUUUGCCAGGCAUCUUUAA |
| | | | UUUAACAUAUUUUUAUUUUUCAGGCUA |
| | | | ACCUACGCCGCCACC |
| 42 | 5′UTR | S065 Ref | GGGAGACCUCAUAUCCAGGCUCAAGAA |
| | | (RNA) | UAGAGCUCAGUGUUUUGUUGUUUAAUC |
| | | | AUUCCGACGUGUUUUGCGAUAUUCGCG |
| | | | CAAAGCAGCCAGUCGCGCGCUUGCUUU |
| | | | UAAGUAGAGUUGUUUUUCCACCCGUUU |
| | | | GCCAGGCAUCUUUAAUUUAACAUAUUU |
| | | | UUAUUUUUCAGGCUAACCUAAAGCAGA |
| | | | GAA |
| 43 | 5′UTR | GCC3-ExtKozak | GGGAAAGCCGCCGCCGCCACC |
| | | (Ref) | |
| 44 | 5′UTR | CrichCR4 + | GGGAAACCCCCCUAAGCCGCCGCCGCC |
| | | GCC3-ExtKozak | GCCACC |
| | | (RNA) | |
| 45 | 5′UTR | V0-UTR (v1.0 | GGGAAAUAAGAGAGAAAAGAAGAGUAA |
| | | Ref) (RNA) | GAAGAAAUAUAAGAGCCACC |
| 46 | 5′UTR | S065 core (RNA) | CCUCAUAUCCAGGCUCAAGAAUAGAGC |
| | | | UCAGUGUUUUGUUGUUUAAUCAUUCCG |
| | | | ACGUGUUUUGCGAUAUUCGCGCAAAGC |
| | | | AGCCAGUCGCGCGCUUGCUUUUAAGUA |
| | | | GAGUUGUUUUUCCACCCGUUUGCCAGG |
| | | | CAUCUUUAAUUUAACAUAUUUUUAUUU |
| | | | UUCAGGCUAACCUA |
| 47 | 5′UTR | 5′UTR-026 (DNA) | TTCCGGTTGGGTGTCACG |
| 48 | 5′UTR | 5′UTR-026 (RNA) | UUCCGUUGGGUGUCACG |
| 49 | 5′UTR | 5′UTR-024 (RNA) | CCCCCCCAACCCGUCACG |
| 50 | 5′UTR | 5UTR-002 (RNA) | GGGAGAUCAGAGAGAAAAGAAGAGUAA |
| | | | GAAGAAAUAUAAGAGCCACC |
| 51 | 5′UTR | 5′UTR-003 (RNA) | GGAAUAAAAGUCUCAACACAACAUAUA |
| | | | CAAAACAAACGAAUCUCAAGCAAUCAA |
| | | | GCAUUCUACUUCUAUUGCAGCAAUUUA |
| | | | AAUCAUUUCUUUUAAAGCAAAAGCAAU |
| | | | UUUCUGAAAAUUUUCACCAUUUACGAA |
| | | | CGAUAGCAAC |
| 52 | 5′UTR | 5′UTR-004 (RNA) | GGGAGACAAGCUUGGCAUUCCGGUACU |
| | | | GUUGGUAAAGCCACC |
| 53 | 5′UTR | 5′UTR-005 (RNA) | GGGAGAUCAGAGAGAAAAGAAGAGUAA |
| | | | GAAGAAAUAUAAAGAGCCACC |
| 54 | 5′UTR | 5′UTR-006 (RNA) | GGAAUAAAAGUCUCAACACAACAUAUA |
| | | | CAAAACAAACGAAUCUCAAGCAAUCAA |
| | | | GCAUUCUACUUCUAUUGCAGCAAUUUA |
| | | | AAUCAUUUCUUUUAAAGCAAAAGCAAU |
| | | | UUUCUGAAAAUUUUCACCAUUUACGAA |
| | | | CGAUAGCAAC |
| 55 | 5′UTR | 5′UTR-007 (RNA) | GGGAGACAAGCUUGGCAUUCCGGUACU |
| | | | GUUGGUAAAGCCACC |
| 56 | 5′UTR | 5′UTR-008 (RNA) | GGGAAUUAACAGAGAAAAGAAGAGUAA |
| | | | GAAGAAAUAUAAGAGCCACC |
| 57 | 5′UTR | 5′UTR-009 (RNA) | GGGAAAUUAGACAGAAAAGAAGAGUAA |
| | | | GAAGAAAUAUAAGAGCCACC |
| 58 | 5′UTR | 5′UTR-010 (RNA) | GGGAAAUAAGAGAGUAAAGAACAGUAA |
| | | | GAAGAAAUAUAAGAGCCACC |
| 59 | 5′UTR | 5′UTR-011 (RNA) | GGGAAAAAAGAGAGAAAAGAAGACUAA |
| | | | GAAGAAAUAUAAGAGCCACC |
| 60 | 5′UTR | 5′UTR-012 (RNA) | GGGAAAUAAGAGAGAAAAGAAGAGUAA |
| | | | GAAGAUAUAUAAGAGCCACC |
| 61 | 5′UTR | 5′UTR-013 (RNA) | GGGAAAUAAGAGACAAAACAAGAGUAA |
| | | | GAAGAAAUAUAAGAGCCACC |
| 62 | 5′UTR | 5′UTR-014 (RNA) | GGGAAAUUAGAGAGUAAAGAACAGUAA |
| | | | GUAGAAUUAAAAGAGCCACC |
| 63 | 5′UTR | 5′UTR-015 (RNA) | GGGAAAUAAGAGAGAAUAGAAGAGUAA |
| | | | GAAGAAAUAUAAGAGCCACC |
| 64 | 5′UTR | 5′UTR-016 (RNA) | GGGAAAUAAGAGAGAAAAGAAGAGUAA |
| | | | GAAGAAAAUUAAGAGCCACC |
| 65 | 5′UTR | 5′UTR-017 (RNA) | GGGAAAUAAGAGAGAAAAGAAGAGUAA |
| | | | GAAGAAAUUUAAGAGCCACC |
| 66 | 5′UTR | 5′UTR-018 (RNA) | GGGAAAUAAGAGAGAAAAGAAGAGUAA |
| | | | GAAGAAAUAUAAGAGCCACC |
| 67 | 5′UTR | 5′UTR-019 (RNA) | UCAAGCUUUUGGACCCUCGUACAGAAG |
| | | | CUAAUACGACUCACUAUAGGGAAAUAA |
| | | | GAGAGAAAAGAAGAGUAAGAAGAAAUA |
| | | | UAAGAGCCACC |
| 68 | 5′UTR | 5′UTR-020 (RNA) | GGACAGAUCGCCUGGAGACGCCAUCCA |
| | | | CGCUGUUUUGACCUCCAUAGAAGACAC |
| | | | CGGGACCGAUCCAGCCUCCGCGGCCGG |
| | | | GAACGGUGCAUUGGAACGCGGAUUCCC |
| | | | CGUGCCAAGAGUGACUCACCGUCCUUG |
| | | | ACACG |
| 69 | 5′UTR | 5′UTR-021 (RNA) | GGCGCUGCCUACGGAGGUGGCAGCCAU |
| | | | CUCCUUCUCGGCAUC |
| 70 | 5′UTR | CG2-UTR (RNA) | GGGAAAUAAGAGAGAAAAGAAGAGUAA |
| | | | GAAGAAAUAUAAGACCCGCCCGCCCCG |
| | | | CCCCGCCGCCACC |
| 71 | 5′UTR | V0-UTR (v1.0 | AGGAAAUAAGAGAGAAAAGAAGAGUAA |
| | | Ref)-A (RNA) | GAAGAAAUAUAAGAGCCACC |
| 72 | 5′UTR | S065-A Ref | AGGAGACCUCAUAUCCAGGCUCAAGAA |
| | | (RNA) | UAGAGCUCAGUGUUUUGUUGUUUAAUC |
| | | | AUUCCGACGUGUUUUGCGAUAUUCGCG |
| | | | CAAAGCAGCCAGUCGCGCGCUUGCUUU |
| | | | UAAGUAGAGUUGUUUUUCCACCCGUUU |
| | | | GCCAGGCAUCUUUAAUUUAACAUAUUU |
| | | | UUAUUUUUCAGGCUAACCUAAAGCAGA |
| | | | GAA |
| 73 | 5′UTR | combo3_S065 | AGGAGACCUCAUAUCCAGGCUCAAGAA |
| | | (S065 | UAGAGCUCAGUGUUUUGUUGUUUAAUC |
| | | ExtKozak)-A | AUUCCGACGUGUUUUGCGAUAUUCGCG |
| | | | CAAAGCAGCCAGUCGCGCGCUUGCUUU |
| | | | UAAGUAGAGUUGUUUUUCCACCCGUUU |
| | | | GCCAGGCAUCUUUAAUUUAACAUAUUU |
| | | | UUAUUUUUCAGGCUAACCUACGCCGCC |
| | | | ACC |
| 74 | 5′UTR | F418 (V1-UTR | AGGAAAUAAGAGAGAAAAGAAGAGUAA |
| | | (v1.1 Ref))-A | GAAGAAAUAUAAGACCCCGGCGCCGCC |
| | | (RNA) | ACC |
| 75 | 5′UTR | V2-UTR-A (RNA) | AGGAAAUAAGAGAGAAAAGAAGAGUAA |
| | | | GAAGAAAUAUAAGACCCCGGCGCCACC |
| 76 | 5′UTR | CG1-UTR-A (RNA) | AGGAAAUAAGAGAGAAAAGAAGAGUAA |
| | | | GAAGAAAUAUAAGAGCGCCCCGCGGCG |
| | | | CCCCGCGGCCACC |
| 77 | 5′UTR | CG2-UTR-A (RNA) | AGGAAAUAAGAGAGAAAAGAAGAGUAA |
| | | | GAAGAAAUAUAAGACCCGCCCGCCCCG |
| | | | CCCCGCCGCCACC |
| 78 | 5′UTR | KT1-UTR-A | AGGCCCGCCGCCAAC |
| 79 | 5′UTR | KT2-UTR-A | AGGCCCGCCGCCACC |
| 80 | 5′UTR | KT3-UTR-A | AGGCCCGCCGCCGAC |
| 81 | 5′UTR | KT4-UTR-A | AGGCCCGCCGCCGCC |
| 82 | 5′UTR | GCC3-ExtKozak | AGGAAAGCCGCCGCCGCCACC |
| | | (Ref)-A | |
| 83 | 5′UTR | combo1_S065 | AGGAAACCCCCCACCCCCGCCUCAUAU |
| | | (RNA)-A | CCAGGCUCAAGAAUAGAGCUCAGUGUU |
| | | | UUGUUGUUUAAUCAUUCCGACGUGUUU |
| | | | UGCGAUAUUCGCGCAAAGCAGCCAGUC |
| | | | GCGCGCUUGCUUUUAAGUAGAGUUGUU |
| | | | UUUCCACCCGUUUGCCAGGCAUCUUUA |
| | | | AUUUAACAUAUUUUUAUUUUUCAGGCU |
| | | | AACCUAAAGCAGAGAA |
| 84 | 5′UTR | combo2_S065 | AGGAAAUCCCCACAACCGCCUCAUAUC |
| | | (RNA)-A | CAGGCUCAAGAAUAGAGCUCAGUGUUU |
| | | | UGUUGUUUAAUCAUUCCGACGUGUUUU |
| | | | GCGAUAUUCGCGCAAAGCAGCCAGUCG |
| | | | CGCGCUUGCUUUUAAGUAGAGUUGUUU |
| | | | UUCCACCCGUUUGCCAGGCAUCUUUAA |
| | | | UUUAACAUAUUUUUAUUUUUCAGGCUA |
| | | | ACCUAAAGCAGAGAA |
| 85 | 5′UTR | combo4_S065 | AGGAAACCCCCCACCCCCGCCUCAUAU |
| | | (RNA)-A | CCAGGCUCAAGAAUAGAGCUCAGUGUU |
| | | | UUGUUGUUUAAUCAUUCCGACGUGUUU |
| | | | UGCGAUAUUCGCGCAAAGCAGCCAGUC |
| | | | GCGCGCUUGCUUUUAAGUAGAGUUGUU |
| | | | UUUCCACCCGUUUGCCAGGCAUCUUUA |
| | | | AUUUAACAUAUUUUUAUUUUUCAGGCU |
| | | | AACCUACGCCGCCACC |
| 86 | 5′UTR | F153 | AGGAAAUCCCCACAACCGCCUCAUAUC |
| | | combo5_S065 | CAGGCUCAAGAAUAGAGCUCAGUGUUU |
| | | (RNA-A | UGUUGUUUAAUCAUUCCGACGUGUUUU |
| | | | GCGAUAUUCGCGCAAAGCAGCCAGUCG |
| | | | CGCGCUUGCUUUUAAGUAGAGUUGUUU |
| | | | UUCCACCCGUUUGCCAGGCAUCUUUAA |
| | | | UUUAACAUAUUUUUAUUUUUCAGGCUA |
| | | | ACCUACGCCGCCACC |
| 87 | 5′UTR | combo1_V1.1-A | AGGAAACCCCCCACCCCCGGGGAAAUA |
| | | (RNA) | AGAGAGAAAAGAAGAGUAAGAAGAAAU |
| | | | AUAAGACCCCGGCGCCGCCACC |
| 88 | 5′UTR | combo2_V1.1-A | AGGAAAUCCCCACAACCGGGGAAAUAA |
| | | (RNA) | GAGAGAAAAGAAGAGUAAGAAGAAAUA |
| | | | UAAGACCCCGGCGCCGCCACC |
| 89 | 5′UTR | CrichCR4 + | AGGAAACCCCCCUAAGCCGCCGCCGCC |
| | | GCC3-ExtKozak-A | GCCACC |
| | | (RNA) | |
| 90 | 3′UTR-001 | Creatine | GCGCCUGCCCACCUGCCACCGACUGCU |
| | | Kinase | GGAACCCAGCCAGUGGGAGGGCCUGGC |
| | | | CCACCAGAGUCCUGCUCCCUCACUCCU |
| | | | CGCCCCGCCCCCUGUCCCAGAGUCCCA |
| | | | CCUGGGGGCUCUCUCCACCCUUCUCAG |
| | | | AGUUCCAGUUUCAACCAGAGUUCCAAC |
| | | | CAAUGGGCUCCAUCCUCUGGAUUCUGG |
| | | | CCAAUGAAAUAUCUCCCUGGCAGGGUC |
| | | | CUCUUCUUUUCCCAGAGCUCCACCCCA |
| | | | ACCAGGAGCUCUAGUUAAUGGAGAGCU |
| | | | CCCAGCACACUCGGAGCUUGUGCUUUG |
| | | | UCUCCACGCAAAGCGAUAAAUAAAAGC |
| | | | AUUGGUGGCCUUUGGUCUUUGAAUAAA |
| | | | GCCUGAGUAGGAAGUCUAGA |
| 91 | 3′UTR-002 | Myoglobin | GCCCCUGCCGCUCCCACCCCCACCCAU |
| | | | CUGGGCCCCGGGUUCAAGAGAGAGCGG |
| | | | GGUCUGAUCUCGUGUAGCCAUAUAGAG |
| | | | UUUGCUUCUGAGUGUCUGCUUUGUUUA |
| | | | GUAGAGGUGGGCAGGAGGAGCUGAGGG |
| | | | GCUGGGGCUGGGGUGUUGAAGUUGGCU |
| | | | UUGCAUGCCCAGCGAUGCGCCUCCCUG |
| | | | UGGGAUGUCAUCACCCUGGGAACCGGG |
| | | | AGUGGCCCUUGGCUCACUGUGUUCUGC |
| | | | AUGGUUUGGAUCUGAAUUAAUUGUCCU |
| | | | UUCUUCUAAAUCCCAACCGAACUUCUU |
| | | | CCAACCUCCAAACUGGCUGUAACCCCA |
| | | | AAUCCAAGCCAUUAACUACACCUGACA |
| | | | GUAGCAAUUGUCUGAUUAAUCACUGGC |
| | | | CCCUUGAAGACAGCAGAAUGUCCCUUU |
| | | | GCAAUGAGGAGGAGAUCUGGGCUGGGC |
| | | | GGGCCAGCUGGGGAAGCAUUUGACUAU |
| | | | CUGGAACUUGUGUGUGCCUCCUCAGGU |
| | | | AUGGCAGUGACUCACCUGGUUUUAAUA |
| | | | AAACAACCUGCAACAUCUCAUGGUCUU |
| | | | UGAAUAAAGCCUGAGUAGGAAGUCUAG |
| | | | A |
| 92 | 3′UTR-003 | α-actin | ACACACUCCACCUCCAGCACGCGACUU |
| | | | CUCAGGACGACGAAUCUUCUCAAUGGG |
| | | | GGGGCGGCUGAGCUCCAGCCACCCCGC |
| | | | AGUCACUUUCUUUGUAACAACUUCCGU |
| | | | UGCUGCCAUCGUAAACUGACACAGUGU |
| | | | UUAUAACGUGUACAUACAUUAACUUAU |
| | | | UACCUCAUUUUGUUAUUUUUCGAAACA |
| | | | AAGCCCUGUGGAAGAAAAUGGAAAACU |
| | | | UGAAGAAGCAUUAAAGUCAUUCUGUUA |
| | | | AGCUGCGUAAAUGGUCUUUGAAUAAAG |
| | | | CCUGAGUAGGAAGUCUAGA |
| 93 | 3′UTR-004 | Albumin | CAUCACAUUUAAAAGCAUCUCAGCCUA |
| | | | CCAUGAGAAUAAGAGAAAGAAAAUGAA |
| | | | GAUCAAAAGCUUAUUCAUCUGUUUUUC |
| | | | UUUUUCGUUGGUGUAAAGCCAACACCC |
| | | | UGUCUAAAAAACAUAAAUUUCUUUAAU |
| | | | CAUUUUGCCUCUUUUCUCUGUGCUUCA |
| | | | AUUAAUAAAAAAUGGAAAGAAUCUAAU |
| | | | AGAGUGGUACAGCACUGUUAUUUUUCA |
| | | | AAGAUGUGUUGCUAUCCUGAAAAUUCU |
| | | | GUAGGUUCUGUGGAAGUUCCAGUGUUC |
| | | | UCUCUUAUUCCACUUCGGUAGAGGAUU |
| | | | UCUAGUUUCUUGUGGGCUAAUUAAAUA |
| | | | AAUCAUUAAUACUCUUCUAAUGGUCUU |
| | | | UGAAUAAAGCCUGAGUAGGAAGUCUAG |
| | | | A |
| 94 | 3′UTR-005 | α-globin | GCUGCCUUCUGCGGGGCUUGCCUUCUG |
| | | | GCCAUGCCCUUCUUCUCUCCCUUGCAC |
| | | | CUGUACCUCUUGGUCUUUGAAUAAAGC |
| | | | CUGAGUAGGAAGGCGGCCGCUCGAGCA |
| | | | UGCAUCUAGA |
| 95 | 3′UTR-006 | G-CSF | GCCAAGCCCUCCCCAUCCCAUGUAUUU |
| | | | AUCUCUAUUUAAUAUUUAUGUCUAUUU |
| | | | AAGCCUCAUAUUUAAAGACAGGGAAGA |
| | | | GCAGAACGGAGCCCCAGGCCUCUGUGU |
| | | | CCUUCCCUGCAUUUCUGAGUUUCAUUC |
| | | | UCCUGCCUGUAGCAGUGAGAAAAAGCU |
| | | | CCUGUCCUCCCAUCCCCUGGACUGGGA |
| | | | GGUAGAUAGGUAAAUACCAAGUAUUUA |
| | | | UUACUAUGACUGCUCCCCAGCCCUGGC |
| | | | UCUGCAAUGGGCACUGGGAUGAGCCGC |
| | | | UGUGAGCCCCUGGUCCUGAGGGUCCCC |
| | | | ACCUGGGACCCUUGAGAGUAUCAGGUC |
| | | | UCCCACGUGGGAGACAAGAAAUCCCUG |
| | | | UUUAAUAUUUAAACAGCAGUGUUCCCC |
| | | | AUCUGGGUCCUUGCACCCCUCACUCUG |
| | | | GCCUCAGCCGACUGCACAGCGGCCCCU |
| | | | GCAUCCCCUUGGCUGUGAGGCCCCUGG |
| | | | ACAAGCAGAGGUGGCCAGAGCUGGGAG |
| | | | GCAUGGCCCUGGGGUCCCACGAAUUUG |
| | | | CUGGGGAAUCUCGUUUUUCUUCUUAAG |
| | | | ACUUUUGGGACAUGGUUUGACUCCCGA |
| | | | ACAUCACCGACGCGUCUCCUGUUUUUC |
| | | | UGGGUGGCCUCGGGACACCUGCCCUGC |
| | | | CCCCACGAGGGUCAGGACUGUGACUCU |
| | | | UUUUAGGGCCAGGCAGGUGCCUGGACA |
| | | | UUUGCCUUGCUGGACGGGGACUGGGGA |
| | | | UGUGGGAGGGAGCAGACAGGAGGAAUC |
| | | | AUGUCAGGCCUGUGUGUGAAAGGAAGC |
| | | | UCCACUGUCACCCUCCACCUCUUCACC |
| | | | CCCCACUCACCAGUGUCCCCUCCACUG |
| | | | UCACAUUGUAACUGAACUUCAGGAUAA |
| | | | UAAAGUGUUUGCCUCCAUGGUCUUUGA |
| | | | AUAAAGCCUGAGUAGGAAGGCGGCCGC |
| | | | UCGAGCAUGCAUCUAGA |
| 96 | 3′UTR-007 | Col1a2; | ACUCAAUCUAAAUUAAAAAAGAAAGAA |
| | | collagen, | AUUUGAAAAAACUUUCUCUUUGCCAUU |
| | | type I, alpha 2 | UCUUCUUCUUCUUUUUUAACUGAAAGC |
| | | | UGAAUCCUUCCAUUUCUUCUGCACAUC |
| | | | UACUUGCUUAAAUUGUGGGCAAAAGAG |
| | | | AAAAAGAAGGAUUGAUCAGAGCAUUGU |
| | | | GCAAUACAGUUUCAUUAACUCCUUCCC |
| | | | CCGCUCCCCCAAAAAUUUGAAUUUUUU |
| | | | UUUCAACACUCUUACACCUGUUAUGGA |
| | | | AAAUGUCAACCUUUGUAAGAAAACCAA |
| | | | AAUAAAAAUUGAAAAAUAAAAACCAUA |
| | | | AACAUUUGCACCACUUGUGGCUUUUGA |
| | | | AUAUCUUCCACAGAGGGAAGUUUAAAA |
| | | | CCCAAACUUCCAAAGGUUUAAACUACC |
| | | | UCAAAACACUUUCCCAUGAGUGUGAUC |
| | | | CACAUUGUUAGGUGCUGACCUAGACAG |
| | | | AGAUGAACUGAGGUCCUUGUUUUGUUU |
| | | | UGUUCAUAAUACAAAGGUGCUAAUUAA |
| | | | UAGUAUUUCAGAUACUUGAAGAAUGUU |
| | | | GAUGGUGCUAGAAGAAUUUGAGAAGAA |
| | | | AUACUCCUGUAUUGAGUUGUAUCGUGU |
| | | | GGUGUAUUUUUUAAAAAAUUUGAUUUA |
| | | | GCAUUCAUAUUUUCCAUCUUAUUCCCA |
| | | | AUUAAAAGUAUGCAGAUUAUUUGCCCA |
| | | | AAUCUUCUUCAGAUUCAGCAUUUGUUC |
| | | | UUUGCCAGUCUCAUUUUCAUCUUCUUC |
| | | | CAUGGUUCCACAGAAGCUUUGUUUCUU |
| | | | GGGCAAGCAGAAAAAUUAAAUUGUACC |
| | | | UAUUUUGUAUAUGUGAGAUGUUUAAAU |
| | | | AAAUUGUGAAAAAAAUGAAAUAAAGCA |
| | | | UGUUUGGUUUUCCAAAAGAACAUAU |
| 97 | 3′UTR-008 | Col6a2; | CGCCGCCGCCCGGGCCCCGCAGUCGAG |
| | | collagen, type | GGUCGUGAGCCCACCCCGUCCAUGGUG |
| | | VI, alpha 2 | CUAAGCGGGCCCGGGUCCCACACGGCC |
| | | | AGCACCGCUGCUCACUCGGACGACGCC |
| | | | CUGGGCCUGCACCUCUCCAGCUCCUCC |
| | | | CACGGGGUCCCCGUAGCCCCGGCCCCC |
| | | | GCCCAGCCCCAGGUCUCCCCAGGCCCU |
| | | | CCGCAGGCUGCCCGGCCUCCCUCCCCC |
| | | | UGCAGCCAUCCCAAGGCUCCUGACCUA |
| | | | CCUGGCCCCUGAGCUCUGGAGCAAGCC |
| | | | CUGACCCAAUAAAGGCUUUGAACCCAU |
| 98 | 3′UTR-009 | RPN1; | GGGGCUAGAGCCCUCUCCGCACAGCGU |
| | | ribophorin I | GGAGACGGGGCAAGGAGGGGGGUUAUU |
| | | | AGGAUUGGUGGUUUUGUUUUGCUUUGU |
| | | | UUAAAGCCGUGGGAAAAUGGCACAACU |
| | | | UUACCUCUGUGGGAGAUGCAACACUGA |
| | | | GAGCCAAGGGGUGGGAGUUGGGAUAAU |
| | | | UUUUAUAUAAAAGAAGUUUUUCCACUU |
| | | | UGAAUUGCUAAAAGUGGCAUUUUUCCU |
| | | | AUGUGCAGUCACUCCUCUCAUUUCUAA |
| | | | AAUAGGGACGUGGCCAGGCACGGUGGC |
| | | | UCAUGCCUGUAAUCCCAGCACUUUGGG |
| | | | AGGCCGAGGCAGGCGGCUCACGAGGUC |
| | | | AGGAGAUCGAGACUAUCCUGGCUAACA |
| | | | CGGUAAAACCCUGUCUCUACUAAAAGU |
| | | | ACAAAAAAUUAGCUGGGCGUGGUGGUG |
| | | | GGCACCUGUAGUCCCAGCUACUCGGGA |
| | | | GGCUGAGGCAGGAGAAAGGCAUGAAUC |
| | | | CAAGAGGCAGAGCUUGCAGUGAGCUGA |
| | | | GAUCACGCCAUUGCACUCCAGCCUGGG |
| | | | CAACAGUGUUAAGACUCUGUCUCAAAU |
| | | | AUAAAUAAAUAAAUAAAUAAAUAAAUA |
| | | | AAUAAAUAAAAAUAAAGCGAGAUGUUG |
| | | | CCCUCAAA |
| 99 | 3′UTR-010 | LRP1; low | GGCCCUGCCCCGUCGGACUGCCCCCAG |
| | | density | AAAGCCUCCUGCCCCCUGCCAGUGAAG |
| | | lipoprotein | UCCUUCAGUGAGCCCCUCCCCAGCCAG |
| | | receptor- | CCCUUCCCUGGCCCCGCCGGAUGUAUA |
| | | related | AAUGUAAAAAUGAAGGAAUUACAUUUU |
| | | protein 1 | AUAUGUGAGCGAGCAAGCCGGCAAGCG |
| | | | AGCACAGUAUUAUUUCUCCAUCCCCUC |
| | | | CCUGCCUGCUCCUUGGCACCCCCAUGC |
| | | | UGCCUUCAGGGAGACAGGCAGGGAGGG |
| | | | CUUGGGGCUGCACCUCCUACCCUCCCA |
| | | | CCAGAACGCACCCCACUGGGAGAGCUG |
| | | | GUGGUGCAGCCUUCCCCUCCCUGUAUA |
| | | | AGACACUUUGCCAAGGCUCUCCCCUCU |
| | | | CGCCCCAUCCCUGCUUGCCCGCUCCCA |
| | | | CAGCUUCCUGAGGGCUAAUUCUGGGAA |
| | | | GGGAGGAUCUUUGCUGCCCCUGUCUGG |
| | | | AAGACGUGGCUCUGGGUGAGGUAGGCG |
| | | | GGAAAGGAUGGAGUGUUUUAGUUCUUG |
| | | | GGGGAGGCCACCCCAAACCCCAGCCCC |
| | | | AACUCCAGGGGCACCUAUGAGAUGGCC |
| | | | AUGCUCAACCCCCCUCCCAGACAGGCC |
| | | | CUCCCUGUCUCCAGGGCCCCCACCGAG |
| | | | GUUCCCAGGGCUGGAGACUUCCUCUGG |
| | | | UAAACAUUCCUCCAGCCUCCCCUCCCC |
| | | | UGGGGACGCCAAGGAGGUGGGCCACAC |
| | | | CCAGGAAGGGAAAGCGGGCAGCCCCGU |
| | | | UUUGGGGACGUGAACGUUUUAAUAAUU |
| | | | UUUGCUGAAUUCCUUUACAACUAAAUA |
| | | | ACACAGAUAUUGUUAUAAAUAAAAUUG |
| | | | U |
| 100 | 3′UTR-011 | Nnt1; | AUAUUAAGGAUCAAGCUGUUAGCUAAU |
| | | cardiotrophin- | AAUGCCACCUCUGCAGUUUUGGGAACA |
| | | like cytokine | GGCAAAUAAAGUAUCAGUAUACAUGGU |
| | | factor 1 | GAUGUACAUCUGUAGCAAAGCUCUUGG |
| | | | AGAAAAUGAAGACUGAAGAAAGCAAAG |
| | | | CAAAAACUGUAUAGAGAGAUUUUUCAA |
| | | | AAGCAGUAAUCCCUCAAUUUUAAAAAA |
| | | | GGAUUGAAAAUUCUAAAUGUCUUUCUG |
| | | | UGCAUAUUUUUUGUGUUAGGAAUCAAA |
| | | | AGUAUUUUAUAAAAGGAGAAAGAACAG |
| | | | CCUCAUUUUAGAUGUAGUCCUGUUGGA |
| | | | UUUUUUAUGCCUCCUCAGUAACCAGAA |
| | | | AUGUUUUAAAAAACUAAGUGUUUAGGA |
| | | | UUUCAAGACAACAUUAUACAUGGCUCU |
| | | | GAAAUAUCUGACACAAUGUAAACAUUG |
| | | | CAGGCACCUGCAUUUUAUGUUUUUUUU |
| | | | UUCAACAAAUGUGACUAAUUUGAAACU |
| | | | UUUAUGAACUUCUGAGCUGUCCCCUUG |
| | | | CAAUUCAACCGCAGUUUGAAUUAAUCA |
| | | | UAUCAAAUCAGUUUUAAUUUUUUAAAU |
| | | | UGUACUUCAGAGUCUAUAUUUCAAGGG |
| | | | CACAUUUUCUCACUACUAUUUUAAUAC |
| | | | AUUAAAGGACUAAAUAAUCUUUCAGAG |
| | | | AUGCUGGAAACAAAUCAUUUGCUUUAU |
| | | | AUGUUUCAUUAGAAUACCAAUGAAACA |
| | | | UACAACUUGAAAAUUAGUAAUAGUAUU |
| | | | UUUGAAGAUCCCAUUUCUAAUUGGAGA |
| | | | UCUCUUUAAUUUCGAUCAACUUAUAAU |
| | | | GUGUAGUACUAUAUUAAGUGCACUUGA |
| | | | GUGGAAUUCAACAUUUGACUAAUAAAA |
| | | | UGAGUUCAUCAUGUUGGCAAGUGAUGU |
| | | | GGCAAUUAUCUCUGGUGACAAAAGAGU |
| | | | AAAAUCAAAUAUUUCUGCCUGUUACAA |
| | | | AUAUCAAGGAAGACCUGCUACUAUGAA |
| | | | AUAGAUGACAUUAAUCUGUCUUCACUG |
| | | | UUUAUAAUACGGAUGGAUUUUUUUUCA |
| | | | AAUCAGUGUGUGUUUUGAGGUCUUAUG |
| | | | UAAUUGAUGACAUUUGAGAGAAAUGGU |
| | | | GGCUUUUUUUAGCUACCUCUUUGUUCA |
| | | | UUUAAGCACCAGUAAAGAUCAUGUCUU |
| | | | UUUAUAGAAGUGUAGAUUUUCUUUGUG |
| | | | ACUUUGCUAUCGUGCCUAAAGCUCUAA |
| | | | AUAUAGGUGAAUGUGUGAUGAAUACUC |
| | | | AGAUUAUUUGUCUCUCUAUAUAAUUAG |
| | | | UUUGGUACUAAGUUUCUCAAAAAAUUA |
| | | | UUAACACAUGAAAGACAAUCUCUAAAC |
| | | | CAGAAAAAGAAGUAGUACAAAUUUUGU |
| | | | UACUGUAAUGCUCGCGUUUAGUGAGUU |
| | | | UAAAACACACAGUAUCUUUUGGUUUUA |
| | | | UAAUCAGUUUCUAUUUUGCUGUGCCUG |
| | | | AGAUUAAGAUCUGUGUAUGUGUGUGUG |
| | | | UGUGUGUGUGCGUUUGUGUGUUAAAGC |
| | | | AGAAAAGACUUUUUUAAAAGUUUUAAG |
| | | | UGAUAAAUGCAAUUUGUUAAUUGAUCU |
| | | | UAGAUCACUAGUAAACUCAGGGCUGAA |
| | | | UUAUACCAUGUAUAUUCUAUUAGAAGA |
| | | | AAGUAAACACCAUCUUUAUUCCUGCCC |
| | | | UUUUUCUUCUCUCAAAGUAGUUGUAGU |
| | | | UAUAUCUAGAAAGAAGCAAUUUUGAUU |
| | | | UCUUGAAAAGGUAGUUCCUGCACUCAG |
| | | | UUUAAACUAAAAAUAAUCAUACUUGGA |
| | | | UUUUAUUUAUUUUUGUCAUAGUAAAAA |
| | | | UUUUAAUUUAUAUAUAUUUUUAUUUAG |
| | | | UAUUAUCUUAUUCUUUGCUAUUUGCCA |
| | | | AUCCUUUGUCAUCAAUUGUGUUAAAUG |
| | | | AAUUGAAAAUUCAUGCCCUGUUCAUUU |
| | | | UAUUUUACUUUAUUGGUUAGGAUAUUU |
| | | | AAAGGAUUUUUGUAUAUAUAAUUUCUU |
| | | | AAAUUAAUAUUCCAAAAGGUUAGUGGA |
| | | | CUUAGAUUAUAAAUUAUGGCAAAAAUC |
| | | | UAAAAACAACAAAAAUGAUUUUUAUAC |
| | | | AUUCUAUUUCAUUAUUCCUCUUUUUCC |
| | | | AAUAAGUCAUACAAUUGGUAGAUAUGA |
| | | | CUUAUUUUAUUUUUGUAUUAUUCACUA |
| | | | UAUCUUUAUGAUAUUUAAGUAUAAAUA |
| | | | AUUAAAAAAAUUUAUUGUACCUUAUAG |
| | | | UCUGUCACCAAAAAAAAAAAAUUAUCU |
| | | | GUAGGUAGUGAAAUGCUAAUGUUGAUU |
| | | | UGUCUUUAAGGGCUUGUUAACUAUCCU |
| | | | UUAUUUUCUCAUUUGUCUUAAAUUAGG |
| | | | AGUUUGUGUUUAAAUUACUCAUCUAAG |
| | | | CAAAAAAUGUAUAUAAAUCCCAUUACU |
| | | | GGGUAUAUACCCAAAGGAUUAUAAAUC |
| | | | AUGCUGCUAUAAAGACACAUGCACACG |
| | | | UAUGUUUAUUGCAGCACUAUUCACAAU |
| | | | AGCAAAGACUUGGAACCAACCCAAAUG |
| | | | UCCAUCAAUGAUAGACUUGAUUAAGAA |
| | | | AAUGUGCACAUAUACACCAUGGAAUAC |
| | | | UAUGCAGCCAUAAAAAAGGAUGAGUUC |
| | | | AUGUCCUUUGUAGGGACAUGGAUAAAG |
| | | | CUGGAAACCAUCAUUCUGAGCAAACUA |
| | | | UUGCAAGGACAGAAAACCAAACACUGC |
| | | | AUGUUCUCACUCAUAGGUGGGAAUUGA |
| | | | ACAAUGAGAACACUUGGACACAAGGUG |
| | | | GGGAACACCACACACCAGGGCCUGUCA |
| | | | UGGGGUGGGGGGAGUGGGGAGGGAUAG |
| | | | CAUUAGGAGAUAUACCUAAUGUAAAUG |
| | | | AUGAGUUAAUGGGUGCAGCACACCAAC |
| | | | AUGGCACAUGUAUACAUAUGUAGCAAA |
| | | | CCUGCACGUUGUGCACAUGUACCCUAG |
| | | | AACUUAAAGUAUAAUUAAAAAAAAAAA |
| | | | GAAAACAGAAGCUAUUUAUAAAGAAGU |
| | | | UAUUUGCUGAAAUAAAUGUGAUCUUUC |
| | | | CCAUUAAAAAAAUAAAGAAAUUUUGGG |
| | | | GUAAAAAAACACAAUAUAUUGUAUUCU |
| | | | UGAAAAAUUCUAAGAGAGUGGAUGUGA |
| | | | AGUGUUCUCACCACAAAAGUGAUAACU |
| | | | AAUUGAGGUAAUGCACAUAUUAAUUAG |
| | | | AAAGAUUUUGUCAUUCCACAAUGUAUA |
| | | | UAUACUUAAAAAUAUGUUAUACACAAU |
| | | | AAAUACAUACAUUAAAAAAUAAGUAAA |
| | | | UGUA |
| 101 | 3′UTR-012 | Col6a1; | CCCACCCUGCACGCCGGCACCAAACCC |
| | | collagen, type | UGUCCUCCCACCCCUCCCCACUCAUCA |
| | | VI, alpha 1 | CUAAACAGAGUAAAAUGUGAUGCGAAU |
| | | | UUUCCCGACCAACCUGAUUCGCUAGAU |
| | | | UUUUUUUAAGGAAAAGCUUGGAAAGCC |
| | | | AGGACACAACGCUGCUGCCUGCUUUGU |
| | | | GCAGGGUCCUCCGGGGCUCAGCCCUGA |
| | | | GUUGGCAUCACCUGCGCAGGGCCCUCU |
| | | | GGGGCUCAGCCCUGAGCUAGUGUCACC |
| | | | UGCACAGGGCCCUCUGAGGCUCAGCCC |
| | | | UGAGCUGGCGUCACCUGUGCAGGGCCC |
| | | | UCUGGGGCUCAGCCCUGAGCUGGCCUC |
| | | | ACCUGGGUUCCCCACCCCGGGCUCUCC |
| | | | UGCCCUGCCCUCCUGCCCGCCCUCCCU |
| | | | CCUGCCUGCGCAGCUCCUUCCCUAGGC |
| | | | ACCUCUGUGCUGCAUCCCACCAGCCUG |
| | | | AGCAAGACGCCCUCUCGGGGCCUGUGC |
| | | | CGCACUAGCCUCCCUCUCCUCUGUCCC |
| | | | CAUAGCUGGUUUUUCCCACCAAUCCUC |
| | | | ACCUAACAGUUACUUUACAAUUAAACU |
| | | | CAAAGCAAGCUCUUCUCCUCAGCUUGG |
| | | | GGCAGCCAUUGGCCUCUGUCUCGUUUU |
| | | | GGGAAACCAAGGUCAGGAGGCCGUUGC |
| | | | AGACAUAAAUCUCGGCGACUCGGCCCC |
| | | | GUCUCCUGAGGGUCCUGCUGGUGACCG |
| | | | GCCUGGACCUUGGCCCUACAGCCCUGG |
| | | | AGGCCGCUGCUGACCAGCACUGACCCC |
| | | | GACCUCAGAGAGUACUCGCAGGGGCGC |
| | | | UGGCUGCACUCAAGACCCUCGAGAUUA |
| | | | ACGGUGCUAACCCCGUCUGCUCCUCCC |
| | | | UCCCGCAGAGACUGGGGCCUGGACUGG |
| | | | ACAUGAGAGCCCCUUGGUGCCACAGAG |
| | | | GGCUGUGUCUUACUAGAAACAACGCAA |
| | | | ACCUCUCCUUCCUCAGAAUAGUGAUGU |
| | | | GUUCGACGUUUUAUCAAAGGCCCCCUU |
| | | | UCUAUGUUCAUGUUAGUUUUGCUCCUU |
| | | | CUGUGUUUUUUUCUGAACCAUAUCCAU |
| | | | GUUGCUGACUUUUCCAAAUAAAGGUUU |
| | | | UCACUCCUCUC |
| 102 | 3′UTR-013 | Calr; | AGAGGCCUGCCUCCAGGGCUGGACUGA |
| | | calreticulin | GGCCUGAGCGCUCCUGCCGCAGAGCUG |
| | | | GCCGCGCCAAAUAAUGUCUCUGUGAGA |
| | | | CUCGAGAACUUUCAUUUUUUUCCAGGC |
| | | | UGGUUCGGAUUUGGGGUGGAUUUUGGU |
| | | | UUUGUUCCCCUCCUCCACUCUCCCCCA |
| | | | CCCCCUCCCCGCCCUUUUUUUUUUUUU |
| | | | UUUUUAAACUGGUAUUUUAUCUUUGAU |
| | | | UCUCCUUCAGCCCUCACCCCUGGUUCU |
| | | | CAUCUUUCUUGAUCAACAUCUUUUCUU |
| | | | GCCUCUGUCCCCUUCUCUCAUCUCUUA |
| | | | GCUCCCCUCCAACCUGGGGGGCAGUGG |
| | | | UGUGGAGAAGCCACAGGCCUGAGAUUU |
| | | | CAUCUGCUCUCCUUCCUGGAGCCCAGA |
| | | | GGAGGGCAGCAGAAGGGGGUGGUGUCU |
| | | | CCAACCCCCCAGCACUGAGGAAGAACG |
| | | | GGGCUCUUCUCAUUUCACCCCUCCCUU |
| | | | UCUCCCCUGCCCCCAGGACUGGGCCAC |
| | | | UUCUGGGUGGGGCAGUGGGUCCCAGAU |
| | | | UGGCUCACACUGAGAAUGUAAGAACUA |
| | | | CAAACAAAAUUUCUAUUAAAUUAAAUU |
| | | | UUGUGUCUCC |
| 103 | 3′UTR-014 | Colla1; | CUCCCUCCAUCCCAACCUGGCUCCCUC |
| | | collagen, type | CCACCCAACCAACUUUCCCCCCAACCC |
| | | I, alpha 1 | GGAAACAGACAAGCAACCCAAACUGAA |
| | | | CCCCCUCAAAAGCCAAAAAAUGGGAGA |
| | | | CAAUUUCACAUGGACUUUGGAAAAUAU |
| | | | UUUUUUCCUUUGCAUUCAUCUCUCAAA |
| | | | CUUAGUUUUUAUCUUUGACCAACCGAA |
| | | | CAUGACCAAAAACCAAAAGUGCAUUCA |
| | | | ACCUUACCAAAAAAAAAAAAAAAAAAA |
| | | | GAAUAAAUAAAUAACUUUUUAAAAAAG |
| | | | GAAGCUUGGUCCACUUGCUUGAAGACC |
| | | | CAUGCGGGGGUAAGUCCCUUUCUGCCC |
| | | | GUUGGGCUUAUGAAACCCCAAUGCUGC |
| | | | CCUUUCUGCUCCUUUCUCCACACCCCC |
| | | | CUUGGGGCCUCCCCUCCACUCCUUCCC |
| | | | AAAUCUGUCUCCCCAGAAGACACAGGA |
| | | | AACAAUGUAUUGUCUGCCCAGCAAUCA |
| | | | AAGGCAAUGCUCAAACACCCAAGUGGC |
| | | | CCCCACCCUCAGCCCGCUCCUGCCCGC |
| | | | CCAGCACCCCCAGGCCCUGGGGGACCU |
| | | | GGGGUUCUCAGACUGCCAAAGAAGCCU |
| | | | UGCCAUCUGGCGCUCCCAUGGCUCUUG |
| | | | CAACAUCUCCCCUUCGUUUUUGAGGGG |
| | | | GUCAUGCCGGGGGAGCCACCAGCCCCU |
| | | | CACUGGGUUCGGAGGAGAGUCAGGAAG |
| | | | GGCCACGACAAAGCAGAAACAUCGGAU |
| | | | UUGGGGAACGCGUGUCAAUCCCUUGUG |
| | | | CCGCAGGGCUGGGCGGGAGAGACUGUU |
| | | | CUGUUCCUUGUGUAACUGUGUUGCUGA |
| | | | AAGACUACCUCGUUCUUGUCUUGAUGU |
| | | | GUCACCGGGGCAACUGCCUGGGGGCGG |
| | | | GGAUGGGGGCAGGGUGGAAGCGGCUCC |
| | | | CCAUUUUAUACCAAAGGUGCUACAUCU |
| | | | AUGUGAUGGGUGGGGUGGGGAGGGAAU |
| | | | CACUGGUGCUAUAGAAAUUGAGAUGCC |
| | | | CCCCCAGGCCAGCAAAUGUUCCUUUUU |
| | | | GUUCAAAGUCUAUUUUUAUUCCUUGAU |
| | | | AUUUUUCUUUUUUUUUUUUUUUUUUUG |
| | | | UGGAUGGGGACUUGUGAAUUUUUCUAA |
| | | | AGGUGCUAUUUAACAUGGGAGGAGAGC |
| | | | GUGUGCGGCUCCAGCCCAGCCCGCUGC |
| | | | UCACUUUCCACCCUCUCUCCACCUGCC |
| | | | UCUGGCUUCUCAGGCCUCUGCUCUCCG |
| | | | ACCUCUCUCCUCUGAAACCCUCCUCCA |
| | | | CAGCUGCAGCCCAUCCUCCCGGCUCCC |
| | | | UCCUAGUCUGUCCUGCGUCCUCUGUCC |
| | | | CCGGGUUUCAGAGACAACUUCCCAAAG |
| | | | CACAAAGCAGUUUUUCCCCCUAGGGGU |
| | | | GGGAGGAAGCAAAAGACUCUGUACCUA |
| | | | UUUUGUAUGUGUAUAAUAAUUUGAGAU |
| | | | GUUUUUAAUUAUUUUGAUUGCUGGAAU |
| | | | AAAGCAUGUGGAAAUGACCCAAACAUA |
| | | | AUCCGCAGUGGCCUCCUAAUUUCCUUC |
| | | | UUUGGAGUUGGGGGAGGGGUAGACAUG |
| | | | GGGAAGGGGCUUUGGGGUGAUGGGCUU |
| | | | GCCUUCCAUUCCUGCCCUUUCCCUCCC |
| | | | CACUAUUCUCUUCUAGAUCCCUCCAUA |
| | | | ACCCCACUCCCCUUUCUCUCACCCUUC |
| | | | UUAUACCGCAAACCUUUCUACUUCCUC |
| | | | UUUCAUUUUCUAUUCUUGCAAUUUCCU |
| | | | UGCACCUUUUCCAAAUCCUCUUCUCCC |
| | | | CUGCAAUACCAUACAGGCAAUCCACGU |
| | | | GCACAACACACACACACACUCUUCACA |
| | | | UCUGGGGUUGUCCAAACCUCAUACCCA |
| | | | CUCCCCUUCAAGCCCAUCCACUCUCCA |
| | | | CCCCCUGGAUGCCCUGCACUUGGUGGC |
| | | | GGUGGGAUGCUCAUGGAUACUGGGAGG |
| | | | GUGAGGGGAGUGGAACCCGUGAGGAGG |
| | | | ACCUGGGGGCCUCUCCUUGAACUGACA |
| | | | UGAAGGGUCAUCUGGCCUCUGCUCCCU |
| | | | UCUCACCCACGCUGACCUCCUGCCGAA |
| | | | GGAGCAACGCAACAGGAGAGGGGUCUG |
| | | | CUGAGCCUGGCGAGGGUCUGGGAGGGA |
| | | | CCAGGAGGAAGGCGUGCUCCCUGCUCG |
| | | | CUGUCCUGGCCCUGGGGGAGUGAGGGA |
| | | | GACAGACACCUGGGAGAGCUGUGGGGA |
| | | | AGGCACUCGCACCGUGCUCUUGGGAAG |
| | | | GAAGGAGACCUGGCCCUGCUCACCACG |
| | | | GACUGGGUGCCUCGACCUCCUGAAUCC |
| | | | CCAGAACACAACCCCCCUGGGCUGGGG |
| | | | UGGUCUGGGGAACCAUCGUGCCCCCGC |
| | | | CUCCCGCCUACUCCUUUUUAAGCUU |
| 104 | 3′UTR-015 | Plod1; | UUGGCCAGGCCUGACCCUCUUGGACCU |
| | | procollagen- | UUCUUCUUUGCCGACAACCACUGCCCA |
| | | lysine, 2- | GCAGCCUCUGGGACCUCGGGGUCCCAG |
| | | oxoglutarate | GGAACCCAGUCCAGCCUCCUGGCUGUU |
| | | 5-dioxygenase 1 | GACUUCCCAUUGCUCUUGGAGCCACCA |
| | | | AUCAAAGAGAUUCAAAGAGAUUCCUGC |
| | | | AGGCCAGAGGCGGAACACACCUUUAUG |
| | | | GCUGGGGCUCUCCGUGGUGUUCUGGAC |
| | | | CCAGCCCCUGGAGACACCAUUCACUUU |
| | | | UACUGCUUUGUAGUGACUCGUGCUCUC |
| | | | CAACCUGUCUUCCUGAAAAACCAAGGC |
| | | | CCCCUUCCCCCACCUCUUCCAUGGGGU |
| | | | GAGACUUGAGCAGAACAGGGGCUUCCC |
| | | | CAAGUUGCCCAGAAAGACUGUCUGGGU |
| | | | GAGAAGCCAUGGCCAGAGCUUCUCCCA |
| | | | GGCACAGGUGUUGCACCAGGGACUUCU |
| | | | GCUUCAAGUUUUGGGGUAAAGACACCU |
| | | | GGAUCAGACUCCAAGGGCUGCCCUGAG |
| | | | UCUGGGACUUCUGCCUCCAUGGCUGGU |
| | | | CAUGAGAGCAAACCGUAGUCCCCUGGA |
| | | | GACAGCGACUCCAGAGAACCUCUUGGG |
| | | | AGACAGAAGAGGCAUCUGUGCACAGCU |
| | | | CGAUCUUCUACUUGCCUGUGGGGAGGG |
| | | | GAGUGACAGGUCCACACACCACACUGG |
| | | | GUCACCCUGUCCUGGAUGCCUCUGAAG |
| | | | AGAGGGACAGACCGUCAGAAACUGGAG |
| | | | AGUUUCUAUUAAAGGUCAUUUAAACCA |
| 105 | 3′UTR-016 | Nucb1; | UCCUCCGGGACCCCAGCCCUCAGGAUU |
| | | nucleobindin 1 | CCUGAUGCUCCAAGGCGACUGAUGGGC |
| | | | GCUGGAUGAAGUGGCACAGUCAGCUUC |
| | | | CCUGGGGGCUGGUGUCAUGUUGGGCUC |
| | | | CUGGGGCGGGGGCACGGCCUGGCAUUU |
| | | | CACGCAUUGCUGCCACCCCAGGUCCAC |
| | | | CUGUCUCCACUUUCACAGCCUCCAAGU |
| | | | CUGUGGCUCUUCCCUUCUGUCCUCCGA |
| | | | GGGGCUUGCCUUCUCUCGUGUCCAGUG |
| | | | AGGUGCUCAGUGAUCGGCUUAACUUAG |
| | | | AGAAGCCCGCCCCCUCCCCUUCUCCGU |
| | | | CUGUCCCAAGAGGGUCUGCUCUGAGCC |
| | | | UGCGUUCCUAGGUGGCUCGGCCUCAGC |
| | | | UGCCUGGGUUGUGGCCGCCCUAGCAUC |
| | | | CUGUAUGCCCACAGCUACUGGAAUCCC |
| | | | CGCUGCUGCUCCGGGCCAAGCUUCUGG |
| | | | UUGAUUAAUGAGGGCAUGGGGUGGUCC |
| | | | CUCAAGACCUUCCCCUACCUUUUGUGG |
| | | | AACCAGUGAUGCCUCAAAGACAGUGUC |
| | | | CCCUCCACAGCUGGGUGCCAGGGGCAG |
| | | | GGGAUCCUCAGUAUAGCCGGUGAACCC |
| | | | UGAUACCAGGAGCCUGGGCCUCCCUGA |
| | | | ACCCCUGGCUUCCAGCCAUCUCAUCGC |
| | | | CAGCCUCCUCCUGGACCUCUUGGCCCC |
| | | | CAGCCCCUUCCCCACACAGCCCCAGAA |
| | | | GGGUCCCAGAGCUGACCCCACUCCAGG |
| | | | ACCUAGGCCCAGCCCCUCAGCCUCAUC |
| | | | UGGAGCCCCUGAAGACCAGUCCCACCC |
| | | | ACCUUUCUGGCCUCAUCUGACACUGCU |
| | | | CCGCAUCCUGCUGUGUGUCCUGUUCCA |
| | | | UGUUCCGGUUCCAUCCAAAUACACUUU |
| | | | CUGGAACAAA |
| 106 | 3′UTR-017 | α-globin | GCUGGAGCCUCGGUGGCCAUGCUUCUU |
| | | | GCCCCUUGGGCCUCCCCCCAGCCCCUC |
| | | | CUCCCCUUCCUGCACCCGUACCCCCGU |
| | | | GGUCUUUGAAUAAAGUCUGAGUGGGCG |
| | | | GC |
| 107 | 3′UTR-018 | Downstream UTR | UAAUAGGCUGGAGCCUCGGUGGCCAUG |
| | | | CUUCUUGCCCCUUGGGCCUCCCCCCAG |
| | | | CCCCUCCUCCCCUUCCUGCACCCGUAC |
| | | | CCCCGUGGUCUUUGAAUAAAGUCUGAG |
| | | | UGGGCGGC |
| 108 | 3′UTR-109 | Downstream UTR | UGAUAAUAGGCUGGAGCCUCGGUGGCC |
| | | | AUGCUUCUUGCCCCUUGGGCCUCCCCC |
| | | | CAGCCCCUCCUCCCCUUCCUGCACCCG |
| | | | UACCCCCUGGUCUUUGAAUAAAGUCUG |
| | | | AGUGGGCGGC |
| 109 | 3′UTR | v1.1 3′UTR | UGAUAAUAGGCUGGAGCCUCGGUGGCC |
| | | (RNA) | UAGCUUCUUGCCCCUUGGGCCUCCCCC |
| | | | CAGCCCCUCCUCCCCUUCCUGCACCCG |
| | | | UACCCCCGUGGUCUUUGAAUAAAGUCU |
| | | | GAGUGGGCGGC |
| 110 | 3′UTR-020 | Downstream UTR | UGAUAAUAGGCUGGAGCCUCGGUGGCC |
| | | | AUGCUUCUUGCCCCUUGGGCCUCCCCC |
| | | | CAGCCCCUCCUCCCCUUCCUGCACCCG |
| | | | UACCCCCGUGGUCUUUGAAUAAAGUCU |
| | | | GAGUGGGCGGC |
| 111 | 3XFLAG | Epitope tag | DYKDHDGDYKDHDIDYKDDDK |
| 112 | Myc | Epitope tag | EQKLISEEDL |
| 113 | V5 | Epitope tag | GKPIPNPLLGLDST |
| 114 | Hemagglutin | Epitope tag | YPYDVPDYA |
| | in A (HA) | | |
| 115 | 6xHis tag | Epitope tag | HHHHHH |
| 116 | HSV | Epitope tag | QPELAPEDPED |
| 117 | VSV-G | Epitope tag | YTDIEMNRLGK |
| 118 | NE | Epitope tag | TKENPRSNQEESYDDNES |
| 119 | AViTag | Epitope tag | GLNDIFEAQKIEWHE |
| 120 | Calmodulin | Epitope tag | KRRWKKNFIAVSAANRFKKISSSGAL |
| 121 | E tag | Epitope tag | GAPVPYPDPLEPR |
| 122 | S tag | Epitope tag | KETAAAKFERQHMDS |
| 123 | SBP tag | Epitope tag | MDEKTTGWRGGHVVEGLAGELEQLRAR |
| | | | LEHHPQGQREP |
| 124 | Softag 1 | Epitope tag | SLAELLNAGLGGS |
| 125 | Softag 3 | Epitope tag | TQDPSRVG |
| 126 | Strep tag | Epitope tag | WSHPQFEK |
| 127 | Ty tag | Epitope tag | EVHTNQDPLD |
| 128 | Xpress tag | Epitope tag | DLYDDDDK |

1. A messenger RNA (mRNA), wherein the mRNA comprises: a 5′cap, a 5′untranslated region (UTR), an initiation codon, a full open reading frame (ORF) encoding a polypeptide, and a 3′ UTR, wherein the 5′ UTR comprises a C-rich RNA element located proximal to the 5′ cap, wherein the C-rich RNA element comprises a sequence of linked nucleotides, or derivatives or analogs thereof, wherein each nucleotide comprises a nucleobase selected from the group consisting of: adenine, guanine, thymine, uracil, and cytosine, linked in any order, and wherein the C-rich RNA element provides a translational regulatory activity selected from:
a. increasing residence time of a 43S pre-initiation complex (PIC) or ribosome at, or proximal to, the initiation codon;
b. increasing initiation of polypeptide synthesis at or from the initiation codon;
c. increasing an amount of polypeptide translated from the full ORF;
d. increasing fidelity of initiation codon decoding by the PIC or ribosome;
e. inhibiting or reducing leaky scanning by the PIC or ribosome;
f. decreasing a rate of decoding the initiation codon by the PIC or ribosome;
g. inhibiting or reducing initiation of polypeptide synthesis at any codon within the mRNA other than the initiation codon;
h. inhibiting or reducing the amount of polypeptide translated from any ORF within the mRNA other than the full ORF;
i. inhibiting or reducing the production of aberrant translation products;
j. increasing ribosomal density on the mRNA; and
k. a combination of any of (a)-(j).

2. The mRNA of claim 1, wherein the C-rich element comprises a sequence of (i) about 95%, about 90%, about 85%, about 80%, about 75%, about 70%, about 65%, about 60%, about 55%, about 50%, or greater than 50% cytosine nucleobases or derivatives or analogs thereof (ii) less than about 25%, less than about 20%, less than about 15%, less than about 10%, or less than about 5% guanosine nucleobases, or derivatives or analogs thereof and/or (iii) about 50% or less adenosine nucleobases and/or uracil nucleobases, or derivatives or analogs thereof.

3.-4. (canceled)

5. The mRNA of claim 1, wherein the C-rich RNA element comprises a sequence of about 3-20 nucleotides, about 4-18 nucleotides, about 6-16 nucleotides, about 6-14 nucleotides, about 6-12 nucleotides, about 6-10 nucleotides, about 8-14 nucleotides, about 8-12 nucleotides, about 8-10 nucleotides, about 10-12 nucleotides, about 10-14 nucleotides, about 14 nucleotides, about 13 nucleotides, about 12 nucleotides, about 11 nucleotides, about 10 nucleotides, or about 20, 19, 18, 17, 16, 15, 14, 13, 12, 11, 10, 9, 8, 7, 6, 5, 4, or 3 nucleotides or derivatives or analogs thereof, linked in any order.

6.-7. (canceled)

8. The mRNA of claim 1, wherein the C-rich RNA element is located (i) downstream of and immediately adjacent to the 5′ cap or the 5′end of the mRNA in the 5′ UTR; and/or (ii) about 45-50, about 40-45, about 35-40, about 30-35, about 25-30, about 20-25, about 15-20, about 10-15, about 6-10 nucleotides, about 1-5 nucleotides, or about 20, 19, 18, 17, 16, 15, 14, 13, 12, 11, 10, 9, 8, 7, 6, 5, 4, 3, 2 or 1 nucleotide downstream of the 5′ cap or the 5′end of the mRNA in the 5′ UTR.

9. (canceled)

10. The mRNA of claim 1, wherein the 5′ UTR comprises a Kozak-like sequence upstream of the initiation codon, and wherein the C-rich RNA element is located: (i) upstream of the Kozak-like sequence in the 5′ UTR; or (ii) about 40-45, about 35-40, about 30-35, about 25-30, about 20-25, about 15-20, about 10-15, about 6-10 nucleotides, about 1-5 nucleotides, or about 20, 19, 18, 17, 16, 15, 14, 13, 12, 11, 10, 9, 8, 7, 6, 5, 4, 3, 2 or 1 nucleotide upstream of the Kozak-like sequence in the 5′ UTR.

11. (canceled)

12. An mRNA comprising:
a 5′ cap;
a 5′ UTR comprising a C-rich RNA element of about 3-20 nucleotides comprising a sequence of greater than 50% cytosine nucleobases and less than 10% guanosine nucleobases, wherein the C-rich RNA element is located about 1-50 nucleotides downstream of the 5′ cap or 5′ end of the mRNA in the 5′ UTR;
an ORF encoding a polypeptide; and
a 3′ UTR,
wherein the C-rich RNA element comprises a sequence of linked nucleotides comprising the formula:
5′-[C1]ᵥ-[N1]_{w}-[N2]ₓ-[N3]_{y}-[C2]_{z}-3′,
wherein C1 and C2 are nucleotides comprising cytidine, or a derivative or analogue thereof, wherein N1, and N2 and N3 if present, are each a nucleotide comprising a nucleobase selected from the group consisting of: adenine, guanine, thymine, uracil, and cytosine, and derivatives or analogues thereof, wherein v, w, x, y and z are integers whose value indicates the number of nucleotides comprising the C-rich RNA element, wherein v=2-15 nucleotides, wherein w=1-5 nucleotides, wherein x=0-5 nucleotides, wherein y=0-5 nucleotides, and wherein z=2-10 nucleotides.

13. The mRNA of claim 12, wherein (i) v=3-12 nucleotides, 5-10 nucleotides, 6-8 nucleotides, 3, 4, 5, 6, 7, 8, 9 or 10 nucleotides; (ii) w=1-3 nucleotides, 1, 2, or 3 nucleotide(s); (iii) x=0-3 nucleotides, 0, 1, 2, or 3 nucleotide(s); (iv) y=0-3 nucleotides, 0, 1, 2, or 3 nucleotide(s); and/or (v) z=2-7 nucleotides, 3-5 nucleotides, 2, 3, 4, 5, 6, or 7 nucleotides.

14.-17. (canceled)

18. The mRNA of claim 12, wherein (i) N1 comprises adenosine, or derivative or analogue thereof; w=1 or 2; x=0, 1, 2, or 3; and y=0, 1, 2, or 3; or (ii) N1 comprises uracil, or derivative or analogue thereof; N2 comprises adenosine, or derivative or analogue thereof; N3 is guanosine, or derivative or analogue thereof w=1 or 2; x=1, 2, or 3; and y=1 or 2.

19.-21. (canceled)

22. The mRNA of claim 12, wherein v=4-10 nucleotides, wherein w=1-3 nucleotides, wherein x=0-3 nucleotides, wherein y=0-3 nucleotides, and wherein z=2-6 nucleotides.

23.-29. (canceled)

30. The mRNA of claim 22, wherein (i) N1 comprises uracil, or derivative or analogue thereof; w=1 or 2; N2 comprises adenosine, or derivative or analogue thereof; x=1, 2, or 3; N3 is guanosine, or derivative or analogue thereof; and y=1 or 2; (ii) wherein N1 comprises uracil, or derivative or analogue thereof w=1; N2 comprises adenosine, or derivative or analogue thereof x=2; N3 is guanosine, or derivative or analogue thereof and y=1; (iii) v=6-8; N1 comprises adenosine, or derivative or analogue thereof, w=1 or 2; x=0; y=0; and z=2-5; or (iv) v=6-8; N1 comprises uracil, or derivative or analogue thereof, w=1; N2 comprises adenosine, or derivative or analogue thereof, x=2; N3 is guanosine, or derivative or analogue thereof y=1; and z=2-5.

31.-33. (canceled)

34. The mRNA of claim 12, wherein the C-rich RNA element comprises a nucleotide sequence selected from: (i) the nucleotide sequence [5′-CCCCCCCCAACC′-3] set forth in SEQ ID NO 30; (ii) the nucleotide sequence [5′-CCCCCCCAACCC′-3] set forth in SEQ ID NO: 29; (iii) the nucleotide sequence [5′-CCCCCCACCCCC′-3] set forth in SEQ ID NO: 31; (iv) the nucleotide sequence [5′-CCCCCCUAAGCC′-3] set forth in SEQ ID NO: 32; (v) the nucleotide sequence [5′-CCCACAACC-3] set forth in SEQ ID NO: 33; and (vi) the nucleotide sequence [5′-CCCCCACAACC-3] set forth in SEQ ID NO: 34.

35.-43. (canceled)

44. The mRNA of claim 1, comprising a Kozak-like sequence in the 5′UTR, wherein the 5′UTR comprises a GC-rich RNA element comprising a sequence of about 20-30, about 10-20, about 10-15, about 5-15, or about 3-15 nucleotides, or derivatives or analogs thereof, wherein the GC-rich RNA element comprises a sequence of cytosine and guanine, wherein the sequence is at least about 50% cytosine, and wherein the GC-rich RNA element is located upstream of the Kozak-like in the 5′ UTR.

45.-46. (canceled)

47. The mRNA of claim 44, wherein the GC-rich RNA element comprises a sequence of about 3-30 guanine and cytosine nucleotides, or derivatives or analogues thereof, wherein the sequence comprises a repeating GC-motif, wherein the repeating GC-motif is [CCG]ₙ or [GCC]ₙ, wherein n=1 to 10, 1-5, 3, 2 or 1.

48. The mRNA of claim 44, wherein the sequence of the GC-rich RNA element comprises the sequence selected from (i) the sequence of EK1 [CCCGCC] set forth in SEQ ID NO: 3; (ii) the sequence of EK2 [GCCGCC] set forth in SEQ ID NO: 18; (iii) the sequence of EK3 [CCGCCG] set forth in SEQ ID NO: 19; (iv) the sequence of V1 [CCCCGGCGCC] set forth in SEQ ID NO: 1; (v) the sequence of V2 [CCCCGGC] set forth in SEQ ID NO: 2; (vi) the sequence of CG1 [GCGCCCCGCGGCGCCCCGCG] set forth in SEQ ID NO: 20; and (vii) the sequence of CG2 [CCCGCCCGCCCCGCCCCGCC] set forth in SEQ ID NO: 21.

49.-52. (canceled)

53. The mRNA of claim 44, wherein the GC-rich RNA element is located about 20-30, about 15-20, about 10-15, about 5-10, or about 1-5 nucleotides upstream of the Kozak-like sequence in the 5′ UTR; or wherein the GC-rich RNA element is upstream of and immediately adjacent to the Kozak-like sequence in the 5′ UTR.

54.-55. (canceled)

56. The mRNA of claim 53, wherein the Kozak-like sequence comprises the sequence [5′-GCCACC-′3] set forth in SEQ ID NO: 148 or [5′-GCCGCC-′3] set forth in SEQ ID NO: 18.

57.-64. (canceled)

65. The mRNA of claim 1,
wherein the 5′UTR comprises:
(i) a C-rich RNA element comprising a nucleotide sequence selected from the group consisting of: SEQ ID NO: 29, SEQ ID NO: 30, SEQ ID NO: 31, SEQ ID NO: 32, SEQ ID NO: 33 and SEQ ID NO: 34; and
(ii) a GC-rich RNA element comprising a nucleotide sequence selected from the group consisting of: SEQ ID NO: 1, SEQ ID NO: 2, SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 and SEQ ID NO: 28.

66.-69. (canceled)

70. The mRNA of claim 65, wherein the C-rich RNA element is located about 15-20, about 10-15, about 5-10 nucleotides, about 1-5 nucleotides, or about 20, 19, 18, 17, 16, 15, 14, 13, 12, 11, 10, 9, 8, 7, 6, 5, 4, 3, 2 or 1 nucleotide downstream of the 5′ cap or 5′end of the mRNA in the 5′ UTR; and wherein the C-rich RNA element is located upstream of the GC-rich RNA element in the 5′ UTR.

71.-75. (canceled)

76. The mRNA of claim 1, wherein the 5′UTR comprises a sequence selected from the group consisting of:
(i) a nucleotide sequence comprising a C-rich RNA element comprising the nucleotide sequence set forth in SEQ ID NO: 31 inserted within a 5′ UTR comprising the nucleotide sequence set forth in SEQ ID NO: 45, 71 or 149;
(ii) a nucleotide sequence comprising a C-rich RNA element comprising the nucleotide sequence set forth in SEQ ID NO: 32 inserted within a 5′ UTR comprising the nucleotide sequence set forth in SEQ ID NO: 45, 71 or 149;
(iii) a nucleotide sequence comprising a C-rich RNA element comprising the nucleotide sequence set forth in SEQ ID NO: 31 inserted within a 5′ UTR comprising the nucleotide sequence set forth in SEQ ID NO: 46 or the nucleotide sequence set forth in SEQ ID NO: 42, 72 or 154;
(iv) a nucleotide sequence comprising a C-rich RNA element comprising the nucleotide sequence set forth in SEQ ID NO: 32 inserted within a 5′ UTR comprising the nucleotide sequence set forth in SEQ ID NO: 46 or the nucleotide sequence set forth in SEQ ID NO: 42, 72 or 154;
(v) a nucleotide sequence comprising a C-rich RNA element comprising the nucleotide sequence set forth in SEQ ID NO: 33 inserted within a 5′ UTR comprising the nucleotide sequence set forth in SEQ ID NO: 46; and
(vi) a nucleotide sequence comprising a C-rich RNA element comprising the nucleotide sequence set forth in SEQ ID NO: 33 inserted within a 5′ UTR comprising the nucleotide sequence set forth in SEQ ID NO: 42, 72 or 154.

77.-78. (canceled)

79. The mRNA of claim 76, comprising a GC-rich RNA element comprising the nucleotide sequence set forth in SEQ ID NO: 1 inserted within the 5′UTR; wherein the C-rich RNA element is located about 45-50, about 40-45, about 35-40, about 30-35, about 25-30, about 20-25, about 15-20, about 10-15, about 6-10 nucleotides upstream of the GC-rich RNA element in the 5′ UTR; and wherein the GC-rich RNA element is located about 20, about 15, about 10 or about 5 nucleotides upstream of the Kozak like sequence in the 5′ UTR or upstream of and immediately adjacent to the Kozak like sequence in the 5′ UTR.

80.-84. (canceled)

85. The mRNA of claim 1, wherein the 5′ UTR comprises a nucleotide sequence selected from the group consisting of:
(i) the nucleotide sequence set forth in SEQ ID NO: 35;
(ii) the nucleotide sequence set forth in SEQ ID NO: 87;
(iii) the nucleotide sequence set forth in SEQ ID NO: 160;
(iv) the nucleotide sequence set forth in SEQ ID NO: 36;
(v) the nucleotide sequence set forth in SEQ ID NO: 88;
(vi) the nucleotide sequence set forth in SEQ ID NO: 161;
(vii) the nucleotide sequence set forth in SEQ ID NO: 40;
(viii) the nucleotide sequence set forth in SEQ ID NO: 85; and
(ix) the nucleotide sequence set forth in SEQ ID NO: 158; and
(x) the nucleotide sequence set forth in SEQ ID NO: 41.

86. The mRNA of claim 1, wherein the mRNA comprises:
(i) a first polynucleotide, wherein the first polynucleotide is chemically synthesized, and wherein the first polynucleotide comprises a 5′ UTR comprising at least one C-rich RNA sequence, and;
(ii) a second polynucleotide, wherein the second polynucleotide is synthesized by in vitro transcription, and, wherein the second polynucleotide comprises an ORF encoding a polypeptide, and a 3′ UTR.

87. The mRNA of claim 86, wherein the first polynucleotide and the second polynucleotide are chemically cross-linked or are enzymatically ligated.

88. (canceled)

89. A pharmaceutical composition comprising the mRNA of claim 1, and a pharmaceutically acceptable carrier.

90. A lipid nanoparticle comprising the mRNA of claim 1.

91.-93. (canceled)

94. A method to (i) inhibit or reduce the amount of polypeptide translated from any open reading frame within an mRNA other than the full open reading frame, or (ii) inhibit or reduce the production of aberrant translation products encoded by an mRNA, the method comprising administering to a subject an mRNA of claim 1.

95. (canceled)

96. A method of identifying an RNA element having translational regulatory activity, the method comprising:
i. providing a population of polynucleotides, wherein each polynucleotide comprises a plurality of open reading frames encoding a plurality of polypeptides, each comprising a peptide epitope tag, wherein each polynucleotide comprises:
a. at least one first AUG codon upstream of, in-frame, and operably linked to, at least one first open reading frame encoding at least one first polypeptide comprising at least one first peptide epitope tag;
b. at least one second AUG codon upstream of, in-frame, and operably linked to, at least one second open reading frame encoding at least one second polypeptide comprising at least one second peptide epitope tag, wherein the second AUG codon is downstream and out-of-frame of the first AUG codon; optionally,
c. at least one third AUG codon upstream of, in-frame, and operably linked to, at least one third open reading frame encoding at least one third polypeptide comprising at least one third peptide epitope tag, wherein the third AUG codon is downstream and out-of-frame with the first and second AUG codons, and;
d. a 5′ UTR and a 3′ UTR, wherein the 5′ UTR of each polynucleotide within the population comprises a unique nucleotide sequence;
e. no stop codons (UAG, UGA, or UAA) within any frame between the first AUG and the stop codon corresponding to the first AUG;
ii. providing conditions suitable for translation of each polynucleotide in the population of polynucleotides;
iii. isolating a complex comprising a nascent translation product comprising the first, second and, if present, third epitope tag, and the 5′ UTR corresponding to the epitope tag and encoded polynucleotide;
iv. determining the sequences of the 5′ UTRs corresponding to each polynucleotide encoding the nascent translation product; and
v. determining which nucleotides are enriched at each position in the 5′UTR of the first polynucleotide compared to the second, and optionally third, polynucleotide.

97.-100. (canceled)


SEQUENCE LISTING (163 sequences)

SEQ ID NO: 1 - DNA, 10 bp, Artificial Sequence
  Synthetic GC-rich RNA element, V1
ccccggcgcc

SEQ ID NO: 2 - DNA, 7 bp, Artificial Sequence
  Synthetic GC-rich RNA element, V2
ccccggc

SEQ ID NO: 3 - DNA, 6 bp, Artificial Sequence
  Synthetic GC-rich RNA element, EK1
cccgcc

SEQ ID NO: 4 - DNA, 41 bp, Artificial Sequence
  Synthetic 5 UTR-022 (DNA)
gggaaataagagagaaaagaagagtaagaagaaatataaga

SEQ ID NO: 5 - RNA, 41 nt, Artificial Sequence
  Synthetic 5 UTR-022 (RNA)
gggaaauaagagagaaaagaagaguaagaagaaauauaaga

SEQ ID NO: 6 - RNA, 57 nt, Artificial Sequence
  Synthetic 5 UTR-023 (RNA)
gggaaauaagagagaaaagaagaguaagaagaaauauaagaccccggcgccgccacc

SEQ ID NO: 7 - DNA, 57 bp, Artificial Sequence
  Synthetic 5 UTR-023 (DNA)
gggaaataagagagaaaagaagagtaagaagaaatataagaccccggcgccgccacc

SEQ ID NO: 8 - RNA, 35 nt, Artificial Sequence
  Synthetic 5 UTR-001 Core (RNA)
uaagagagaaaagaagaguaagaagaaauauaaga

SEQ ID NO: 9 - DNA, 57 bp, Artificial Sequence; identical to SEQ ID NO: 7
  Synthetic F418 (V1-UTR (v1.1 Ref)) (DNA)

SEQ ID NO: 10 - DNA, 54 bp, Artificial Sequence
  Synthetic V2-UTR (DNA)
gggaaataagagagaaaagaagagtaagaagaaatataagaccccggcgccacc

SEQ ID NO: 11 - DNA, 67 bp, Artificial Sequence
  Synthetic CG1-UTR (DNA)
gggaaataagagagaaaagaagagtaagaagaaatataagagcgccccgcggcgccccgcggccacc

SEQ ID NO: 12 - DNA, 67 bp, Artificial Sequence
  Synthetic CG2-UTR (DNA)
gggaaataagagagaaaagaagagtaagaagaaatataagacccgcccgccccgccccgccgccacc

SEQ ID NO: 13 - DNA, 15 bp, Artificial Sequence
  Synthetic KT1-UTR
gggcccgccgccaac

SEQ ID NO: 14 - DNA, 15 bp, Artificial Sequence
  Synthetic KT2-UTR
gggcccgccgccacc

SEQ ID NO: 15 - DNA, 15 bp, Artificial Sequence
  Synthetic KT3-UTR
gggcccgccgccgac

SEQ ID NO: 16 - DNA, 15 bp, Artificial Sequence
  Synthetic KT4-UT4
gggcccgccgccgcc

SEQ ID NO: 17 - DNA, 6 bp, Artificial Sequence
  Synthetic Traditional Kozak consensus, K0
gccrcc

SEQ ID NO: 18 - DNA, 6 bp, Artificial Sequence
  Synthetic GC-rich RNA element, EK2
gccgcc

SEQ ID NO: 19 - DNA, 6 bp, Artificial Sequence
  Synthetic GC-rich RNA element, EK3
ccgccg

SEQ ID NO: 20 - DNA, 20 bp, Artificial Sequence
  Synthetic GC-rich RNA element, CG1
gcgccccgcggcgccccgcg

SEQ ID NO: 21 - DNA, 20 bp, Artificial Sequence
  Synthetic GC-rich RNA element, CG2
cccgcccgccccgccccgcc

SEQ ID NO: 22 - DNA, 30 bp, Artificial Sequence
  Synthetic GC-rich RNA element
  misc_feature (4)..(30): ccg may or may not be present
ccgccgccgccgccgccgccgccgccgccg

SEQ ID NO: 23 - DNA, 30 bp, Artificial Sequence
  Synthetic GC-rich RNA element
  misc_feature (4)..(30): gcc may or may not be present
gccgccgccgccgccgccgccgccgccgcc

SEQ ID NO: 24 - DNA, 16 bp, Artificial Sequence
  Synthetic Stable RNA structures, SL1
ccgcggcgccccgcgg

SEQ ID NO: 25 - RNA, 17 nt, Artificial Sequence
  Synthetic Stable RNA structures, SL2
gcgcgcauauagcgcgc

SEQ ID NO: 26 - DNA, 26 bp, Artificial Sequence
  Synthetic Stable RNA structures, SL3
catggtggcggcccgccgccaccatg

SEQ ID NO: 27 - DNA, 23 bp, Artificial Sequence
  Synthetic Stable RNA structures, SL4
catggtggcccgccgccaccatg

SEQ ID NO: 28 - DNA, 22 bp, Artificial Sequence
  Synthetic Stable RNA structures, SL5
catggtgcccgccgccaccatg

SEQ ID NO: 29 - DNA, 12 bp, Artificial Sequence
  Synthetic C-Rich RNA element, CR2
cccccccaaccc

SEQ ID NO: 30 - DNA, 12 bp, Artificial Sequence
  Synthetic C-Rich RNA element, CR1
ccccccccaacc

SEQ ID NO: 31 - DNA, 12 bp, Artificial Sequence
  Synthetic C-Rich RNA element, CR3
ccccccaccccc

SEQ ID NO: 32 - RNA, 12 nt, Artificial Sequence
  Synthetic C-Rich RNA element, CR4
ccccccuaagcc

SEQ ID NO: 33 - DNA, 10 bp, Artificial Sequence
  Synthetic C-Rich RNA element, CR5
ccccacaacc

SEQ ID NO: 34 - DNA, 11 bp, Artificial Sequence
  Synthetic C-Rich RNA element, CR6
cccccacaacc

SEQ ID NO: 35 - RNA, 76 nt, Artificial Sequence
  Synthetic 5 UTR, combo1_V1.1 (RNA)
gggaaaccccccacccccggggaaauaagagagaaaagaagaguaagaagaaauauaagaccccggcgccgccacc

SEQ ID NO: 36 - RNA, 75 nt, Artificial Sequence
  Synthetic 5 UTR, combo2_V1.1 (RNA)
gggaaauccccacaaccggggaaauaagagagaaaagaagaguaagaagaaauauaagaccccggcgccgccacc

SEQ ID NO: 37 - RNA, 205 nt, Artificial Sequence
  Synthetic 5 UTR, combo1_S065 (RNA)
gggaaaccccccacccccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 38 - RNA, 204 nt, Artificial Sequence
  Synthetic 5 UTR, combo2_S065 (RNA)
gggaaauccccacaaccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 39 - RNA, 192 nt, Artificial Sequence
  Synthetic 5 UTR, combo3_S065 (S065 ExtKozak) (RNA)
gggagaccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 40 - RNA, 205 nt, Artificial Sequence
  Synthetic 5 UTR, combo4_S065 (RNA)
gggaaaccccccacccccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 41 - RNA, 204 nt, Artificial Sequence
  Synthetic 5 UTR, F153 combo5_S065 (RNA)
gggaaauccccacaaccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 42 - RNA, 192 nt, Artificial Sequence
  Synthetic 5 UTR, S065 Ref (RNA)
gggagaccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 43 - DNA, 21 bp, Artificial Sequence
  Synthetic 5 UTR, GCC3-ExtKozak (Ref)
gggaaagccgccgccgccacc

SEQ ID NO: 44 - RNA, 33 nt, Artificial Sequence
  Synthetic 5 UTR, CrichCR4 + GCC3-ExtKozak (RNA)
gggaaaccccccuaagccgccgccgccgccacc

SEQ ID NO: 45 - RNA, 47 nt, Artificial Sequence
  Synthetic 5 UTR, V0-UTR (v1.0 Ref) (RNA)
gggaaauaagagagaaaagaagaguaagaagaaauauaagagccacc

SEQ ID NO: 46 - RNA, 176 nt, Artificial Sequence
  Synthetic 5 UTR, S065 core (RNA)
ccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccua

SEQ ID NO: 47 - DNA, 18 bp, Artificial Sequence
  Synthetic 5 UTR-026 (DNA)
ttccggttgggtgtcacg

SEQ ID NO: 48 - RNA, 18 nt, Artificial Sequence
  Synthetic 5 UTR-026 (RNA)
uuccgguugggugucacg

SEQ ID NO: 49 - RNA, 18 nt, Artificial Sequence
  Synthetic 5 UTR-024 (RNA)
cccccccaacccgucacg

SEQ ID NO: 50 - RNA, 47 nt, Artificial Sequence
  Synthetic 5UTR-002 (RNA)
gggagaucagagagaaaagaagaguaagaagaaauauaagagccacc

SEQ ID NO: 51 - RNA, 145 nt, Artificial Sequence
  Synthetic 5 UTR-003 (RNA)
ggaauaaaagucucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagcaac

SEQ ID NO: 52 - RNA, 42 nt, Artificial Sequence
  Synthetic 5 UTR-004 (RNA)
gggagacaagcuuggcauuccgguacuguugguaaagccacc

SEQ ID NO: 53 - RNA, 47 nt, Artificial Sequence; identical to SEQ ID NO: 50
  Synthetic 5 UTR-005 (RNA)

SEQ ID NO: 54 - RNA, 145 nt, Artificial Sequence; identical to SEQ ID NO: 51
  Synthetic 5 UTR-006 (RNA)

SEQ ID NO: 55 - RNA, 42 nt, Artificial Sequence; identical to SEQ ID NO: 52
  Synthetic 5 UTR-007 (RNA)

SEQ ID NO: 56 - RNA, 47 nt, Artificial Sequence
  Synthetic 5 UTR-008 (RNA)
gggaauuaacagagaaaagaagaguaagaagaaauauaagagccacc

SEQ ID NO: 57 - RNA, 47 nt, Artificial Sequence
  Synthetic 5 UTR-009 (RNA)
gggaaauuagacagaaaagaagaguaagaagaaauauaagagccacc

SEQ ID NO: 58 - RNA, 47 nt, Artificial Sequence
  Synthetic 5 UTR-010 (RNA)
gggaaauaagagaguaaagaacaguaagaagaaauauaagagccacc

SEQ ID NO: 59 - RNA, 47 nt, Artificial Sequence
  Synthetic 5 UTR-011 (RNA)
gggaaaaaagagagaaaagaagacuaagaagaaauauaagagccacc

SEQ ID NO: 60 - RNA, 47 nt, Artificial Sequence
  Synthetic 5 UTR-012 (RNA)
gggaaauaagagagaaaagaagaguaagaagauauauaagagccacc

SEQ ID NO: 61 - RNA, 47 nt, Artificial Sequence
  Synthetic 5 UTR-013 (RNA)
gggaaauaagagacaaaacaagaguaagaagaaauauaagagccacc

SEQ ID NO: 62 - RNA, 47 nt, Artificial Sequence
  Synthetic 5 UTR-014 (RNA)
gggaaauuagagaguaaagaacaguaaguagaauuaaaagagccacc

SEQ ID NO: 63 - RNA, 47 nt, Artificial Sequence
  Synthetic 5 UTR-015 (RNA)
gggaaauaagagagaauagaagaguaagaagaaauauaagagccacc

SEQ ID NO: 64 - RNA, 47 nt, Artificial Sequence
  Synthetic 5 UTR-016 (RNA)
gggaaauaagagagaaaagaagaguaagaagaaaauuaagagccacc

SEQ ID NO: 65 - RNA, 47 nt, Artificial Sequence
  Synthetic 5 UTR-017 (RNA)
gggaaauaagagagaaaagaagaguaagaagaaauuuaagagccacc

SEQ ID NO: 66 - RNA, 47 nt, Artificial Sequence; identical to SEQ ID NO: 45
  Synthetic 5 UTR-018 (RNA)

SEQ ID NO: 67 - RNA, 92 nt, Artificial Sequence
  Synthetic 5 UTR-019 (RNA)
ucaagcuuuuggacccucguacagaagcuaauacgacucacuauagggaaauaagagagaaaagaagaguaagaagaaauauaagagccacc

SEQ ID NO: 68 - RNA, 140 nt, Artificial Sequence
  Synthetic 5 UTR-020 (RNA)
ggacagaucgccuggagacgccauccacgcuguuuugaccuccauagaagacaccgggaccgauccagccuccgcggccgggaacggugcauuggaacgcggauuccccgugccaagagugacucaccguccuugacacg

SEQ ID NO: 69 - RNA, 42 nt, Artificial Sequence
  Synthetic 5 UTR-021 (RNA)
ggcgcugccuacggagguggcagccaucuccuucucggcauc

SEQ ID NO: 70 - RNA, 67 nt, Artificial Sequence
  Synthetic 5 UTR, CG2-UTR (RNA)
gggaaauaagagagaaaagaagaguaagaagaaauauaagacccgcccgccccgccccgccgccacc

SEQ ID NO: 71 - RNA, 47 nt, Artificial Sequence
  Synthetic 5 UTR, V0-UTR (v1.0 Ref)-A (RNA)
aggaaauaagagagaaaagaagaguaagaagaaauauaagagccacc

SEQ ID NO: 72 - RNA, 192 nt, Artificial Sequence
  Synthetic 5 UTR, S065-A Ref (RNA)
aggagaccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 73 - RNA, 192 nt, Artificial Sequence
  Synthetic 5 UTR, combo3_S065 (S065 ExtKozak)-A
aggagaccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 74 - RNA, 57 nt, Artificial Sequence
  Synthetic 5 UTR, F418 (V1-UTR (v1.1 Ref))-A (RNA)
aggaaauaagagagaaaagaagaguaagaagaaauauaagaccccggcgccgccacc

SEQ ID NO: 75 - RNA, 54 nt, Artificial Sequence
  Synthetic 5 UTR, V2-UTR-A (RNA)
aggaaauaagagagaaaagaagaguaagaagaaauauaagaccccggcgccacc

SEQ ID NO: 76 - RNA, 67 nt, Artificial Sequence
  Synthetic 5 UTR, CG1-UTR-A (RNA)
aggaaauaagagagaaaagaagaguaagaagaaauauaagagcgccccgcggcgccccgcggccacc

SEQ ID NO: 77 - RNA, 67 nt, Artificial Sequence
  Synthetic 5 UTR,  CG2-UTR-A (RNA)
aggaaauaagagagaaaagaagaguaagaagaaauauaagacccgcccgccccgccccgccgccacc

SEQ ID NO: 78 - DNA, 15 bp, Artificial Sequence
  Synthetic 5 UTR,  KT1-UTR-A
aggcccgccgccaac

SEQ ID NO: 79 - DNA, 15 bp, Artificial Sequence
  Synthetic 5 UTR,  KT2-UTR-A
aggcccgccgccacc

SEQ ID NO: 80 - DNA, 15 bp, Artificial Sequence
  Synthetic 5 UTR,  KT3-UTR-A
aggcccgccgccgac

SEQ ID NO: 81 - DNA, 15 bp, Artificial Sequence
  Synthetic 5 UTR,  KT4-UTR-A
aggcccgccgccgcc

SEQ ID NO: 82 - DNA, 21 bp, Artificial Sequence
  Synthetic 5 UTR,  GCC3-ExtKozak (Ref)-A
aggaaagccgccgccgccacc

SEQ ID NO: 83 - RNA, 205 nt, Artificial Sequence
  Synthetic 5 UTR,  combo1_S065 (RNA)-A
aggaaaccccccacccccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 84 - RNA, 204 nt, Artificial Sequence
  Synthetic 5 UTR,  combo2_S065 (RNA)-A
aggaaauccccacaaccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 85 - RNA, 205 nt, Artificial Sequence
  Synthetic 5 UTR,  combo4_S065 (RNA)-A
aggaaaccccccacccccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 86 - RNA, 204 nt, Artificial Sequence
  Synthetic 5 UTR,  F153 combo5_S065 (RNA-A
aggaaauccccacaaccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 87 - RNA, 76 nt, Artificial Sequence
  Synthetic 5 UTR,  combo1_V1.1-A (RNA)
aggaaaccccccacccccggggaaauaagagagaaaagaagaguaagaagaaauauaagaccccggcgccgccacc

SEQ ID NO: 88 - RNA, 75 nt, Artificial Sequence
  Synthetic 5 UTR,  combo2_V1.1-A (RNA)
aggaaauccccacaaccggggaaauaagagagaaaagaagaguaagaagaaauauaagaccccggcgccgccacc

SEQ ID NO: 89 - RNA, 33 nt, Artificial Sequence
  Synthetic 5 UTR,  CrichCR4 + GCC3-ExtKozak-A (RNA)
aggaaaccccccuaagccgccgccgccgccacc

SEQ ID NO: 90 - RNA, 371 nt, Artificial Sequence
  Synthetic 3 UTR-001, Creatine Kinase
gcgccugcccaccugccaccgacugcuggaacccagccagugggagggccuggcccaccagaguccugcucccucacuccucgccccgcccccugucccagagucccaccugggggcucucuccacccuucucagaguuccaguuucaaccagaguuccaaccaaugggcuccauccucuggauucuggccaaugaaauaucucccuggcaggguccucuucuuuucccagagcuccaccccaaccaggagcucuaguuaauggagagcucccagcacacucggagcuugugcuuugucuccacgcaaagcgauaaauaaaagcauugguggccuuuggucuuugaauaaagccugaguaggaagucuaga

SEQ ID NO: 91 - RNA, 568 nt, Artificial Sequence
  Synthetic 3 UTR-002, Myoglobin
gccccugccgcucccacccccacccaucugggccccggguucaagagagagcggggucugaucucguguagccauauagaguuugcuucugagugucugcuuuguuuaguagaggugggcaggaggagcugaggggcuggggcugggguguugaaguuggcuuugcaugcccagcgaugcgccucccugugggaugucaucacccugggaaccgggaguggcccuuggcucacuguguucugcaugguuuggaucugaauuaauuguccuuucuucuaaaucccaaccgaacuucuuccaaccuccaaacuggcuguaaccccaaauccaagccauuaacuacaccugacaguagcaauugucugauuaaucacuggccccuugaagacagcagaaugucccuuugcaaugaggaggagaucugggcugggcgggccagcuggggaagcauuugacuaucuggaacuugugugugccuccucagguauggcagugacucaccugguuuuaauaaaacaaccugcaacaucucauggucuuugaauaaagccugaguaggaagucuaga

SEQ ID NO: 92 - RNA, 289 nt, Artificial Sequence
  Synthetic 3 UTR-003, alpha-actin
acacacuccaccuccagcacgcgacuucucaggacgacgaaucuucucaaugggggggcggcugagcuccagccaccccgcagucacuuucuuuguaacaacuuccguugcugccaucguaaacugacacaguguuuauaacguguacauacauuaacuuauuaccucauuuuguuauuuuucgaaacaaagcccuguggaagaaaauggaaaacuugaagaagcauuaaagucauucuguuaagcugcguaaauggucuuugaauaaagccugaguaggaagucuaga

SEQ ID NO: 93 - RNA, 379 nt, Artificial Sequence
  Synthetic 3 UTR-004, Albumin
caucacauuuaaaagcaucucagccuaccaugagaauaagagaaagaaaaugaagaucaaaagcuuauucaucuguuuuucuuuuucguugguguaaagccaacacccugucuaaaaaacauaaauuucuuuaaucauuuugccucuuuucucugugcuucaauuaauaaaaaauggaaagaaucuaauagagugguacagcacuguuauuuuucaaagauguguugcuauccugaaaauucuguagguucuguggaaguuccaguguucucucuuauuccacuucgguagaggauuucuaguuucuugugggcuaauuaaauaaaucauuaauacucuucuaauggucuuugaauaaagccugaguaggaagucuaga

SEQ ID NO: 94 - RNA, 118 nt, Artificial Sequence
  Synthetic 3 UTR-005, alpha-globin
gcugccuucugcggggcuugccuucuggccaugcccuucuucucucccuugcaccuguaccucuuggucuuugaauaaagccugaguaggaaggcggccgcucgagcaugcaucuaga

SEQ ID NO: 95 - RNA, 908 nt, Artificial Sequence
  Synthetic 3 UTR-006, G-CSF
gccaagcccuccccaucccauguauuuaucucuauuuaauauuuaugucuauuuaagccucauauuuaaagacagggaagagcagaacggagccccaggccucuguguccuucccugcauuucugaguuucauucuccugccuguagcagugagaaaaagcuccuguccucccauccccuggacugggagguagauagguaaauaccaaguauuuauuacuaugacugcuccccagcccuggcucugcaaugggcacugggaugagccgcugugagccccugguccugaggguccccaccugggacccuugagaguaucaggucucccacgugggagacaagaaaucccuguuuaauauuuaaacagcaguguuccccaucuggguccuugcaccccucacucuggccucagccgacugcacagcggccccugcauccccuuggcugugaggccccuggacaagcagagguggccagagcugggaggcauggcccuggggucccacgaauuugcuggggaaucucguuuuucuucuuaagacuuuugggacaugguuugacucccgaacaucaccgacgcgucuccuguuuuucuggguggccucgggacaccugcccugcccccacgagggucaggacugugacucuuuuuagggccaggcaggugccuggacauuugccuugcuggacggggacuggggaugugggagggagcagacaggaggaaucaugucaggccugugugugaaaggaagcuccacugucacccuccaccucuucaccccccacucaccaguguccccuccacugucacauuguaacugaacuucaggauaauaaaguguuugccuccauggucuuugaauaaagccugaguaggaaggcggccgcucgagcaugcaucuaga

SEQ ID NO: 96 - RNA, 835 nt, Artificial Sequence
  Synthetic 3 UTR-007, Col1a2; collagen, type I, alpha 2
acucaaucuaaauuaaaaaagaaagaaauuugaaaaaacuuucucuuugccauuucuucuucuucuuuuuuaacugaaagcugaauccuuccauuucuucugcacaucuacuugcuuaaauugugggcaaaagagaaaaagaaggauugaucagagcauugugcaauacaguuucauuaacuccuucccccgcucccccaaaaauuugaauuuuuuuuucaacacucuuacaccuguuauggaaaaugucaaccuuuguaagaaaaccaaaauaaaaauugaaaaauaaaaaccauaaacauuugcaccacuuguggcuuuugaauaucuuccacagagggaaguuuaaaacccaaacuuccaaagguuuaaacuaccucaaaacacuuucccaugagugugauccacauuguuaggugcugaccuagacagagaugaacugagguccuuguuuuguuuuguucauaauacaaaggugcuaauuaauaguauuucagauacuugaagaauguugauggugcuagaagaauuugagaagaaauacuccuguauugaguuguaucgugugguguauuuuuuaaaaaauuugauuuagcauucauauuuuccaucuuauucccaauuaaaaguaugcagauuauuugcccaaaucuucuucagauucagcauuuguucuuugccagucucauuuucaucuucuuccaugguuccacagaagcuuuguuucuugggcaagcagaaaaauuaaauuguaccuauuuuguauaugugagauguuuaaauaaauugugaaaaaaaugaaauaaagcauguuugguuuuccaaaagaacauau

SEQ ID NO: 97 - RNA, 297 nt, Artificial Sequence
  Synthetic 3 UTR-008, Col6a2; collagen, type VI, alpha 2
cgccgccgcccgggccccgcagucgagggucgugagcccaccccguccauggugcuaagcgggcccgggucccacacggccagcaccgcugcucacucggacgacgcccugggccugcaccucuccagcuccucccacgggguccccguagccccggcccccgcccagccccaggucuccccaggcccuccgcaggcugcccggccucccucccccugcagccaucccaaggcuccugaccuaccuggccccugagcucuggagcaagcccugacccaauaaaggcuuugaacccau

SEQ ID NO: 98 - RNA, 602 nt, Artificial Sequence
  Synthetic 3 UTR-009, RPN1; ribophorin I
ggggcuagagcccucuccgcacagcguggagacggggcaaggagggggguuauuaggauuggugguuuuguuuugcuuuguuuaaagccgugggaaaauggcacaacuuuaccucugugggagaugcaacacugagagccaaggggugggaguugggauaauuuuuauauaaaagaaguuuuuccacuuugaauugcuaaaaguggcauuuuuccuaugugcagucacuccucucauuucuaaaauagggacguggccaggcacgguggcucaugccuguaaucccagcacuuugggaggccgaggcaggcggcucacgaggucaggagaucgagacuauccuggcuaacacgguaaaacccugucucuacuaaaaguacaaaaaauuagcugggcgugguggugggcaccuguagucccagcuacucgggaggcugaggcaggagaaaggcaugaauccaagaggcagagcuugcagugagcugagaucacgccauugcacuccagccugggcaacaguguuaagacucugucucaaauauaaauaaauaaauaaauaaauaaauaaauaaauaaaaauaaagcgagauguugcccucaaa

SEQ ID NO: 99 - RNA, 785 nt, Artificial Sequence
  Synthetic 3 UTR-010, LRP1; low density lipoprotein receptor-related protein 1
ggcccugccccgucggacugcccccagaaagccuccugcccccugccagugaaguccuucagugagccccuccccagccagcccuucccuggccccgccggauguauaaauguaaaaaugaaggaauuacauuuuauaugugagcgagcaagccggcaagcgagcacaguauuauuucuccauccccucccugccugcuccuuggcacccccaugcugccuucagggagacaggcagggagggcuuggggcugcaccuccuacccucccaccagaacgcaccccacugggagagcugguggugcagccuuccccucccuguauaagacacuuugccaaggcucuccccucucgccccaucccugcuugcccgcucccacagcuuccugagggcuaauucugggaagggagaguucuuugcugccccugucuggaagacguggcucugggugagguaggcgggaaaggauggaguguuuuaguucuugggggaggccaccccaaaccccagccccaacuccaggggcaccuaugagauggccaugcucaaccccccucccagacaggcccucccugucuccagggcccccaccgagguucccagggcuggagacuuccucugguaaacauuccuccagccuccccuccccuggggacgccaaggaggugggccacacccaggaagggaaagcgggcagccccguuuuggggacgugaacguuuuaauaauuuuugcugaauuccuuuacaacuaaauaacacagauauuguuauaaauaaaauugu

SEQ ID NO: 100 - RNA, 3001 nt, Artificial Sequence
  Synthetic 3 UTR-011, Nnt1; cardiotrophin-like cytokine factor 1
auauuaaggaucaagcuguuagcuaauaaugccaccucugcaguuuugggaacaggcaaauaaaguaucaguauacauggugauguacaucuguagcaaagcucuuggagaaaaugaagacugaagaaagcaaagcaaaaacuguauagagagauuuuucaaaagcaguaaucccucaauuuuaaaaaaggauugaaaauucuaaaugucuuucugugcauauuuuuuguguuaggaaucaaaaguauuuuauaaaaggagaaagaacagccucauuuuagauguaguccuguuggauuuuuuaugccuccucaguaaccagaaauguuuuaaaaaacuaaguguuuaggauuucaagacaacauuauacauggcucugaaauaucugacacaauguaaacauugcaggcaccugcauuuuauguuuuuuuuuucaacaaaugugacuaauuugaaacuuuuaugaacuucugagcuguccccuugcaauucaaccgcaguuugaauuaaucauaucaaaucaguuuuaauuuuuuaaauuguacuucagagucuauauuucaagggcacauuuucucacuacuauuuuaauacauuaaaggacuaaauaaucuuucagagaugcuggaaacaaaucauuugcuuuauauguuucauuagaauaccaaugaaacauacaacuugaaaauuaguaauaguauuuuugaagaucccauuucuaauuggagaucucuuuaauuucgaucaacuuauaauguguaguacuauauuaagugcacuugaguggaauucaacauuugacuaauaaaaugaguucaucauguuggcaagugauguggcaauuaucucuggugacaaaagaguaaaaucaaauauuucugccuguuacaaauaucaaggaagaccugcuacuaugaaauagaugacauuaaucugucuucacuguuuauaauacggauggauuuuuuuucaaaucaguguguguuuugaggucuuauguaauugaugacauuugagagaaaugguggcuuuuuuuagcuaccucuuuguucauuuaagcaccaguaaagaucaugucuuuuuauagaaguguagauuuucuuugugacuuugcuaucgugccuaaagcucuaaauauaggugaaugugugaugaauacucagauuauuugucucucuauauaauuaguuugguacuaaguuucucaaaaaauuauuaacacaugaaagacaaucucuaaaccagaaaaagaaguaguacaaauuuuguuacuguaaugcucgcguuuagugaguuuaaaacacacaguaucuuuugguuuuauaaucaguuucuauuuugcugugccugagauuaagaucuguguaugugugugugugugugugugcguuuguguguuaaagcagaaaagacuuuuuuaaaaguuuuaagugauaaaugcaauuuguuaauugaucuuagaucacuaguaaacucagggcugaauuauaccauguauauucuauuagaagaaaguaaacaccaucuuuauuccugcccuuuuucuucucucaaaguaguuguaguuauaucuagaaagaagcaauuuugauuucuugaaaagguaguuccugcacucaguuuaaacuaaaaauaaucauacuuggauuuuauuuauuuuugucauaguaaaaauuuuaauuuauauauauuuuuauuuaguauuaucuuauucuuugcuauuugccaauccuuugucaucaauuguguuaaaugaauugaaaauucaugcccuguucauuuuauuuuacuuuauugguuaggauauuuaaaggauuuuuguauauauaauuucuuaaauuaauauuccaaaagguuaguggacuuagauuauaaauuauggcaaaaaucuaaaaacaacaaaaaugauuuuuauacauucuauuucauuauuccucuuuuuccaauaagucauacaauugguagauaugacuuauuuuauuuuuguauuauucacuauaucuuuaugauauuuaaguauaaauaauuaaaaaaauuuauuguaccuuauagucugucaccaaaaaaaaaaaauuaucuguagguagugaaaugcuaauguugauuugucuuuaagggcuuguuaacuauccuuuauuuucucauuugucuuaaauuaggaguuuguguuuaaauuacucaucuaagcaaaaaauguauauaaaucccauuacuggguauauacccaaaggauuauaaaucaugcugcuauaaagacacaugcacacguauguuuauugcagcacuauucacaauagcaaagacuuggaaccaacccaaauguccaucaaugauagacuugauuaagaaaaugugcacauauacaccauggaauacuaugcagccauaaaaaaggaugaguucauguccuuuguagggacauggauaaagcuggaaaccaucauucugagcaaacuauugcaaggacagaaaaccaaacacugcauguucucacucauaggugggaauugaacaaugagaacacuuggacacaagguggggaacaccacacaccagggccugucaugggguggggggaguggggagggauagcauuaggagauauaccuaauguaaaugaugaguuaaugggugcagcacaccaacauggcacauguauacauauguagcaaaccugcacguugugcacauguacccuagaacuuaaaguauaauuaaaaaaaaaaagaaaacagaagcuauuuauaaagaaguuauuugcugaaauaaaugugaucuuucccauuaaaaaaauaaagaaauuuugggguaaaaaaacacaauauauuguauucuugaaaaauucuaagagaguggaugugaaguguucucaccacaaaagugauaacuaauugagguaaugcacauauuaauuagaaagauuuugucauuccacaauguauauauacuuaaaaauauguuauacacaauaaauacauacauuaaaaaauaaguaaaugua

SEQ ID NO: 101 - RNA, 1037 nt, Artificial Sequence
  Synthetic 3 UTR-012, Col6a1; collagen, type VI, alpha 1
cccacccugcacgccggcaccaaacccuguccucccaccccuccccacucaucacuaaacagaguaaaaugugaugcgaauuuucccgaccaaccugauucgcuagauuuuuuuuaaggaaaagcuuggaaagccaggacacaacgcugcugccugcuuugugcaggguccuccggggcucagcccugaguuggcaucaccugcgcagggcccucuggggcucagcccugagcuagugucaccugcacagggcccucugaggcucagcccugagcuggcgucaccugugcagggcccucuggggcucagcccugagcuggccucaccuggguuccccaccccgggcucuccugcccugcccuccugcccgcccucccuccugccugcgcagcuccuucccuaggcaccucugugcugcaucccaccagccugagcaagacgcccucucggggccugugccgcacuagccucccucuccucuguccccauagcugguuuuucccaccaauccucaccuaacaguuacuuuacaauuaaacucaaagcaagcucuucuccucagcuuggggcagccauuggccucugucucguuuugggaaaccaaggucaggaggccguugcagacauaaaucucggcgacucggccccgucuccugaggguccugcuggugaccggccuggaccuuggcccuacagcccuggaggccgcugcugaccagcacugaccccgaccucagagaguacucgcaggggcgcuggcugcacucaagacccucgagauuaacggugcuaaccccgucugcuccucccucccgcagagacuggggccuggacuggacaugagagccccuuggugccacagagggcugugucuuacuagaaacaacgcaaaccucuccuuccucagaauagugauguguucgacguuuuaucaaaggcccccuuucuauguucauguuaguuuugcuccuucuguguuuuuuucugaaccauauccauguugcugacuuuuccaaauaaagguuuucacuccucuc

SEQ ID NO: 102 - RNA, 577 nt, Artificial Sequence
  Synthetic 3 UTR-013, Calr; calreticulin
agaggccugccuccagggcuggacugaggccugagcgcuccugccgcagagcuggccgcgccaaauaaugucucugugagacucgagaacuuucauuuuuuuccaggcugguucggauuugggguggauuuugguuuuguuccccuccuccacucucccccacccccuccccgcccuuuuuuuuuuuuuuuuuuaaacugguauuuuaucuuugauucuccuucagcccucaccccugguucucaucuuucuugaucaacaucuuuucuugccucuguccccuucucucaucucuuagcuccccuccaaccuggggggcagugguguggagaagccacaggccugagauuucaucugcucuccuuccuggagcccagaggagggcagcagaaggggguggugucuccaaccccccagcacugaggaagaacggggcucuucucauuucaccccucccuuucuccccugcccccaggacugggccacuucuggguggggcagugggucccagauuggcucacacugagaauguaagaacuacaaacaaaauuucuauuaaauuaaauuuugugucucc

SEQ ID NO: 103 - RNA, 2212 nt, Artificial Sequence
  Synthetic 3 UTR-014, Colla1; collagen, type I, alpha 1
cucccuccaucccaaccuggcucccucccacccaaccaacuuuccccccaacccggaaacagacaagcaacccaaacugaacccccucaaaagccaaaaaaugggagacaauuucacauggacuuuggaaaauauuuuuuuccuuugcauucaucucucaaacuuaguuuuuaucuuugaccaaccgaacaugaccaaaaaccaaaagugcauucaaccuuaccaaaaaaaaaaaaaaaaaaagaauaaauaaauaacuuuuuaaaaaaggaagcuugguccacuugcuugaagacccaugcggggguaagucccuuucugcccguugggcuuaugaaaccccaaugcugcccuuucugcuccuuucuccacaccccccuuggggccuccccuccacuccuucccaaaucugucuccccagaagacacaggaaacaauguauugucugcccagcaaucaaaggcaaugcucaaacacccaaguggcccccacccucagcccgcuccugcccgcccagcacccccaggcccugggggaccugggguucucagacugccaaagaagccuugccaucuggcgcucccauggcucuugcaacaucuccccuucguuuuugagggggucaugccgggggagccaccagccccucacuggguucggaggagagucaggaagggccacgacaaagcagaaacaucggauuuggggaacgcgugucaaucccuugugccgcagggcugggcgggagagacuguucuguuccuuguguaacuguguugcugaaagacuaccucguucuugucuugaugugucaccggggcaacugccugggggcggggaugggggcaggguggaagcggcuccccauuuuauaccaaaggugcuacaucuaugugaugggugggguggggagggaaucacuggugcuauagaaauugagaugcccccccaggccagcaaauguuccuuuuuguucaaagucuauuuuuauuccuugauauuuuucuuuuuuuuuuuuuuuuuuuguggauggggacuugugaauuuuucuaaaggugcuauuuaacaugggaggagagcgugugcggcuccagcccagcccgcugcucacuuuccacccucucuccaccugccucuggcuucucaggccucugcucuccgaccucucuccucugaaacccuccuccacagcugcagcccauccucccggcucccuccuagucuguccugcguccucuguccccggguuucagagacaacuucccaaagcacaaagcaguuuuucccccuaggggugggaggaagcaaaagacucuguaccuauuuuguauguguauaauaauuugagauguuuuuaauuauuuugauugcuggaauaaagcauguggaaaugacccaaacauaauccgcaguggccuccuaauuuccuucuuuggaguugggggagggguagacauggggaaggggcuuuggggugaugggcuugccuuccauuccugcccuuucccuccccacuauucucuucuagaucccuccauaaccccacuccccuuucucucacccuucuuauaccgcaaaccuuucuacuuccucuuucauuuucuauucuugcaauuuccuugcaccuuuuccaaauccucuucuccccugcaauaccauacaggcaauccacgugcacaacacacacacacacucuucacaucugggguuguccaaaccucauacccacuccccuucaagcccauccacucuccacccccuggaugcccugcacuugguggcggugggaugcucauggauacugggagggugaggggaguggaacccgugaggaggaccugggggccucuccuugaacugacaugaagggucaucuggccucugcucccuucucacccacgcugaccuccugccgaaggagcaacgcaacaggagaggggucugcugagccuggcgagggucugggagggaccaggaggaaggcgugcucccugcucgcuguccuggcccugggggagugagggagacagacaccugggagagcuguggggaaggcacucgcaccgugcucuugggaaggaaggagaccuggcccugcucaccacggacugggugccucgaccuccugaauccccagaacacaaccccccugggcugggguggucuggggaaccaucgugcccccgccucccgccuacuccuuuuuaagcuu

SEQ ID NO: 104 - RNA, 729 nt, Artificial Sequence
  Synthetic 3 UTR-015, Plod1; procollagen-lysine, 2-oxoglutarate 5-dioxygenase 1
uuggccaggccugacccucuuggaccuuucuucuuugccgacaaccacugcccagcagccucugggaccucggggucccagggaacccaguccagccuccuggcuguugacuucccauugcucuuggagccaccaaucaaagagauucaaagagauuccugcaggccagaggcggaacacaccuuuauggcuggggcucuccgugguguucuggacccagccccuggagacaccauucacuuuuacugcuuuguagugacucgugcucuccaaccugucuuccugaaaaaccaaggcccccuucccccaccucuuccauggggugagacuugagcagaacaggggcuuccccaaguugcccagaaagacugucugggugagaagccauggccagagcuucucccaggcacagguguugcaccagggacuucugcuucaaguuuugggguaaagacaccuggaucagacuccaagggcugcccugagucugggacuucugccuccauggcuggucaugagagcaaaccguaguccccuggagacagcgacuccagagaaccucuugggagacagaagaggcaucugugcacagcucgaucuucuacuugccuguggggaggggagugacagguccacacaccacacugggucacccuguccuggaugccucugaagagagggacagaccgucagaaacuggagaguuucuauuaaaggucauuuaaacca

SEQ ID NO: 105 - RNA, 847 nt, Artificial Sequence
  Synthetic 3 UTR-016, Nucb1; nucleobindin 1
uccuccgggaccccagcccucaggauuccugaugcuccaaggcgacugaugggcgcuggaugaaguggcacagucagcuucccugggggcuggugucauguugggcuccuggggcgggggcacggccuggcauuucacgcauugcugccaccccagguccaccugucuccacuuucacagccuccaagucuguggcucuucccuucuguccuccgaggggcuugccuucucucguguccagugaggugcucagugaucggcuuaacuuagagaagcccgcccccuccccuucuccgucugucccaagagggucugcucugagccugcguuccuagguggcucggccucagcugccuggguuguggccgcccuagcauccuguaugcccacagcuacuggaauccccgcugcugcuccgggccaagcuucugguugauuaaugagggcaugggguggucccucaagaccuuccccuaccuuuuguggaaccagugaugccucaaagacaguguccccuccacagcugggugccaggggcaggggauccucaguauagccggugaacccugauaccaggagccugggccucccugaaccccuggcuuccagccaucucaucgccagccuccuccuggaccucuuggcccccagccccuuccccacacagccccagaagggucccagagcugaccccacuccaggaccuaggcccagccccucagccucaucuggagccccugaagaccagucccacccaccuuucuggccucaucugacacugcuccgcauccugcuguguguccuguuccauguuccgguuccauccaaauacacuuucuggaacaaa

SEQ ID NO: 106 - RNA, 110 nt, Artificial Sequence
  Synthetic 3 UTR-017, alpha-globin
gcuggagccucgguggccaugcuucuugccccuugggccuccccccagccccuccuccccuuccugcacccguacccccguggucuuugaauaaagucugagugggcggc

SEQ ID NO: 107 - RNA, 116 nt, Artificial Sequence
  Synthetic 3 UTR-018, Downstream UTR
uaauaggcuggagccucgguggccaugcuucuugccccuugggccuccccccagccccuccuccccuuccugcacccguacccccguggucuuugaauaaagucugagugggcggc

SEQ ID NO: 108 - RNA, 118 nt, Artificial Sequence
  Synthetic 3 UTR-019, Downstream UTR
ugauaauaggcuggagccucgguggccaugcuucuugccccuugggccuccccccagccccuccuccccuuccugcacccguacccccuggucuuugaauaaagucugagugggcggc

SEQ ID NO: 109 - RNA, 119 nt, Artificial Sequence
  Synthetic v1.1 3UTR (RNA)
ugauaauaggcuggagccucgguggccuagcuucuugccccuugggccuccccccagccccuccuccccuuccugcacccguacccccguggucuuugaauaaagucugagugggcggc

SEQ ID NO: 110 - RNA, 119 nt, Artificial Sequence
  Synthetic 3 UTR-020, Downstream UTR
ugauaauaggcuggagccucgguggccaugcuucuugccccuugggccuccccccagccccuccuccccuuccugcacccguacccccguggucuuugaauaaagucugagugggcggc

SEQ ID NO: 111 - protein, 21 aa, Artificial Sequence
  Synthetic 3XFLAG, Epitope tag
DYKDHDGDYKDHDIDYKDDDK

SEQ ID NO: 112 - protein, 10 aa, Artificial Sequence
  Synthetic Myc, Epitope tag
EQKLISEEDL

SEQ ID NO: 113 - protein, 14 aa, Artificial Sequence
  Synthetic V5, Epitope tag
GKPIPNPLLGLDST

SEQ ID NO: 114 - protein, 9 aa, Artificial Sequence
  Synthetic Hemagglutinin A (HA), Epitope tag
YPYDVPDYA

SEQ ID NO: 115 - protein, 6 aa, Artificial Sequence
  Synthetic 6xHis tag, Epitope tag
HHHHHH

SEQ ID NO: 116 - protein, 11 aa, Artificial Sequence
  Synthetic HSV, Epitope tag
QPELAPEDPED

SEQ ID NO: 117 - protein, 11 aa, Artificial Sequence
  Synthetic VSV-G, Epitope tag
YTDIEMNRLGK

SEQ ID NO: 118 - protein, 18 aa, Artificial Sequence
  Synthetic NE, Epitope tag
TKENPRSNQEESYDDNES

SEQ ID NO: 119 - protein, 15 aa, Artificial Sequence
  Synthetic AViTag, Epitope tag
GLNDIFEAQKIEWHE

SEQ ID NO: 120 - protein, 26 aa, Artificial Sequence
  Synthetic Calmodulin, Epitope tag
KRRWKKNFIAVSAANRFKKISSSGAL

SEQ ID NO: 121 - protein, 13 aa, Artificial Sequence
  Synthetic E tag, Epitope tag
GAPVPYPDPLEPR

SEQ ID NO: 122 - protein, 15 aa, Artificial Sequence
  Synthetic S tag, Epitope tag
KETAAAKFERQHMDS

SEQ ID NO: 123 - protein, 38 aa, Artificial Sequence
  Synthetic SBP tag, Epitope tag
MDEKTTGWRGGHVVEGLAGELEQLRARLEHHPQGQREP

SEQ ID NO: 124 - protein, 13 aa, Artificial Sequence
  Synthetic Softag 1, Epitope tag
SLAELLNAGLGGS

SEQ ID NO: 125 - protein, 8 aa, Artificial Sequence
  Synthetic Softag 3, Epitope tag
TQDPSRVG

SEQ ID NO: 126 - protein, 8 aa, Artificial Sequence
  Synthetic Strep tag, Epitope tag
WSHPQFEK

SEQ ID NO: 127 - protein, 10 aa, Artificial Sequence
  Synthetic Ty tag, Epitope tag
EVHTNQDPLD

SEQ ID NO: 128 - protein, 8 aa, Artificial Sequence
  Synthetic Xpress tag, Epitope tag
DLYDDDDK

SEQ ID NO: 129 - RNA, 18 nt, Artificial Sequence
  Synthetic 5 UTR-025 (RNA) [EXAMPLE 4]
uauggggguuaugucacg

SEQ ID NO: 130 - RNA, 54 nt, Artificial Sequence
  Synthetic 5 UTR, V2-UTR (RNA)
gggaaauaagagagaaaagaagaguaagaagaaauauaagaccccggcgccacc

SEQ ID NO: 131 - RNA, 67 nt, Artificial Sequence
  Synthetic 5 UTR, CG1-UTR (RNA)
gggaaauaagagagaaaagaagaguaagaagaaauauaagagcgccccgcggcgccccgcggccacc

SEQ ID NO: 132 - RNA, 57 nt, Artificial Sequence; identical to SEQ ID NO: 6
  Synthetic 5 UTR, F418 (V1-UTR (v1.1 Ref)) (RNA)

SEQ ID NO: 133 - protein, 8 aa, Artificial Sequence
  Synthetic FLAG, Epitope tag
DYKDDDDK

SEQ ID NO: 134 - RNA, 138 nt, Artificial Sequence
  Synthetic 3 UTR-018 + miR-122-5p binding site
uaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 135 - RNA, 138 nt, Artificial Sequence
  Synthetic 3UTR-018 + miR-122-3p binding site
uaauaggcuggagccucgguggccaugcuucuugccccuugggccuccccccagccccuccuccccuuccugcacccguacccccuauuuagugugauaauggcguuguggucuuugaauaaagucugagugggcggc

SEQ ID NO: 136 - RNA, 141 nt, Artificial Sequence
  Synthetic 3UTR-019 + miR-122 binding site
ugauaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 137 - RNA, 133 nt, Artificial Sequence
  Synthetic 3UTR + miR-142-3p binding site
gcuggagccucgguggccaugcuucuugccccuugggccuccccccagccccuccuccccuuccugcacccguacccccuccauaaaguaggaaacacuacaguggucuuugaauaaagucugagugggcggc

SEQ ID NO: 138 - RNA, 87 nt, Artificial Sequence
  Synthetic mmiR-142
gacagugcagucacccauaaaguagaaagcacuacuaacagcacuggaggguguaguguuuccuacuuuauggaugaguguacugug

SEQ ID NO: 139 - RNA, 23 nt, Artificial Sequence
  Synthetic mmiR-142-3p
uguaguguuuccuacuuuaugga

SEQ ID NO: 140 - RNA, 23 nt, Artificial Sequence
  Synthetic mmiR-142-3p binding site
uccauaaaguaggaaacacuaca

SEQ ID NO: 141 - RNA, 21 nt, Artificial Sequence
  Synthetic mmiR-142-5p
cauaaaguagaaagcacuacu

SEQ ID NO: 142 - RNA, 21 nt, Artificial Sequence
  Synthetic mmiR-142-5p binding site
aguagugcuuucuacuuuaug

SEQ ID NO: 143 - RNA, 85 nt, Artificial Sequence
  Synthetic miR-122
ccuuagcagagcuguggagugugacaaugguguuugugucuaaacuaucaaacgccauuaucacacuaaauagcuacugcuaggc

SEQ ID NO: 144 - RNA, 22 nt, Artificial Sequence
  Synthetic miR-122-3p
aacgccauuaucacacuaaaua

SEQ ID NO: 145 - RNA, 22 nt, Artificial Sequence
  Synthetic miR-122-3p binding site
uauuuagugugauaauggcguu

SEQ ID NO: 146 - RNA, 22 nt, Artificial Sequence
  Synthetic miR-122-5p
uggagugugacaaugguguuug

SEQ ID NO: 147 - RNA, 22 nt, Artificial Sequence
  Synthetic miR-122-5p binding site
caaacaccauugucacacucca

SEQ ID NO: 148 - DNA, 6 bp, Artificial Sequence
  Synthetic Kozak-like sequence, K1
gccacc

SEQ ID NO: 149 - RNA, 41 nt, Artificial Sequence
  Synthetic 5 UTR, V0-UTR (v1.0 Ref)-0
uaagagagaaaagaagaguaagaagaaauauaagagccacc

SEQ ID NO: 150 - RNA, 51 nt, Artificial Sequence
  Synthetic 5 UTR, F418-0 (V1-UTR (v1.1 Ref)) (RNA)
uaagagagaaaagaagaguaagaagaaauauaagaccccggcgccgccacc

SEQ ID NO: 151 - RNA, 61 nt, Artificial Sequence
  Synthetic 5 UTR, CG1-UTR-0
uaagagagaaaagaagaguaagaagaaauauaagagcgccccgcggcgccccgcggccacc

SEQ ID NO: 152 - RNA, 61 nt, Artificial Sequence
  Synthetic 5 UTR, CG2-UTR-0
uaagagagaaaagaagaguaagaagaaauauaagacccgcccgccccgccccgccgccacc

SEQ ID NO: 153 - DNA, 15 bp, Artificial Sequence
  Synthetic 5 UTR, GCC3-ExtKozak (Ref)-0
gccgccgccgccacc

SEQ ID NO: 154 - RNA, 186 nt, Artificial Sequence
  Synthetic 5 UTR, S065-0
ccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 155 - RNA, 186 nt, Artificial Sequence
  Synthetic 5 UTR, combo3_S065-0 (S065 ExtKozak)
ccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 156 - RNA, 199 nt, Artificial Sequence
  Synthetic 5 UTR, combo1_S065-0
ccccccacccccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 157 - RNA, 198 nt, Artificial Sequence
  Synthetic 5 UTR, combo2_S065-0
uccccacaaccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 158 - RNA, 199 nt, Artificial Sequence
  Synthetic 5 UTR, combo4_S065-0
ccccccacccccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 159 - RNA, 198 nt, Artificial Sequence
  Synthetic 5 UTR, combo5_S065-0
uccccacaaccgccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 160 - RNA, 70 nt, Artificial Sequence
  Synthetic 5 UTR, combo1_V1.1-0
ccccccacccccggggaaauaagagagaaaagaagaguaagaagaaauauaagaccccggcgccgccacc

SEQ ID NO: 161 - RNA, 69 nt, Artificial Sequence
  Synthetic 5 UTR, combo2_V1.1-0
uccccacaaccggggaaauaagagagaaaagaagaguaagaagaaauauaagaccccggcgccgccacc

SEQ ID NO: 162 - RNA, 27 nt, Artificial Sequence
  Synthetic 5 UTR, CrichCR4 + GCC3-ExtKozak-0
ccccccuaagccgccgccgccgccacc

SEQ ID NO: 163 - RNA, 48 nt, Artificial Sequence
  Synthetic 5 UTR, V2-UTR-0
uaagagagaaaagaagaguaagaagaaauauaagaccccggcgccacc